(19) United States
(12) Patent Application Publication
(10) Pub. No.: US 2010/0280085 A1
(43) Pub. Date: Nov. 4, 2010

(54) CHEMICAL COMPOUNDS

(75) Inventors: Christoph Luthy, Basel (CH); Anthony Cornelius O'Sullivan, Basel (CH); Jurgen Harry Schaetzer, Basel (CH)

Correspondence Address: SYNGENTA CROP PROTECTION , INC.;PATENT AND TRADEMARK DEPARTMENT, 410 SWING ROAD, GREENSBORO, NC, 27409, US

(73) Assignee: SYNGENTA CROP PROTECTION, INC., Greensboro (US)

(21) Appl. No.: 12/376,626

(22) PCT Filed: Aug. 6, 2007
(86) PCT No.: PCT/GB07/02969

§ 371 Date: May 21, 2010

(30) Foreign Application Priority Data
Aug. 7, 2006 (GB) 0615670.7

(51) Int. Cl. A01N 43/76; C07D 263/28; C07C 275/26; A01N 47/32; A01P 7/04; A01P 7/02; A01P 9/00; A01P 5/00

(52) U.S. Cl. 514/377; 548/233; 564/47; 514/595

(57) ABSTRACT

The use of a compound of formula (I) wherein X is (i), (ii) or (iii), where the substituents have the meanings assigned to them in claim 1, or compositions containing them in controlling insects, acarines, nematodes or molluscs. Novel compounds are also provided.

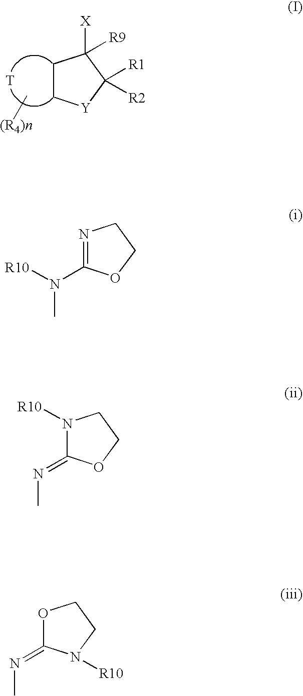

The present invention relates to oxazoline derivatives, to processes for preparing them, to insecticidal, acaricidal, molluscicidal and nematicidal compositions comprising them and to methods of using them to combat and control insect, acarine, mollusc and nematode pests.

Oxazoline derivatives with pharmaceutical properties are disclosed for example in U.S. Pat. No. 3,679,798, U.S. Pat. No. 3,624,092, U.S. Pat. No. 3,509,170, U.S. Pat. No. 2,870,161, U.S. Pat. No. 2,870,159.

1-Indanyl-2-oxazoline with ectoparasiticidal activity has been disclosed in DE 1963192.

It has now surprisingly been found that certain oxazolines derivatives have good insecticidal properties.

The present invention therefore provides a method of combating and controlling insects, acarines, nematodes or molluscs which comprises applying to a pest, to a locus of a pest, or to a plant susceptible to attack by a pest an insecticidally, acaricidally, nematicidally or molluscicidally effective amount of a compound of formula (I)

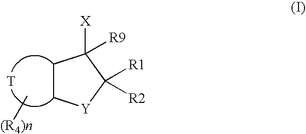

wherein

X is (i), (ii) or (iii)

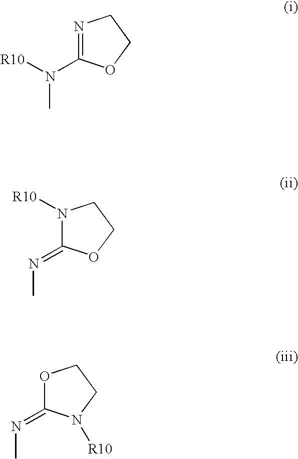

R¹⁰ is hydrogen, hydroxy, cyano, formyl, G-, G-O—, G-S—, G-S—S—, G-A-, R²⁴R²⁵N—, G-A-NR¹⁷—, R²⁴R²⁵N—S—, R²⁴R²⁵N-A-, R¹⁸N═C(R¹⁹)—, G-O-A- or G-S-A-; where R²⁴ and R²⁵ are independently H or G-, or R²⁴ and R²⁵ together with the N atom to which they are attached, form a group N═CRaRb where Ra and Rb are H, C₁₋₆ alkyl or phenyl; or R²⁴ and R²⁵ together with the N atom to which they are attached form a five, six or seven membered ring which may contain one or two further hetero atoms selected from O, N or S and which may be optionally substituted by one to four C₁₋₆ alkyl groups or phenyl; R¹⁷ is H, G-, G-C(O)— or G-OC(O); R¹⁸ is H, OH, cyano, nitro, G-, G-O— or R³⁸R³⁹N—, where R³⁸ and R³⁹ are independently H or G-, or R³⁸ and R³⁹ together with the N atom to which they are attached, form a group N═CRaRb where Ra and Rb are H, C₁₋₆ alkyl or phenyl; or R³⁸ and R³⁹ together with the N atom to which they are attached form a five, six or seven membered ring which may contain one or two further hetero atoms selected from O, N or S and which may be optionally substituted by one to four C₁₋₆ alkyl groups; R¹⁹ is H, cyano, G-, G-O—, G-S— or R⁴²R⁴³N—, where R⁴² and R⁴³ are independently H or G; or R⁴² and R⁴³ together with the N atom to which they are attached, form a group N═CRaRb where Ra and Rb are H, C₁₋₆ alkyl or phenyl; or R⁴² and R⁴³ together with the N atom to which they are attached form a five, six or seven membered ring which may contain one or two further hetero atoms selected from O, N or S and which may be optionally substituted by one to four C₁₋₆ alkyl groups or phenyl;

Y is O, S(O)ₘ, where m is 0, 1 or 2, NR³, SO₂—NR³, NR³—SO₂, NR³—O or O—NR³, where R³ is H, OH, cyano, formyl, G-, G-O—, G-S—, G-A-, R²⁷R²⁸N—, R²⁷R²⁸N-A-, G-O-A-, G-S-A-, G-A-NR²⁹—, R²⁷R²⁸N-A-NR²⁹—, G-O-A-NR²⁹— or G-S-A-NR²⁹—, where R²⁷ and R²⁸ are independently H or G-, or R²⁷ and R²⁸ together with the N atom to which they are attached, form a group N═CRaRb where Ra and Rb are H, C₁₋₆ alkyl or phenyl; or R²⁷ and R²⁸ together with the N atom to which they are attached form a five, six or seven membered ring which may contain one or two further hetero atoms selected from O, N or S and which may be optionally substituted by one to four C₁₋₆ alkyl groups or phenyl; R²⁹ is H or G-; or Y is CR⁵R⁶, CR⁵R⁶—CR⁷R⁸, O—CR⁷R⁸, S(O)ₘ—CR⁷R⁸, NR³—CR⁷R⁸, CR⁵R⁶—O, CR⁵R⁶—S(O)ₘ, CR⁵R⁶—NR³, where R³ and m have the meanings assigned to them above, and R⁵, R⁶, R⁷ and R⁸ are each independently H, OH, halogen, nitro, cyano, rhodano, carboxy, formyl, formyloxy, G-, G-O—, G-S—, G-A-, R²¹R²²N—, R²¹R²²N-A-, G-O-A-, G-S-A-, G-A-O—, G-A-S—, G-A-NR²³—, R²¹R²²N-A-O—, R²¹R²²N-A-S—, R²¹R²²N-A-NR²³—, G-O-A-O—, G-O-A-S—, G-O-A-NR²³—, G-S-A-O—, G-S-A-NR²³— or R²⁰S(O)(═NR¹⁷)—, where R²¹ and R²² are independently H or G-, or R²¹ and R²² together with the N atom to which they are attached, form a group N═CRaRb where Ra and Rb are H, C₁₋₆ alkyl or phenyl; or R²¹ and R²² together with the N atom to which they are attached form a five, six or seven membered ring which may contain one or two further hetero atoms selected from O, N or S and which may be optionally substituted by one to four C₁₋₆ alkyl groups or phenyl; R²³ is H or G- and R¹⁷ is as defined above; R²⁰ is C₁₋₆ alkyl, optionally substituted phenyl, optionally substituted benzyl; or two of the groups R⁵, R⁶, R⁷ and R⁸ attached to the same carbon atom are ═O, ═S, ═NR¹¹ or ═CR¹²R¹³, where R¹¹ is H, OH, nitro, cyano, formyl, formyloxy, G-, G-O—, G-A-, R³⁶R³⁷N—, G-C(O)—O—, G-C(O)—NR²⁶—, R³⁶R³⁷N—C(O)O—, G-O—C(O)O—, G-O—C(O)—NR²⁶—, where R³⁶, R³⁷ and R²⁶ are independently H or G-, or R³⁶ and R³⁷ together with the N atom to which they are attached, form a group N═CRaRb where Ra and Rb are H, C₁₋₆ alkyl or phenyl; or R³⁶ and R³⁷ together with the N atom to which they are attached form a five, six or seven membered ring which may contain one or two further hetero atoms selected from O, N or S and which may be optionally substituted by one to four C₁₋₆ alkyl groups or phenyl, and R¹² and R¹³ are each independently H, halogen, nitro, cyano, formyl, formyloxy, G-, G-O—, G-S—, G-A-, R⁴⁰R⁴¹N—, R⁴⁰R⁴¹N-A-, G-O-A-, G-A-O—, R⁴⁰R⁴¹N-A-O—, R⁴⁰R⁴¹N-A-S—, G-O-A-O—, G-O-A-S—, G-O-A-NR³⁰—, where R⁴⁰R⁴¹ and R³⁰ are independently H or G-, or R⁴⁰ and R⁴¹ together with the N atom to which they are attached, form a group N═CRaRb where Ra and Rb are H, C₁₋₆ alkyl or phenyl; or R⁴⁰ and R⁴¹ together with the N atom to which they are attached form a five, six or seven membered ring which may contain one or two further hetero atoms selected from O, N or S and which may be optionally substituted by one to four C₁₋₆ alkyl groups or phenyl, or R¹² and R¹³ together with the carbon atom to which they are attached form a 3 to 6 membered carbocyclic ring; or the groups R⁵ and R⁶ or R⁷ and R⁸ together with the carbon atom to which they are attached form a three to six membered ring, containing at least 2 carbon atoms and optionally containing one or two sulfur and/or one or two non-adjacent oxygen atoms or a group NR¹⁴, where R¹⁴ is H, OH, cyano, formyl, G-, G-O—, G-S—, G-A-, R²⁷R²⁸N—, R²⁷R²⁸N-A-, G-O-A-, G-S-A-, G-A-NR²⁹—, R²⁷R²⁸N-A-NR²⁹—, G-O-A-NR²⁹— or G-S-A-NR²⁹—, where R²⁷, R²⁸ and R²⁹ have the meanings assigned to them above, the ring being optionally substituted by one to four C₁₋₆ alkyl groups or phenyl; or two of the groups R⁵, R⁶, R⁷ and R⁸ attached to different atoms together with the atoms they are attached form a three to seven membered ring, that optionally contains one or two sulfur and/or one or two non-adjacent oxygen atoms or a group NR¹⁴, where R¹⁴ is as defined above, or two of the groups R⁵, R⁶, R⁷ and R⁸ attached to adjacent atoms combine to form a bond;

the ring (T)

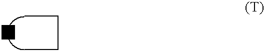

is a 5- or 6-membered aromatic or heteroaromatic ring;

R¹ and R² are each independently H, OH, halogen, nitro, cyano, rhodano, carboxy, formyl, formyloxy, G-, G-O—, G-S—, G-A-, R²¹R²²N—, R²¹R²²N-A-, G-O-A-, G-S-A-, G-A-O—, O-A-S—, G-A-NR²³—, R²¹R²²N-A-O—, R²¹R²²N-A-S—, R²¹R²²N-A-NR²³—, G-O-A-O—, G-O-A-S—, 0-O-A-NR²³—, G-S-A-O—, G-S-A-NR²³— or R²⁰S(O)(═NR¹⁷)—, where R¹⁷, R²⁰, R²¹, R²² and R²³ are as defined above, or two of the groups R¹ and R² attached to the same carbon atom are ═O, ═S, ═NR¹¹ or ═CR¹²R¹³, where R¹¹, R¹² and R¹³ are defined as above, or the groups R¹ and R² together with the same carbon atom to which they are attached form a three to six membered ring, containing at least 2 carbon atoms and optionally containing one or two sulfur and/or one or two non-adjacent oxygen atoms or a group NR¹⁴, where R¹⁴ is as defined above, the ring being optionally substituted by C₁₋₆ alkyl; or two of the groups R¹, R² and R⁷, R⁸ attached to different atoms together with the atoms they are attached form a three to seven membered ring, that optionally contains one or two sulfur and/or one or two non-adjacent oxygen atoms or a group NR¹⁴, where R¹⁴ is defined as above, the ring being optionally substituted by one or four C₁₋₆ alkyl groups or phenyl; or two of the groups R¹, R², R⁵, R⁶, R⁷ and R⁸ attached to adjacent atoms combine to form a bond;

each R⁴ is independently OH, halogen, nitro, cyano, azido, rhodano, isothiocyanato, carboxy, formyl, formyloxy, G-, G-O—, G-S—, G-A-, R³¹R³²N—, R³¹R³²N-A-, G-O-A-, G-S-A-, G-A-O—, G-A-S—, G-A-NR³³—, R³¹R³²N-A-O—, R³¹R³²N-A-S—, R³¹R³²N-A-NR³³—, G-O-A-O—, G-O-A-S—, G-O-A-NR³³—, G-S-A-O, G-S-A-NR³³—, R²⁰S(O)(═NR¹⁷)—, R¹⁸N═C(R¹⁹)—, R⁴⁴R⁴⁵P(O)— or R⁴⁴R⁴⁵P(S)—, where R¹⁷, R¹⁸, R¹⁹ and R²⁰ have the meanings assigned to them above, and R³¹, R³² and R³³ are independently H or G-, or R³¹ and R³² together with the N atom to which they are attached, form a group N═CRaRb where Ra and Rb are H, C₁₋₆ alkyl or phenyl; or R³¹ and R³² together with the N atom to which they are attached form a five, six or seven membered ring which may contain one or two further hetero atoms selected from O, N or S and which may be optionally substituted by one to four C₁₋₆ alkyl groups or phenyl, and R⁴¹ and R⁴⁵ are independently H, C₁₋₆ alkyl, C₁₋₆ alkoxy, phenyl, phenoxy; or 2 adjacent groups R⁴ together with the carbon atoms to which they are attached form a 4, 5, 6 or 7 membered carbocyclic or heterocyclic ring which may be optionally substituted by C₁₋₆alkyl or halogen; or a group R⁴ together with a group R³, R⁵, R⁶ or R⁹ and the atoms to which they are attached form a 5-7 membered ring optionally containing an NR¹⁵ group S-A-, G-A-NR²⁹—, R²⁷R²⁸N-A-NR²⁹—, G-O-A-NR²⁹— or G-S-A-NR²⁹—, where R²⁷, R²⁸ and R²⁹ have the meanings assigned to them above, or containing an S or O atom, the ring being optionally substituted by one to four C₁₋₆ alkyl groups or phenyl;

n is 0, 1, 2, 3 or 4;

R⁹ is H, formyl, G-, G-A-, G-O-A-, R³⁴R³⁵N-A-, where R³⁴ and R³⁵ are independently H or G-, or R³⁴ and R³⁵ together with the N atom to which they are attached, form a group N═CRaRb where Ra and Rb are H, C₁₋₆ alkyl or phenyl; or R³⁴ and R³⁵ together with the N atom to which they are attached form a five, six or seven membered ring which may contain one or two further hetero atoms selected from O, N or S and which may be optionally substituted by one to four C₁₋₆ alkyl groups or phenyl; or R⁹ is G-O-A- or G-S-A-; or R⁹ together with a group R¹, R², R³, R⁵, R⁶, R⁷ or R⁸ and the atoms to which they are attached may form a three to seven membered ring, that optionally may contain one or two sulfur and/or one or two non-adjacent oxygen atoms or a group NR¹⁶; where R¹⁶ is H, OH, cyano, formyl, G-, G-O—, G-S—, G-A-, R²⁷R²⁸N—, R²⁷R²⁸N-A-, G-O-A-, G-S-A-, G-A-NR²⁹—, R²⁷R²⁸N-A-NR²⁹—, G-O-A-NR²⁹— or G-S-A-NR²⁹—, where R²⁷, R²⁸ and R²⁹ have the meanings assigned to them above,

G is optionally substituted C₁₋₁₂ alkyl, optionally substituted C₂₋₁₂ alkenyl, optionally substituted C₂₋₁₂ alkynyl, optionally substituted C₃₋₈ cycloalkyl, optionally substituted C₃₋₈ cycloalkenyl, optionally substituted aryl, optionally substituted heteroaryl or optionally substituted heterocyclyl;

A is S(O), SO₂, C(O) or C(S);

or salts or N-oxides thereof, and where at least one of R¹, R², R⁴, R⁵, R⁶, or R⁹ is different to hydrogen, when R¹⁰ is hydrogen or methyl, T is benzene and Y is CR⁵R⁶. In some embodiments, when the compound of formula I is a compound of formula Iα or Iβ

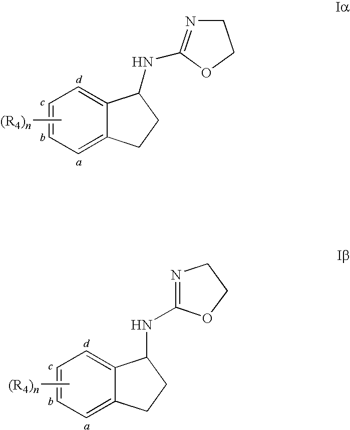

wherein R⁴ is as defined above, and (i) n is 2 or 3, and R⁴ at position “c” is selected from CH₃, Cl, F, Br, CF₃ and OCF₃, then at least one further R⁴ is different to that at position “c”, and when a further R⁴ is present at position “a”, then the R⁴ at position “a” is not selected from CH₃, Cl, F, Br, CF₃ or OCF₃, or (ii) when the compound of formula I is a compound of formula Iα or Iβ as defined above, and n is 1, then R⁴ is not CH₃, Cl, F, Br, CF₃ or OCF₃.

The compounds of formula (I) may exist in different geometric or optical isomers or different tautomeric forms. One or more centres of chirality may be present, for example on the chiral carbon atoms CR¹R², CR⁵R⁶, CR⁷R⁸, and CR⁹ or a chiral carbon unit in the group G, or a chiral —S(O)— unit, in which case compounds of the formula (I) may be present as pure enantiomers, mixtures of enantiomers, pure diastereomers or mixtures of diastereomers. There may be double bonds present in the molecule, such as C═C or C═N bonds, in which case compounds of formula (I) may exist as single isomers of mixtures of isomers. Centres of tautomerisation may be present. This invention covers all such isomers and tautomers and mixtures thereof in all proportions as well as isotopic forms such as deuterated compounds.

The compounds of formula (I) contain an amidine moiety, which can exist in two tautomeric forms when R¹⁰ is hydrogen. One of these forms contains an exocyclic C═N double bond, and one of them contains an endocyclic C═N double bond. When the C═N double bond is exocyclic this double bond can exist in two geometric forms E and Z as shown by formula (I′) and (I″). Each form can be latentiated with a group R¹⁰. This group R¹⁰ is selected to allow its removal by one or a combination of biochemical, chemical or physical processes to afford compounds of formula I where R¹⁰ is hydrogen before, during or following application to the treated area or plants. Examples of these processes include enzymatic cleavage, chemical hydrolysis and photolysis. Compounds bearing groups R¹⁰ may offer certain advantages, such as improved penetration of the cuticula of the plants treated, increased tolerance of crops, improved compatibility or stability in formulated mixtures containing other herbicides, herbicide safeners, plant growth regulators, fungicides or insecticides, increased movement in soils or reduced leaching in soils.

Suitable acid addition salts include those with an inorganic acid such as hydrochloric, hydrobromic, sulfuric, nitric and phosphoric acids, or an organic carboxylic acid such as oxalic, tartaric, lactic, butyric, toluic, hexanoic and phthalic acids, or sulphonic acids such as methane, benzene and toluene sulphonic acids. Other examples of organic carboxylic acids include haloacids such as trifluoroacetic acid.

N-oxides are oxidised forms of tertiary amines or oxidised forms of nitrogen containing heteroaromatic compounds. They are described in many books for example in “Heterocyclic N-oxides” by Angelo Albini and Silvio Pietra, CRC Press, Boca Raton, Fla., 1991.

Each alkyl moiety either alone or as part of a larger group (such as G, alkoxy, alkoxycarbonyl, alkylcarbonyl, alkylaminocarbonyl, dialkylaminocarbonyl) is a straight or branched chain and is, for example, methyl, ethyl, n-propyl, n-butyl, n-pentyl, n-hexyl, iso-propyl, n-butyl, sec-butyl, iso-butyl, tert-butyl or neo-pentyl. The alkyl groups are suitably C₁ to C₁₂ alkyl groups, but are preferably C₁-C₁₀, more preferably C₁-C₈, even more preferably C₁₋₆ and most preferably C₁₋₄ alkyl groups.

Ring or chain forming alkylen, alkenylen and alkinyl groups can optionally be further substituted by one or more halogen, C₁₋₃ alkyl and/or C₁₋₃ alkoxy group.

When present, the optional substituents on an alkyl moiety (alone or as part of a larger group such as G, alkoxy, alkoxycarbonyl, alkylcarbonyl, alkylaminocarbonyl, dialkylaminocarbonyl) include one or more of halogen, nitro, cyano, rhodano, isothiocyanato, C₃₋₇ cycloalkyl (itself optionally substituted with C₁₋₆ alkyl or halogen), C₅₋₇ cycloalkenyl (itself optionally substituted with C₁₋₆ alkyl or halogen), hydroxy, C₁₋₁₀ alkoxy, C₁₋₁₀ alkoxy(C₁₋₁₀)alkoxy, tri(C₁₋₄alkylsilyl(C₁₋₆)alkoxy, C₁₋₆ alkoxycarbonyl(C₁₋₁₀)alkoxy, C₁₋₁₀ haloalkoxy, aryl(C₁₋₄)-alkoxy (where the aryl group is optionally substituted), C₃₋₇ cycloalkyloxy (where the cycloalkyl group is optionally substituted with C₁₋₆ alkyl or halogen), C₂₋₁₀ alkenyloxy, C₂₋₁₀ alkynyloxy, mercapto, C₁₋₁₀ alkylthio, C₁₋₁₀ haloalkylthio, aryl(C₁₋₄)alkylthio (where the aryl group is optionally substituted), C₃₋₇ cycloalkylthio (where the cycloalkyl group is optionally substituted with C₁₋₆ alkyl or halogen), tri(C₁₋₄)alkylsilyl(C₁₋₆)alkylthio, arylthio (where the aryl group is optionally substituted), C₁₋₆ alkylsulfonyl, C₁₋₆ haloalkylsulfonyl, C₁₋₆ alkylsulfinyl, C₁₋₆ haloalkylsulfinyl, arylsulfonyl (where the aryl group may be optionally substituted), tri(C₁₋₄)alkylsilyl, (C₁₋₄)alkyldiarylsilyl, triarylsilyl, aryl(C₁₋₄)alkylthio(C₁₋₄)alkyl, aryloxy(C₁₋₄)alkyl, formyl, C₁₋₁₀ alkylcarbonyl, HO₂C, C₁₋₁₀ alkoxycarbonyl, aminocarbonyl, C₁₋₆ alkylaminocarbonyl, di(C₁₋₆ alkyl)aminocarbonyl, N—(C₁₋₃ alkyl)-N—(C₁₋₃ alkoxy)aminocarbonyl, C₁₋₆ alkylcarbonyloxy, arylcarbonyloxy (where the aryl group is optionally substituted), di(C₁₋₆)alkylaminocarbonyloxy, oximes and oximethers such as ═NOalkyl, ═NOhaloalkyl and ═NOaryl (itself optionally substituted), aryl (itself optionally substituted), heteroaryl (itself optionally substituted), heterocyclyl (itself optionally substituted with C₁₋₆ alkyl or halogen), aryloxy (where the aryl group is optionally substituted), heteroaryloxy, (where the heteroaryl group is optionally substituted), heterocyclyloxy (where the heterocyclyl group is optionally substituted with C₁₋₆ alkyl or halogen), amino, C₁₋₆ alkylamino, di(C₁₋₆)alkylamino, C₁₋₆ alkylcarbonylamino, N—(C₁₋₆)alkylcarbonyl-N—(C₁₋₆)alkylamino, C₂₋₆ alkenylcarbonyl, C₂₋₆ alkynylcarbonyl, C₃₋₆ alkenyloxycarbonyl, C₃₋₆ alkynyloxycarbonyl, aryloxycarbonyl (where the aryl group is optionally substituted) and arylcarbonyl (where the aryl group is optionally substituted).

Alkenyl and alkynyl moieties can be in the form of straight or branched chains, and the alkenyl moieties, where appropriate, can be of either the (E)- or (Z)-configuration. Examples are vinyl, allyl and propargyl. Alkenyl and alkynyl moieties can contain one or more double and/or triple bonds in any combination. It is understood, that allenyl and alkylinylalkenyl are included in these terms.

When present, the optional substituents on alkenyl or alkynyl include those optional substituents given above for an alkyl moiety.

In the context of this specification acyl is optionally substituted C₁₋₆ alkylcarbonyl (for example acetyl), optionally substituted C₂₋₆ alkenylcarbonyl, optionally substituted C₃₋₆ cycloalkylcarbonyl (for example cyclopropylcarbonyl, optionally substituted C₂₋₆ alkynylcarbonyl, optionally substituted arylcarbonyl (for example benzoyl) or optionally substituted heteroarylcarbonyl.

Halogen is fluorine, chlorine, bromine or iodine.

Haloalkyl groups are alkyl groups which are substituted with one or more of the same or different halogen atoms and are, for example, CF₃, CF₂C₁, CF₂H, CCl₂H, FCH₂, ClCH₂, BrCH₂, CH₃CHF, (CH₃)₂CF, CF₃CH₂ or CHF₂CH₂.

In the context of the present specification the terms “aryl”, “aromatic ring” and “aromatic ring system” refer to ring systems which may be mono-, bi- or tricyclic. Examples of such rings include phenyl, naphthalenyl, anthracenyl, indenyl or phenanthrenyl. A preferred aryl group is phenyl. In addition, the terms “heteroaryl”, “heteroaromatic ring” or “heteroaromatic ring system” refer to an aromatic ring system containing at least one heteroatom and consisting either of a single ring or of two or more fused rings. Preferably, single rings will contain up to three and bicyclic systems up to four heteroatoms which will preferably be chosen from nitrogen, oxygen and sulphur. Examples of such groups include furyl, thienyl, pyrrolyl, pyrazolyl, imidazolyl, 1,2,3-triazolyl, 1,2,4-triazolyl, oxazolyl, isoxazolyl, thiazolyl, isothiazolyl, 1,2,3-oxadiazolyl, 1,2,4-oxadiazolyl, 1,3,4-oxadiazolyl, 1,2,5-oxadiazolyl, 1,2,3-thiadiazolyl, 1,2,4-thiadiazolyl, 1,3,4-thiadiazolyl, 1,2,5-thiadiazolyl, pyridyl, pyrimidinyl, pyridazinyl, pyrazinyl, 1,2,3-triazinyl, 1,2,4-triazinyl, 1,3,5-triazinyl, benzofuryl, benzisofuryl, benzothienyl, benzisothienyl, indolyl, isoindolyl, indazolyl, benzothiazolyl, benzisothiazolyl, benzoxazolyl, benzisoxazolyl, benzimidazolyl, 2,1,3-benzoxadiazole, quinolinyl, isoquinolinyl, cinnolinyl, phthalazinyl, quinazolinyl, quinoxalinyl, naphthyridinyl, benzotriazinyl, purinyl, pteridinyl and indolizinyl. Preferred examples of heteroaromatic radicals include pyridyl, pyrimidyl, triazinyl, thienyl, furyl, oxazolyl, isoxazolyl, 2,1,3-benzoxadiazole and thiazolyl.

The terms heterocycle and heterocyclyl refer to a non-aromatic preferably monocyclic or bicyclic ring systems containing up to 10 atoms including one or more (preferably one or two) heteroatoms selected from O, S and N. Examples of such rings include 1,3-dioxolane, oxetane, tetrahydrofuran, morpholine, thiomorpholin and piperazine.

When present, the optional substituents on heterocyclyl include C₁₋₆ alkyl and C₁₋₆ haloalkyl as well as those optional substituents given above for an alkyl moiety.

Cycloalkyl includes cyclopropyl, cyclobutyl, cyclopentyl and cyclohexyl. Cycloalkylalkyl is preferentially cyclopropylmethyl. Cycloalkenyl includes cyclopentenyl and cyclohexenyl.

When present, the optional substituents on cycloalkyl or cycloalkenyl include C₁₋₃ alkyl as well as those optional substituents given above for an alkyl moiety.

Carbocyclic rings include aryl, cycloalkyl and cycloalkenyl groups.

When present, the optional substituents on aryl or heteroaryl are selected independently, from halogen, nitro, cyano, rhodano, isothiocyanato, C₁₋₆ alkyl, C₁₋₆ haloalkyl, C₁₋₆ alkoxy-(C₁₋₆)alkyl, C₂₋₆ alkenyl, C₂₋₆ haloalkenyl, C₂₋₆ alkynyl, C₃₋₇ cycloalkyl (itself optionally substituted with C₁₋₆ alkyl or halogen), C₅₋₇ cycloalkenyl (itself optionally substituted with C₁₋₆ alkyl or halogen), hydroxy, C₁₋₁₀ alkoxy, C₁₋₁₀ alkoxy(C₁₋₁₀)alkoxy, tri(C₁₋₄)alkyl-silyl(C₁₋₆)alkoxy, C₁₋₆ alkoxycarbonyl(C₁₋₁₀)alkoxy, C₁₋₁₀ haloalkoxy, aryl(C₁₋₄)alkoxy (where the aryl group is optionally substituted with halogen or C₁₋₆ alkyl), C₃₋₇ cycloalkyloxy (where the cycloalkyl group is optionally substituted with C₁₋₆ alkyl or halogen), C₂₋₁₀ alkenyloxy, C₂₋₁₀ alkynyloxy, mercapto, C₁₋₁₀ alkylthio, C₁₋₁₀ haloalkylthio, aryl(C₁₋₄)alkylthio, C₃₋₇ cycloalkylthio (where the cycloalkyl group is optionally substituted with C₁₋₆ alkyl or halogen), tri(C₁₋₄)-alkylsilyl(C₁₋₆)alkylthio, arylthio, C₁₋₆ alkylsulfonyl, C₁₋₆ haloalkylsulfonyl, C₁₋₆ alkylsulfinyl, C₁₋₆ haloalkylsulfinyl, arylsulfonyl, aryldi(C₁₋₄)-alkylsilyl, (C₁₋₄)alkyldiarylsilyl, triarylsilyl, C₁₋₁₀ alkylcarbonyl, HO₂C, C₁₋₁₀ alkoxycarbonyl, aminocarbonyl, C₁₋₆ alkylaminocarbonyl, di(C₁₋₆ alkyl)-aminocarbonyl, N—(C₁₋₃ alkyl)-N—(C₁₋₃ alkoxy)aminocarbonyl, C₁₋₆ alkylcarbonyloxy, arylcarbonyloxy, di(C₁₋₆)alkylamino-carbonyloxy, aryl (itself optionally substituted with C₁₋₆ alkyl or halogen), heteroaryl (itself optionally substituted with C₁₋₆ alkyl or halogen), heterocyclyl (itself optionally substituted with C₁₋₆ alkyl or halogen), aryloxy (where the aryl group is optionally substituted with C₁₋₆ alkyl or halogen), heteroaryloxy (where the heteroaryl group is optionally substituted with C₁₋₆ alkyl or halogen), heterocyclyloxy (where the heterocyclyl group is optionally substituted with C₁₋₆ alkyl or halogen), amino, C₁₋₆ alkylamino, di(C₁₋₆)alkylamino, C₁₋₆ alkylcarbonylamino, N—(C₁₋₆)alkylcarbonyl-N—(C₁₋₆)alkylamino, arylcarbonyl, (where the aryl group is itself optionally substituted with halogen or C₁₋₆ alkyl) or two adjacent positions on an aryl or heteroaryl system may be cyclised to form a 5, 6 or 7 membered carbocyclic or heterocyclic ring, itself optionally substituted with halogen or C₁₋₆ alkyl. Further substituents for aryl or heteroaryl include aryl carbonyl amino (where the aryl group is substituted by C₁₋₆ alkyl or halogen), C₁₋₆alkoxycarbonylamino C₁₋₆alkoxycarbonyl-N—(C₁₋₆)alkylamino, aryloxycarbonylamino (where the aryl group is substituted by C₁₋₆ alkyl or halogen), aryloxycarbonyl-N—(C₁₋₆)alkylamino (where the aryl group is substituted by C₁₋₆ alkyl or halogen), arylsulphonylamino (where the aryl group is substituted by C₁₋₆ alkyl or halogen), arylsulphonyl-N—(C₁₋₆)alkylamino (where the aryl group is substituted by C₁₋₆ alkyl or halogen), aryl-N—(C₁₋₆)alkylamino (where the aryl group is substituted by C₁₋₆ alkyl or halogen), arylamino (where the aryl group is substituted by C₁₋₆ alkyl or halogen), heteroaryl amino (where the heteroaryl group is substituted by C₁₋₆ alkyl or halogen), heterocyclylamino (where the heterocyclyl group is substituted by C₁₋₆ alkyl or halogen), aminocarbonylamino, C₁₋₆ alkylaminocarbonyl amino, di(C₁₋₆)alkylaminocarbonyl amino, arylaminocarbonyl amino where the aryl group is substituted by C₁₋₆ alkyl or halogen), aryl-N—(C₁₋₆)alkylaminocarbonylamino (where the aryl group is substituted by C₁₋₆ alkyl or halogen), C₁₋₆alkylaminocarbonyl-N—(C₁₋₆)alkyl amino, di(C₁₋₆)alkylaminocarbonyl-N—(C₁₋₆)alkyl amino, arylaminocarbonyl-N—(C₁₋₆)alkyl amino (where the aryl group is substituted by C₁₋₆ alkyl or halogen) and aryl-N—(C₁₋₆)alkylaminocarbonyl-N—(C₁₋₆)alkyl amino (where the aryl group is substituted by C₁₋₆ alkyl or halogen).

For substituted phenyl moieties, heterocyclyl and heteroaryl groups it is preferred that one or more substituents are independently selected from halogen, C₁₋₆ alkyl, C₁₋₆ haloalkyl, C₁₋₆ alkoxy(C₁₋₆)alkyl, C₁₋₆ alkoxy, C₁₋₆ haloalkoxy, C₁₋₆ alkylthio, C₁₋₆ haloalkylthio, C₁₋₆ alkylsulfinyl, C₁₋₆ haloalkylsulfinyl, C₁₋₆ alkylsulfonyl, C₁₋₆ haloalkylsulfonyl, C₂₋₆ alkenyl, C₂₋₆ haloalkenyl, C₂₋₆ alkynyl, C₃₋₇ cycloalkyl, nitro, cyano, CO₂H, C₁₋₆ alkylcarbonyl, C₁₋₆ alkoxycarbonyl, aryl, heteroaryl, R⁵⁰R⁵¹N or R⁵²R⁵³NC(O), wherein R⁵⁰, R⁵¹, R⁵² and R⁵³ are, independently, hydrogen or C₁₋₆ alkyl.

Haloalkenyl groups are alkenyl groups which are substituted with one or more of the same or different halogen atoms.

It is to be understood that dialkylamino substituents include those where the dialkyl groups together with the N atom to which they are attached form a five, six or seven-membered heterocyclic ring which may contain one or two further heteroatoms selected from O, N or S and which is optionally substituted by one or two independently selected (C₁₋₆)alkyl groups. When heterocyclic rings are formed by joining two groups on an N atom, the resulting rings are suitably pyrrolidine, piperidine, thiomorpholine and morpholine each of which may be substituted by one or two independently selected (C₁₋₆) alkyl groups.

Preferably the optional substituents on an alkyl moiety include one or more of halogen, nitro, cyano, HO₂C, C₁₋₁₀ alkoxy (itself optionally substituted by C₁₋₁₀ alkoxy), aryl(C₁₋₄)alkoxy, C₁₋₁₀ alkylthio, C₁₋₁₀ alkylcarbonyl, C₃₋₅ cycloalkylcarbonyl, C₁₋₁₀ alkoxycarbonyl, C₁₋₆ alkylaminocarbonyl, di(C₁₋₆ alkyl)aminocarbonyl, (C₁₋₆)alkylcarbonyloxy, optionally substituted phenyl, heteroaryl, aryloxy, arylcarbonyloxy, heteroaryloxy, heterocyclyl, heterocyclyloxy, C₃₋₇ cycloalkyl (itself optionally substituted with (C₁₋₆)alkyl or halogen), C₃₋₇ cycloalkyloxy, C₆₋₇ cycloalkenyl, C₁₋₆ alkylsulfonyl, C₁₋₆ alkylsulfinyl, tri(C₁₋₄)alkylsilyl, tri(C₁₋₄alkylsilyl(C₁₋₆)alkoxy, aryldi(C₁₋₄)alkylsilyl, (C₁₋₄)alkyldiarylsilyl and triarylsilyl.

Preferably the optional substituents on alkenyl or alkynyl include one or more of halogen, aryl and C₃₋₇ cycloalkyl.

A preferred optional substituent for heterocyclyl is C₁₋₃ alkyl.

Preferably the optional substituents for cycloalkyl include halogen, cyano and C₁₋₆ alkyl.

The optional substituents for cycloalkenyl preferably include C₁₋₃ alkyl, halogen and cyano.

In preferred compounds R⁵, R⁶, R⁷ and/or R⁸ is/are independently hydrogen, halogen, hydroxy, amino, nitro, cyano, C₁₋₆ alkyl, C₁₋₆ alkenyl, C₁₋₆ haloalkyl, C₁₋₆ alkoxy(C₁₋₆)alkyl, phenyl(C₁₋₃)alkyl (wherein the phenyl group may be optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino, dialkylamino, C₁₋₆ alkylsulfonyl or C₁₋₆ alkoxycarbonyl, or two adjacent positions on the phenyl ring may be cyclised to form a 5, 6 or 7 membered carbocyclic or heterocyclic ring, itself optionally substituted with halogen), C₃₋₅ cycloalkyl, 1,3-dioxolan-2-yl, phenyl (which may be optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino, dialkylamino, C₁₋₆ alkylsulfonyl or C₁₋₆ alkoxycarbonyl, or two adjacent positions on the phenyl ring may be cyclised to form a 5, 6 or 7 membered carbocyclic or heterocyclic ring, itself optionally substituted with halogen), heteroaryl (which may be optionally substituted by halo, nitro, cyano, C₁₋₆ alkyl, C₁₋₆ haloalkyl, C₁₋₆ alkoxy or C₁₋₆ haloalkoxy), C₁₋₆ alkoxy, C₁₋₆ haloalkoxy, C₁₋₆ alkenyloxy, C₁₋₆ alkynyloxy, C₁₋₃ alkoxy(C₁₋₃)alkoxy, benzyloxy (where phenyl is optionally substituted by halogen, C₁₋₄ alkyl, C₁₋₄ alkoxy, C₁₋₄ haloalkyl, C₁₋₄ haloalkoxy, CN, NO₂), phenoxy (where phenyl may be optionally substituted by halo, nitro, cyano, C₁₋₆ alkyl, C₁₋₆ haloalkyl, C₁₋₆ alkoxy or C₁₋₆ haloalkoxy), C₁₋₆ alkylthio, C₁₋₆ haloalkylthio, formyl, C₁₋₆ alkylcarbonyl, C₁₋₆ alkoxycarbonyl, carboxy, C₁₋₆ alkoxythionocarbonyl, carbamoyl, C₁₋₆ alkylaminocarbonyl, di-C₁₋₆ alkylaminocarbonyl, thiocarbamoyl, C₁₋₆ alkylaminothionocarbonyl, alkylaminothionocarbonyl, C₁₋₆ alkylcarbonyloxy, C₁₋₆ alkylthionocarbonyloxy, C₁₋₆ alkoxycarbonyloxy, C₁₋₆ alkylaminocarbonyloxy, di-C₁₋₆ alkylaminocarbonyloxy, C₁₋₆ alkylcarbonylthio, C₁₋₆ alkylaminothionocarbonylthio, di-(C₁₋₆)-alkylaminothionocarbonylthio, C₁₋₆ alkoxycarbonylamino, aminocarbonylamino, C₁₋₆ alkylaminocarbonylamino, di-C₁₋₆alkylaminocarbonylamino, NR⁵⁴R⁵⁵ (where R⁵⁴ and R⁵⁵ are independently hydrogen, C₁₋₆ alkyl, C₁₋₆ haloalkyl, C₁₋₄ alkoxy(C₁₋₄)alkyl, formyl, C₁₋₆ alkylcarbonyl or phenylcarbonyl (where the phenyl is optionally substituted by halogen, C₁₋₄ alkyl, C₁₋₄ alkoxy, C₁₋₄ haloalkyl, C₁₋₄ haloalkoxy, CN or NO₂), or R⁵ and R⁶ or R⁷ and R⁸ together with the carbon atom to which they are attached form a three to six membered ring, that optionally may contain one or two sulfur or one or two not adjacent oxygen atoms or a group NR⁵⁶ (where R⁵⁶ is hydrogen, C₁₋₆ alkyl, C₁₋₆ haloalkyl, C₁₋₄ alkoxy(C₁₋₄)alkyl, formyl, C₁₋₆ alkylcarbonyl), or R⁵ or R⁷ when attached to a carbon atom adjacent to the R¹ bearing carbon atom, together with R¹ form a bond; or R⁵ and R¹, or R⁷ and R¹, together with the carbon atoms to which they are attached form a three to six membered ring optionally comprising one or two non-adjacent oxygen atoms; or R⁵ and R⁶, or R⁷ and R⁸, form ═O, ═S, ═NR⁵⁷ or ═CR⁵⁸R⁵⁹, wherein R⁵⁷ is OH, optionally substituted C₁₋₆ alkoxy or C₁₋₄ alkylcarbonylamino, and R⁵⁸ and R⁵⁹ are independently H or C₁₋₆ alkyl.

Y is preferably O, S, S(O), SO₂, NR³ or CR⁵R⁶ where R³, R⁵ and R⁶ are defined above. R³ is especially hydrogen, formyl, C₁₋₆ alkylcarbonyl, cyclopropylcarbonyl, C₁₋₆ alkoxycarbonyl, C₁₋₆ alkylsulfonyl, C₁₋₆ alkyl, C₁₋₆ haloalkyl, C₃₋₄ alkenyl, C₃₋₄ haloalkenyl, C₃₋₄ alkynyl, benzyl or phenyl (where the phenyl containing groups are optionally substituted by halogen, C₁₋₄ alkyl, C₁₋₄ alkoxy, C₁₋₄ haloalkyl, C₁₋₄ haloalkoxy, CN or NO₂). Especially R⁵ and R⁶ are independently hydrogen, hydroxy, halogen, cyano, C₁₋₆ alkyl, C₁₋₆ haloalkyl, C₁₋₆ alkoxy(C₁₋₆)alkyl, phenyl(C₁₋₃)alkyl (wherein the phenyl group may be optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino, dialkylamino, C₁₋₆ alkylsulfonyl, C₁₋₆ alkoxycarbonyl, or two adjacent positions on the phenyl ring may be cyclised to form a 5, 6 or 7 membered carbocyclic or heterocyclic ring, itself optionally substituted with halogen), C₃₋₅ cycloalkyl, 1,3-dioxolan-2-yl, phenyl (which may be optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino, dialkylamino, C₁₋₆ alkylsulfonyl, C₁₋₆ alkoxycarbonyl, or two adjacent positions on the phenyl ring may be cyclised to form a 5, 6 or 7 membered carbocyclic or heterocyclic ring, itself optionally substituted with halogen), C₁₋₆ alkoxy, C₁₋₆ haloalkoxy, C₁₋₆ alkenyloxy, C₁₋₆ alkynyloxy, C₁₋₃ alkoxy(C₁₋₃)alkoxy, benzyloxy (where phenyl is optionally substituted by halogen, C₁₋₄ alkyl, C₁₋₄ alkoxy, C₁₋₄ haloalkyl, C₁₋₄ haloalkoxy, CN, NO₂), C₁₋₆ alkylthio, C₁₋₆ haloalkylthio, NR⁵⁴R⁵⁵ (where R⁵⁴ and R⁵⁵ are independently hydrogen, C₁₋₆ alkyl, C₁₋₆ haloalkyl, C₁₋₄ alkoxy(C₁₋₄)alkyl, formyl, C₁₋₆ alkylcarbonyl or phenylcarbonyl (where the phenyl is optionally substituted by halogen, C₁₋₄ alkyl, C₁₋₄ alkoxy, C₁₋₄ haloalkyl, C₁₋₄ haloalkoxy, CN or NO₂), or R⁵ and R⁶ together with the carbon atom to which they are attached form a three to six membered ring, that optionally may contain one or two sulfur or one or two not adjacent oxygen atoms or a group NR⁵⁶ (where R⁵⁶ is hydrogen, C₁₋₆ alkyl, C₁₋₆ haloalkyl, C₁₋₄ alkoxy(C₁₋₄)alkyl, formyl, C₁₋₆ alkylcarbonyl), or when R⁵ together with R¹ forms a bond, or either R⁵ or R⁶ together with R¹ or R² and the carbon atom they are attached form a three to six membered ring, that optionally may contain one or two not adjacent oxygen atoms, or R⁵ and R⁶ together form ═O, ═S, ═NR⁵⁷ or ═CR⁵⁸R⁵⁹, wherein R⁵⁷ is OH, optionally substituted C₁₋₆ alkoxy or C₁₋₄ alkylcarbonylamino, and R⁵⁸ and R⁵⁹ are independently H or C₁₋₆ alkyl.

More preferably Y is O or CR⁵R⁶ where R⁵ and R⁶ are hydrogen, hydroxy, fluoro, chloro, C₁₋₆ alkyl, C₁₋₆ haloalkyl, C₁₋₆ alkoxy, C₁₋₆ haloalkoxy, C₁₋₆ alkenyloxy, C₁₋₆ alkynyloxy, C₁₋₃ alkoxy(C₁₋₃)alkoxy or benzyloxy, or R⁵ and R⁶ together with the carbon atom to which they are attached form a three to six membered carbocyclic ring, or R⁵ and R¹ together form a bond, or R⁵ together with R¹ and the carbon atoms to which they are attached form a three to six membered carbocyclic ring, or R⁵ and R⁶ together form ═CR⁵⁸R⁵⁹, wherein R⁵⁸ and R⁵⁹ are independently H or C₁₋₆ alkyl.

Even more preferably Y is CR⁵R⁶, where R⁵ and R⁶ are independently hydrogen, fluorine or methyl. Even more preferably Y is CR⁵R⁶, where R⁵ and R⁶ are independently hydrogen, or methyl and most preferably R⁵ and R⁶ are hydrogen.

In some embodiments R¹ and R² are independently hydrogen, hydroxy, halogen, amino, nitro, cyano, C₁₋₆ alkyl, C₁₋₆ alkenyl, C₉₋₆ haloalkyl, C₁₋₆ alkoxy(C₁₋₆)alkyl, phenyl(C₁₋₃)alkyl (wherein the phenyl group may be optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino, dialkylamino, alkylsulfonyl or C₁₋₆ alkoxycarbonyl), C₃₋₆ cycloalkyl, 1,3-dioxolan-2-yl, phenyl (which may be optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino, dialkylamino, C₁₋₆ alkylsulfonyl or C₁₋₆ alkoxycarbonyl), heteroaryl (which may be optionally substituted by halo, nitro, cyano, C₁₋₆ alkyl, C₁₋₆ haloalkyl, C₁₋₆ alkoxy or C₁₋₆ haloalkoxy), C₁₋₆ alkoxy, C₁₋₆ haloalkoxy, C₂₋₆ alkenyloxy, C₂₋₆ alkynyloxy, phenoxy (where phenyl may be optionally substituted by halo, nitro, cyano, alkyl, C₁₋₆ haloalkyl, C₁₋₆ alkoxy or C₁₋₆ haloalkoxy), C₁₋₆ alkylthio, C₁₋₆ haloalkylthio, formyl, C₁₋₆ alkylcarbonyl, phenylcarbonyl (where the phenyl is optionally substituted by halogen, C₁₋₄ alkyl, C₁₋₄ alkoxy, C₁₋₄ haloalkyl, C₁₋₄ haloalkoxy, CN, or NO₂), C₁₋₆ alkoxycarbonyl, carboxy, C₁₋₆ alkoxythionocarbonyl, carbamoyl, C₁₋₆ alkylaminocarbonyl, di-C₁₋₆ alkylaminocarbonyl, thiocarbamoyl, C₁₋₆ alkylaminothionocarbonyl, di-C₁₋₆ alkylaminothionocarbonyl, C₁₋₆ alkylcarbonyloxy, C₁₋₆ alkylthionocarbonyloxy, C₁₋₆ alkoxycarbonyloxy, C₁₋₆ alkylaminocarbonyloxy, di-C₁₋₆ alkylaminocarbonyloxy, C₁₋₆ alkylcarbonylthio, C₁₋₆ alkylaminothionocarbonylthio, di-(C₁₋₆)-alkylaminothionocarbonylthio, C₁₋₈ alkylamino, di-(C″)-alkylamino, formylamino, C₁₋₆ alkylcarbonylamino, C₁₋₆ alkoxycarbonylamino, aminocarbonylamino, C₁₋₆ alkylaminocarbonylamino, di-C₁₋₆alkylaminocarbonylamino, or R¹ and R² together are ═O, ═S, ═NR¹¹ or ═CR¹²R¹³, wherein R¹¹ is OH, C₁₋₆ alkoxy or C₁₋₆ alkylcarbonylamino, and R¹² and R¹³ are independently H, C₁₋₆ alkyl, or C₁₋₆ haloalkyl; or R¹ and R⁹ together with the carbon atom they are attached form a three to six membered ring, that optionally may contain one or two not adjacent oxygen atoms; or Wand R² together form a three to six membered ring, that optionally may contain one or two non-adjacent oxygen atoms.

Preferably each R¹ and R² group is independently hydrogen, hydroxy, halogen, cyano, C₁₋₆ alkyl, C₁₋₆ haloalkyl, C₁₋₆ alkoxy(C₁₋₆)alkyl, phenyl(C₁₋₃)alkyl (wherein the phenyl group may be optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino, dialkylamino, C₁₋₆ alkylsulfonyl, C₁₋₆ alkoxycarbonyl), C₃₋₅ cycloalkyl, 1,3-dioxolan-2-yl, phenyl (which may be optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino, dialkylamino, C₁₋₆ alkylsulfonyl, C₁₋₆ alkoxycarbonyl), C₁₋₆ alkoxy, C₁₋₆ haloalkoxy, C₂₋₆ alkenyloxy, C₂₋₆ alkinyloxy, C₁₋₆ alkylthio, C₁₋₆ haloalkylthio, formyl, C₂₋₆ alkylcarbonyl, phenylcarbonyl (where the phenyl is optionally substituted by halogen, C₁₋₄ alkyl, C₁₋₄ alkoxy, C₁₋₄ haloalkyl, C₁₋₄ haloalkoxy, CN, NO₂), or R¹ and R² together are ═O, ═S, ═NR¹¹ or ═CR¹²R¹³, R¹¹ is OH, C₁₋₆ alkoxy or C₁₋₆ alkylcarbonylamino, and R¹² and R¹³ are independently H, C₁₋₆ alkyl, or C₁₋₆ haloalkyl; or R¹ and R⁹ together with the carbon atom they are attached form a three to six membered ring, that optionally may contain one or two not adjacent oxygen atoms; or R¹ and R² together form a three to six membered ring, that optionally may contain one or two non-adjacent oxygen atoms.

More preferably each R¹ and R² group is independently hydrogen, hydroxy, halogen, C₁₋₆ alkyl, C₁₋₆ haloalkyl, C₁₋₆ alkoxy, C₁₋₆ haloalkoxy, C₁₋₆ alkenyloxy, C₁₋₆ alkynyloxy, C₁₋₃ alkoxy(C₁₋₃)alkoxy or benzyloxy.

Even more preferably each R¹ and R² group is independently hydrogen, fluorine or methyl, most preferably hydrogen or methyl.

In some embodiments R⁹ is hydrogen, C₁₋₆ alkyl, C₁₋₆ cyanoalkyl, C₁₋₆ haloalkyl, C₃₋₇ cycloalkyl(C₁₋₄)alkyl, C₁₋₆ alkoxy(C₁₋₆)alkyl, aryl(C₁₋₆)alkyl (wherein the aryl group may be optionally substituted by halo, nitro, cyano, C₁₋₆ alkyl, C₁₋₆ haloalkyl, C₁₋₆ alkoxy, C₁₋₆ haloalkoxy, C₁₋₆ alkylsulfonyl, C₁₋₆ alkylsulfinyl, C₁₋₆ alkylthio, C₁₋₆ alkoxycarbonyl, C₁₋₆ alkylcarbonylamino or arylcarbonyl), C₁₋₆ alkylcarbonyl, phenylcarbonyl (where the phenyl is optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino or dialkylamino), C₁₋₆ alkoxycarbonyl, carboxy, alkylaminothionocarbonyl or C(O)NR³⁴R³⁵ (where R³⁴ and R³⁵ are independently hydrogen, C₁₋₆ alkyl or C₁₋₆ haloalkyl or C₁₋₆ alkoxy(C₁₋₆)alkyl; or R³⁴ and R³⁵ together with the N atom to which they are attached form a five, six or seven-membered ring containing an O or S atom); or R⁹ and R¹ or R⁹ and R² together with the carbon atoms to which they are attached form a three to six membered ring, that may optionally contain one or two sulphur and/or one or two non-adjacent oxygen atoms.

R⁹ is preferably hydrogen, C₁₋₆ alkyl, C₁₋₆ cyanoalkyl, C₁₋₆ haloalkyl, C₃₋₇ cycloalkyl(C₁₋₄)alkyl, C₁₋₆ alkoxy(C₁₋₆)alkyl, aryl(C₁₋₆)alkyl (wherein the aryl group may be optionally substituted by halo, nitro, cyano, C₁₋₆ alkyl, C₁₋₆ haloalkyl, C₁₋₆ alkoxy, C₁₋₆ haloalkoxy, C₁₋₆ alkylsulfonyl, C₁₋₆ alkylsulfinyl, C₁₋₆ alkylthio, C₁₋₆ alkoxycarbonyl, C₁₋₆ alkylcarbonylamino, arylcarbonyl,), C₁₋₆ alkylcarbonyl, phenylcarbonyl (where the phenyl is optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino or dialkylamino), C₁₋₆ alkoxycarbonyl, C(O)NR³⁴R³⁵ (where R³⁴ and R³⁵ are independently hydrogen, C₁₋₆ alkyl or C₁₋₆ haloalkyl or C₁₋₆ alkoxy(C₁₋₆)alkyl or R³⁴ and R³⁵ together with the N atom to which they are attached form a five, six or seven-membered ring containing an O or S atom), or R⁹ and R¹ together with the carbon atoms to which they are attached form a three to six membered ring, that may optionally contain one or two sulphur and/or one or two non-adjacent oxygen atoms.

More preferably R⁹ is independently hydrogen, C₁₋₆ alkyl, C₁₋₆ haloalkyl, C₃₋₇ cycloalkyl(C₁₋₄)alkyl, C₁₋₆ alkoxy(C₁₋₆)alkyl, aryl(C₁₋₆)alkyl (wherein the aryl group may be optionally substituted by halo, nitro, cyano, C₁₋₆ alkyl, C₁₋₆ haloalkyl, C₁₋₆ alkoxy), C₂₋₆ alkylcarbonyl, phenylcarbonyl (where the phenyl is optionally substituted by halogen, C₁₋₄ alkyl, C₁₋₄ alkoxy, C₁₋₄ haloalkyl), C₁₋₆ alkoxycarbonyl, or R⁹ and R¹ together with the carbon atoms to which they are attached form a three to six membered ring.

Most preferably R₉ is independently hydrogen or methyl.

In some embodiments each R⁴ is independently halogen, hydroxy, amino, nitro, cyano, C₁₋₈ alkyl, C₁₋₈ haloalkyl, cyano(C₁₋₆)alkyl, C₁₋₃ alkoxy(C₁₋₃)alkyl, C₂₋₆ alkenyl, C₂₋₆ alkynyl, C₃₋₆ cycloalkyl, C₁₋₃ alkyl-(C₃₋₆)-cycloalkyl, phenyl (optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino or dialkylamino), heteroaryl (optionally substituted by halo, nitro, cyano, C₁₋₆ alkyl, C₁₋₆ haloalkyl, C₁₋₆ alkoxy or C₁₋₆ haloalkoxy), heterocyclyl (optionally substituted by halo, nitro, cyano, C₁₋₆ alkyl, C₁₋₆ haloalkyl, C₁₋₆ alkoxy or C₁₋₆ haloalkoxy), formyl, C₁₋₆ alkylcarbonyl, carboxy, C₁₋₆ alkoxycarbonyl, C₁₋₆ alkylthiocarbonyl, C₁₋₆ alkylthionocarbonyloxy, C₁₋₆ alkoxythionocarbonyl, carbamoyl, C₁₋₆ alkylaminocarbonyl, alkylaminocarbonyl, thiocarbamoyl, C₁₋₆ alkylamino-thionocarbonyl, di-C₁₋₆ alkylaminothionocarbonyl, C₁₋₈ alkoxy, C₁₋₆ haloalkoxy, phenoxy (optionally substituted by halogen, C₁₋₄ alkyl, C₁₋₄ alkoxy, C₁₋₄ haloalkyl, C₁₋₄ haloalkoxy, CN, NO₂, phenyl), heteroaryloxy (optionally substituted by halo, nitro, cyano, C₁₋₃ alkyl, C₁₋₃ haloalkyl, C₁₋₃ alkoxy or C₁₋₃ haloalkoxy), C₁₋₆ alkylcarbonyloxy, C₁₋₆ alkoxycarbonyloxy, C₁₋₆ alkylaminocarbonyloxy, di-C₁₋₆ alkylaminocarbonyloxy, alkylaminothionocarbonyloxy, alkylaminothionocarbonyloxy, C₁₋₈ alkylthio, C₁₋₆ haloalkylthio, arylthio or heteroarylthio (where the aryl is optionally substituted by halogen, C₁₋₄ alkyl, C₁₋₄ alkoxy, C₁₋₄ haloalkyl, C₁₋₄ haloalkoxy, CN, NO₂ or phenyl), C₁₋₆ alkylcarbonylthio, C₁₋₆ alkylaminocarbonylthio, alkylaminocarbonylthio, C₁₋₆ alkylaminothionocarbonylthio, di-(C₁₋₆)-alkylamino-thionocarbonylthio, C₁₋₈ alkylamino, di-(C₁₋₈)-alkylamino, formylamino, C₁₋₆ alkylcarbonylamino, C₁₋₆ alkoxycarbonylamino, aminocarbonylamino, C₁₋₆ alkylaminocarbonylamino, di-C₁₋₆alkylaminocarbonylamino, aminothionocarbonylamino, C₁₋₆ alkylaminothionocarbonylamino, di-C₁₋₆ alkylaminothionocarbonylamino, or 2 adjacent groups R⁴ together with the carbon atoms to which they are attached form a 4, 5, 6 or 7 membered carbocylic or heterocyclic ring which may be optionally substituted by halogen.

In some preferred embodiments at least one R⁴ is independently phenyl that is substituted by dialkylamino, heterocyclyl (optionally substituted by halo, nitro, cyano, C₁₋₆ alkyl, C₁₋₆ haloalkyl, C₁₋₆ alkoxy or C₁₋₆ haloalkoxy), C₁₋₆ alkylthiocarbonyl, C₁₋₆ alkylthionocarbonyloxy, C₁₋₆ alkoxycarbonyloxy, C₁₋₆ alkylaminocarbonyloxy, alkylaminocarbonyloxy, C₁₋₆ alkylaminothionocarbonyloxy, alkylaminothionocarbonyloxy, C₁₋₆ alkylcarbonylthio, C₁₋₆ alkylaminocarbonylthio, di-C₁₋₆ alkylaminocarbonylthio, C₁₋₆ alkylamino-thionocarbonylthio, di-(C₁₋₆)-alkylaminothionocarbonylthio, formylamino, C₁₋₆ alkylcarbonylamino, C₁₋₆ alkoxycarbonylamino, aminocarbonylamino, C₁₋₆ alkylaminocarbonylamino, di-C₁₋₆ alkylaminocarbonylamino, aminothionocarbonylamino, C₁₋₆ alkylaminothionocarbonylamino, di-C₁₋₆ alkylaminothionocarbonylamino, or 2 adjacent groups R⁴ together with the carbon atoms to which they are attached form a 4 membered carbocylic or heterocyclic ring which may be optionally substituted by halogen.

In other embodiments each R⁴ is independently halogen, nitro, cyano, C₁₋₈ alkyl, C₁₋₈ haloalkyl, cyano(C₁₋₄alkyl, C₁₋₃ alkoxy(C₁₋₃)alkyl, C₂₋₆ alkynyl, C₃₋₆ cycloalkyl, C₁₋₃ alkyl-(C₃₋₆)-cycloalkyl, phenyl (optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino or dialkylamino), heterocyclyl (optionally substituted by halo, nitro, cyano, C₁₋₆ alkyl, C₁₋₆ haloalkyl, C₁₋₆ alkoxy or C₁₋₆ haloalkoxy), formyl, C₁₋₆ alkylcarbonyl, C₁₋₆ alkoxycarbonyl, C₁₋₆ alkylthiocarbonyl, C₁₋₆ alkoxythionocarbonyl, carbamoyl, C₁₋₆ alkylaminocarbonyl, alkylaminocarbonyl, thiocarbamoyl, C₁₋₆ alkylaminothionocarbonyl, di-C₁₋₆ alkylaminothionocarbonyl, C₁₋₈ alkoxy, C₁₋₆ haloalkoxy, phenoxy (optionally substituted by halogen, C₁₋₄ alkyl, C₁₋₄ alkoxy, C₁₋₄ haloalkyl, C₁₋₄ haloalkoxy, CN, NO₂, phenyl), heteroaryloxy (optionally substituted by halo, nitro, cyano, C₁₋₃ alkyl, C₁₋₃ haloalkyl, C₁₋₃ alkoxy or C₁₋₃ haloalkoxy), C₁₋₆ alkylcarbonyloxy, C₁₋₆ alkoxycarbonyloxy, C₁₋₆ alkylaminocarbonyloxy, di-C₁₋₆ alkylaminocarbonyloxy, C₁₋₆ alkylaminothionocarbonyloxy, di-C₁₋₆ alkylaminothionocarbonyloxy, C₁₋₄ alkylthio, C₁₋₆ haloalkylthio, arylthio or heteroarylthio (where the aryl is optionally substituted by halogen, C₁₋₄ alkyl, C₁₋₄ alkoxy, C₁₋₄ haloalkyl, C₁₋₄ haloalkoxy, CN, NO₂ or phenyl), C₁₋₆ alkylcarbonylthio, C₁₋₆ alkylaminocarbonylthio, di-C₁₋₆ alkylaminocarbonylthio, di(C₁₋₈)alkylamino, C₁₋₆ alkylcarbonylamino, C₁₋₆ alkoxycarbonylamino, C₁₋₆ alkylaminocarbonylamino, di-C₁₋₆ alkylaminocarbonylamino, aminothiocarbonylamino, C₁₋₆ alkylaminothiocarbonylamino, di-C₁₋₆ alkylaminothiocarbonylamino, or 2 adjacent groups. R⁴ together with the carbon atoms to which they are attached form a 4, 5, 6 or 7 membered carbocyclic or heterocyclic ring which may be optionally substituted by halogen.

Preferably each R⁴ is independently halogen, nitro, cyano, C₁₋₈ alkyl, C₁₋₈ haloalkyl, cyano(C₁₋₆)alkyl, C₁₋₆ alkoxy(C₁₋₆)alkyl, C₂₋₆ alkynyl, heterocyclyl (optionally substituted by C₁₋₆ alkyl), C₁₋₈ alkoxy, C₁₋₆ haloalkoxy, phenoxy (optionally substituted by halo, cyano, C₁₋₃ alkyl or C₁₋₃ haloalkyl), heteroaryloxy (optionally substituted by halo, cyano, C₁₋₃ alkyl or C₁₋₃ haloalkyl), C₁₋₃ alkoxy, C₁₋₃ haloalkoxy, C₁₋₃ alkylthio, C₁₋₃ haloalkylthio, C₁₋₃ alksulfonyl, di(C₁₋₈)alkylamino, or 2 adjacent groups R⁴ together with the carbon atoms to which they are attached form a 4, 5, 6 or 7 membered carbocyclic or heterocyclic ring which may be optionally substituted by halogen.

Most preferably each R⁴ is independently fluoro, chloro, bromo, C₁₋₃ alkyl or C₁₋₃ haloalkyl.

In some embodiments at least one R⁴ is independently selected from fluoro, C₁₋₄alkyl and C₁₋₄haloalkyl.

Preferably n is 0, 1, 2 or 3. More preferably n is 1, 2 or 3. Most preferably n is 1 or 2.

In certain preferred embodiments n is 1, 2, or 3 and at least one R⁴ is independently selected from fluorine, methyl, fluoromethyl, difluoromethyl and trifluoromethyl.

In some preferred embodiments at least one group R⁴ is positioned adjacent to the group Y and in particularly preferred embodiments the R⁴ adjacent to the group Y is selected from fluorine, methyl, fluoromethyl, difluoromethyl, or trifluoromethyl.

In some embodiments R¹⁰ is hydrogen, hydroxy, amino, cyano, C₁₋₆ alkyl, C₁₋₆ haloalkyl, C₁₋₆ alkoxy(C₁₋₆)alkyl, C₃₋₆ alkenyl, C₃₋₆ alkinyl, phenyl(C₁₋₃)alkyl (wherein the phenyl group is optionally substituted by halogen, C₁₋₄ alkyl, C₁₋₄ alkoxy, C₁₋₄ haloalkyl, C₁₋₄ haloalkoxy, cyano, nitro, C₁₋₆ alkylsulfonyl or C₁₋₆ alkoxycarbonyl), cycloalkyl-C₁₋₃alkyl, C₃₋₆ cycloalkyl, aryl or heteroaryl (wherein aryl or heteroaryl is optionally substituted by halogen, C₁₋₄ alkyl, C₁₋₄ alkoxy, C₁₋₄ haloalkyl, C₁₋₄ haloalkoxy, cyano, nitro, C₁₋₆ alkylsulfonyl, C₁₋₆ alkoxycarbonyl), C₁₋₆ alkylthio, C₁₋₆ haloalkylthio, C₁₋₆ alkyldithio, C₁₋₆ alkylthiosulfinyl, formyl, C₁₋₆ alkylcarbonyl, aryl- or heteroarylcarbonyl (wherein aryl or heteroaryl is optionally substituted by halogen, C₁₋₄ alkyl, C₁₋₄ alkoxy, C₁₋₄ haloalkyl, C₁₋₄ haloalkoxy, cyano or nitro), C₁₋₆ alkoxycarbonyl, C₁₋₆ alkylthiocarbonyl, C₁₋₆ alkylcarbonylamino, R²⁴R²⁵N—, R²⁴R²⁵N—S— or R²⁴R²⁵N-A-, wherein R²⁴ and R²⁵ are preferably hydrogen or C₁₋₆alkyl and A is SO₂, C(O) or C(S).

In some embodiments R¹⁰ is hydroxy, phenyl(C₂₋₃)alkyl (wherein the phenyl group is optionally substituted by halogen, C₁₋₄ alkyl, C₁₋₄ alkoxy, C₁₋₄ haloalkyl, C₁₋₄ haloalkoxy, cyano, nitro, C₁₋₆ alkylsulfonyl or C₁₋₆ alkoxycarbonyl), C₃₋₅ cycloalkyl-C₁₋₃alkyl, heteroaryl (wherein heteroaryl is optionally substituted by halogen, C₁₋₄ alkyl, C₁₋₄ alkoxy, C₁₋₄ haloalkyl, C₁₋₄ haloalkoxy, cyano, nitro, C₁₋₆ alkylsulfonyl or C₁₋₆ alkoxycarbonyl), C₁₋₆ alkylthio, C₁₋₆ haloalkylthio, G-S—S—, aryl- or heteroarylcarbonyl (wherein aryl or heteroaryl is substituted by halogen, C₁₋₄ alkyl, C₁₋₄ alkoxy, C₁₋₄ haloalkyl, C₁₋₄ haloalkoxy, cyano and/or nitro), G-C(S)—, G-O—C(S)—, G-S—C(S)—, R²⁴R²⁵N—, G-C(O)—NR¹⁷—, G-C(S)—NR¹⁷—, G-SO₂—NR¹⁷—, R²⁴R²⁵N—S—, R²⁴R²⁵N—S(O)—, R²⁴R²⁵N—C(S)—, R¹⁸N═C(R¹⁹)—, where G, R¹⁷, R¹⁸, R¹⁹, R²⁴ and R²⁵ are as defined above.

Preferably R¹⁹ is hydrogen, amino, cyano, C₁₋₆, alkyl, C₁₋₆ haloalkyl, C₁₋₆ alkoxy(C₁₋₃)alkyl, C₃₋₆ alkenyl, C₃₋₆ alkinyl, phenyl-(C₁₋₂) alkyl (wherein phenyl is optionally substituted by halogen, C₁₋₄ alkyl, C₁₋₃ alkoxy, C₁₋₃ haloalkyl, C₁₋₃ haloalkoxy, cyano, nitro, C₁₋₃ alkylsulfonyl or C₁₋₄ alkoxycarbonyl), C₃₋₅ cycloalkyl-(C₁₋₃)alkyl, C₃₋₆ cycloalkyl, furyl, phenyl (which may be optionally substituted by halogen, C₁₋₄ alkyl, C₁₋₃ alkoxy, C₁₋₃ haloalkyl, C₁₋₃ haloalkoxy, cyano, nitro, C₁₋₃ alkylsulfonyl, or C₁₋₄ alkoxycarbonyl), C₁₋₆ alkylthio, C₁₋₆ haloalkylthio, C₁₋₆ alkyldithio, C₁₋₆ alkylthiosulfinyl, formyl, C₁₋₆ alkylcarbonyl, phenylcarbonyl (where phenyl is optionally substituted by halogen, C₁₋₄ alkyl, C₁₋₄ alkoxy, C₁₋₄ haloalkyl, C₁₋₄ haloalkoxy, cyano or nitro), C₁₋₆ alkoxycarbonyl, C₁₋₆ alkylthiocarbonyl, C₁₋₆ alkylcarbonylamino, R²⁴R²⁵N—, R²⁴R²⁵N—S— or R²⁴R²⁵N-A-, wherein R²⁴ and R²⁵ are preferably hydrogen or C₁₋₆ alkyl, and A is SO₂, C(O) or C(S).

Most preferably R¹⁹ is hydrogen.

It is preferred that the ring (T)

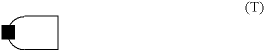

is a 6-membered aromatic ring or is 5 or 6 membered heteroaromatic ring wherein the ring members are each independently CH, S, N, NR⁴, O or CR⁴ provided that there are no more than one O or S atoms present in the ring.

More preferably the ring (T)

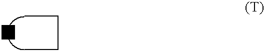

is a benzene, thiophene, furan, pyridine, pyrimidine, pyrazine, pyridazine, triazine, pyrrole, imidazole, pyrazole, oxazole, thiazole, isoxazole, isothiazole, [1,2,3]triazole, [1,2,3]oxadiazole or [1,2,3]thiadiazole ring.

Most preferably the ring (T)

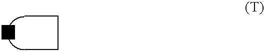

is a benzene, pyridine or thiophene ring, especially a benzene ring.

Another especially preferred group of compounds are those compounds of formula (IA)

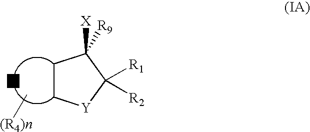

where the absolute chirality on the X and R⁹ bearing carbon atom is that shown in the structure above, and T, X, Y, R¹, R², R⁴, R⁹, R¹⁰ and n are as defined in relation to formula (I).

Certain compounds of formula (I) are novel and as such form a further aspect of the invention. One group of novel compounds are compounds of formula IB

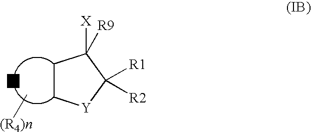

wherein T, X, Y, R¹, R², R⁴, R⁹, and n are as defined in claim 1 or salts or N-oxides thereof, with the proviso that the following compounds (IC1) to (IC5) are excluded:

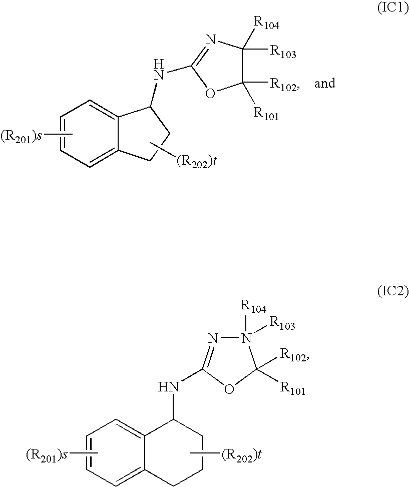

wherein R¹⁰¹ R¹⁰², R¹⁰³ and R¹⁰⁴ are hydrogen, R²⁰¹ and R²⁰² independently of each other are halogen, C₁₋₄ alkyl, C₁₋₄ alkoxy, C₁₋₄ alkylthio, trifluoromethyl or trifluoromethoxy, where the sum of s and t is 0, 1, 2 or 3.

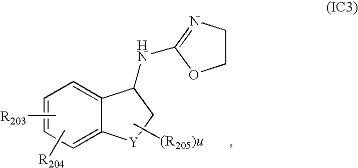

wherein Y is OCH₂, SCH₂, N(Me)CH₂, CH₂O, CH₂S or CH₂N(Me), R²⁰³ is hydrogen or C₁₋₄ alkyl, R²⁰⁴ is hydrogen, fluorine, chlorine, bromine, C₁₋₄ alkyl, C₁₋₄ alkoxy, C₁₋₄ alkylthio or dimethylamino, and R²⁰⁵ is C₁₋₄ alkyl, where u is 0, 1 or 2.

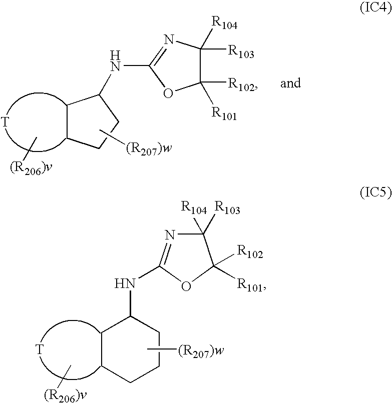

wherein T is thienyl or furyl, R¹⁰¹ R¹⁰², R¹⁰³ and R¹⁰⁴ are hydrogen, R²⁰⁶ and R²⁰⁷ independently of each other are hydrogen, chlorine or C₁₋₃ alkyl, and the sum of v and w is 0, 1, 2 or 3.

Another preferred group of compounds are those of formula IB above, wherein T is a benzene ring, and in particular where also at least one of R¹, R², R⁵, R⁶, R⁷, R⁸ or R⁹ is not hydrogen.

Another preferred group of novel compounds of the formula IB are those wherein Y is CR⁵R⁶ or CR⁵R⁶CR⁷R⁸, and R¹, R², R⁴, R⁵, R⁶, R⁷, R⁸, R⁹, X and n are defined above with respect to formula I, in particular where at least one of R¹, R², R⁵, R⁶, R⁷, R⁸ or R⁹ is other than hydrogen. Particularly preferred compounds within this group are those wherein T is a benzene ring; and those compounds wherein Y is CR⁵R⁶, T is a benzene ring and at least one of R¹, R², R⁵, R⁶ or R⁹ is fluorine are especially preferred.

Another preferred group of compounds of formula I are those, wherein Y is O, S(O)ₘ, NR³, SO₂—NR³, NR³—SO₂, NR³—O, O—NR³, O—CR⁷R⁸, S(O)ₘ—CR⁷R⁸, NR³—CR⁷R⁸, CR⁵R⁶—O, CR⁵R⁶—S(O)ₘ, or CR⁵R⁶—NR³, and R³, R⁵, R⁶, R⁷ and R⁸ are as defined herein.

The compounds in Tables 1 to 3 below illustrate the compounds of the invention.

Table I provides 612 compounds of formula Ia

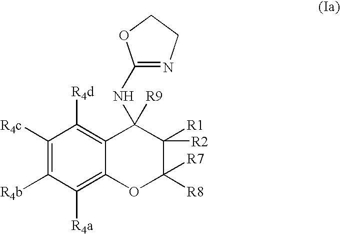

wherein the values of R¹, R², R^{4a}, R^{4b}, R^{4c}, R^{4d}, R⁷, R⁸ and R⁹ are given in Table 1.

TABLE 1

| Compound | | | | | | | | | |
|---|---|---|---|---|---|---|---|---|---|
| No | R9 | R1 | R2 | R7 | R8 | R4a | R4b | R4c | R4d |
| I-1 | Me | H | H | H | H | H | H | H | H |
| I-2 | Et | H | H | H | H | H | H | H | H |
| I-3 | vinyl | H | H | H | H | H | H | H | H |
| I-4 | allyl | H | H | H | H | H | H | H | H |
| I-5 | cyclopropyl | H | H | H | H | H | H | H | H |
| I-6 | CN | H | H | H | H | H | H | H | H |
| I-7 | CONH₂ | H | H | H | H | H | H | H | H |
| I-8 | CONHMe | H | H | H | H | H | H | H | H |
| I-9 | CONMe₂ | H | H | H | H | H | H | H | H |
| I-10 | COMe | H | H | H | H | H | H | H | H |
| I-11 | COOH | H | H | H | H | H | H | H | H |
| I-12 | COOMe | H | H | H | H | H | H | H | H |
| I-13 | CSNHMe | H | H | H | H | H | H | H | H |
| I-14 | H | Me | H | H | H | H | H | H | H |
| I-15 | H | Et | H | H | H | H | H | H | H |
| I-16 | H | vinyl | H | H | H | H | H | H | H |
| I-17 | H | allyl | H | H | H | H | H | H | H |
| I-18 | H | cyclopropyl | H | H | H | H | H | H | H |
| I-19 | H | F | H | H | H | H | H | H | H |
| I-20 | H | Cl | H | H | H | H | H | H | H |
| I-21 | H | NO₂ | H | H | H | H | H | H | H |
| I-22 | H | CN | H | H | H | H | H | H | H |
| I-23 | H | CONH₂ | H | H | H | H | H | H | H |
| I-24 | H | CONHMe | H | H | H | H | H | H | H |
| I-25 | H | CONMe₂ | H | H | H | H | H | H | H |
| I-26 | H | COMe | H | H | H | H | H | H | H |
| I-27 | H | COOH | H | H | H | H | H | H | H |
| I-28 | H | COOMe | H | H | H | H | H | H | H |
| I-29 | H | CSOMe | H | H | H | H | H | H | H |
| I-30 | H | CSNH₂ | H | H | H | H | H | H | H |
| I-31 | H | CSNMe₂ | H | H | H | H | H | H | H |
| I-32 | H | CSNHMe | H | H | H | H | H | H | H |
| I-33 | H | OMe | H | H | H | H | H | H | H |
| I-34 | H | OEt | H | H | H | H | H | H | H |
| I-35 | H | OCOMe | H | H | H | H | H | H | H |
| I-36 | H | OCOOMe | H | H | H | H | H | H | H |
| I-37 | H | OCONHMe | H | H | H | H | H | H | H |
| I-38 | H | OCONMe₂ | H | H | H | H | H | H | H |
| I-39 | H | OCSMe | H | H | H | H | H | H | H |
| I-40 | H | OCSNMe₂ | H | H | H | H | H | H | H |
| I-41 | H | SMe | H | H | H | H | H | H | H |
| I-42 | H | SEt | H | H | H | H | H | H | H |
| I-43 | H | SCOMe | H | H | H | H | H | H | H |
| I-44 | H | SCSNMe₂ | H | H | H | H | H | H | H |
| I-45 | H | SCSNHMe | H | H | H | H | H | H | H |
| I-46 | H | NHMe | H | H | H | H | H | H | H |
| I-47 | H | NH₂ | H | H | H | H | H | H | H |
| I-48 | H | NMe₂ | H | H | H | H | H | H | H |
| I-49 | H | NHCOMe | H | H | H | H | H | H | H |
| I-50 | H | NHCONH₂ | H | H | H | H | H | H | H |
| I-51 | H | NHCONHMe | H | H | H | H | H | H | H |
| I-52 | H | NHCONMe₂ | H | H | H | H | H | H | H |
| I-53 | H | phenyl | H | H | H | H | H | H | H |
| I-54 | H | 2-chloro- | H | H | H | H | H | H | H |
| | | phenyl | | | | | | | |
| I-55 | H | 4-nitrophenyl | H | H | H | H | H | H | H |
| I-56 | H | 2-pyridyl | H | H | H | H | H | H | H |
| I-57 | H | 3-pyridyl | H | H | H | H | H | H | H |
| I-58 | H | 4-pyridyl | H | H | H | H | H | H | H |
| I-59 | H | 2-furyl | H | H | H | H | H | H | H |
| I-60 | H | PhO | H | H | H | H | H | H | H |
| I-61 | CH₂ | | H | H | H | H | H | H | H |
| I-62 | H | ═O | | H | H | H | H | H | H |
| I-63 | H | ═NOH | | H | H | H | H | H | H |
| I-64 | H | ═NOMe | | H | H | H | H | H | H |
| I-65 | H | ═CH₂ | | H | H | H | H | H | H |
| I-66 | H | ═CHMe | | H | H | H | H | H | H |
| I-67 | H | H | H | H | H | H | H | H | H |
| I-68 | H | H | H | Me | H | H | H | H | H |
| I-69 | H | H | H | Et | H | H | H | H | H |
| I-70 | H | H | H | Vinyl | H | H | H | H | H |
| I-71 | H | H | H | Allyl | H | H | H | H | H |
| I-72 | H | H | H | cyclopropyl | H | H | H | H | H |
| I-73 | H | H | H | F | H | H | H | H | H |
| I-74 | H | H | H | Cl | H | H | H | H | H |
| I-75 | H | H | H | NO₂ | H | H | H | H | H |
| I-76 | H | H | H | CN | H | H | H | H | H |
| I-77 | H | H | H | CONH₂ | H | H | H | H | H |
| I-78 | H | H | H | CONHMe | H | H | H | H | H |
| I-79 | H | H | H | CONMe₂ | H | H | H | H | H |
| I-80 | H | H | H | COMe | H | H | H | H | H |
| I-81 | H | H | H | COOH | H | H | H | H | H |
| I-82 | H | H | H | COOMe | H | H | H | H | H |
| I-83 | H | H | H | CSOMe | H | H | H | H | H |
| I-84 | H | H | H | CSNH₂ | H | H | H | H | H |
| I-85 | H | H | H | CSNMe₂ | H | H | H | H | H |
| I-86 | H | H | H | CSNHMe | H | H | H | H | H |
| I-87 | H | H | H | OMe | H | H | H | H | H |
| I-88 | H | H | H | OEt | H | H | H | H | H |
| I-89 | H | H | H | OCOMe | H | H | H | H | H |
| I-90 | H | H | H | OCOOMe | H | H | H | H | H |
| I-91 | H | H | H | OCONHMe | H | H | H | H | H |
| I-92 | H | H | H | OCONMe₂ | H | H | H | H | H |
| I-93 | H | H | H | OCSMe | H | H | H | H | H |
| I-94 | H | H | H | OCSNMe₂ | H | H | H | H | H |
| I-95 | H | H | H | SMe | H | H | H | H | H |
| I-96 | H | H | H | SEt | H | H | H | H | H |
| I-97 | H | H | H | SCOMe | H | H | H | H | H |
| I-98 | H | H | H | SCSNMe₂ | H | H | H | H | H |
| I-99 | H | H | H | SCSNHMe | H | H | H | H | H |
| I-100 | H | H | H | NHMe | H | H | H | H | H |
| I-101 | H | H | H | NH₂ | H | H | H | H | H |
| I-102 | H | H | H | NMe₂ | H | H | H | H | H |
| I-103 | H | H | H | NHCOMe | H | H | H | H | H |
| I-104 | H | H | H | NHCONH₂ | H | H | H | H | H |
| I-105 | H | H | H | NHCONHMe | H | H | H | H | H |
| I-106 | H | H | H | NHCONMe₂ | H | H | H | H | H |
| I-107 | H | H | H | Phenyl | H | H | H | H | H |
| I-108 | H | H | H | 2-chloro- | H | H | H | H | H |
| | | | | phenyl | | | | | |
| I-109 | H | H | H | 4-nitrophenyl | H | H | H | H | H |
| I-110 | H | H | H | 2-pyridyl | H | H | H | H | H |
| I-111 | H | H | H | 3-pyridyl | H | H | H | H | H |
| I-112 | H | H | H | 4-pyridyl | H | H | H | H | H |
| I-113 | H | H | H | 2-furyl | H | H | H | H | H |
| I-114 | H | H | H | PhO | H | H | H | H | H |
| I-115 | H | H | H | ═O | | H | H | H | H |
| I-116 | H | H | H | ═NOH | | H | H | H | H |
| I-117 | H | H | H | ═NOMe | | H | H | H | H |
| I-118 | H | H | H | ═CH₂ | | H | H | H | H |
| I-119 | H | H | H | ═CHMe | | H | H | H | H |
| I-120 | H | Me | H | Me | H | H | H | H | H |
| I-121 | H | Me | F | Me | H | H | H | H | H |
| I-122 | H | Me | H | F | H | H | H | H | H |
| I-123 | H | Me | F | H | H | H | H | H | H |
| I-124 | H | Me | Me | H | H | H | H | H | H |
| I-125 | H | F | H | Me | H | H | H | H | H |
| I-126 | H | F | H | F | H | H | H | H | H |
| I-127 | Me | H | H | H | H | F | H | H | H |
| I-128 | Et | H | H | H | H | F | H | H | H |
| I-129 | vinyl | H | H | H | H | F | H | H | H |
| I-130 | allyl | H | H | H | H | F | H | H | H |
| I-131 | cyclopropyl | H | H | H | H | F | H | H | H |
| I-132 | CN | H | H | H | H | F | H | H | H |
| I-133 | CONH₂ | H | H | H | H | F | H | H | H |
| I-134 | CONHMe | H | H | H | H | F | H | H | H |
| I-135 | CONMe₂ | H | H | H | H | F | H | H | H |
| I-136 | COMe | H | H | H | H | F | H | H | H |
| I-137 | COOH | H | H | H | H | F | H | H | H |
| I-138 | COOMe | H | H | H | H | F | H | H | H |
| I-139 | CSNHMe | H | H | H | H | F | H | H | H |
| I-140 | H | Me | H | H | H | F | H | H | H |
| I-141 | H | Et | H | H | H | F | H | H | H |
| I-142 | H | vinyl | H | H | H | F | H | H | H |
| I-143 | H | allyl | H | H | H | F | H | H | H |
| I-144 | H | cyclopropyl | H | H | H | F | H | H | H |
| I-145 | H | F | H | H | H | F | H | H | H |
| I-146 | H | Cl | H | H | H | F | H | H | H |
| I-147 | H | NO₂ | H | H | H | F | H | H | H |
| I-148 | H | CN | H | H | H | F | H | H | H |
| I-149 | H | CONH₂ | H | H | H | F | H | H | H |
| I-150 | H | CONHMe | H | H | H | F | H | H | H |
| I-151 | H | CONMe₂ | H | H | H | F | H | H | H |
| I-152 | H | COMe | H | H | H | F | H | H | H |
| I-153 | H | COOH | H | H | H | F | H | H | H |
| I-154 | H | COOMe | H | H | H | F | H | H | H |
| I-155 | H | CSOMe | H | H | H | F | H | H | H |
| I-156 | H | CSNH₂ | H | H | H | F | H | H | H |
| I-157 | H | CSNMe₂ | H | H | H | F | H | H | H |
| I-158 | H | CSNHMe | H | H | H | F | H | H | H |
| I-159 | H | OMe | H | H | H | F | H | H | H |
| I-160 | H | OEt | H | H | H | F | H | H | H |
| I-161 | H | OCOMe | H | H | H | F | H | H | H |
| I-162 | H | OCOOMe | H | H | H | F | H | H | H |
| I-163 | H | OCONHMe | H | H | H | F | H | H | H |
| I-164 | H | OCONMe₂ | H | H | H | F | H | H | H |
| I-165 | H | OCSMe | H | H | H | F | H | H | H |
| I-166 | H | OCSNMe₂ | H | H | H | F | H | H | H |
| I-167 | H | SMe | H | H | H | F | H | H | H |
| I-168 | H | SEt | H | H | H | F | H | H | H |
| I-169 | H | SCOMe | H | H | H | F | H | H | H |
| I-170 | H | SCSNMe₂ | H | H | H | F | H | H | H |
| I-171 | H | SCSNHMe | H | H | H | F | H | H | H |
| I-172 | H | NHMe | H | H | H | F | H | H | H |
| I-173 | H | NH₂ | H | H | H | F | H | H | H |
| I-174 | H | NMe₂ | H | H | H | F | H | H | H |
| I-175 | H | NHCOMe | H | H | H | F | H | H | H |
| I-176 | H | NHCONH₂ | H | H | H | F | H | H | H |
| I-177 | H | NHCONHMe | H | H | H | F | H | H | H |
| I-178 | H | NHCONMe₂ | H | H | H | F | H | H | H |
| I-179 | H | phenyl | H | H | H | F | H | H | H |
| I-180 | H | 2-chloro- | H | H | H | F | H | H | H |
| | | phenyl | | | | | | | |
| I-181 | H | 4-nitrophenyl | H | H | H | F | H | H | H |
| I-182 | H | 2-pyridyl | H | H | H | F | H | H | H |
| I-183 | H | 3-pyridyl | H | H | H | F | H | H | H |
| I-184 | H | 4-pyridyl | H | H | H | F | H | H | H |
| I-185 | H | 2-furyl | H | H | H | F | H | H | H |
| I-186 | H | PhO | H | H | H | F | H | H | H |
| I-187 | CH₂ | | H | H | H | F | H | H | H |
| I-188 | H | H | H | Me | H | F | H | H | H |
| I-190 | H | H | H | Vinyl | H | F | H | H | H |
| I-191 | H | H | H | Allyl | H | F | H | H | H |
| I-192 | H | H | H | Cyclopropyl | H | F | H | H | H |
| I-193 | H | H | H | F | H | F | H | H | H |
| I-194 | H | H | H | Cl | H | F | H | H | H |
| I-195 | H | H | H | NO₂ | H | F | H | H | H |
| I-196 | H | H | H | CN | H | F | H | H | H |
| I-197 | H | H | H | CONH₂ | H | F | H | H | H |
| I-198 | H | H | H | CONHMe | H | F | H | H | H |
| I-199 | H | H | H | CONMe₂ | H | F | H | H | H |
| I-200 | H | H | H | COMe | H | F | H | H | H |
| I-201 | H | H | H | COOH | H | F | H | H | H |
| I-202 | H | H | H | COOMe | H | F | H | H | H |
| I-203 | H | H | H | CSOMe | H | F | H | H | H |
| I-204 | H | H | H | CSNH₂ | H | F | H | H | H |
| I-205 | H | H | H | CSNMe₂ | H | F | H | H | H |
| I-206 | H | H | H | CSNHMe | H | F | H | H | H |
| I-207 | H | H | H | OMe | H | F | H | H | H |
| I-208 | H | H | H | OEt | H | F | H | H | H |
| I-209 | H | H | H | OCOMe | H | F | H | H | H |
| I-210 | H | H | H | OCOOMe | H | F | H | H | H |
| I-211 | H | H | H | OCONHMe | H | F | H | H | H |
| I-212 | H | H | H | OCONMe₂ | H | F | H | H | H |
| I-213 | H | H | H | OCSMe | H | F | H | H | H |
| I-214 | H | H | H | OCSNMe₂ | H | F | H | H | H |
| I-215 | H | H | H | SMe | H | F | H | H | H |
| I-216 | H | H | H | SEt | H | F | H | H | H |
| I-217 | H | H | H | SCOMe | H | F | H | H | H |
| I-218 | H | H | H | SCSNMe₂ | H | F | H | H | H |
| I-219 | H | H | H | SCSNHMe | H | F | H | H | H |
| I-220 | H | H | H | NHMe | H | F | H | H | H |
| I-221 | H | H | H | NH₂ | H | F | H | H | H |
| I-222 | H | H | H | NMe₂ | H | F | H | H | H |
| I-223 | H | H | H | NHCOMe | H | F | H | H | H |
| I-224 | H | H | H | NHCONH₂ | H | F | H | H | H |
| I-225 | H | H | H | NHCONHMe | H | F | H | H | H |
| I-226 | H | H | H | NHCONMe₂ | H | F | H | H | H |
| I-227 | H | H | H | Phenyl | H | F | H | H | H |
| I-228 | H | H | H | 2-chloro- | H | F | H | H | H |
| | | | | phenyl | | | | | |
| I-229 | H | H | H | 4-nitrophenyl | H | F | H | H | H |
| I-230 | H | H | H | 2-pyridyl | H | F | H | H | H |
| I-231 | H | H | H | 3-pyridyl | H | F | H | H | H |
| I-232 | H | H | H | 4-pyridyl | H | F | H | H | H |
| I-233 | H | H | H | 2-furyl | H | F | H | H | H |
| I-234 | H | H | H | PhO | H | F | H | H | H |
| I-235 | H | Me | H | Me | H | F | H | H | H |
| I-236 | H | Me | F | Me | H | F | H | H | H |
| I-237 | H | Me | H | F | H | F | H | H | H |
| I-238 | H | Me | F | H | H | F | H | H | H |
| I-239 | H | Me | Me | H | H | F | H | H | H |
| I-240 | H | F | H | Me | H | F | H | H | H |
| I-241 | H | F | H | F | H | F | H | H | H |
| I-242 | Me | H | H | H | H | Me | H | H | H |
| I-243 | Et | H | H | H | H | Me | H | H | H |
| I-244 | vinyl | H | H | H | H | Me | H | H | H |
| I-245 | allyl | H | H | H | H | Me | H | H | H |
| I-246 | cyclopropyl | H | H | H | H | Me | H | H | H |
| I-247 | CN | H | H | H | H | Me | H | H | H |
| I-248 | CONH₂ | H | H | H | H | Me | H | H | H |
| I-249 | CONHMe | H | H | H | H | Me | H | H | H |
| I-250 | CONMe₂ | H | H | H | H | Me | H | H | H |
| I-251 | COMe | H | H | H | H | Me | H | H | H |
| I-252 | COOH | H | H | H | H | Me | H | H | H |
| I-253 | COOMe | H | H | H | H | Me | H | H | H |
| I-254 | CSNHMe | H | H | H | H | Me | H | H | H |
| I-255 | H | Me | H | H | H | Me | H | H | H |
| I-256 | H | Et | H | H | H | Me | H | H | H |
| I-257 | H | vinyl | H | H | H | Me | H | H | H |
| I-258 | H | allyl | H | H | H | Me | H | H | H |
| I-259 | H | cyclopropyl | H | H | H | Me | H | H | H |
| I-260 | H | F | H | H | H | Me | H | H | H |
| I-261 | H | Cl | H | H | H | Me | H | H | H |
| I-262 | H | NO₂ | H | H | H | Me | H | H | H |
| I-263 | H | CN | H | H | H | Me | H | H | H |
| I-264 | H | CONH₂ | H | H | H | Me | H | H | H |
| I-265 | H | CONHMe | H | H | H | Me | H | H | H |
| I-266 | H | CONMe₂ | H | H | H | Me | H | H | H |
| I-267 | H | COMe | H | H | H | Me | H | H | H |
| I-268 | H | COOH | H | H | H | Me | H | H | H |
| I-269 | H | COOMe | H | H | H | Me | H | H | H |
| I-270 | H | CSOMe | H | H | H | Me | H | H | H |
| I-271 | H | CSNH₂ | H | H | H | Me | H | H | H |
| I-272 | H | CSNMe₂ | H | H | H | Me | H | H | H |
| I-273 | H | CSNHMe | H | H | H | Me | H | H | H |
| I-274 | H | OMe | H | H | H | Me | H | H | H |
| I-275 | H | OEt | H | H | H | Me | H | H | H |
| I-276 | H | OCOMe | H | H | H | Me | H | H | H |
| I-277 | H | OCOOMe | H | H | H | Me | H | H | H |
| I-278 | H | OCONHMe | H | H | H | Me | H | H | H |
| I-279 | H | OCONMe₂ | H | H | H | Me | H | H | H |
| I-280 | H | OCSMe | H | H | H | Me | H | H | H |
| I-281 | H | OCSNMe₂ | H | H | H | Me | H | H | H |
| I-282 | H | SMe | H | H | H | Me | H | H | H |
| I-283 | H | SEt | H | H | H | Me | H | H | H |
| I-284 | H | SCOMe | H | H | H | Me | H | H | H |
| I-285 | H | SCSNMe₂ | H | H | H | Me | H | H | H |
| I-286 | H | SCSNHMe | H | H | H | Me | H | H | H |
| I-287 | H | NHMe | H | H | H | Me | H | H | H |
| I-288 | H | NH₂ | H | H | H | Me | H | H | H |
| I-289 | H | NMe₂ | H | H | H | Me | H | H | H |
| I-290 | H | NHCOMe | H | H | H | Me | H | H | H |
| I-291 | H | NHCONH₂ | H | H | H | Me | H | H | H |
| I-292 | H | NHCONHMe | H | H | H | Me | H | H | H |
| I-293 | H | NHCONMe₂ | H | H | H | Me | H | H | H |
| I-294 | H | phenyl | H | H | H | Me | H | H | H |
| I-295 | H | 2-chloro- | H | H | H | Me | H | H | H |
| | | phenyl | | | | | | | |
| I-296 | H | 4-nitrophenyl | H | H | H | Me | H | H | H |
| I-297 | H | 2-pyridyl | H | H | H | Me | H | H | H |
| I-298 | H | 3-pyridyl | H | H | H | Me | H | H | H |
| I-299 | H | 4-pyridyl | H | H | H | Me | H | H | H |
| I-300 | H | 2-furyl | H | H | H | Me | H | H | H |
| I-301 | H | PhO | H | H | H | Me | H | H | H |
| I-302 | CH₂ | | H | H | H | Me | H | H | H |
| I-303 | H | H | H | Me | H | Me | H | H | H |
| I-304 | H | H | H | Et | H | Me | H | H | H |
| I-305 | H | H | H | Vinyl | H | Me | H | H | H |
| I-306 | H | H | H | Allyl | H | Me | H | H | H |
| I-307 | H | H | H | Cyclopropyl | H | Me | H | H | H |
| I-308 | H | H | H | F | H | Me | H | H | H |
| I-309 | H | H | H | Cl | H | Me | H | H | H |
| I-310 | H | H | H | NO₂ | H | Me | H | H | H |
| I-311 | H | H | H | CN | H | Me | H | H | H |
| I-312 | H | H | H | CONH₂ | H | Me | H | H | H |
| I-313 | H | H | H | CONHMe | H | Me | H | H | H |
| I-314 | H | H | H | CONMe₂ | H | Me | H | H | H |
| I-315 | H | H | H | COMe | H | Me | H | H | H |
| I-316 | H | H | H | COOH | H | Me | H | H | H |
| I-317 | H | H | H | COOMe | H | Me | H | H | H |
| I-318 | H | H | H | CSOMe | H | Me | H | H | H |
| I-319 | H | H | H | CSNH₂ | H | Me | H | H | H |
| I-320 | H | H | H | CSNMe₂ | H | Me | H | H | H |
| I-321 | H | H | H | CSNHMe | H | Me | H | H | H |
| I-322 | H | H | H | OMe | H | Me | H | H | H |
| I-323 | H | H | H | OEt | H | Me | H | H | H |
| I-324 | H | H | H | OCOMe | H | Me | H | H | H |
| I-325 | H | H | H | OCOOMe | H | Me | H | H | H |
| I-326 | H | H | H | OCONHMe | H | Me | H | H | H |
| I-327 | H | H | H | OCONMe₂ | H | Me | H | H | H |
| I-328 | H | H | H | OCSMe | H | Me | H | H | H |
| I-329 | H | H | H | OCSNMe₂ | H | Me | H | H | H |
| I-330 | H | H | H | SMe | H | Me | H | H | H |
| I-331 | H | H | H | SEt | H | Me | H | H | H |
| I-332 | H | H | H | SCOMe | H | Me | H | H | H |
| I-333 | H | H | H | SCSNMe₂ | H | Me | H | H | H |
| I-334 | H | H | H | SCSNHMe | H | Me | H | H | H |
| I-335 | H | H | H | NHMe | H | Me | H | H | H |
| I-336 | H | H | H | NH₂ | H | Me | H | H | H |
| I-337 | H | H | H | NMe₂ | H | Me | H | H | H |
| I-338 | H | H | H | NHCOMe | H | Me | H | H | H |
| I-339 | H | H | H | NHCONH₂ | H | Me | H | H | H |
| I-340 | H | H | H | NHCONHMe | H | Me | H | H | H |
| I-341 | H | H | H | NHCONMe₂ | H | Me | H | H | H |
| I-342 | H | H | H | Phenyl | H | Me | H | H | H |
| I-343 | H | H | H | 2-chloro- | H | Me | H | H | H |
| | | | | phenyl | | | | | |
| I-344 | H | H | H | 4-nitrophenyl | H | Me | H | H | H |
| I-345 | H | H | H | 2-pyridyl | H | Me | H | H | H |
| I-346 | H | H | H | 3-pyridyl | H | Me | H | H | H |
| I-347 | H | H | H | 4-pyridyl | H | Me | H | H | H |
| I-348 | H | H | H | 2-furyl | H | Me | H | H | H |
| I-349 | H | H | H | PhO | H | Me | H | H | H |
| I-350 | H | Me | H | Me | H | Me | H | H | H |
| I-351 | H | Me | F | Me | H | Me | H | H | H |
| I-352 | H | Me | H | F | H | Me | H | H | H |
| I-353 | H | Me | F | H | H | Me | H | H | H |
| I-354 | H | Me | Me | H | H | Me | H | H | H |
| I-355 | H | F | H | Me | H | Me | H | H | H |
| I-356 | H | F | H | F | H | Me | H | H | H |
| I-357 | Me | H | H | H | H | F | H | F | H |
| I-358 | Et | H | H | H | H | F | H | F | H |
| I-359 | vinyl | H | H | H | H | F | H | F | H |
| I-360 | allyl | H | H | H | H | F | H | F | H |
| I-361 | cyclopropyl | H | H | H | H | F | H | F | H |
| I-362 | CN | H | H | H | H | F | H | F | H |
| I-363 | CONH₂ | H | H | H | H | F | H | F | H |
| I-364 | CONHMe | H | H | H | H | F | H | F | H |
| I-365 | CONMe₂ | H | H | H | H | F | H | F | H |
| I-366 | COMe | H | H | H | H | F | H | F | H |
| I-367 | COOH | H | H | H | H | F | H | F | H |
| I-368 | COOMe | H | H | H | H | F | H | F | H |
| I-369 | CSNHMe | H | H | H | H | F | H | F | H |
| I-370 | H | Me | H | H | H | F | H | F | H |
| I-371 | H | Et | H | H | H | F | H | F | H |
| I-372 | H | vinyl | H | H | H | F | H | F | H |
| I-373 | H | allyl | H | H | H | F | H | F | H |
| I-374 | H | cyclopropyl | H | H | H | F | H | F | H |
| I-375 | H | F | H | H | H | F | H | F | H |
| I-376 | H | Cl | H | H | H | F | H | F | H |
| I-377 | H | NO₂ | H | H | H | F | H | F | H |
| I-378 | H | CN | H | H | H | F | H | F | H |
| I-379 | H | CONH₂ | H | H | H | F | H | F | H |
| I-380 | H | CONHMe | H | H | H | F | H | F | H |
| I-381 | H | CONMe₂ | H | H | H | F | H | F | H |
| I-382 | H | COMe | H | H | H | F | H | F | H |
| I-383 | H | COOH | H | H | H | F | H | F | H |
| I-384 | H | COOMe | H | H | H | F | H | F | H |
| I-385 | H | CSOMe | H | H | H | F | H | F | H |
| I-386 | H | CSNH₂ | H | H | H | F | H | F | H |
| I-387 | H | CSNMe₂ | H | H | H | F | H | F | H |
| I-388 | H | CSNHMe | H | H | H | F | H | F | H |
| I-389 | H | OMe | H | H | H | F | H | F | H |
| I-390 | H | OEt | H | H | H | F | H | F | H |
| I-391 | H | OCOMe | H | H | H | F | H | F | H |
| I-392 | H | OCOOMe | H | H | H | F | H | F | H |
| I-393 | H | OCONHMe | H | H | H | F | H | F | H |
| I-394 | H | OCONMe₂ | H | H | H | F | H | F | H |
| I-395 | H | OCSMe | H | H | H | F | H | F | H |
| I-396 | H | OCSNMe₂ | H | H | H | F | H | F | H |
| I-397 | H | SMe | H | H | H | F | H | F | H |
| I-398 | H | SEt | H | H | H | F | H | F | H |
| I-399 | H | SCOMe | H | H | H | F | H | F | H |
| I-400 | H | SCSNMe₂ | H | H | H | F | H | F | H |
| I-401 | H | SCSNHMe | H | H | H | F | H | F | H |
| I-402 | H | NHMe | H | H | H | F | H | F | H |
| I-403 | H | NH₂ | H | H | H | F | H | F | H |
| I-404 | H | NMe₂ | H | H | H | F | H | F | H |
| I-405 | H | NHCOMe | H | H | H | F | H | F | H |
| I-406 | H | NHCONH₂ | H | H | H | F | H | F | H |
| I-407 | H | NHCONHMe | H | H | H | F | H | F | H |
| I-408 | H | NHCONMe₂ | H | H | H | F | H | F | H |
| I-409 | H | phenyl | H | H | H | F | H | F | H |
| I-410 | H | 2-chloro- | H | H | H | F | H | F | H |
| | | phenyl | | | | | | | |
| I-411 | H | 4-nitrophenyl | H | H | H | F | H | F | H |
| I-412 | H | 2-pyridyl | H | H | H | F | H | F | H |
| I-413 | H | 3-pyridyl | H | H | H | F | H | F | H |
| I-414 | H | 4-pyridyl | H | H | H | F | H | F | H |
| I-415 | H | 2-furyl | H | H | H | F | H | F | H |
| I-416 | H | PhO | H | H | H | F | H | F | H |
| I-417 | CH₂ | | H | H | H | F | H | F | H |
| I-418 | H | H | H | Me | H | F | H | F | H |
| I-419 | H | H | H | Et | H | F | H | F | H |
| I-420 | H | H | H | Vinyl | H | F | H | F | H |
| I-421 | H | H | H | Allyl | H | F | H | F | H |
| I-422 | H | H | H | Cyclopropyl | H | F | H | F | H |
| I-423 | H | H | H | F | H | F | H | F | H |
| I-424 | H | H | H | Cl | H | F | H | F | H |
| I-425 | H | H | H | NO₂ | H | F | H | F | H |
| I-426 | H | H | H | CN | H | F | H | F | H |
| I-427 | H | H | H | CONH₂ | H | F | H | F | H |
| I-428 | H | H | H | CONHMe | H | F | H | F | H |
| I-429 | H | H | H | CONMe₂ | H | F | H | F | H |
| I-430 | H | H | H | COMe | H | F | H | F | H |
| I-431 | H | H | H | COOH | H | F | H | F | H |
| I-432 | H | H | H | COOMe | H | F | H | F | H |
| I-433 | H | H | H | CSOMe | H | F | H | F | H |
| I-434 | H | H | H | CSNH₂ | H | F | H | F | H |
| I-435 | H | H | H | CSNMe₂ | H | F | H | F | H |
| I-436 | H | H | H | CSNHMe | H | F | H | F | H |
| I-437 | H | H | H | OMe | H | F | H | F | H |
| I-438 | H | H | H | OEt | H | F | H | F | H |
| I-439 | H | H | H | OCOMe | H | F | H | F | H |
| I-440 | H | H | H | OCOOMe | H | F | H | F | H |
| I-441 | H | H | H | OCONHMe | H | F | H | F | H |
| I-442 | H | H | H | OCONMe₂ | H | F | H | F | H |
| I-443 | H | H | H | OCSMe | H | F | H | F | H |
| I-444 | H | H | H | OCSNMe₂ | H | F | H | F | H |
| I-445 | H | H | H | SMe | H | F | H | F | H |
| I-446 | H | H | H | SEt | H | F | H | F | H |
| I-447 | H | H | H | SCOMe | H | F | H | F | H |
| I-448 | H | H | H | SCSNMe₂ | H | F | H | F | H |
| I-449 | H | H | H | SCSNHMe | H | F | H | F | H |
| I-450 | H | H | H | NHMe | H | F | H | F | H |
| I-451 | H | H | H | NH₂ | H | F | H | F | H |
| I-452 | H | H | H | NMe₂ | H | F | H | F | H |
| I-453 | H | H | H | NHCOMe | H | F | H | F | H |
| I-454 | H | H | H | NHCONH₂ | H | F | H | F | H |
| I-455 | H | H | H | NHCONHMe | H | F | H | F | H |
| I-456 | H | H | H | NHCONMe₂ | H | F | H | F | H |
| I-457 | H | H | H | Phenyl | H | F | H | F | H |
| I-458 | H | H | H | 2-chloro- | H | F | H | F | H |
| | | | | phenyl | | | | | |
| I-459 | H | H | H | 4-nitrophenyl | H | F | H | F | H |
| I-460 | H | H | H | 2-pyridyl | H | F | H | F | H |
| I-461 | H | H | H | 3-pyridyl | H | F | H | F | H |
| I-462 | H | H | H | 4-pyridyl | H | F | H | F | H |
| I-463 | H | H | H | 2-furyl | H | F | H | F | H |
| I-464 | H | H | H | PhO | H | F | H | F | H |
| I-465 | H | Me | H | Me | H | F | H | F | H |
| I-466 | H | Me | F | Me | H | F | H | F | H |
| I-467 | H | Me | H | F | H | F | H | F | H |
| I-468 | H | Me | F | H | H | F | H | F | H |
| I-469 | H | Me | Me | H | H | F | H | F | H |
| I-470 | H | F | H | Me | H | F | H | F | H |
| I-471 | H | F | H | F | H | F | H | F | H |
| I-472 | H | H | H | H | H | Me | H | H | H |
| I-473 | H | H | H | H | H | Et | H | H | H |
| I-474 | H | H | H | H | H | vinyl | H | H | H |
| I-475 | H | H | H | H | H | allyl | H | H | H |
| I-476 | H | H | H | H | H | cyclopropyl | H | H | H |
| I-477 | H | H | H | H | H | F | H | H | H |
| I-478 | H | H | H | H | H | Cl | H | H | H |
| I-479 | H | H | H | H | H | NO₂ | H | H | H |
| I-480 | H | H | H | H | H | CN | H | H | H |
| I-481 | H | H | H | H | H | CONH₂ | H | H | H |
| I-482 | H | H | H | H | H | CONHMe | H | H | H |
| I-483 | H | H | H | H | H | CONMe₂ | H | H | H |
| I-484 | H | H | H | H | H | COMe | H | H | H |
| I-485 | H | H | H | H | H | COOH | H | H | H |
| I-486 | H | H | H | H | H | COOMe | H | H | H |
| I-487 | H | H | H | H | H | CSOMe | H | H | H |
| I-488 | H | H | H | H | H | CSNH₂ | H | H | H |
| I-489 | H | H | H | H | H | CSNMe₂ | H | H | H |
| I-490 | H | H | H | H | H | CSNHMe | H | H | H |
| I-491 | H | H | H | H | H | OMe | H | H | H |
| I-492 | H | H | H | H | H | OEt | H | H | H |
| I-493 | H | H | H | H | H | OCOMe | H | H | H |
| I-494 | H | H | H | H | H | OCOOMe | H | H | H |
| I-495 | H | H | H | H | H | OCONHMe | H | H | H |
| I-496 | H | H | H | H | H | OCONMe₂ | H | H | H |
| I-497 | H | H | H | H | H | OCSMe | H | H | H |
| I-498 | H | H | H | H | H | OCSNMe₂ | H | H | H |
| I-499 | H | H | H | H | H | SMe | H | H | H |
| I-500 | H | H | H | H | H | SEt | H | H | H |
| I-501 | H | H | H | H | H | SCOMe | H | H | H |
| I-502 | H | H | H | H | H | SCSNMe₂ | H | H | H |
| I-503 | H | H | H | H | H | SCSNHMe | H | H | H |
| I-504 | H | H | H | H | H | NHMe | H | H | H |
| I-505 | H | H | H | H | H | NH₂ | H | H | H |
| I-506 | H | H | H | H | H | NMe₂ | H | H | H |
| I-507 | H | H | H | H | H | NHCOMe | H | H | H |
| I-508 | H | H | H | H | H | NHCONH₂ | H | H | H |
| I-509 | H | H | H | H | H | NHCONHMe | H | H | H |
| I-510 | H | H | H | H | H | NHCONMe₂ | H | H | H |
| I-511 | H | H | H | H | H | phenyl | H | H | H |
| I-512 | H | H | H | H | H | 2-chloro- | H | H | H |
| | | | | | | phenyl | | | |
| I-513 | H | H | H | H | H | 4-nitrophenyl | H | H | H |
| I-514 | H | H | H | H | H | 2-pyridyl | H | H | H |
| I-515 | H | H | H | H | H | 3-pyridyl | H | H | H |
| I-516 | H | H | H | H | H | 4-pyridyl | H | H | H |
| I-517 | H | H | H | H | H | 2-furyl | H | H | H |
| I-518 | H | H | H | H | H | PhO | H | H | H |
| I-519 | H | H | H | H | H | H | H | Me | H |
| I-520 | H | H | H | H | H | H | H | Et | H |
| I-521 | H | H | H | H | H | H | H | vinyl | H |
| I-522 | H | H | H | H | H | H | H | allyl | H |
| I-523 | H | H | H | H | H | H | H | cyclopropyl | H |
| I-524 | H | H | H | H | H | H | H | F | H |
| I-525 | H | H | H | H | H | H | H | Cl | H |
| I-526 | H | H | H | H | H | H | H | NO₂ | H |
| I-527 | H | H | H | H | H | H | H | CN | H |
| I-528 | H | H | H | H | H | H | H | CONH₂ | H |
| I-529 | H | H | H | H | H | H | H | CONHMe | H |
| I-530 | H | H | H | H | H | H | H | CONMe₂ | H |
| I-531 | H | H | H | H | H | H | H | COMe | H |
| I-532 | H | H | H | H | H | H | H | COOH | H |
| I-533 | H | H | H | H | H | H | H | COOMe | H |
| I-534 | H | H | H | H | H | H | H | CSOMe | H |
| I-535 | H | H | H | H | H | H | H | CSNH₂ | H |
| I-536 | H | H | H | H | H | H | H | CSNMe₂ | H |
| I-537 | H | H | H | H | H | H | H | CSNHMe | H |
| I-538 | H | H | H | H | H | H | H | OMe | H |
| I-539 | H | H | H | H | H | H | H | OEt | H |
| I-540 | H | H | H | H | H | H | H | OCOMe | H |
| I-541 | H | H | H | H | H | H | H | OCOOMe | H |
| I-542 | H | H | H | H | H | H | H | OCONHMe | H |
| I-543 | H | H | H | H | H | H | H | OCONMe₂ | H |
| I-544 | H | H | H | H | H | H | H | OCSMe | H |
| I-545 | H | H | H | H | H | H | H | OCSNMe₂ | H |
| I-546 | H | H | H | H | H | H | H | SMe | H |
| I-547 | H | H | H | H | H | H | H | SEt | H |
| I-548 | H | H | H | H | H | H | H | SCOMe | H |
| I-549 | H | H | H | H | H | H | H | SCSNMe₂ | H |
| I-550 | H | H | H | H | H | H | H | SCSNHMe | H |
| I-551 | H | H | H | H | H | H | H | NHMe | H |
| I-552 | H | H | H | H | H | H | H | NH₂ | H |
| I-553 | H | H | H | H | H | H | H | NMe₂ | H |
| I-554 | H | H | H | H | H | H | H | NHCOMe | H |
| I-555 | H | H | H | H | H | H | H | NHCONH₂ | H |
| I-556 | H | H | H | H | H | H | H | NHCONHMe | H |
| I-557 | H | H | H | H | H | H | H | NHCONMe₂ | H |
| I-558 | H | H | H | H | H | H | H | phenyl | H |
| I-559 | H | H | H | H | H | H | H | 2-chloro- | H |
| | | | | | | | | phenyl | |
| I-560 | H | H | H | H | H | H | H | 4-nitrophenyl | H |
| I-561 | H | H | H | H | H | H | H | 2-pyridyl | H |
| I-562 | H | H | H | H | H | H | H | 3-pyridyl | H |
| I-563 | H | H | H | H | H | H | H | 4-pyridyl | H |
| I-564 | H | H | H | H | H | H | H | 2-furyl | H |
| I-565 | H | H | H | H | H | H | H | PhO | H |
| I-566 | H | H | H | H | H | H | H | H | Me |
| I-567 | H | H | H | H | H | H | H | H | Et |
| I-568 | H | H | H | H | H | H | H | H | vinyl |
| I-569 | H | H | H | H | H | H | H | H | allyl |
| I-570 | H | H | H | H | H | H | H | H | cyclopropyl |
| I-571 | H | H | H | H | H | H | H | H | F |
| I-572 | H | H | H | H | H | H | H | H | Cl |
| I-573 | H | H | H | H | H | H | H | H | NO₂ |
| I-574 | H | H | H | H | H | H | H | H | CN |
| I-575 | H | H | H | H | H | H | H | H | CONH₂ |
| I-576 | H | H | H | H | H | H | H | H | CONHMe |
| I-577 | H | H | H | H | H | H | H | H | CONMe₂ |
| I-578 | H | H | H | H | H | H | H | H | COMe |
| I-579 | H | H | H | H | H | H | H | H | COOH |
| I-580 | H | H | H | H | H | H | H | H | COOMe |
| I-581 | H | H | H | H | H | H | H | H | CSOMe |
| I-582 | H | H | H | H | H | H | H | H | CSNH₂ |
| I-583 | H | H | H | H | H | H | H | H | CSNMe₂ |
| I-584 | H | H | H | H | H | H | H | H | CSNHMe |
| I-585 | H | H | H | H | H | H | H | H | OMe |
| I-586 | H | H | H | H | H | H | H | H | OEt |
| I-587 | H | H | H | H | H | H | H | H | OCOMe |
| I-588 | H | H | H | H | H | H | H | H | OCOOMe |
| I-589 | H | H | H | H | H | H | H | H | OCONHMe |
| I-590 | H | H | H | H | H | H | H | H | OCONMe₂ |
| I-591 | H | H | H | H | H | H | H | H | OCSMe |
| I-592 | H | H | H | H | H | H | H | H | OCSNMe₂ |
| I-593 | H | H | H | H | H | H | H | H | SMe |
| I-594 | H | H | H | H | H | H | H | H | SEt |
| I-595 | H | H | H | H | H | H | H | H | SCOMe |
| I-596 | H | H | H | H | H | H | H | H | SCSNMe₂ |
| I-597 | H | H | H | H | H | H | H | H | SCSNHMe |
| I-598 | H | H | H | H | H | H | H | H | NHMe |
| I-599 | H | H | H | H | H | H | H | H | NH₂ |
| I-600 | H | H | H | H | H | H | H | H | NMe₂ |
| I-601 | H | H | H | H | H | H | H | H | NHCOMe |
| I-602 | H | H | H | H | H | H | H | H | NHCONH₂ |
| I-603 | H | H | H | H | H | H | H | H | NHCONHMe |
| I-604 | H | H | H | H | H | H | H | H | NHCONMe₂ |
| I-605 | H | H | H | H | H | H | H | H | phenyl |
| I-606 | H | H | H | H | H | H | H | H | 2-chloro- |
| | | | | | | | | | phenyl |
| I-607 | H | H | H | H | H | H | H | H | 4-nitrophenyl |
| I-608 | H | H | H | H | H | H | H | H | 2-pyridyl |
| I-609 | H | H | H | H | H | H | H | H | 3-pyridyl |
| I-610 | H | H | H | H | H | H | H | H | 4-pyridyl |
| I-611 | H | H | H | H | H | H | H | H | 2-furyl |
| I-612 | H | H | H | H | H | H | H | H | PhO |

Table II provides 612 compounds of formula Ib

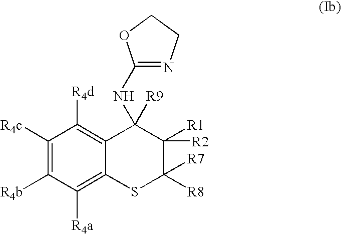

wherein the values of R¹, R², R^{4a}, R^{4b}, R^{4c}, R^{4d}, R⁷, R⁸ and R⁹ are given in Table 1.

Table III provides 612 compounds of formula Ic

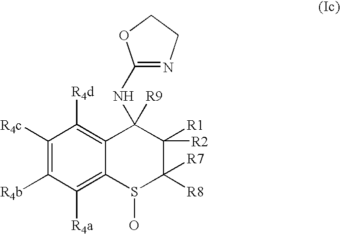

wherein the values of R¹, R², R^{4a}, R^{4b}, R^{4c}, R^{4d}, R⁷, R⁸ and R⁹ are given in Table 1.

Table IV provides 612 compounds of formula Id

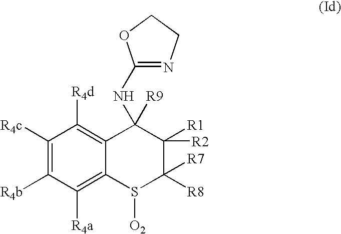

wherein the values of R¹, R², R^{4a}, R^{4b}, R^{4c}, R^{4d}, R⁷, R⁸ and R⁹ are given in Table 1.

Table V provides 612 compounds of formula Ie

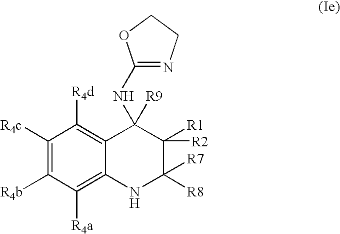

wherein the values of R¹, R², R^{4a}, R^{4b}, R^{4c}, R^{4d}, R⁷, R⁸ and R⁹ are given in Table 1.

Table VI provides 612 compounds of formula If

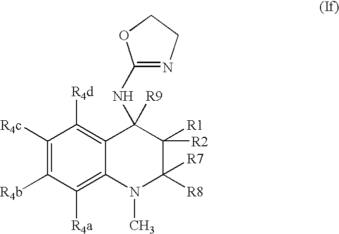

wherein the values of R¹, R², R^{4a}, R^{4b}, R^{4c}, R^{4d}, R⁷, R⁸ and R⁹ are given in Table 1.

Table VII provides 612 compounds of formula Ig

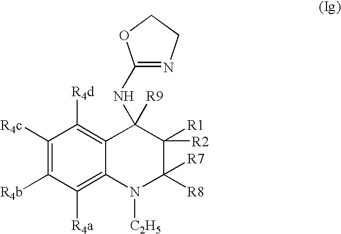

wherein the values of R¹, R², R^{4a}, R^{4b}, R^{4c}, R^{4d}, R⁷, R⁸ and R⁹ are given in Table 1.

Table VIII provides 612 compounds of formula Ih

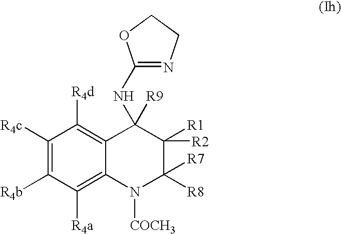

wherein the values of R¹, R², R^{4a}, R^{4b}, R^{4c}, R^{4d}, R⁷, and R⁸ and R⁹ are given in Table 1.

Table IX provides 612 compounds of formula Ii

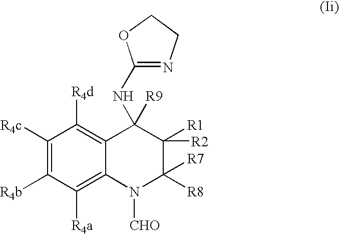

wherein the values of R¹, R², R^{4a}, R^{4b}, R^{4c}, R^{4d}, R⁷, R⁸ and R⁹ are given in Table 1.

Table X provides 612 compounds of formula Ij

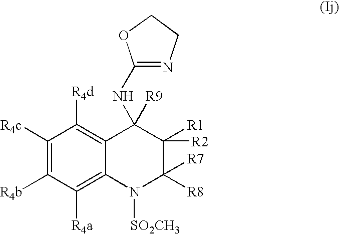

wherein the values of R¹, R², R^{4a}, R^{4b}, R^{4c}, R^{4d}, R⁷, R⁸ and R⁹ are given in Table 1.

Table XI provides 612 compounds of formula Ik

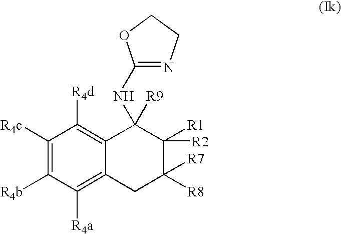

wherein the values of R¹, R², R^{4a}, R^{4b}, R^{4c}, R^{4d}, R⁷, R⁸ and R⁹ are given in Table 1.

Table XII provides 612 compounds of formula II

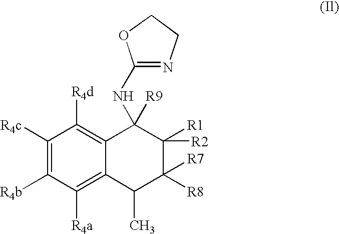

wherein the values of R¹, R², R^{4a}, R^{4b}, R^{4c}, R4d, R⁷, R⁸ and R⁹ are given in Table 1.

Table XIII provides 612 compounds of formula Im

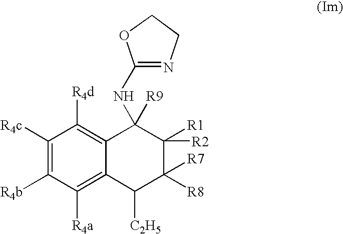

wherein the values of R¹, R², R^{4a}, R^{4b}, R^{4c}, R^{4d}, R⁷, R⁸ and R⁹ are given in Table 1.

Table XIV provides 612 compounds of formula In

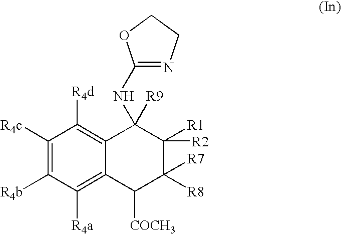

wherein the values of R¹, R², R^{4a}, R^{4b}, R^{4c}, R^{4d}, R⁷, R⁸ and R⁹ are given in Table 1.

Table XV provides 612 compounds of formula Io

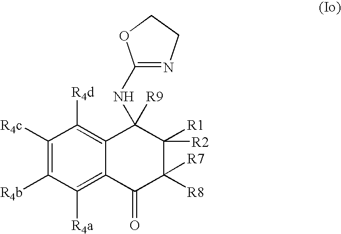

wherein the values of R¹, R², R^{4a}, R^{4b}, R^{4c}, R^{4d}, R⁷, R⁸ and R⁹ are given in Table 1.

Table XVI provides 612 compounds of formula Ip

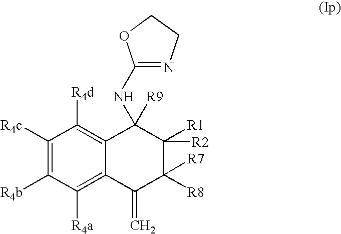

wherein the values of R¹, R², R^{4a}, R^{4b}, R^{4c}, R^{4d}, R⁷, R⁸ and R⁹ are given in Table 1.

Table XVII provides 612 compounds of formula Iq

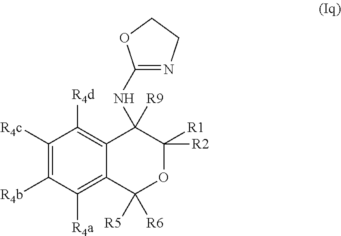

wherein the values of R¹, R², R^{4a}, R^{4b}, R^{4c}, R^{4d}, R⁵, R⁶ and R⁹ are given in Table 2.

TABLE 2

| Compound | | | | | | | | | |
|---|---|---|---|---|---|---|---|---|---|
| No | R9 | R1 | R2 | R5 | R6 | R4a | R4b | R4c | R4d |
| XVII-1 | Me | H | H | H | H | H | H | H | H |
| XVII-2 | Et | H | H | H | H | H | H | H | H |
| XVII-3 | vinyl | H | H | H | H | H | H | H | H |
| XVII-4 | allyl | H | H | H | H | H | H | H | H |
| XVII-5 | cyclopropyl | H | H | H | H | H | H | H | H |
| XVII-6 | CN | H | H | H | H | H | H | H | H |
| XVII-7 | CONH₂ | H | H | H | H | H | H | H | H |
| XVII-8 | CONHMe | H | H | H | H | H | H | H | H |
| XVII-9 | CONMe₂ | H | H | H | H | H | H | H | H |
| XVII-10 | COMe | H | H | H | H | H | H | H | H |
| XVII-11 | COOH | H | H | H | H | H | H | H | H |
| XVII-12 | COOMe | H | H | H | H | H | H | H | H |
| XVII-13 | CSNHMe | H | H | H | H | H | H | H | H |
| XVII-14 | H | Me | H | H | H | H | H | H | H |
| XVII-15 | H | Et | H | H | H | H | H | H | H |
| XVII-16 | H | vinyl | H | H | H | H | H | H | H |
| XVII-17 | H | allyl | H | H | H | H | H | H | H |
| XVII-18 | H | cyclopropyl | H | H | H | H | H | H | H |
| XVII-19 | H | F | H | H | H | H | H | H | H |
| XVII-20 | H | Cl | H | H | H | H | H | H | H |
| XVII-21 | H | NO₂ | H | H | H | H | H | H | H |
| XVII-22 | H | CN | H | H | H | H | H | H | H |
| XVII-23 | H | CONH₂ | H | H | H | H | H | H | H |
| XVII-24 | H | CONHMe | H | H | H | H | H | H | H |
| XVII-25 | H | CONMe₂ | H | H | H | H | H | H | H |
| XVII-26 | H | COMe | H | H | H | H | H | H | H |
| XVII-27 | H | COOH | H | H | H | H | H | H | H |
| XVII-28 | H | COOMe | H | H | H | H | H | H | H |
| XVII-29 | H | CSOMe | H | H | H | H | H | H | H |
| XVII-30 | H | CSNH₂ | H | H | H | H | H | H | H |
| XVII-31 | H | CSNMe₂ | H | H | H | H | H | H | H |
| XVII-32 | H | CSNHMe | H | H | H | H | H | H | H |
| XVII-33 | H | OMe | H | H | H | H | H | H | H |
| XVII-34 | H | OEt | H | H | H | H | H | H | H |
| XVII-35 | H | OCOMe | H | H | H | H | H | H | H |
| XVII-36 | H | OCOOMe | H | H | H | H | H | H | H |
| XVII-37 | H | OCONHMe | H | H | H | H | H | H | H |
| XVII-38 | H | OCONMe₂ | H | H | H | H | H | H | H |
| XVII-39 | H | OCSMe | H | H | H | H | H | H | H |
| XVII-40 | H | OCSNMe₂ | H | H | H | H | H | H | H |
| XVII-41 | H | SMe | H | H | H | H | H | H | H |
| XVII-42 | H | SEt | H | H | H | H | H | H | H |
| XVII-43 | H | SCOMe | H | H | H | H | H | H | H |
| XVII-44 | H | SCSNMe₂ | H | H | H | H | H | H | H |
| XVII-45 | H | SCSNHMe | H | H | H | H | H | H | H |
| XVII-46 | H | NHMe | H | H | H | H | H | H | H |
| XVII-47 | H | NH₂ | H | H | H | H | H | H | H |
| XVII-48 | H | NMe₂ | H | H | H | H | H | H | H |
| XVII-49 | H | NHCOMe | H | H | H | H | H | H | H |
| XVII-50 | H | NHCONH₂ | H | H | H | H | H | H | H |
| XVII-51 | H | NHCONHMe | H | H | H | H | H | H | H |
| XVII-52 | H | NHCONMe₂ | H | H | H | H | H | H | H |
| XVII-53 | H | phenyl | H | H | H | H | H | H | H |
| XVII-54 | H | 2-chloro- | H | H | H | H | H | H | H |
| | | phenyl | | | | | | | |
| XVII-55 | H | 4-nitrophenyl | H | H | H | H | H | H | H |
| XVII-56 | H | 2-pyridyl | H | H | H | H | H | H | H |
| XVII-57 | H | 3-pyridyl | H | H | H | H | H | H | H |
| XVII-58 | H | 4-pyridyl | H | H | H | H | H | H | H |
| XVII-59 | H | 2-furyl | H | H | H | H | H | H | H |
| XVII-60 | H | PhO | H | H | H | H | H | H | H |
| XVII-61 | CH₂ | | H | H | H | H | H | H | H |
| XVII-62 | H | ═O | | H | H | H | H | H | H |
| XVII-63 | H | ═NOH | | H | H | H | H | H | H |
| XVII-64 | H | ═NOMe | | H | H | H | H | H | H |
| XVII-65 | H | ═CH₂ | | H | H | H | H | H | H |
| XVII-66 | H | ═CHMe | | H | H | H | H | H | H |
| XVII-67 | H | H | H | H | H | H | H | H | H |
| XVII-68 | H | H | H | Me | H | H | H | H | H |
| XVII-69 | H | H | H | Et | H | H | H | H | H |
| XVII-70 | H | H | H | vinyl | H | H | H | H | H |
| XVII-71 | H | H | H | allyl | H | H | H | H | H |
| XVII-72 | H | H | H | cyclopropyl | H | H | H | H | H |
| XVII-73 | H | H | H | F | H | H | H | H | H |
| XVII-74 | H | H | H | Cl | H | H | H | H | H |
| XVII-75 | H | H | H | NO₂ | H | H | H | H | H |
| XVII-76 | H | H | H | CN | H | H | H | H | H |
| XVII-77 | H | H | H | CONH₂ | H | H | H | H | H |
| XVII-78 | H | H | H | CONHMe | H | H | H | H | H |
| XVII-79 | H | H | H | CONMe₂ | H | H | H | H | H |
| XVII-80 | H | H | H | COMe | H | H | H | H | H |
| XVII-81 | H | H | H | COOH | H | H | H | H | H |
| XVII-82 | H | H | H | COOMe | H | H | H | H | H |
| XVII-83 | H | H | H | CSOMe | H | H | H | H | H |
| XVII-84 | H | H | H | CSNH₂ | H | H | H | H | H |
| XVII-85 | H | H | H | CSNMe₂ | H | H | H | H | H |
| XVII-86 | H | H | H | CSNHMe | H | H | H | H | H |
| XVII-87 | H | H | H | OMe | H | H | H | H | H |
| XVII-88 | H | H | H | OEt | H | H | H | H | H |
| XVII-89 | H | H | H | OCOMe | H | H | H | H | H |
| XVII-90 | H | H | H | OCOOMe | H | H | H | H | H |
| XVII-91 | H | H | H | OCONHMe | H | H | H | H | H |
| XVII-92 | H | H | H | OCONMe₂ | H | H | H | H | H |
| XVII-93 | H | H | H | OCSMe | H | H | H | H | H |
| XVII-94 | H | H | H | OCSNMe₂ | H | H | H | H | H |
| XVII-95 | H | H | H | SMe | H | H | H | H | H |
| XVII-96 | H | H | H | SEt | H | H | H | H | H |
| XVII-97 | H | H | H | SCOMe | H | H | H | H | H |
| XVII-98 | H | H | H | SCSNMe₂ | H | H | H | H | H |
| XVII-99 | H | H | H | SCSNHMe | H | H | H | H | H |
| XVII-100 | H | H | H | NHMe | H | H | H | H | H |
| XVII-101 | H | H | H | NH₂ | H | H | H | H | H |
| XVII-102 | H | H | H | NMe₂ | H | H | H | H | H |
| XVII-103 | H | H | H | NHCOMe | H | H | H | H | H |
| XVII-104 | H | H | H | NHCONH₂ | H | H | H | H | H |
| XVII-105 | H | H | H | NHCONHMe | H | H | H | H | H |
| XVII-106 | H | H | H | NHCONMe₂ | H | H | H | H | H |
| XVII-107 | H | H | H | phenyl | H | H | H | H | H |
| XVII-108 | H | H | H | 2-chloro- | H | H | H | H | H |
| | | | | phenyl | | | | | |
| XVII-109 | H | H | H | 4-nitrophenyl | H | H | H | H | H |
| XVII-110 | H | H | H | 2-pyridyl | H | H | H | H | H |
| XVII-111 | H | H | H | 3-pyridyl | H | H | H | H | H |
| XVII-112 | H | H | H | 4-pyridyl | H | H | H | H | H |
| XVII-113 | H | H | H | 2-furyl | H | H | H | H | H |
| XVII-114 | H | H | H | PhO | H | H | H | H | H |
| XVII-115 | H | H | H | ═O | | H | H | H | H |
| XVII-116 | H | H | H | ═NOH | | H | H | H | H |
| XVII-117 | H | H | H | ═NOMe | | H | H | H | H |
| XVII-118 | H | H | H | ═CH₂ | | H | H | H | H |
| XVII-119 | H | H | H | ═CHMe | | H | H | H | H |
| XVII-120 | H | Me | H | Me | H | H | H | H | H |
| XVII-121 | H | Me | F | Me | H | H | H | H | H |
| XVII-122 | H | Me | H | F | H | H | H | H | H |
| XVII-123 | H | Me | F | H | H | H | H | H | H |
| XVII-124 | H | Me | Me | H | H | H | H | H | H |
| XVII-125 | H | F | H | Me | H | H | H | H | H |
| XVII-126 | H | F | H | F | H | H | H | H | H |
| XVII-127 | Me | H | H | H | H | F | H | H | H |
| XVII-128 | Et | H | H | H | H | F | H | H | H |
| XVII-129 | vinyl | H | H | H | H | F | H | H | H |
| XVII-130 | allyl | H | H | H | H | F | H | H | H |
| XVII-131 | cyclopropyl | H | H | H | H | F | H | H | H |
| XVII-132 | CN | H | H | H | H | F | H | H | H |
| XVII-133 | CONH₂ | H | H | H | H | F | H | H | H |
| XVII-134 | CONHMe | H | H | H | H | F | H | H | H |
| XVII-135 | CONMe₂ | H | H | H | H | F | H | H | H |
| XVII-136 | COMe | H | H | H | H | F | H | H | H |
| XVII-137 | COOH | H | H | H | H | F | H | H | H |
| XVII-138 | COOMe | H | H | H | H | F | H | H | H |
| XVII-139 | CSNHMe | H | H | H | H | F | H | H | H |
| XVII-140 | H | Me | H | H | H | F | H | H | H |
| XVII-141 | H | Et | H | H | H | F | H | H | H |
| XVII-142 | H | vinyl | H | H | H | F | H | H | H |
| XVII-143 | H | allyl | H | H | H | F | H | H | H |
| XVII-144 | H | cyclopropyl | H | H | H | F | H | H | H |
| XVII-145 | H | F | H | H | H | F | H | H | H |
| XVII-146 | H | Cl | H | H | H | F | H | H | H |
| XVII-147 | H | NO₂ | H | H | H | F | H | H | H |
| XVII-148 | H | CN | H | H | H | F | H | H | H |
| XVII-149 | H | CONH₂ | H | H | H | F | H | H | H |
| XVII-150 | H | CONHMe | H | H | H | F | H | H | H |
| XVII-151 | H | CONMe₂ | H | H | H | F | H | H | H |
| XVII-152 | H | COMe | H | H | H | F | H | H | H |
| XVII-153 | H | COOH | H | H | H | F | H | H | H |
| XVII-154 | H | COOMe | H | H | H | F | H | H | H |
| XVII-155 | H | CSOMe | H | H | H | F | H | H | H |
| XVII-156 | H | CSNH₂ | H | H | H | F | H | H | H |
| XVII-157 | H | CSNMe₂ | H | H | H | F | H | H | H |
| XVII-158 | H | CSNHMe | H | H | H | F | H | H | H |
| XVII-159 | H | OMe | H | H | H | F | H | H | H |
| XVII-160 | H | OEt | H | H | H | F | H | H | H |
| XVII-161 | H | OCOMe | H | H | H | F | H | H | H |
| XVII-162 | H | OCOOMe | H | H | H | F | H | H | H |
| XVII-163 | H | OCONHMe | H | H | H | F | H | H | H |
| XVII-164 | H | OCONMe₂ | H | H | H | F | H | H | H |
| XVII-165 | H | OCSMe | H | H | H | F | H | H | H |
| XVII-166 | H | OCSNMe₂ | H | H | H | F | H | H | H |
| XVII-167 | H | SMe | H | H | H | F | H | H | H |
| XVII-168 | H | SEt | H | H | H | F | H | H | H |
| XVII-169 | H | SCOMe | H | H | H | F | H | H | H |
| XVII-170 | H | SCSNMe₂ | H | H | H | F | H | H | H |
| XVII-171 | H | SCSNHMe | H | H | H | F | H | H | H |
| XVII-172 | H | NHMe | H | H | H | F | H | H | H |
| XVII-173 | H | NH₂ | H | H | H | F | H | H | H |
| XVII-174 | H | NMe₂ | H | H | H | F | H | H | H |
| XVII-175 | H | NHCOMe | H | H | H | F | H | H | H |
| XVII-176 | H | NHCONH₂ | H | H | H | F | H | H | H |
| XVII-177 | H | NHCONHMe | H | H | H | F | H | H | H |
| XVII-178 | H | NHCONMe₂ | H | H | H | F | H | H | H |
| XVII-179 | H | phenyl | H | H | H | F | H | H | H |
| XVII-180 | H | 2-chloro- | H | H | H | F | H | H | H |
| | | phenyl | | | | | | | |
| XVII-181 | H | 4-nitrophenyl | H | H | H | F | H | H | H |
| XVII-182 | H | 2-pyridyl | H | H | H | F | H | H | H |
| XVII-183 | H | 3-pyridyl | H | H | H | F | H | H | H |
| XVII-184 | H | 4-pyridyl | H | H | H | F | H | H | H |
| XVII-185 | H | 2-furyl | H | H | H | F | H | H | H |
| XVII-186 | H | PhO | H | H | H | F | H | H | H |
| XVII-187 | CH₂ | | H | H | H | F | H | H | H |
| XVII-188 | H | H | H | Me | H | F | H | H | H |
| XVII-189 | H | H | H | Et | H | F | H | H | H |
| XVII-190 | H | H | H | vinyl | H | F | H | H | H |
| XVII-191 | H | H | H | allyl | H | F | H | H | H |
| XVII-192 | H | H | H | cyclopropyl | H | F | H | H | H |
| XVII-193 | H | H | H | F | H | F | H | H | H |
| XVII-194 | H | H | H | Cl | H | F | H | H | H |
| XVII-195 | H | H | H | NO₂ | H | F | H | H | H |
| XVII-196 | H | H | H | CN | H | F | H | H | H |
| XVII-197 | H | H | H | CONH₂ | H | F | H | H | H |
| XVII-198 | H | H | H | CONHMe | H | F | H | H | H |
| XVII-199 | H | H | H | CONMe₂ | H | F | H | H | H |
| XVII-200 | H | H | H | COMe | H | F | H | H | H |
| XVII-201 | H | H | H | COOH | H | F | H | H | H |
| XVII-202 | H | H | H | COOMe | H | F | H | H | H |
| XVII-203 | H | H | H | CSOMe | H | F | H | H | H |
| XVII-204 | H | H | H | CSNH₂ | H | F | H | H | H |
| XVII-205 | H | H | H | CSNMe₂ | H | F | H | H | H |
| XVII-206 | H | H | H | CSNHMe | H | F | H | H | H |
| XVII-207 | H | H | H | OMe | H | F | H | H | H |
| XVII-208 | H | H | H | OEt | H | F | H | H | H |
| XVII-209 | H | H | H | OCOMe | H | F | H | H | H |
| XVII-210 | H | H | H | OCOOMe | H | F | H | H | H |
| XVII-211 | H | H | H | OCONHMe | H | F | H | H | H |
| XVII-212 | H | H | H | OCONMe₂ | H | F | H | H | H |
| XVII-213 | H | H | H | OCSMe | H | F | H | H | H |
| XVII-214 | H | H | H | OCSNMe₂ | H | F | H | H | H |
| XVII-215 | H | H | H | SMe | H | F | H | H | H |
| XVII-216 | H | H | H | SEt | H | F | H | H | H |
| XVII-217 | H | H | H | SCOMe | H | F | H | H | H |
| XVII-218 | H | H | H | SCSNMe₂ | H | F | H | H | H |
| XVII-219 | H | H | H | SCSNHMe | H | F | H | H | H |
| XVII-220 | H | H | H | NHMe | H | F | H | H | H |
| XVII-221 | H | H | H | NH₂ | H | F | H | H | H |
| XVII-222 | H | H | H | NMe₂ | H | F | H | H | H |
| XVII-223 | H | H | H | NHCOMe | H | F | H | H | H |
| XVII-224 | H | H | H | NHCONH₂ | H | F | H | H | H |
| XVII-225 | H | H | H | NHCONHMe | H | F | H | H | H |
| XVII-226 | H | H | H | NHCONMe₂ | H | F | H | H | H |
| XVII-227 | H | H | H | phenyl | H | F | H | H | H |
| XVII-228 | H | H | H | 2-chloro- | H | F | H | H | H |
| | | | | phenyl | | | | | |
| XVII-229 | H | H | H | 4-nitrophenyl | H | F | H | H | H |
| XVII-230 | H | H | H | 2-pyridyl | H | F | H | H | H |
| XVII-231 | H | H | H | 3-pyridyl | H | F | H | H | H |
| XVII-232 | H | H | H | 4-pyridyl | H | F | H | H | H |
| XVII-233 | H | H | H | 2-furyl | H | F | H | H | H |
| XVII-234 | H | H | H | PhO | H | F | H | H | H |
| XVII-235 | H | Me | H | Me | H | F | H | H | H |
| XVII-236 | H | Me | F | Me | H | F | H | H | H |
| XVII-237 | H | Me | H | F | H | F | H | H | H |
| XVII-238 | H | Me | F | H | H | F | H | H | H |
| XVII-239 | H | Me | Me | H | H | F | H | H | H |
| XVII-240 | H | F | H | Me | H | F | H | H | H |
| XVII-241 | H | F | H | F | H | F | H | H | H |
| XVII-242 | Me | H | H | H | H | Me | H | H | H |
| XVII-243 | Et | H | H | H | H | Me | H | H | H |
| XVII-244 | vinyl | H | H | H | H | Me | H | H | H |
| XVII-245 | allyl | H | H | H | H | Me | H | H | H |
| XVII-246 | cyclopropyl | H | H | H | H | Me | H | H | H |
| XVII-247 | CN | H | H | H | H | Me | H | H | H |
| XVII-248 | CONH₂ | H | H | H | H | Me | H | H | H |
| XVII-249 | CONHMe | H | H | H | H | Me | H | H | H |
| XVII-250 | CONMe₂ | H | H | H | H | Me | H | H | H |
| XVII-251 | COMe | H | H | H | H | Me | H | H | H |
| XVII-252 | COOH | H | H | H | H | Me | H | H | H |
| XVII-253 | COOMe | H | H | H | H | Me | H | H | H |
| XVII-254 | CSNHMe | H | H | H | H | Me | H | H | H |
| XVII-255 | H | Me | H | H | H | Me | H | H | H |
| XVII-256 | H | Et | H | H | H | Me | H | H | H |
| XVII-257 | H | vinyl | H | H | H | Me | H | H | H |
| XVII-258 | H | allyl | H | H | H | Me | H | H | H |
| XVII-259 | H | cyclopropyl | H | H | H | Me | H | H | H |
| XVII-260 | H | F | H | H | H | Me | H | H | H |
| XVII-261 | H | Cl | H | H | H | Me | H | H | H |
| XVII-262 | H | NO₂ | H | H | H | Me | H | H | H |
| XVII-263 | H | CN | H | H | H | Me | H | H | H |
| XVII-264 | H | CONH₂ | H | H | H | Me | H | H | H |
| XVII-265 | H | CONHMe | H | H | H | Me | H | H | H |
| XVII-266 | H | CONMe₂ | H | H | H | Me | H | H | H |
| XVII-267 | H | COMe | H | H | H | Me | H | H | H |
| XVII-268 | H | COOH | H | H | H | Me | H | H | H |
| XVII-269 | H | COOMe | H | H | H | Me | H | H | H |
| XVII-270 | H | CSOMe | H | H | H | Me | H | H | H |
| XVII-271 | H | CSNH₂ | H | H | H | Me | H | H | H |
| XVII-272 | H | CSNMe₂ | H | H | H | Me | H | H | H |
| XVII-273 | H | CSNHMe | H | H | H | Me | H | H | H |
| XVII-274 | H | OMe | H | H | H | Me | H | H | H |
| XVII-275 | H | OEt | H | H | H | Me | H | H | H |
| XVII-276 | H | OCOMe | H | H | H | Me | H | H | H |
| XVII-277 | H | OCOOMe | H | H | H | Me | H | H | H |
| XVII-278 | H | OCONHMe | H | H | H | Me | H | H | H |
| XVII-279 | H | OCONMe₂ | H | H | H | Me | H | H | H |
| XVII-280 | H | OCSMe | H | H | H | Me | H | H | H |
| XVII-281 | H | OCSNMe₂ | H | H | H | Me | H | H | H |
| XVII-282 | H | SMe | H | H | H | Me | H | H | H |
| XVII-283 | H | SEt | H | H | H | Me | H | H | H |
| XVII-284 | H | SCOMe | H | H | H | Me | H | H | H |
| XVII-285 | H | SCSNMe₂ | H | H | H | Me | H | H | H |
| XVII-286 | H | SCSNHMe | H | H | H | Me | H | H | H |
| XVII-287 | H | NHMe | H | H | H | Me | H | H | H |
| XVII-288 | H | NH₂ | H | H | H | Me | H | H | H |
| XVII-289 | H | NMe₂ | H | H | H | Me | H | H | H |
| XVII-290 | H | NHCOMe | H | H | H | Me | H | H | H |
| XVII-291 | H | NHCONH₂ | H | H | H | Me | H | H | H |
| XVII-292 | H | NHCONHMe | H | H | H | Me | H | H | H |
| XVII-293 | H | NHCONMe₂ | H | H | H | Me | H | H | H |
| XVII-294 | H | phenyl | H | H | H | Me | H | H | H |
| XVII-295 | H | 2-chloro- | H | H | H | Me | H | H | H |
| | | phenyl | | | | | | | |
| XVII-296 | H | 4-nitrophenyl | H | H | H | Me | H | H | H |
| XVII-297 | H | 2-pyridyl | H | H | H | Me | H | H | H |
| XVII-298 | H | 3-pyridyl | H | H | H | Me | H | H | H |
| XVII-299 | H | 4-pyridyl | H | H | H | Me | H | H | H |
| XVII-300 | H | 2-furyl | H | H | H | Me | H | H | H |
| XVII-301 | H | PhO | H | H | H | Me | H | H | H |
| XVII-302 | CH₂ | | H | H | H | Me | H | H | H |
| XVII-303 | H | H | H | Me | H | Me | H | H | H |
| XVII-304 | H | H | H | Et | H | Me | H | H | H |
| XVII-305 | H | H | H | vinyl | H | Me | H | H | H |
| XVII-306 | H | H | H | allyl | H | Me | H | H | H |
| XVII-307 | H | H | H | cyclopropyl | H | Me | H | H | H |
| XVII-308 | H | H | H | F | H | Me | H | H | H |
| XVII-309 | H | H | H | Cl | H | Me | H | H | H |
| XVII-310 | H | H | H | NO₂ | H | Me | H | H | H |
| XVII-311 | H | H | H | CN | H | Me | H | H | H |
| XVII-312 | H | H | H | CONH₂ | H | Me | H | H | H |
| XVII-313 | H | H | H | CONHMe | H | Me | H | H | H |
| XVII-314 | H | H | H | CONMe₂ | H | Me | H | H | H |
| XVII-315 | H | H | H | COMe | H | Me | H | H | H |
| XVII-316 | H | H | H | COOH | H | Me | H | H | H |
| XVII-317 | H | H | H | COOMe | H | Me | H | H | H |
| XVII-318 | H | H | H | CSOMe | H | Me | H | H | H |
| XVII-319 | H | H | H | CSNH₂ | H | Me | H | H | H |
| XVII-320 | H | H | H | CSNMe₂ | H | Me | H | H | H |
| XVII-321 | H | H | H | CSNHMe | H | Me | H | H | H |
| XVII-322 | H | H | H | OMe | H | Me | H | H | H |
| XVII-323 | H | H | H | OEt | H | Me | H | H | H |
| XVII-324 | H | H | H | OCOMe | H | Me | H | H | H |
| XVII-325 | H | H | H | OCOOMe | H | Me | H | H | H |
| XVII-326 | H | H | H | OCONHMe | H | Me | H | H | H |
| XVII-327 | H | H | H | OCONMe₂ | H | Me | H | H | H |
| XVII-328 | H | H | H | OCSMe | H | Me | H | H | H |
| XVII-329 | H | H | H | OCSNMe₂ | H | Me | H | H | H |
| XVII-330 | H | H | H | SMe | H | Me | H | H | H |
| XVII-331 | H | H | H | SEt | H | Me | H | H | H |
| XVII-332 | H | H | H | SCOMe | H | Me | H | H | H |
| XVII-333 | H | H | H | SCSNMe₂ | H | Me | H | H | H |
| XVII-334 | H | H | H | SCSNHMe | H | Me | H | H | H |
| XVII-335 | H | H | H | NHMe | H | Me | H | H | H |
| XVII-336 | H | H | H | NH₂ | H | Me | H | H | H |
| XVII-337 | H | H | H | NMe₂ | H | Me | H | H | H |
| XVII-338 | H | H | H | NHCOMe | H | Me | H | H | H |
| XVII-339 | H | H | H | NHCONH₂ | H | Me | H | H | H |
| XVII-340 | H | H | H | NHCONHMe | H | Me | H | H | H |
| XVII-341 | H | H | H | NHCONMe₂ | H | Me | H | H | H |
| XVII-342 | H | H | H | phenyl | H | Me | H | H | H |
| XVII-343 | H | H | H | 2-chloro- | H | Me | H | H | H |
| | | | | phenyl | | | | | |
| XVII-344 | H | H | H | 4-nitrophenyl | H | Me | H | H | H |
| XVII-345 | H | H | H | 2-pyridyl | H | Me | H | H | H |
| XVII-346 | H | H | H | 3-pyridyl | H | Me | H | H | H |
| XVII-347 | H | H | H | 4-pyridyl | H | Me | H | H | H |
| XVII-348 | H | H | H | 2-furyl | H | Me | H | H | H |
| XVII-349 | H | H | H | PhO | H | Me | H | H | H |
| XVII-350 | H | Me | H | Me | H | Me | H | H | H |
| XVII-351 | H | Me | F | Me | H | Me | H | H | H |
| XVII-352 | H | Me | H | F | H | Me | H | H | H |
| XVII-353 | H | Me | F | H | H | Me | H | H | H |
| XVII-354 | H | Me | Me | H | H | Me | H | H | H |
| XVII-355 | H | F | H | Me | H | Me | H | H | H |
| XVII-356 | H | F | H | F | H | Me | H | H | H |
| XVII-357 | Me | H | H | H | H | F | H | F | H |
| XVII-358 | Et | H | H | H | H | F | H | F | H |
| XVII-359 | vinyl | H | H | H | H | F | H | F | H |
| XVII-360 | allyl | H | H | H | H | F | H | F | H |
| XVII-361 | cyclopropyl | H | H | H | H | F | H | F | H |
| XVII-362 | CN | H | H | H | H | F | H | F | H |
| XVII-363 | CONH₂ | H | H | H | H | F | H | F | H |
| XVII-364 | CONHMe | H | H | H | H | F | H | F | H |
| XVII-365 | CONMe₂ | H | H | H | H | F | H | F | H |
| XVII-366 | COMe | H | H | H | H | F | H | F | H |
| XVII-367 | COOH | H | H | H | H | F | H | F | H |
| XVII-368 | COOMe | H | H | H | H | F | H | F | H |
| XVII-369 | CSNHMe | H | H | H | H | F | H | F | H |
| XVII-370 | H | Me | H | H | H | F | H | F | H |
| XVII-371 | H | Et | H | H | H | F | H | F | H |
| XVII-372 | H | vinyl | H | H | H | F | H | F | H |
| XVII-373 | H | allyl | H | H | H | F | H | F | H |
| XVII-374 | H | cyclopropyl | H | H | H | F | H | F | H |
| XVII-375 | H | F | H | H | H | F | H | F | H |
| XVII-376 | H | Cl | H | H | H | F | H | F | H |
| XVII-377 | H | NO₂ | H | H | H | F | H | F | H |
| XVII-378 | H | CN | H | H | H | F | H | F | H |
| XVII-379 | H | CONH₂ | H | H | H | F | H | F | H |
| XVII-380 | H | CONHMe | H | H | H | F | H | F | H |
| XVII-381 | H | CONMe₂ | H | H | H | F | H | F | H |
| XVII-382 | H | COMe | H | H | H | F | H | F | H |
| XVII-383 | H | COOH | H | H | H | F | H | F | H |
| XVII-384 | H | COOMe | H | H | H | F | H | F | H |
| XVII-385 | H | CSOMe | H | H | H | F | H | F | H |
| XVII-386 | H | CSNH₂ | H | H | H | F | H | F | H |
| XVII-387 | H | CSNMe₂ | H | H | H | F | H | F | H |
| XVII-388 | H | CSNHMe | H | H | H | F | H | F | H |
| XVII-389 | H | OMe | H | H | H | F | H | F | H |
| XVII-390 | H | OEt | H | H | H | F | H | F | H |
| XVII-391 | H | OCOMe | H | H | H | F | H | F | H |
| XVII-392 | H | OCOOMe | H | H | H | F | H | F | H |
| XVII-393 | H | OCONHMe | H | H | H | F | H | F | H |
| XVII-394 | H | OCONMe₂ | H | H | H | F | H | F | H |
| XVII-395 | H | OCSMe | H | H | H | F | H | F | H |
| XVII-396 | H | OCSNMe₂ | H | H | H | F | H | F | H |
| XVII-397 | H | SMe | H | H | H | F | H | F | H |
| XVII-398 | H | SEt | H | H | H | F | H | F | H |
| XVII-399 | H | SCOMe | H | H | H | F | H | F | H |
| XVII-400 | H | SCSNMe₂ | H | H | H | F | H | F | H |
| XVII-401 | H | SCSNHMe | H | H | H | F | H | F | H |
| XVII-402 | H | NHMe | H | H | H | F | H | F | H |
| XVII-403 | H | NH₂ | H | H | H | F | H | F | H |
| XVII-404 | H | NMe₂ | H | H | H | F | H | F | H |
| XVII-405 | H | NHCOMe | H | H | H | F | H | F | H |
| XVII-406 | H | NHCONH₂ | H | H | H | F | H | F | H |
| XVII-407 | H | NHCONHMe | H | H | H | F | H | F | H |
| XVII-408 | H | NHCONMe₂ | H | H | H | F | H | F | H |
| XVII-409 | H | phenyl | H | H | H | F | H | F | H |
| XVII-410 | H | 2-chloro- | H | H | H | F | H | F | H |
| | | phenyl | | | | | | | |
| XVII-411 | H | 4-nitrophenyl | H | H | H | F | H | F | H |
| XVII-412 | H | 2-pyridyl | H | H | H | F | H | F | H |
| XVII-413 | H | 3-pyridyl | H | H | H | F | H | F | H |
| XVII-414 | H | 4-pyridyl | H | H | H | F | H | F | H |
| XVII-415 | H | 2-furyl | H | H | H | F | H | F | H |
| XVII-416 | H | PhO | H | H | H | F | H | F | H |
| XVII-417 | CH₂ | | H | H | H | F | H | F | H |
| XVII-418 | H | H | H | Me | H | F | H | F | H |
| XVII-419 | H | H | H | Et | H | F | H | F | H |
| XVII-420 | H | H | H | vinyl | H | F | H | F | H |
| XVII-421 | H | H | H | allyl | H | F | H | F | H |
| XVII-422 | H | H | H | cyclopropyl | H | F | H | F | H |
| XVII-423 | H | H | H | F | H | F | H | F | H |
| XVII-424 | H | H | H | Cl | H | F | H | F | H |
| XVII-425 | H | H | H | NO₂ | H | F | H | F | H |
| XVII-426 | H | H | H | CN | H | F | H | F | H |
| XVII-427 | H | H | H | CONH₂ | H | F | H | F | H |
| XVII-428 | H | H | H | CONHMe | H | F | H | F | H |
| XVII-429 | H | H | H | CONMe₂ | H | F | H | F | H |
| XVII-430 | H | H | H | COMe | H | F | H | F | H |
| XVII-431 | H | H | H | COOH | H | F | H | F | H |
| XVII-432 | H | H | H | COOMe | H | F | H | F | H |
| XVII-433 | H | H | H | CSOMe | H | F | H | F | H |
| XVII-434 | H | H | H | CSNH₂ | H | F | H | F | H |
| XVII-435 | H | H | H | CSNMe₂ | H | F | H | F | H |
| XVII-436 | H | H | H | CSNHMe | H | F | H | F | H |
| XVII-437 | H | H | H | OMe | H | F | H | F | H |
| XVII-438 | H | H | H | OEt | H | F | H | F | H |
| XVII-439 | H | H | H | OCOMe | H | F | H | F | H |
| XVII-440 | H | H | H | OCOOMe | H | F | H | F | H |
| XVII-441 | H | H | H | OCONHMe | H | F | H | F | H |
| XVII-442 | H | H | H | OCONMe₂ | H | F | H | F | H |
| XVII-443 | H | H | H | OCSMe | H | F | H | F | H |
| XVII-444 | H | H | H | OCSNMe₂ | H | F | H | F | H |
| XVII-445 | H | H | H | SMe | H | F | H | F | H |
| XVII-446 | H | H | H | SEt | H | F | H | F | H |
| XVII-447 | H | H | H | SCOMe | H | F | H | F | H |
| XVII-448 | H | H | H | SCSNMe₂ | H | F | H | F | H |
| XVII-449 | H | H | H | SCSNHMe | H | F | H | F | H |
| XVII-450 | H | H | H | NHMe | H | F | H | F | H |
| XVII-451 | H | H | H | NH₂ | H | F | H | F | H |
| XVII-452 | H | H | H | NMe₂ | H | F | H | F | H |
| XVII-453 | H | H | H | NHCOMe | H | F | H | F | H |
| XVII-454 | H | H | H | NHCONH₂ | H | F | H | F | H |
| XVII-455 | H | H | H | NHCONHMe | H | F | H | F | H |
| XVII-456 | H | H | H | NHCONMe₂ | H | F | H | F | H |
| XVII-457 | H | H | H | phenyl | H | F | H | F | H |
| XVII-458 | H | H | H | 2-chloro- | H | F | H | F | H |
| | | | | phenyl | | | | | |
| XVII-459 | H | H | H | 4-nitrophenyl | H | F | H | F | H |
| XVII-460 | H | H | H | 2-pyridyl | H | F | H | F | H |
| XVII-461 | H | H | H | 3-pyridyl | H | F | H | F | H |
| XVII-462 | H | H | H | 4-pyridyl | H | F | H | F | H |
| XVII-463 | H | H | H | 2-furyl | H | F | H | F | H |
| XVII-464 | H | H | H | PhO | H | F | H | F | H |
| XVII-465 | H | Me | H | Me | H | F | H | F | H |
| XVII-466 | H | Me | F | Me | H | F | H | F | H |
| XVII-467 | H | Me | H | F | H | F | H | F | H |
| XVII-468 | H | Me | F | H | H | F | H | F | H |
| XVII-469 | H | Me | Me | H | H | F | H | F | H |
| XVII-470 | H | F | H | Me | H | F | H | F | H |
| XVII-471 | H | F | H | F | H | F | H | F | H |
| XVII-472 | H | H | H | H | H | Me | H | H | H |
| XVII-473 | H | H | H | H | H | Et | H | H | H |
| XVII-474 | H | H | H | H | H | vinyl | H | H | H |
| XVII-475 | H | H | H | H | H | allyl | H | H | H |
| XVII-476 | H | H | H | H | H | cyclopropyl | H | H | H |
| XVII-477 | H | H | H | H | H | F | H | H | H |
| XVII-478 | H | H | H | H | H | Cl | H | H | H |
| XVII-479 | H | H | H | H | H | NO₂ | H | H | H |
| XVII-480 | H | H | H | H | H | CN | H | H | H |
| XVII-481 | H | H | H | H | H | CONH₂ | H | H | H |
| XVII-482 | H | H | H | H | H | CONHMe | H | H | H |
| XVII-483 | H | H | H | H | H | CONMe₂ | H | H | H |
| XVII-484 | H | H | H | H | H | COMe | H | H | H |
| XVII-485 | H | H | H | H | H | COOH | H | H | H |
| XVII-486 | H | H | H | H | H | COOMe | H | H | H |
| XVII-487 | H | H | H | H | H | CSOMe | H | H | H |
| XVII-488 | H | H | H | H | H | CSNH₂ | H | H | H |
| XVII-489 | H | H | H | H | H | CSNMe₂ | H | H | H |
| XVII-490 | H | H | H | H | H | CSNHMe | H | H | H |
| XVII-491 | H | H | H | H | H | OMe | H | H | H |
| XVII-492 | H | H | H | H | H | OEt | H | H | H |
| XVII-493 | H | H | H | H | H | OCOMe | H | H | H |
| XVII-494 | H | H | H | H | H | OCOOMe | H | H | H |
| XVII-495 | H | H | H | H | H | OCONHMe | H | H | H |
| XVII-496 | H | H | H | H | H | OCONMe₂ | H | H | H |
| XVII-497 | H | H | H | H | H | OCSMe | H | H | H |
| XVII-498 | H | H | H | H | H | OCSNMe₂ | H | H | H |
| XVII-499 | H | H | H | H | H | SMe | H | H | H |
| XVII-500 | H | H | H | H | H | SEt | H | H | H |
| XVII-501 | H | H | H | H | H | SCOMe | H | H | H |
| XVII-502 | H | H | H | H | H | SCSNMe₂ | H | H | H |
| XVII-503 | H | H | H | H | H | SCSNHMe | H | H | H |
| XVII-504 | H | H | H | H | H | NHMe | H | H | H |
| XVII-505 | H | H | H | H | H | NH₂ | H | H | H |
| XVII-506 | H | H | H | H | H | NMe₂ | H | H | H |
| XVII-507 | H | H | H | H | H | NHCOMe | H | H | H |
| XVII-508 | H | H | H | H | H | NHCONH₂ | H | H | H |
| XVII-509 | H | H | H | H | H | NHCONHMe | H | H | H |
| XVII-510 | H | H | H | H | H | NHCONMe₂ | H | H | H |
| XVII-511 | H | H | H | H | H | phenyl | H | H | H |
| XVII-512 | H | H | H | H | H | 2-chloro- | H | H | H |
| | | | | | | phenyl | | | |
| XVII-513 | H | H | H | H | H | 4-nitrophenyl | H | H | H |
| XVII-514 | H | H | H | H | H | 2-pyridyl | H | H | H |
| XVII-515 | H | H | H | H | H | 3-pyridyl | H | H | H |
| XVII-516 | H | H | H | H | H | 4-pyridyl | H | H | H |
| XVII-517 | H | H | H | H | H | 2-furyl | H | H | H |
| XVII-518 | H | H | H | H | H | PhO | H | H | H |
| XVII-519 | H | H | H | H | H | H | H | Me | H |
| XVII-520 | H | H | H | H | H | H | H | Et | H |
| XVII-521 | H | H | H | H | H | H | H | vinyl | H |
| XVII-522 | H | H | H | H | H | H | H | allyl | H |
| XVII-523 | H | H | H | H | H | H | H | cyclopropyl | H |
| XVII-524 | H | H | H | H | H | H | H | F | H |
| XVII-525 | H | H | H | H | H | H | H | Cl | H |
| XVII-526 | H | H | H | H | H | H | H | NO₂ | H |
| XVII-527 | H | H | H | H | H | H | H | CN | H |
| XVII-528 | H | H | H | H | H | H | H | CONH₂ | H |
| XVII-529 | H | H | H | H | H | H | H | CONHMe | H |
| XVII-530 | H | H | H | H | H | H | H | CONMe₂ | H |
| XVII-531 | H | H | H | H | H | H | H | COMe | H |
| XVII-532 | H | H | H | H | H | H | H | COOH | H |
| XVII-533 | H | H | H | H | H | H | H | COOMe | H |
| XVII-534 | H | H | H | H | H | H | H | CSOMe | H |
| XVII-535 | H | H | H | H | H | H | H | CSNH₂ | H |
| XVII-536 | H | H | H | H | H | H | H | CSNMe₂ | H |
| XVII-537 | H | H | H | H | H | H | H | CSNHMe | H |
| XVII-538 | H | H | H | H | H | H | H | OMe | H |
| XVII-539 | H | H | H | H | H | H | H | OEt | H |
| XVII-540 | H | H | H | H | H | H | H | OCOMe | H |
| XVII-541 | H | H | H | H | H | H | H | OCOOMe | H |
| XVII-542 | H | H | H | H | H | H | H | OCONHMe | H |
| XVII-543 | H | H | H | H | H | H | H | OCONMe₂ | H |
| XVII-544 | H | H | H | H | H | H | H | OCSMe | H |
| XVII-545 | H | H | H | H | H | H | H | OCSNMe₂ | H |
| XVII-546 | H | H | H | H | H | H | H | SMe | H |
| XVII-547 | H | H | H | H | H | H | H | SEt | H |
| XVII-548 | H | H | H | H | H | H | H | SCOMe | H |
| XVII-549 | H | H | H | H | H | H | H | SCSNMe₂ | H |
| XVII-550 | H | H | H | H | H | H | H | SCSNHMe | H |
| XVII-551 | H | H | H | H | H | H | H | NHMe | H |
| XVII-552 | H | H | H | H | H | H | H | NH₂ | H |
| XVII-553 | H | H | H | H | H | H | H | NMe₂ | H |
| XVII-554 | H | H | H | H | H | H | H | NHCOMe | H |
| XVII-555 | H | H | H | H | H | H | H | NHCONH₂ | H |
| XVII-556 | H | H | H | H | H | H | H | NHCONHMe | H |
| XVII-557 | H | H | H | H | H | H | H | NHCONMe₂ | H |
| XVII-558 | H | H | H | H | H | H | H | phenyl | H |
| XVII-559 | H | H | H | H | H | H | H | 2-chloro- | H |
| | | | | | | | | phenyl | |
| XVII-560 | H | H | H | H | H | H | H | 4-nitrophenyl | H |
| XVII-561 | H | H | H | H | H | H | H | 2-pyridyl | H |
| XVII-562 | H | H | H | H | H | H | H | 3-pyridyl | H |
| XVII-563 | H | H | H | H | H | H | H | 4-pyridyl | H |
| XVII-564 | H | H | H | H | H | H | H | 2-furyl | H |
| XVII-565 | H | H | H | H | H | H | H | PhO | H |
| XVII-566 | H | H | H | H | H | H | H | H | Me |
| XVII-567 | H | H | H | H | H | H | H | H | Et |
| XVII-568 | H | H | H | H | H | H | H | H | vinyl |
| XVII-569 | H | H | H | H | H | H | H | H | allyl |
| XVII-570 | H | H | H | H | H | H | H | H | cyclopropyl |
| XVII-571 | H | H | H | H | H | H | H | H | F |
| XVII-572 | H | H | H | H | H | H | H | H | Cl |
| XVII-573 | H | H | H | H | H | H | H | H | NO₂ |
| XVII-574 | H | H | H | H | H | H | H | H | CN |
| XVII-575 | H | H | H | H | H | H | H | H | CONH₂ |
| XVII-576 | H | H | H | H | H | H | H | H | CONHMe |
| XVII-577 | H | H | H | H | H | H | H | H | CONMe₂ |
| XVII-578 | H | H | H | H | H | H | H | H | COMe |
| XVII-579 | H | H | H | H | H | H | H | H | COOH |
| XVII-580 | H | H | H | H | H | H | H | H | COOMe |
| XVII-581 | H | H | H | H | H | H | H | H | CSOMe |
| XVII-582 | H | H | H | H | H | H | H | H | CSNH₂ |
| XVII-583 | H | H | H | H | H | H | H | H | CSNMe₂ |
| XVII-584 | H | H | H | H | H | H | H | H | CSNHMe |
| XVII-585 | H | H | H | H | H | H | H | H | OMe |
| XVII-586 | H | H | H | H | H | H | H | H | OEt |
| XVII-587 | H | H | H | H | H | H | H | H | OCOMe |
| XVII-588 | H | H | H | H | H | H | H | H | OCOOMe |
| XVII-589 | H | H | H | H | H | H | H | H | OCONHMe |
| XVII-590 | H | H | H | H | H | H | H | H | OCONMe₂ |
| XVII-591 | H | H | H | H | H | H | H | H | OCSMe |
| XVII-592 | H | H | H | H | H | H | H | H | OCSNMe₂ |
| XVII-593 | H | H | H | H | H | H | H | H | SMe |
| XVII-594 | H | H | H | H | H | H | H | H | SEt |
| XVII-595 | H | H | H | H | H | H | H | H | SCOMe |
| XVII-596 | H | H | H | H | H | H | H | H | SCSNMe₂ |
| XVII-597 | H | H | H | H | H | H | H | H | SCSNHMe |
| XVII-598 | H | H | H | H | H | H | H | H | NHMe |
| XVII-599 | H | H | H | H | H | H | H | H | NH₂ |
| XVII-600 | H | H | H | H | H | H | H | H | NMe₂ |
| XVII-601 | H | H | H | H | H | H | H | H | NHCOMe |
| XVII-602 | H | H | H | H | H | H | H | H | NHCONH₂ |
| XVII-603 | H | H | H | H | H | H | H | H | NHCONHMe |
| XVII-604 | H | H | H | H | H | H | H | H | NHCONMe₂ |
| XVII-605 | H | H | H | H | H | H | H | H | phenyl |
| XVII-606 | H | H | H | H | H | H | H | H | 2-chloro- |
| | | | | | | | | | phenyl |
| XVII-607 | H | H | H | H | H | H | H | H | 4-nitrophenyl |
| XVII-608 | H | H | H | H | H | H | H | H | 2-pyridyl |
| XVII-609 | H | H | H | H | H | H | H | H | 3-pyridyl |
| XVII-610 | H | H | H | H | H | H | H | H | 4-pyridyl |
| XVII-611 | H | H | H | H | H | H | H | H | 2-furyl |
| XVII-612 | H | H | H | H | H | H | H | H | PhO |

Table XVIII provides 612 compounds of formula Ir

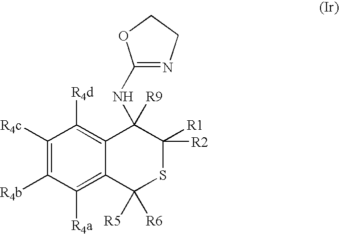

wherein the values of R¹, R², R^{4a}, R^{4b}, R^{4c}, R^{4d}, R⁵, R⁶ and R⁹ are given in Table 2.

Table XIX provides 612 compounds of formula Is

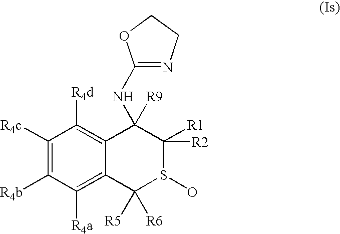

wherein the values of R¹, R², R^{4a}, R^{4b}, R^{4c}, R^{4d}, R⁵, R⁶ and R⁹ are given in Table 2.

Table XX provides 612 compounds of formula It

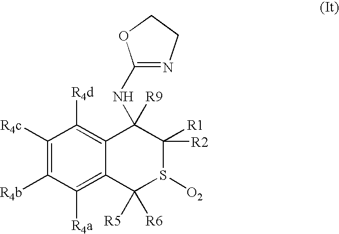

wherein the values of R¹, R², R^{4a}, R^{4b}, R^{4c}, R^{4d}, R⁵, R⁶ and R⁹ are given in Table 2.

Table XXI provides 612 compounds of formula Iu

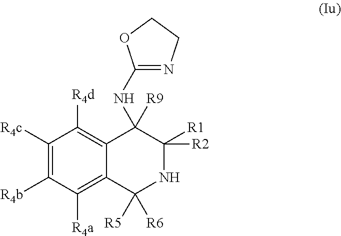

wherein the values of R¹, R², R^{4a}, R^{4b}, R^{4c}, R^{4d}, R⁵, R⁶ and R⁹ are given in Table 2.

Table XXII provides 612 compounds of formula Iv

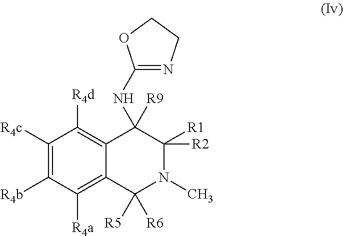

wherein the values of R¹, R², R^{4a}, R^{4b}, R^{4c}, R^{4d}, R⁵, R⁶ and R⁹ are given in Table 2.

Table XXIII provides 612 compounds of formula Iw

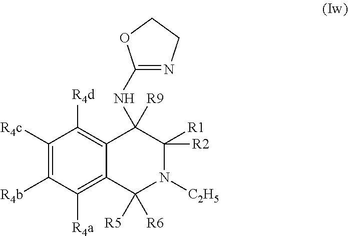

wherein the values of R¹, R², R^{4a}, R^{4b}, R^{4c}, R^{4d}, R⁵, and R⁹ are given in Table 2.

Table XXIV provides 612 compounds of formula Ix

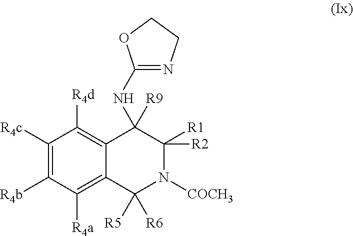

wherein the values of R¹, R², R^{4a}, R^{4b}, R^{4c}, R^{4d}, R⁵, R⁶ and R⁹ are given in Table 2.

Table XXV provides 612 compounds of formula Iy

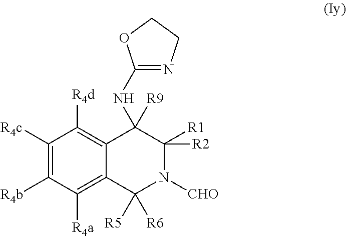

wherein the values of R¹, R², R^{4a}, R^{4b}, R^{4c}, R^{4d}, R⁵, R⁶ and R⁹ are given in Table 2.

Table XXVI provides 612 compounds of formula Iz

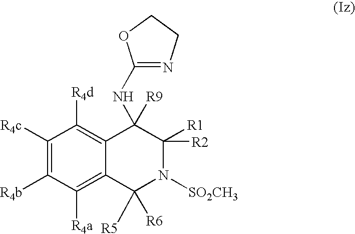

wherein the values of R¹, R², R^{4a}, R^{4b}, R^{4c}, R^{4d}, R⁵, R⁶ and R⁹ are given in Table 2.

Table XXVII provides 612 compounds of formula Iaa

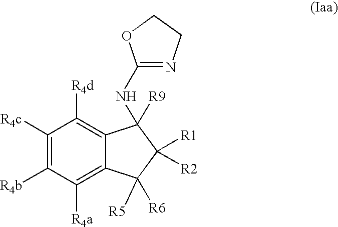

wherein the values of R¹, R², R^{4a}, R^{4b}, R^{4c}, R^{4d}, R⁵, R⁶ and R⁹ are given in Table 3.

TABLE 3

| Compound | | | | | | | | | |
|---|---|---|---|---|---|---|---|---|---|
| No | R9 | R1 | R2 | R5 | R6 | R4a | R4b | R4c | R4d |
| XXVII-1 | Me | H | H | H | H | H | H | H | H |
| XXVII-2 | Et | H | H | H | H | H | H | H | H |
| XXVII-3 | vinyl | H | H | H | H | H | H | H | H |
| XXVII-4 | allyl | H | H | H | H | H | H | H | H |
| XXVII-5 | cyclopropyl | H | H | H | H | H | H | H | H |
| XXVII-6 | CN | H | H | H | H | H | H | H | H |
| XXVII-7 | CONH₂ | H | H | H | H | H | H | H | H |
| XXVII-8 | CONHMe | H | H | H | H | H | H | H | H |
| XXVII-9 | CONMe₂ | H | H | H | H | H | H | H | H |
| XXVII-10 | COMe | H | H | H | H | H | H | H | H |
| XXVII-11 | COOH | H | H | H | H | H | H | H | H |
| XXVII-12 | COOMe | H | H | H | H | H | H | H | H |
| XXVII-13 | CSNHMe | H | H | H | H | H | H | H | H |
| XXVII-14 | H | Me | H | H | H | H | H | H | H |
| XXVII-15 | H | Et | H | H | H | H | H | H | H |
| XXVII-16 | H | vinyl | H | H | H | H | H | H | H |
| XXVII-17 | H | allyl | H | H | H | H | H | H | H |
| XXVII-18 | H | cyclopropyl | H | H | H | H | H | H | H |
| XXVII-19 | H | F | H | H | H | H | H | H | H |
| XXVII-20 | H | Cl | H | H | H | H | H | H | H |
| XXVII-21 | H | NO₂ | H | H | H | H | H | H | H |
| XXVII-22 | H | CN | H | H | H | H | H | H | H |
| XXVII-23 | H | CONH₂ | H | H | H | H | H | H | H |
| XXVII-24 | H | CONHMe | H | H | H | H | H | H | H |
| XXVII-25 | H | CONMe₂ | H | H | H | H | H | H | H |
| XXVII-26 | H | COMe | H | H | H | H | H | H | H |
| XXVII-27 | H | COOH | H | H | H | H | H | H | H |
| XXVII-28 | H | COOMe | H | H | H | H | H | H | H |
| XXVII-29 | H | CSOMe | H | H | H | H | H | H | H |
| XXVII-30 | H | CSNH₂ | H | H | H | H | H | H | H |
| XXVII-31 | H | CSNMe₂ | H | H | H | H | H | H | H |
| XXVII-32 | H | CSNHMe | H | H | H | H | H | H | H |
| XXVII-33 | H | OMe | H | H | H | H | H | H | H |
| XXVII-34 | H | OEt | H | H | H | H | H | H | H |
| XXVII-35 | H | OCOMe | H | H | H | H | H | H | H |
| XXVII-36 | H | OCOOMe | H | H | H | H | H | H | H |
| XXVII-37 | H | OCONHMe | H | H | H | H | H | H | H |
| XXVII-38 | H | OCONMe₂ | H | H | H | H | H | H | H |
| XXVII-39 | H | OCSMe | H | H | H | H | H | H | H |
| XXVII-40 | H | OCSNMe₂ | H | H | H | H | H | H | H |
| XXVII-41 | H | SMe | H | H | H | H | H | H | H |
| XXVII-42 | H | SEt | H | H | H | H | H | H | H |
| XXVII-43 | H | SCOMe | H | H | H | H | H | H | H |
| XXVII-44 | H | SCSNMe₂ | H | H | H | H | H | H | H |
| XXVII-45 | H | SCSNHMe | H | H | H | H | H | H | H |
| XXVII-46 | H | NHMe | H | H | H | H | H | H | H |
| XXVII-47 | H | NH₂ | H | H | H | H | H | H | H |
| XXVII-48 | H | NMe₂ | H | H | H | H | H | H | H |
| XXVII-49 | H | NHCOMe | H | H | H | H | H | H | H |
| XXVII-50 | H | NHCONH₂ | H | H | H | H | H | H | H |
| XXVII-51 | H | NHCONHMe | H | H | H | H | H | H | H |
| XXVII-52 | H | NHCONMe₂ | H | H | H | H | H | H | H |
| XXVII-53 | H | phenyl | H | H | H | H | H | H | H |
| XXVII-54 | H | 2-chloro- | H | H | H | H | H | H | H |
| | | phenyl | | | | | | | |
| XXVII-55 | H | 4-nitrophenyl | H | H | H | H | H | H | H |
| XXVII-56 | H | 2-pyridyl | H | H | H | H | H | H | H |
| XXVII-57 | H | 3-pyridyl | H | H | H | H | H | H | H |
| XXVII-58 | H | 4-pyridyl | H | H | H | H | H | H | H |
| XXVII-59 | H | 2-furyl | H | H | H | H | H | H | H |
| XXVII-60 | H | PhO | H | H | H | H | H | H | H |
| XXVII-61 | CH₂ | | H | H | H | H | H | H | H |
| XXVII-62 | H | ═O | | H | H | H | H | H | H |
| XXVII-63 | H | ═NOH | | H | H | H | H | H | H |
| XXVII-64 | H | ═NOMe | | H | H | H | H | H | H |
| XXVII-65 | H | ═CH₂ | | H | H | H | H | H | H |
| XXVII-66 | H | ═CHMe | | H | H | H | H | H | H |
| XXVII-67 | H | H | H | H | H | H | H | H | H |
| XXVII-68 | H | H | H | Me | H | H | H | H | H |
| XXVII-69 | H | H | H | Et | H | H | H | H | H |
| XXVII-70 | H | H | H | vinyl | H | H | H | H | H |
| XXVII-71 | H | H | H | allyl | H | H | H | H | H |
| XXVII-72 | H | H | H | cyclopropyl | H | H | H | H | H |
| XXVII-73 | H | H | H | F | H | H | H | H | H |
| XXVII-74 | H | H | H | Cl | H | H | H | H | H |
| XXVII-75 | H | H | H | NO₂ | H | H | H | H | H |
| XXVII-76 | H | H | H | CN | H | H | H | H | H |
| XXVII-77 | H | H | H | CONH₂ | H | H | H | H | H |
| XXVII-78 | H | H | H | CONHMe | H | H | H | H | H |
| XXVII-79 | H | H | H | CONMe₂ | H | H | H | H | H |
| XXVII-80 | H | H | H | COMe | H | H | H | H | H |
| XXVII-81 | H | H | H | COOH | H | H | H | H | H |
| XXVII-82 | H | H | H | COOMe | H | H | H | H | H |
| XXVII-83 | H | H | H | CSOMe | H | H | H | H | H |
| XXVII-84 | H | H | H | CSNH₂ | H | H | H | H | H |
| XXVII-85 | H | H | H | CSNMe₂ | H | H | H | H | H |
| XXVII-86 | H | H | H | CSNHMe | H | H | H | H | H |
| XXVII-87 | H | H | H | OMe | H | H | H | H | H |
| XXVII-88 | H | H | H | OEt | H | H | H | H | H |
| XXVII-89 | H | H | H | OCOMe | H | H | H | H | H |
| XXVII-90 | H | H | H | OCOOMe | H | H | H | H | H |
| XXVII-91 | H | H | H | OCONHMe | H | H | H | H | H |
| XXVII-92 | H | H | H | OCONMe₂ | H | H | H | H | H |
| XXVII-93 | H | H | H | OCSMe | H | H | H | H | H |
| XXVII-94 | H | H | H | OCSNMe₂ | H | H | H | H | H |
| XXVII-95 | H | H | H | SMe | H | H | H | H | H |
| XXVII-96 | H | H | H | SEt | H | H | H | H | H |
| XXVII-97 | H | H | H | SCOMe | H | H | H | H | H |
| XXVII-98 | H | H | H | SCSNMe₂ | H | H | H | H | H |
| XXVII-99 | H | H | H | SCSNHMe | H | H | H | H | H |
| XXVII-100 | H | H | H | NHMe | H | H | H | H | H |
| XXVII-101 | H | H | H | NH₂ | H | H | H | H | H |
| XXVII-102 | H | H | H | NMe₂ | H | H | H | H | H |
| XXVII-103 | H | H | H | NHCOMe | H | H | H | H | H |
| XXVII-104 | H | H | H | NHCONH₂ | H | H | H | H | H |
| XXVII-105 | H | H | H | NHCONHMe | H | H | H | H | H |
| XXVII-106 | H | H | H | NHCONMe₂ | H | H | H | H | H |
| XXVII-107 | H | H | H | phenyl | H | H | H | H | H |
| XXVII-108 | H | H | H | 2-chloro- | H | H | H | H | H |
| | | | | phenyl | | | | | |
| XXVII-109 | H | H | H | 4-nitrophenyl | H | H | H | H | H |
| XXVII-110 | H | H | H | 2-pyridyl | H | H | H | H | H |
| XXVII-111 | H | H | H | 3-pyridyl | H | H | H | H | H |
| XXVII-112 | H | H | H | 4-pyridyl | H | H | H | H | H |
| XXVII-113 | H | H | H | 2-furyl | H | H | H | H | H |
| XXVII-114 | H | H | H | PhO | H | H | H | H | H |
| XXVII-115 | H | H | H | ═O | | H | H | H | H |
| XXVII-116 | H | H | H | ═NOH | | H | H | H | H |
| XXVII-117 | H | H | H | ═NOMe | | H | H | H | H |
| XXVII-118 | H | H | H | ═CH₂ | | H | H | H | H |
| XXVII-119 | H | H | H | ═CHMe | | H | H | H | H |
| XXVII-120 | H | Me | H | Me | H | H | H | H | H |
| XXVII-121 | H | Me | F | Me | H | H | H | H | H |
| XXVII-122 | H | Me | H | F | H | H | H | H | H |
| XXVII-123 | H | Me | F | H | H | H | H | H | H |
| XXVII-124 | H | Me | Me | H | H | H | H | H | H |
| XXVII-125 | H | F | H | Me | H | H | H | H | H |
| XXVII-126 | H | F | H | F | H | H | H | H | H |
| XXVII-127 | Me | H | H | H | H | F | H | H | H |
| XXVII-128 | Et | H | H | H | H | F | H | H | H |
| XXVII-129 | vinyl | H | H | H | H | F | H | H | H |
| XXVII-130 | allyl | H | H | H | H | F | H | H | H |
| XXVII-131 | cyclopropyl | H | H | H | H | F | H | H | H |
| XXVII-132 | CN | H | H | H | H | F | H | H | H |
| XXVII-133 | CONH₂ | H | H | H | H | F | H | H | H |
| XXVII-134 | CONHMe | H | H | H | H | F | H | H | H |
| XXVII-135 | CONMe₂ | H | H | H | H | F | H | H | H |
| XXVII-136 | COMe | H | H | H | H | F | H | H | H |
| XXVII-137 | COOH | H | H | H | H | F | H | H | H |
| XXVII-138 | COOMe | H | H | H | H | F | H | H | H |
| XXVII-139 | CSNHMe | H | H | H | H | F | H | H | H |
| XXVII-140 | H | Me | H | H | H | F | H | H | H |
| XXVII-141 | H | Et | H | H | H | F | H | H | H |
| XXVII-142 | H | vinyl | H | H | H | F | H | H | H |
| XXVII-143 | H | allyl | H | H | H | F | H | H | H |
| XXVII-144 | H | cyclopropyl | H | H | H | F | H | H | H |
| XXVII-145 | H | F | H | H | H | F | H | H | H |
| XXVII-146 | H | Cl | H | H | H | F | H | H | H |
| XXVII-147 | H | NO₂ | H | H | H | F | H | H | H |
| XXVII-148 | H | CN | H | H | H | F | H | H | H |
| XXVII-149 | H | CONH₂ | H | H | H | F | H | H | H |
| XXVII-150 | H | CONHMe | H | H | H | F | H | H | H |
| XXVII-151 | H | CONMe₂ | H | H | H | F | H | H | H |
| XXVII-152 | H | COMe | H | H | H | F | H | H | H |
| XXVII-153 | H | COOH | H | H | H | F | H | H | H |
| XXVII-154 | H | COOMe | H | H | H | F | H | H | H |
| XXVII-155 | H | CSOMe | H | H | H | F | H | H | H |
| XXVII-156 | H | CSNH₂ | H | H | H | F | H | H | H |
| XXVII-157 | H | CSNMe₂ | H | H | H | F | H | H | H |
| XXVII-158 | H | CSNHMe | H | H | H | F | H | H | H |
| XXVII-159 | H | OMe | H | H | H | F | H | H | H |
| XXVII-160 | H | OEt | H | H | H | F | H | H | H |
| XXVII-161 | H | OCOMe | H | H | H | F | H | H | H |
| XXVII-162 | H | OCOOMe | H | H | H | F | H | H | H |
| XXVII-163 | H | OCONHMe | H | H | H | F | H | H | H |
| XXVII-164 | H | OCONMe₂ | H | H | H | F | H | H | H |
| XXVII-165 | H | OCSMe | H | H | H | F | H | H | H |
| XXVII-166 | H | OCSNMe₂ | H | H | H | F | H | H | H |
| XXVII-167 | H | SMe | H | H | H | F | H | H | H |
| XXVII-168 | H | SEt | H | H | H | F | H | H | H |
| XXVII-169 | H | SCOMe | H | H | H | F | H | H | H |
| XXVII-170 | H | SCSNMe₂ | H | H | H | F | H | H | H |
| XXVII-171 | H | SCSNHMe | H | H | H | F | H | H | H |
| XXVII-172 | H | NHMe | H | H | H | F | H | H | H |
| XXVII-173 | H | NH₂ | H | H | H | F | H | H | H |
| XXVII-174 | H | NMe₂ | H | H | H | F | H | H | H |
| XXVII-175 | H | NHCOMe | H | H | H | F | H | H | H |
| XXVII-176 | H | NHCONH₂ | H | H | H | F | H | H | H |
| XXVII-177 | H | NHCONHMe | H | H | H | F | H | H | H |
| XXVII-178 | H | NHCONMe₂ | H | H | H | F | H | H | H |
| XXVII-179 | H | phenyl | H | H | H | F | H | H | H |
| XXVII-180 | H | 2-chloro- | H | H | H | F | H | H | H |
| | | phenyl | | | | | | | |
| XXVII-181 | H | 4-nitrophenyl | H | H | H | F | H | H | H |
| XXVII-182 | H | 2-pyridyl | H | H | H | F | H | H | H |
| XXVII-183 | H | 3-pyridyl | H | H | H | F | H | H | H |
| XXVII-184 | H | 4-pyridyl | H | H | H | F | H | H | H |
| XXVII-185 | H | 2-furyl | H | H | H | F | H | H | H |
| XXVII-186 | H | PhO | H | H | H | F | H | H | H |
| XXVII-187 | CH₂ | | H | H | H | F | H | H | H |
| XXVII-188 | H | H | H | Me | H | F | H | H | H |
| XXVII-189 | H | H | H | Et | H | F | H | H | H |
| XXVII-190 | H | H | H | vinyl | H | F | H | H | H |
| XXVII-191 | H | H | H | allyl | H | F | H | H | H |
| XXVII-192 | H | H | H | cyclopropyl | H | F | H | H | H |
| XXVII-193 | H | H | H | F | H | F | H | H | H |
| XXVII-194 | H | H | H | Cl | H | F | H | H | H |
| XXVII-195 | H | H | H | NO₂ | H | F | H | H | H |
| XXVII-196 | H | H | H | CN | H | F | H | H | H |
| XXVII-197 | H | H | H | CONH₂ | H | F | H | H | H |
| XXVII-198 | H | H | H | CONHMe | H | F | H | H | H |
| XXVII-199 | H | H | H | CONMe₂ | H | F | H | H | H |
| XXVII-200 | H | H | H | COMe | H | F | H | H | H |
| XXVII-201 | H | H | H | COOH | H | F | H | H | H |
| XXVII-202 | H | H | H | COOMe | H | F | H | H | H |
| XXVII-203 | H | H | H | CSOMe | H | F | H | H | H |
| XXVII-204 | H | H | H | CSNH₂ | H | F | H | H | H |
| XXVII-205 | H | H | H | CSNMe₂ | H | F | H | H | H |
| XXVII-206 | H | H | H | CSNHMe | H | F | H | H | H |
| XXVII-207 | H | H | H | OMe | H | F | H | H | H |
| XXVII-208 | H | H | H | OEt | H | F | H | H | H |
| XXVII-209 | H | H | H | OCOMe | H | F | H | H | H |
| XXVII-210 | H | H | H | OCOOMe | H | F | H | H | H |
| XXVII-211 | H | H | H | OCONHMe | H | F | H | H | H |
| XXVII-212 | H | H | H | OCONMe₂ | H | F | H | H | H |
| XXVII-213 | H | H | H | OCSMe | H | F | H | H | H |
| XXVII-214 | H | H | H | OCSNMe₂ | H | F | H | H | H |
| XXVII-215 | H | H | H | SMe | H | F | H | H | H |
| XXVII-216 | H | H | H | SEt | H | F | H | H | H |
| XXVII-217 | H | H | H | SCOMe | H | F | H | H | H |
| XXVII-218 | H | H | H | SCSNMe₂ | H | F | H | H | H |
| XXVII-219 | H | H | H | SCSNHMe | H | F | H | H | H |
| XXVII-220 | H | H | H | NHMe | H | F | H | H | H |
| XXVII-221 | H | H | H | NH₂ | H | F | H | H | H |
| XXVII-222 | H | H | H | NMe₂ | H | F | H | H | H |
| XXVII-223 | H | H | H | NHCOMe | H | F | H | H | H |
| XXVII-224 | H | H | H | NHCONH₂ | H | F | H | H | H |
| XXVII-225 | H | H | H | NHCONHMe | H | F | H | H | H |
| XXVII-226 | H | H | H | NHCONMe₂ | H | F | H | H | H |
| XXVII-227 | H | H | H | phenyl | H | F | H | H | H |
| XXVII-228 | H | H | H | 2-chloro- | H | F | H | H | H |
| | | | | phenyl | | | | | |
| XXVII-229 | H | H | H | 4-nitrophenyl | H | F | H | H | H |
| XXVII-230 | H | H | H | 2-pyridyl | H | F | H | H | H |
| XXVII-231 | H | H | H | 3-pyridyl | H | F | H | H | H |
| XXVII-232 | H | H | H | 4-pyridyl | H | F | H | H | H |
| XXVII-233 | H | H | H | 2-furyl | H | F | H | H | H |
| XXVII-234 | H | H | H | PhO | H | F | H | H | H |
| XXVII-235 | H | Me | H | Me | H | F | H | H | H |
| XXVII-236 | H | Me | F | Me | H | F | H | H | H |
| XXVII-237 | H | Me | H | F | H | F | H | H | H |
| XXVII-238 | H | Me | F | H | H | F | H | H | H |
| XXVII-239 | H | Me | Me | H | H | F | H | H | H |
| XXVII-240 | H | F | H | Me | H | F | H | H | H |
| XXVII-241 | H | F | H | F | H | F | H | H | H |
| XXVII-242 | Me | H | H | H | H | Me | H | H | H |
| XXVII-243 | Et | H | H | H | H | Me | H | H | H |
| XXVII-244 | vinyl | H | H | H | H | Me | H | H | H |
| XXVII-245 | allyl | H | H | H | H | Me | H | H | H |
| XXVII-246 | cyclopropyl | H | H | H | H | Me | H | H | H |
| XXVII-247 | CN | H | H | H | H | Me | H | H | H |
| XXVII-248 | CONH₂ | H | H | H | H | Me | H | H | H |
| XXVII-249 | CONHMe | H | H | H | H | Me | H | H | H |
| XXVII-250 | CONMe₂ | H | H | H | H | Me | H | H | H |
| XXVII-251 | COMe | H | H | H | H | Me | H | H | H |
| XXVII-252 | COOH | H | H | H | H | Me | H | H | H |
| XXVII-253 | COOMe | H | H | H | H | Me | H | H | H |
| XXVII-254 | CSNHMe | H | H | H | H | Me | H | H | H |
| XXVII-255 | H | Me | H | H | H | Me | H | H | H |
| XXVII-256 | H | Et | H | H | H | Me | H | H | H |
| XXVII-257 | H | vinyl | H | H | H | Me | H | H | H |
| XXVII-258 | H | allyl | H | H | H | Me | H | H | H |
| XXVII-259 | H | cyclopropyl | H | H | H | Me | H | H | H |
| XXVII-260 | H | F | H | H | H | Me | H | H | H |
| XXVII-261 | H | Cl | H | H | H | Me | H | H | H |
| XXVII-262 | H | NO₂ | H | H | H | Me | H | H | H |
| XXVII-263 | H | CN | H | H | H | Me | H | H | H |
| XXVII-264 | H | CONH₂ | H | H | H | Me | H | H | H |
| XXVII-265 | H | CONHMe | H | H | H | Me | H | H | H |
| XXVII-266 | H | CONMe₂ | H | H | H | Me | H | H | H |
| XXVII-267 | H | COMe | H | H | H | Me | H | H | H |
| XXVII-268 | H | COOH | H | H | H | Me | H | H | H |
| XXVII-269 | H | COOMe | H | H | H | Me | H | H | H |
| XXVII-270 | H | CSOMe | H | H | H | Me | H | H | H |
| XXVII-271 | H | CSNH₂ | H | H | H | Me | H | H | H |
| XXVII-272 | H | CSNMe₂ | H | H | H | Me | H | H | H |
| XXVII-273 | H | CSNHMe | H | H | H | Me | H | H | H |
| XXVII-274 | H | OMe | H | H | H | Me | H | H | H |
| XXVII-275 | H | OEt | H | H | H | Me | H | H | H |
| XXVII-276 | H | OCOMe | H | H | H | Me | H | H | H |
| XXVII-277 | H | OCOOMe | H | H | H | Me | H | H | H |
| XXVII-278 | H | OCONHMe | H | H | H | Me | H | H | H |
| XXVII-279 | H | OCONMe₂ | H | H | H | Me | H | H | H |
| XXVII-280 | H | OCSMe | H | H | H | Me | H | H | H |
| XXVII-281 | H | OCSNMe₂ | H | H | H | Me | H | H | H |
| XXVII-282 | H | SMe | H | H | H | Me | H | H | H |
| XXVII-283 | H | SEt | H | H | H | Me | H | H | H |
| XXVII-284 | H | SCOMe | H | H | H | Me | H | H | H |
| XXVII-285 | H | SCSNMe₂ | H | H | H | Me | H | H | H |
| XXVII-286 | H | SCSNHMe | H | H | H | Me | H | H | H |
| XXVII-287 | H | NHMe | H | H | H | Me | H | H | H |
| XXVII-288 | H | NH₂ | H | H | H | Me | H | H | H |
| XXVII-289 | H | NMe₂ | H | H | H | Me | H | H | H |
| XXVII-290 | H | NHCOMe | H | H | H | Me | H | H | H |
| XXVII-291 | H | NHCONH₂ | H | H | H | Me | H | H | H |
| XXVII-292 | H | NHCONHMe | H | H | H | Me | H | H | H |
| XXVII-293 | H | NHCONMe₂ | H | H | H | Me | H | H | H |
| XXVII-294 | H | phenyl | H | H | H | Me | H | H | H |
| XXVII-295 | H | 2-chloro- | H | H | H | Me | H | H | H |
| | | phenyl | | | | | | | |
| XXVII-296 | H | 4-nitrophenyl | H | H | H | Me | H | H | H |
| XXVII-297 | H | 2-pyridyl | H | H | H | Me | H | H | H |
| XXVII-298 | H | 3-pyridyl | H | H | H | Me | H | H | H |
| XXVII-299 | H | 4-pyridyl | H | H | H | Me | H | H | H |
| XXVII-300 | H | 2-furyl | H | H | H | Me | H | H | H |
| XXVII-301 | H | PhO | H | H | H | Me | H | H | H |
| XXVII-302 | CH₂ | | H | H | H | Me | H | H | H |
| XXVII-303 | H | H | H | Me | H | Me | H | H | H |
| XXVII-304 | H | H | H | Et | H | Me | H | H | H |
| XXVII-305 | H | H | H | vinyl | H | Me | H | H | H |
| XXVII-306 | H | H | H | allyl | H | Me | H | H | H |
| XXVII-307 | H | H | H | cyclopropyl | H | Me | H | H | H |
| XXVII-308 | H | H | H | F | H | Me | H | H | H |
| XXVII-309 | H | H | H | Cl | H | Me | H | H | H |
| XXVII-310 | H | H | H | NO₂ | H | Me | H | H | H |
| XXVII-311 | H | H | H | CN | H | Me | H | H | H |
| XXVII-312 | H | H | H | CONH₂ | H | Me | H | H | H |
| XXVII-313 | H | H | H | CONHMe | H | Me | H | H | H |
| XXVII-314 | H | H | H | CONMe₂ | H | Me | H | H | H |
| XXVII-315 | H | H | H | COMe | H | Me | H | H | H |
| XXVII-316 | H | H | H | COOH | H | Me | H | H | H |
| XXVII-317 | H | H | H | COOMe | H | Me | H | H | H |
| XXVII-318 | H | H | H | CSOMe | H | Me | H | H | H |
| XXVII-319 | H | H | H | CSNH₂ | H | Me | H | H | H |
| XXVII-320 | H | H | H | CSNMe₂ | H | Me | H | H | H |
| XXVII-321 | H | H | H | CSNHMe | H | Me | H | H | H |
| XXVII-322 | H | H | H | OMe | H | Me | H | H | H |
| XXVII-323 | H | H | H | OEt | H | Me | H | H | H |
| XXVII-324 | H | H | H | OCOMe | H | Me | H | H | H |
| XXVII-325 | H | H | H | OCOOMe | H | Me | H | H | H |
| XXVII-326 | H | H | H | OCONHMe | H | Me | H | H | H |
| XXVII-327 | H | H | H | OCONMe₂ | H | Me | H | H | H |
| XXVII-328 | H | H | H | OCSMe | H | Me | H | H | H |
| XXVII-329 | H | H | H | OCSNMe₂ | H | Me | H | H | H |
| XXVII-330 | H | H | H | SMe | H | Me | H | H | H |
| XXVII-331 | H | H | H | SEt | H | Me | H | H | H |
| XXVII-332 | H | H | H | SCOMe | H | Me | H | H | H |
| XXVII-333 | H | H | H | SCSNMe₂ | H | Me | H | H | H |
| XXVII-334 | H | H | H | SCSNHMe | H | Me | H | H | H |
| XXVII-335 | H | H | H | NHMe | H | Me | H | H | H |
| XXVII-336 | H | H | H | NH₂ | H | Me | H | H | H |
| XXVII-337 | H | H | H | NMe₂ | H | Me | H | H | H |
| XXVII-338 | H | H | H | NHCOMe | H | Me | H | H | H |
| XXVII-339 | H | H | H | NHCONH₂ | H | Me | H | H | H |
| XXVII-340 | H | H | H | NHCONHMe | H | Me | H | H | H |
| XXVII-341 | H | H | H | NHCONMe₂ | H | Me | H | H | H |
| XXVII-342 | H | H | H | phenyl | H | Me | H | H | H |
| XXVII-343 | H | H | H | 2-chloro- | H | Me | H | H | H |
| | | | | phenyl | | | | | |
| XXVII-344 | H | H | H | 4-nitrophenyl | H | Me | H | H | H |
| XXVII-345 | H | H | H | 2-pyridyl | H | Me | H | H | H |
| XXVII-346 | H | H | H | 3-pyridyl | H | Me | H | H | H |
| XXVII-347 | H | H | H | 4-pyridyl | H | Me | H | H | H |
| XXVII-348 | H | H | H | 2-furyl | H | Me | H | H | H |
| XXVII-349 | H | H | H | PhO | H | Me | H | H | H |
| XXVII-350 | H | Me | H | Me | H | Me | H | H | H |
| XXVII-351 | H | Me | F | Me | H | Me | H | H | H |
| XXVII-352 | H | Me | H | F | H | Me | H | H | H |
| XXVII-353 | H | Me | F | H | H | Me | H | H | H |
| XXVII-354 | H | Me | Me | H | H | Me | H | H | H |
| XXVII-355 | H | F | H | Me | H | Me | H | H | H |
| XXVII-356 | H | F | H | F | H | Me | H | H | H |
| XXVII-357 | Me | H | H | H | H | F | H | F | H |
| XXVII-358 | Et | H | H | H | H | F | H | F | H |
| XXVII-359 | vinyl | H | H | H | H | F | H | F | H |
| XXVII-360 | allyl | H | H | H | H | F | H | F | H |
| XXVII-361 | cyclopropyl | H | H | H | H | F | H | F | H |
| XXVII-362 | CN | H | H | H | H | F | H | F | H |
| XXVII-363 | CONH₂ | H | H | H | H | F | H | F | H |
| XXVII-364 | CONHMe | H | H | H | H | F | H | F | H |
| XXVII-365 | CONMe₂ | H | H | H | H | F | H | F | H |
| XXVII-366 | COMe | H | H | H | H | F | H | F | H |
| XXVII-367 | COOH | H | H | H | H | F | H | F | H |
| XXVII-368 | COOMe | H | H | H | H | F | H | F | H |
| XXVII-369 | CSNHMe | H | H | H | H | F | H | F | H |
| XXVII-370 | H | Me | H | H | H | F | H | F | H |
| XXVII-371 | H | Et | H | H | H | F | H | F | H |
| XXVII-372 | H | vinyl | H | H | H | F | H | F | H |
| XXVII-373 | H | allyl | H | H | H | F | H | F | H |
| XXVII-374 | H | cyclopropyl | H | H | H | F | H | F | H |
| XXVII-375 | H | F | H | H | H | F | H | F | H |
| XXVII-376 | H | Cl | H | H | H | F | H | F | H |
| XXVII-377 | H | NO₂ | H | H | H | F | H | F | H |
| XXVII-378 | H | CN | H | H | H | F | H | F | H |
| XXVII-379 | H | CONH₂ | H | H | H | F | H | F | H |
| XXVII-380 | H | CONHMe | H | H | H | F | H | F | H |
| XXVII-381 | H | CONMe₂ | H | H | H | F | H | F | H |
| XXVII-382 | H | COMe | H | H | H | F | H | F | H |
| XXVII-383 | H | COOH | H | H | H | F | H | F | H |
| XXVII-384 | H | COOMe | H | H | H | F | H | F | H |
| XXVII-385 | H | CSOMe | H | H | H | F | H | F | H |
| XXVII-386 | H | CSNH₂ | H | H | H | F | H | F | H |
| XXVII-387 | H | CSNMe₂ | H | H | H | F | H | F | H |
| XXVII-388 | H | CSNHMe | H | H | H | F | H | F | H |
| XXVII-389 | H | OMe | H | H | H | F | H | F | H |
| XXVII-390 | H | OEt | H | H | H | F | H | F | H |
| XXVII-391 | H | OCOMe | H | H | H | F | H | F | H |
| XXVII-392 | H | OCOOMe | H | H | H | F | H | F | H |
| XXVII-393 | H | OCONHMe | H | H | H | F | H | F | H |
| XXVII-394 | H | OCONMe₂ | H | H | H | F | H | F | H |
| XXVII-395 | H | OCSMe | H | H | H | F | H | F | H |
| XXVII-396 | H | OCSNMe₂ | H | H | H | F | H | F | H |
| XXVII-397 | H | SMe | H | H | H | F | H | F | H |
| XXVII-398 | H | SEt | H | H | H | F | H | F | H |
| XXVII-399 | H | SCOMe | H | H | H | F | H | F | H |
| XXVII-400 | H | SCSNMe₂ | H | H | H | F | H | F | H |
| XXVII-401 | H | SCSNHMe | H | H | H | F | H | F | H |
| XXVII-402 | H | NHMe | H | H | H | F | H | F | H |
| XXVII-403 | H | NH₂ | H | H | H | F | H | F | H |
| XXVII-404 | H | NMe₂ | H | H | H | F | H | F | H |
| XXVII-405 | H | NHCOMe | H | H | H | F | H | F | H |
| XXVII-406 | H | NHCONH₂ | H | H | H | F | H | F | H |
| XXVII-407 | H | NHCONHMe | H | H | H | F | H | F | H |
| XXVII-408 | H | NHCONMe₂ | H | H | H | F | H | F | H |
| XXVII-409 | H | phenyl | H | H | H | F | H | F | H |
| XXVII-410 | H | 2-chloro- | H | H | H | F | H | F | H |
| | | phenyl | | | | | | | |
| XXVII-411 | H | 4-nitrophenyl | H | H | H | F | H | F | H |
| XXVII-412 | H | 2-pyridyl | H | H | H | F | H | F | H |
| XXVII-413 | H | 3-pyridyl | H | H | H | F | H | F | H |
| XXVII-414 | H | 4-pyridyl | H | H | H | F | H | F | H |
| XXVII-415 | H | 2-furyl | H | H | H | F | H | F | H |
| XXVII-416 | H | PhO | H | H | H | F | H | F | H |
| XXVII-417 | CH₂ | | H | H | H | F | H | F | H |
| XXVII-418 | H | H | H | Me | H | F | H | F | H |
| XXVII-419 | H | H | H | Et | H | F | H | F | H |
| XXVII-420 | H | H | H | vinyl | H | F | H | F | H |
| XXVII-421 | H | H | H | allyl | H | F | H | F | H |
| XXVII-422 | H | H | H | cyclopropyl | H | F | H | F | H |
| XXVII-423 | H | H | H | F | H | F | H | F | H |
| XXVII-424 | H | H | H | Cl | H | F | H | F | H |
| XXVII-425 | H | H | H | NO₂ | H | F | H | F | H |
| XXVII-426 | H | H | H | CN | H | F | H | F | H |
| XXVII-427 | H | H | H | CONH₂ | H | F | H | F | H |
| XXVII-428 | H | H | H | CONHMe | H | F | H | F | H |
| XXVII-429 | H | H | H | CONMe₂ | H | F | H | F | H |
| XXVII-430 | H | H | H | COMe | H | F | H | F | H |
| XXVII-431 | H | H | H | COOH | H | F | H | F | H |
| XXVII-432 | H | H | H | COOMe | H | F | H | F | H |
| XXVII-433 | H | H | H | CSOMe | H | F | H | F | H |
| XXVII-434 | H | H | H | CSNH₂ | H | F | H | F | H |
| XXVII-435 | H | H | H | CSNMe₂ | H | F | H | F | H |
| XXVII-436 | H | H | H | CSNHMe | H | F | H | F | H |
| XXVII-437 | H | H | H | OMe | H | F | H | F | H |
| XXVII-438 | H | H | H | OEt | H | F | H | F | H |
| XXVII-439 | H | H | H | OCOMe | H | F | H | F | H |
| XXVII-440 | H | H | H | OCOOMe | H | F | H | F | H |
| XXVII-441 | H | H | H | OCONHMe | H | F | H | F | H |
| XXVII-442 | H | H | H | OCONMe₂ | H | F | H | F | H |
| XXVII-443 | H | H | H | OCSMe | H | F | H | F | H |
| XXVII-444 | H | H | H | OCSNMe₂ | H | F | H | F | H |
| XXVII-445 | H | H | H | SMe | H | F | H | F | H |
| XXVII-446 | H | H | H | SEt | H | F | H | F | H |
| XXVII-447 | H | H | H | SCOMe | H | F | H | F | H |
| XXVII-448 | H | H | H | SCSNMe₂ | H | F | H | F | H |
| XXVII-449 | H | H | H | SCSNHMe | H | F | H | F | H |
| XXVII-450 | H | H | H | NHMe | H | F | H | F | H |
| XXVII-451 | H | H | H | NH₂ | H | F | H | F | H |
| XXVII-452 | H | H | H | NMe₂ | H | F | H | F | H |
| XXVII-453 | H | H | H | NHCOMe | H | F | H | F | H |
| XXVII-454 | H | H | H | NHCONH₂ | H | F | H | F | H |
| XXVII-455 | H | H | H | NHCONHMe | H | F | H | F | H |
| XXVII-456 | H | H | H | NHCONMe₂ | H | F | H | F | H |
| XXVII-457 | H | H | H | phenyl | H | F | H | F | H |
| XXVII-458 | H | H | H | 2-chloro- | H | F | H | F | H |
| | | | | phenyl | | | | | |
| XXVII-459 | H | H | H | 4-nitrophenyl | H | F | H | F | H |
| XXVII-460 | H | H | H | 2-pyridyl | H | F | H | F | H |
| XXVII-461 | H | H | H | 3-pyridyl | H | F | H | F | H |
| XXVII-462 | H | H | H | 4-pyridyl | H | F | H | F | H |
| XXVII-463 | H | H | H | 2-furyl | H | F | H | F | H |
| XXVII-464 | H | H | H | PhO | H | F | H | F | H |
| XXVII-465 | H | Me | H | Me | H | F | H | F | H |
| XXVII-466 | H | Me | F | Me | H | F | H | F | H |
| XXVII-467 | H | Me | H | F | H | F | H | F | H |
| XXVII-468 | H | Me | F | H | H | F | H | F | H |
| XXVII-469 | H | Me | Me | H | H | F | H | F | H |
| XXVII-470 | H | F | H | Me | H | F | H | F | H |
| XXVII-471 | H | F | H | F | H | F | H | F | H |
| XXVII-472 | H | H | H | H | H | Me | H | H | H |
| XXVII-473 | H | H | H | H | H | Et | H | H | H |
| XXVII-474 | H | H | H | H | H | vinyl | H | H | H |
| XXVII-475 | H | H | H | H | H | allyl | H | H | H |
| XXVII-476 | H | H | H | H | H | cyclopropyl | H | H | H |
| XXVII-477 | H | H | H | H | H | F | H | H | H |
| XXVII-478 | H | H | H | H | H | Cl | H | H | H |
| XXVII-479 | H | H | H | H | H | NO₂ | H | H | H |
| XXVII-480 | H | H | H | H | H | CN | H | H | H |
| XXVII-481 | H | H | H | H | H | CONH₂ | H | H | H |
| XXVII-482 | H | H | H | H | H | CONHMe | H | H | H |
| XXVII-483 | H | H | H | H | H | CONMe₂ | H | H | H |
| XXVII-484 | H | H | H | H | H | COMe | H | H | H |
| XXVII-485 | H | H | H | H | H | COOH | H | H | H |
| XXVII-486 | H | H | H | H | H | COOMe | H | H | H |
| XXVII-487 | H | H | H | H | H | CSOMe | H | H | H |
| XXVII-488 | H | H | H | H | H | CSNH₂ | H | H | H |
| XXVII-489 | H | H | H | H | H | CSNMe₂ | H | H | H |
| XXVII-490 | H | H | H | H | H | CSNHMe | H | H | H |
| XXVII-491 | H | H | H | H | H | OMe | H | H | H |
| XXVII-492 | H | H | H | H | H | OEt | H | H | H |
| XXVII-493 | H | H | H | H | H | OCOMe | H | H | H |
| XXVII-494 | H | H | H | H | H | OCOOMe | H | H | H |
| XXVII-495 | H | H | H | H | H | OCONHMe | H | H | H |
| XXVII-496 | H | H | H | H | H | OCONMe₂ | H | H | H |
| XXVII-497 | H | H | H | H | H | OCSMe | H | H | H |
| XXVII-498 | H | H | H | H | H | OCSNMe₂ | H | H | H |
| XXVII-499 | H | H | H | H | H | SMe | H | H | H |
| XXVII-500 | H | H | H | H | H | SEt | H | H | H |
| XXVII-501 | H | H | H | H | H | SCOMe | H | H | H |
| XXVII-502 | H | H | H | H | H | SCSNMe₂ | H | H | H |
| XXVII-503 | H | H | H | H | H | SCSNHMe | H | H | H |
| XXVII-504 | H | H | H | H | H | NHMe | H | H | H |
| XXVII-505 | H | H | H | H | H | NH₂ | H | H | H |
| XXVII-506 | H | H | H | H | H | NMe₂ | H | H | H |
| XXVII-507 | H | H | H | H | H | NHCOMe | H | H | H |
| XXVII-508 | H | H | H | H | H | NHCONH₂ | H | H | H |
| XXVII-509 | H | H | H | H | H | NHCONHMe | H | H | H |
| XXVII-510 | H | H | H | H | H | NHCONMe₂ | H | H | H |
| XXVII-511 | H | H | H | H | H | phenyl | H | H | H |
| XXVII-512 | H | H | H | H | H | 2-chloro- | H | H | H |
| | | | | | | phenyl | | | |
| XXVII-513 | H | H | H | H | H | 4-nitrophenyl | H | H | H |
| XXVII-514 | H | H | H | H | H | 2-pyridyl | H | H | H |
| XXVII-515 | H | H | H | H | H | 3-pyridyl | H | H | H |
| XXVII-516 | H | H | H | H | H | 4-pyridyl | H | H | H |
| XXVII-517 | H | H | H | H | H | 2-furyl | H | H | H |
| XXVII-518 | H | H | H | H | H | PhO | H | H | H |
| XXVII-519 | H | H | H | H | H | H | H | Me | H |
| XXVII-520 | H | H | H | H | H | H | H | Et | H |
| XXVII-521 | H | H | H | H | H | H | H | vinyl | H |
| XXVII-522 | H | H | H | H | H | H | H | allyl | H |
| XXVII-523 | H | H | H | H | H | H | H | cyclopropyl | H |
| XXVII-524 | H | H | H | H | H | H | H | F | H |
| XXVII-525 | H | H | H | H | H | H | H | Cl | H |
| XXVII-526 | H | H | H | H | H | H | H | NO₂ | H |
| XXVII-527 | H | H | H | H | H | H | H | CN | H |
| XXVII-528 | H | H | H | H | H | H | H | CONH₂ | H |
| XXVII-529 | H | H | H | H | H | H | H | CONHMe | H |
| XXVII-530 | H | H | H | H | H | H | H | CONMe₂ | H |
| XXVII-531 | H | H | H | H | H | H | H | COMe | H |
| XXVII-532 | H | H | H | H | H | H | H | COOH | H |
| XXVII-533 | H | H | H | H | H | H | H | COOMe | H |
| XXVII-534 | H | H | H | H | H | H | H | CSOMe | H |
| XXVII-535 | H | H | H | H | H | H | H | CSNH₂ | H |
| XXVII-536 | H | H | H | H | H | H | H | CSNMe₂ | H |
| XXVII-537 | H | H | H | H | H | H | H | CSNHMe | H |
| XXVII-538 | H | H | H | H | H | H | H | OMe | H |
| XXVII-539 | H | H | H | H | H | H | H | OEt | H |
| XXVII-540 | H | H | H | H | H | H | H | OCOMe | H |
| XXVII-541 | H | H | H | H | H | H | H | OCOOMe | H |
| XXVII-542 | H | H | H | H | H | H | H | OCONHMe | H |
| XXVII-543 | H | H | H | H | H | H | H | OCONMe₂ | H |
| XXVII-544 | H | H | H | H | H | H | H | OCSMe | H |
| XXVII-545 | H | H | H | H | H | H | H | OCSNMe₂ | H |
| XXVII-546 | H | H | H | H | H | H | H | SMe | H |
| XXVII-547 | H | H | H | H | H | H | H | SEt | H |
| XXVII-548 | H | H | H | H | H | H | H | SCOMe | H |
| XXVII-549 | H | H | H | H | H | H | H | SCSNMe₂ | H |
| XXVII-550 | H | H | H | H | H | H | H | SCSNHMe | H |
| XXVII-551 | H | H | H | H | H | H | H | NHMe | H |
| XXVII-552 | H | H | H | H | H | H | H | NH₂ | H |
| XXVII-553 | H | H | H | H | H | H | H | NMe₂ | H |
| XXVII-554 | H | H | H | H | H | H | H | NHCOMe | H |
| XXVII-555 | H | H | H | H | H | H | H | NHCONH₂ | H |
| XXVII-556 | H | H | H | H | H | H | H | NHCONHMe | H |
| XXVII-557 | H | H | H | H | H | H | H | NHCONMe₂ | H |
| XXVII-558 | H | H | H | H | H | H | H | phenyl | H |
| XXVII-559 | H | H | H | H | H | H | H | 2-chloro- | H |
| | | | | | | | | phenyl | |
| XXVII-560 | H | H | H | H | H | H | H | 4-nitrophenyl | H |
| XXVII-561 | H | H | H | H | H | H | H | 2-pyridyl | H |
| XXVII-562 | H | H | H | H | H | H | H | 3-pyridyl | H |
| XXVII-563 | H | H | H | H | H | H | H | 4-pyridyl | H |
| XXVII-564 | H | H | H | H | H | H | H | 2-furyl | H |
| XXVII-565 | H | H | H | H | H | H | H | PhO | H |
| XXVII-566 | H | H | H | H | H | H | H | H | Me |
| XXVII-567 | H | H | H | H | H | H | H | H | Et |
| XXVII-568 | H | H | H | H | H | H | H | H | vinyl |
| XXVII-569 | H | H | H | H | H | H | H | H | allyl |
| XXVII-570 | H | H | H | H | H | H | H | H | cyclopropyl |
| XXVII-571 | H | H | H | H | H | H | H | H | F |
| XXVII-572 | H | H | H | H | H | H | H | H | Cl |
| XXVII-573 | H | H | H | H | H | H | H | H | NO₂ |
| XXVII-574 | H | H | H | H | H | H | H | H | CN |
| XXVII-575 | H | H | H | H | H | H | H | H | CONH₂ |
| XXVII-576 | H | H | H | H | H | H | H | H | CONHMe |
| XXVII-577 | H | H | H | H | H | H | H | H | CONMe₂ |
| XXVII-578 | H | H | H | H | H | H | H | H | COMe |
| XXVII-579 | H | H | H | H | H | H | H | H | COOH |
| XXVII-580 | H | H | H | H | H | H | H | H | COOMe |
| XXVII-581 | H | H | H | H | H | H | H | H | CSOMe |
| XXVII-582 | H | H | H | H | H | H | H | H | CSNH₂ |
| XXVII-583 | H | H | H | H | H | H | H | H | CSNMe₂ |
| XXVII-584 | H | H | H | H | H | H | H | H | CSNHMe |
| XXVII-585 | H | H | H | H | H | H | H | H | OMe |
| XXVII-586 | H | H | H | H | H | H | H | H | OEt |
| XXVII-587 | H | H | H | H | H | H | H | H | OCOMe |
| XXVII-588 | H | H | H | H | H | H | H | H | OCOOMe |
| XXVII-589 | H | H | H | H | H | H | H | H | OCONHMe |
| XXVII-590 | H | H | H | H | H | H | H | H | OCONMe₂ |
| XXVII-591 | H | H | H | H | H | H | H | H | OCSMe |
| XXVII-592 | H | H | H | H | H | H | H | H | OCSNMe₂ |
| XXVII-593 | H | H | H | H | H | H | H | H | SMe |
| XXVII-594 | H | H | H | H | H | H | H | H | SEt |
| XXVII-595 | H | H | H | H | H | H | H | H | SCOMe |
| XXVII-596 | H | H | H | H | H | H | H | H | SCSNMe₂ |
| XXVII-597 | H | H | H | H | H | H | H | H | SCSNHMe |
| XXVII-598 | H | H | H | H | H | H | H | H | NHMe |
| XXVII-599 | H | H | H | H | H | H | H | H | NH₂ |
| XXVII-600 | H | H | H | H | H | H | H | H | NMe₂ |
| XXVII-601 | H | H | H | H | H | H | H | H | NHCOMe |
| XXVII-602 | H | H | H | H | H | H | H | H | NHCONH₂ |
| XXVII-603 | H | H | H | H | H | H | H | H | NHCONHMe |
| XXVII-604 | H | H | H | H | H | H | H | H | NHCONMe₂ |
| XXVII-605 | H | H | H | H | H | H | H | H | phenyl |
| XXVII-606 | H | H | H | H | H | H | H | H | 2-chloro- |
| | | | | | | | | | phenyl |
| XXVII-607 | H | H | H | H | H | H | H | H | 4-nitrophenyl |
| XXVII-608 | H | H | H | H | H | H | H | H | 2-pyridyl |
| XXVII-609 | H | H | H | H | H | H | H | H | 3-pyridyl |
| XXVII-610 | H | H | H | H | H | H | H | H | 4-pyridyl |
| XXVII-611 | H | H | H | H | H | H | H | H | 2-furyl |
| XXVII-612 | H | H | H | H | H | H | H | H | PhO |

Table XXVIII provides 422 compounds of formula Iab

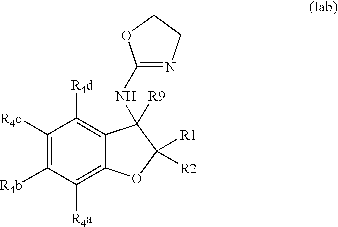

wherein the values of R¹, R², R^{4a}, R^{4b}, R^{4c}, R^{4d} and R⁹ are given in Table 4

TABLE 4

| Compound | | | | | | | |
|---|---|---|---|---|---|---|---|
| No | R9 | R1 | R2 | R4a | R4b | R4c | R4d |
| XXVIII-1 | Me | H | H | H | H | H | H |
| XXVIII-2 | Et | H | H | H | H | H | H |
| XXVIII-3 | vinyl | H | H | H | H | H | H |
| XXVIII-4 | allyl | H | H | H | H | H | H |
| XXVIII-5 | cyclopropyl | H | H | H | H | H | H |
| XXVIII-6 | CN | H | H | H | H | H | H |
| XXVIII-7 | CONH₂ | H | H | H | H | H | H |
| XXVIII-8 | CONHMe | H | H | H | H | H | H |
| XXVIII-9 | CONMe₂ | H | H | H | H | H | H |
| XXVIII-10 | COMe | H | H | H | H | H | H |
| XXVIII-11 | COOH | H | H | H | H | H | H |
| XXVIII-12 | COOMe | H | H | H | H | H | H |
| XXVIII-13 | CSNHMe | H | H | H | H | H | H |
| XXVIII-14 | H | Me | H | H | H | H | H |
| XXVIII-15 | H | Et | H | H | H | H | H |
| XXVIII-16 | H | Vinyl | H | H | H | H | H |
| XXVIII-17 | H | Allyl | H | H | H | H | H |
| XXVIII-18 | H | Cyclopropyl | H | H | H | H | H |
| XXVIII-19 | H | F | H | H | H | H | H |
| XXVIII-20 | H | Cl | H | H | H | H | H |
| XXVIII-21 | H | NO₂ | H | H | H | H | H |
| XXVIII-22 | H | CN | H | H | H | H | H |
| XXVIII-23 | H | CONH₂ | H | H | H | H | H |
| XXVIII-24 | H | CONHMe | H | H | H | H | H |
| XXVIII-25 | H | CONMe₂ | H | H | H | H | H |
| XXVIII-26 | H | COMe | H | H | H | H | H |
| XXVIII-27 | H | COOH | H | H | H | H | H |
| XXVIII-28 | H | COOMe | H | H | H | H | H |
| XXVIII-29 | H | CSOMe | H | H | H | H | H |
| XXVIII-30 | H | CSNH₂ | H | H | H | H | H |
| XXVIII-31 | H | CSNMe₂ | H | H | H | H | H |
| XXVIII-32 | H | CSNHMe | H | H | H | H | H |
| XXVIII-33 | H | OMe | H | H | H | H | H |
| XXVIII-34 | H | OEt | H | H | H | H | H |
| XXVIII-35 | H | OCOMe | H | H | H | H | H |
| XXVIII-36 | H | OCOOMe | H | H | H | H | H |
| XXVIII-37 | H | OCONHMe | H | H | H | H | H |
| XXVIII-38 | H | OCONMe₂ | H | H | H | H | H |
| XXVIII-39 | H | OCSMe | H | H | H | H | H |
| XXVIII-40 | H | OCSNMe₂ | H | H | H | H | H |
| XXVIII-41 | H | SMe | H | H | H | H | H |
| XXVIII-42 | H | SEt | H | H | H | H | H |
| XXVIII-43 | H | SCOMe | H | H | H | H | H |
| XXVIII-44 | H | SCSNMe₂ | H | H | H | H | H |
| XXVIII-45 | H | SCSNHMe | H | H | H | H | H |
| XXVIII-46 | H | NHMe | H | H | H | H | H |
| XXVIII-47 | H | NH₂ | H | H | H | H | H |
| XXVIII-48 | H | NMe₂ | H | H | H | H | H |
| XXVIII-49 | H | NHCOMe | H | H | H | H | H |
| XXVIII-50 | H | NHCONH₂ | H | H | H | H | H |
| XXVIII-51 | H | NHCONHMe | H | H | H | H | H |
| XXVIII-52 | H | NHCONMe₂ | H | H | H | H | H |
| XXVIII-53 | H | Phenyl | H | H | H | H | H |
| XXVIII-54 | H | 2-chloro- | H | H | H | H | H |
| | | phenyl | | | | | |
| XXVIII-55 | H | 4-nitrophenyl | H | H | H | H | H |
| XXVIII-56 | H | 2-pyridyl | H | H | H | H | H |
| XXVIII-57 | H | 3-pyridyl | H | H | H | H | H |
| XXVIII-58 | H | 4-pyridyl | H | H | H | H | H |
| XXVIII-59 | H | 2-furyl | H | H | H | H | H |
| XXVIII-60 | H | PhO | H | H | H | H | H |
| XXVIII-61 | CH₂ | | H | H | H | H | H |
| XXVIII-62 | H | ═O | | H | H | H | H |
| XXVIII-63 | H | ═NOH | | H | H | H | H |
| XXVIII-64 | H | ═NOMe | | H | H | H | H |
| XXVIII-65 | H | ═CH₂ | | H | H | H | H |
| XXVIII-66 | H | ═CHMe | | H | H | H | H |
| XXVIII-67 | H | H | H | H | H | H | H |
| XXVIII-68 | H | Me | H | H | H | H | H |
| XXVIII-69 | H | Me | F | H | H | H | H |
| XXVIII-70 | H | Et | H | H | H | H | H |
| XXVIII-71 | H | Me | Me | H | H | H | H |
| XXVIII-72 | Me | H | Me | H | H | H | H |
| XXVIII-73 | H | F | H | H | H | H | H |
| XXVIII-74 | Me | F | H | H | H | H | H |
| XXVIII-75 | Me | H | H | F | H | H | H |
| XXVIII-76 | Et | H | H | F | H | H | H |
| XXVIII-77 | vinyl | H | H | F | H | H | H |
| XXVIII-78 | allyl | H | H | F | H | H | H |
| XXVIII-79 | cyclopropyl | H | H | F | H | H | H |
| XXVIII-80 | CN | H | H | F | H | H | H |
| XXVIII-81 | CONH₂ | H | H | F | H | H | H |
| XXVIII-82 | CONHMe | H | H | F | H | H | H |
| XXVIII-83 | CONMe₂ | H | H | F | H | H | H |
| XXVIII-84 | COMe | H | H | F | H | H | H |
| XXVIII-85 | COOH | H | H | F | H | H | H |
| XXVIII-86 | COOMe | H | H | F | H | H | H |
| XXVIII-87 | CSNHMe | H | H | F | H | H | H |
| XXVIII-88 | H | Me | H | F | H | H | H |
| XXVIII-89 | H | Et | H | F | H | H | H |
| XXVIII-90 | H | vinyl | H | F | H | H | H |
| XXVIII-91 | H | allyl | H | F | H | H | H |
| XXVIII-92 | H | cyclopropyl | H | F | H | H | H |
| XXVIII-93 | H | F | H | F | H | H | H |
| XXVIII-94 | H | Cl | H | F | H | H | H |
| XXVIII-95 | H | NO₂ | H | F | H | H | H |
| XXVIII-96 | H | CN | H | F | H | H | H |
| XXVIII-97 | H | CONH₂ | H | F | H | H | H |
| XXVIII-98 | H | CONHMe | H | F | H | H | H |
| XXVIII-99 | H | CONMe₂ | H | F | H | H | H |
| XXVIII-100 | H | COMe | H | F | H | H | H |
| XXVIII-101 | H | COOH | H | F | H | H | H |
| XXVIII-102 | H | COOMe | H | F | H | H | H |
| XXVIII-103 | H | CSOMe | H | F | H | H | H |
| XXVIII-104 | H | CSNH₂ | H | F | H | H | H |
| XXVIII-105 | H | CSNMe₂ | H | F | H | H | H |
| XXVIII-106 | H | CSNHMe | H | F | H | H | H |
| XXVIII-107 | H | OMe | H | F | H | H | H |
| XXVIII-108 | H | OEt | H | F | H | H | H |
| XXVIII-109 | H | OCOMe | H | F | H | H | H |
| XXVIII-110 | H | OCOOMe | H | F | H | H | H |
| XXVIII-111 | H | OCONHMe | H | F | H | H | H |
| XXVIII-112 | H | OCONMe₂ | H | F | H | H | H |
| XXVIII-113 | H | OCSMe | H | F | H | H | H |
| XXVIII-114 | H | OCSNMe₂ | H | F | H | H | H |
| XXVIII-115 | H | SMe | H | F | H | H | H |
| XXVIII-116 | H | SEt | H | F | H | H | H |
| XXVIII-117 | H | SCOMe | H | F | H | H | H |
| XXVIII-118 | H | SCSNMe₂ | H | F | H | H | H |
| XXVIII-119 | H | SCSNHMe | H | F | H | H | H |
| XXVIII-120 | H | NHMe | H | F | H | H | H |
| XXVIII-121 | H | NH₂ | H | F | H | H | H |
| XXVIII-122 | H | NMe₂ | H | F | H | H | H |
| XXVIII-123 | H | NHCOMe | H | F | H | H | H |
| XXVIII-124 | H | NHCONH₂ | H | F | H | H | H |
| XXVIII-125 | H | NHCONHMe | H | F | H | H | H |
| XXVIII-126 | H | NHCONMe₂ | H | F | H | H | H |
| XXVIII-127 | H | phenyl | H | F | H | H | H |
| XXVIII-128 | H | 2-chloro- | H | F | H | H | H |
| | | phenyl | | | | | |
| XXVIII-129 | H | 4-nitrophenyl | H | F | H | H | H |
| XXVIII-130 | H | 2-pyridyl | H | F | H | H | H |
| XXVIII-131 | H | 3-pyridyl | H | F | H | H | H |
| XXVIII-132 | H | 4-pyridyl | H | F | H | H | H |
| XXVIII-133 | H | 2-furyl | H | F | H | H | H |
| XXVIII-134 | H | PhO | H | F | H | H | H |
| XXVIII-135 | CH₂ | | H | F | H | H | H |
| XXVIII-136 | H | H | H | F | H | H | H |
| XXVIII-137 | H | Me | H | F | H | H | H |
| XXVIII-138 | H | Me | F | F | H | H | H |
| XXVIII-139 | H | Et | H | F | H | H | H |
| XXVIII-140 | H | Me | Me | F | H | H | H |
| XXVIII-141 | Me | H | Me | F | H | H | H |
| XXVIII-142 | H | F | H | F | H | H | H |
| XXVIII-143 | Me | F | H | F | H | H | H |
| XXVIII-144 | Me | H | H | Me | H | H | H |
| XXVIII-145 | Et | H | H | Me | H | H | H |
| XXVIII-146 | vinyl | H | H | Me | H | H | H |
| XXVIII-147 | allyl | H | H | Me | H | H | H |
| XXVIII-148 | cyclopropyl | H | H | Me | H | H | H |
| XXVIII-149 | CN | H | H | Me | H | H | H |
| XXVIII-150 | CONH₂ | H | H | Me | H | H | H |
| XXVIII-151 | CONHMe | H | H | Me | H | H | H |
| XXVIII-152 | CONMe₂ | H | H | Me | H | H | H |
| XXVIII-153 | COMe | H | H | Me | H | H | H |
| XXVIII-154 | COOH | H | H | Me | H | H | H |
| XXVIII-155 | COOMe | H | H | Me | H | H | H |
| XXVIII-156 | CSNHMe | H | H | Me | H | H | H |
| XXVIII-157 | H | Me | H | Me | H | H | H |
| XXVIII-158 | H | Et | H | Me | H | H | H |
| XXVIII-159 | H | vinyl | H | Me | H | H | H |
| XXVIII-160 | H | allyl | H | Me | H | H | H |
| XXVIII-161 | H | cyclopropyl | H | Me | H | H | H |
| XXVIII-162 | H | F | H | Me | H | H | H |
| XXVIII-163 | H | Cl | H | Me | H | H | H |
| XXVIII-164 | H | NO₂ | H | Me | H | H | H |
| XXVIII-165 | H | CN | H | Me | H | H | H |
| XXVIII-166 | H | CONH₂ | H | Me | H | H | H |
| XXVIII-167 | H | CONHMe | H | Me | H | H | H |
| XXVIII-168 | H | CONMe₂ | H | Me | H | H | H |
| XXVIII-169 | H | COMe | H | Me | H | H | H |
| XXVIII-170 | H | COOH | H | Me | H | H | H |
| XXVIII-171 | H | COOMe | H | Me | H | H | H |
| XXVIII-172 | H | CSOMe | H | Me | H | H | H |
| XXVIII-173 | H | CSNH₂ | H | Me | H | H | H |
| XXVIII-174 | H | CSNMe₂ | H | Me | H | H | H |
| XXVIII-175 | H | CSNHMe | H | Me | H | H | H |
| XXVIII-176 | H | OMe | H | Me | H | H | H |
| XXVIII-177 | H | OEt | H | Me | H | H | H |
| XXVIII-178 | H | OCOMe | H | Me | H | H | H |
| XXVIII-179 | H | OCOOMe | H | Me | H | H | H |
| XXVIII-180 | H | OCONHMe | H | Me | H | H | H |
| XXVIII-181 | H | OCONMe₂ | H | Me | H | H | H |
| XXVIII-182 | H | OCSMe | H | Me | H | H | H |
| XXVIII-183 | H | OCSNMe₂ | H | Me | H | H | H |
| XXVIII-184 | H | SMe | H | Me | H | H | H |
| XXVIII-185 | H | SEt | H | Me | H | H | H |
| XXVIII-186 | H | SCOMe | H | Me | H | H | H |
| XXVIII-187 | H | SCSNMe₂ | H | Me | H | H | H |
| XXVIII-188 | H | SCSNHMe | H | Me | H | H | H |
| XXVIII-189 | H | NHMe | H | Me | H | H | H |
| XXVIII-190 | H | NH₂ | H | Me | H | H | H |
| XXVIII-191 | H | NMe₂ | H | Me | H | H | H |
| XXVIII-192 | H | NHCOMe | H | Me | H | H | H |
| XXVIII-193 | H | NHCONH₂ | H | Me | H | H | H |
| XXVIII-194 | H | NHCONHMe | H | Me | H | H | H |
| XXVIII-195 | H | NHCONMe₂ | H | Me | H | H | H |
| XXVIII-196 | H | phenyl | H | Me | H | H | H |
| XXVIII-197 | H | 2-chloro- | H | Me | H | H | H |
| | | phenyl | | | | | |
| XXVIII-198 | H | 4-nitrophenyl | H | Me | H | H | H |
| XXVIII-199 | H | 2-pyridyl | H | Me | H | H | H |
| XXVIII-200 | H | 3-pyridyl | H | Me | H | H | H |
| XXVIII-201 | H | 4-pyridyl | H | Me | H | H | H |
| XXVIII-202 | H | 2-furyl | H | Me | H | H | H |
| XXVIII-203 | H | PhO | H | Me | H | H | H |
| XXVIII-204 | CH₂ | | H | Me | H | H | H |
| XXVIII-205 | H | H | H | Me | H | H | H |
| XXVIII-206 | H | Me | H | Me | H | H | H |
| XXVIII-207 | H | Me | F | Me | H | H | H |
| XXVIII-208 | H | Et | H | Me | H | H | H |
| XXVIII-209 | H | Me | Me | Me | H | H | H |
| XXVIII-210 | Me | H | Me | Me | H | H | H |
| XXVIII-211 | H | F | H | Me | H | H | H |
| XXVIII-212 | Me | F | H | Me | H | H | H |
| XXVIII-213 | Me | H | H | F | H | F | H |
| XXVIII-214 | Et | H | H | F | H | F | H |
| XXVIII-215 | vinyl | H | H | F | H | F | H |
| XXVIII-216 | allyl | H | H | F | H | F | H |
| XXVIII-217 | cyclopropyl | H | H | F | H | F | H |
| XXVIII-218 | CN | H | H | F | H | F | H |
| XXVIII-219 | CONH₂ | H | H | F | H | F | H |
| XXVIII-220 | CONHMe | H | H | F | H | F | H |
| XXVIII-221 | CONMe₂ | H | H | F | H | F | H |
| XXVIII-222 | COMe | H | H | F | H | F | H |
| XXVIII-223 | COOH | H | H | F | H | F | H |
| XXVIII-224 | COOMe | H | H | F | H | F | H |
| XXVIII-225 | CSNHMe | H | H | F | H | F | H |
| XXVIII-226 | H | Me | H | F | H | F | H |
| XXVIII-227 | H | Et | H | F | H | F | H |
| XXVIII-228 | H | vinyl | H | F | H | F | H |
| XXVIII-229 | H | allyl | H | F | H | F | H |
| XXVIII-230 | H | cyclopropyl | H | F | H | F | H |
| XXVIII-231 | H | F | H | F | H | F | H |
| XXVIII-232 | H | Cl | H | F | H | F | H |
| XXVIII-233 | H | NO₂ | H | F | H | F | H |
| XXVIII-234 | H | CN | H | F | H | F | H |
| XXVIII-235 | H | CONH₂ | H | F | H | F | H |
| XXVIII-236 | H | CONHMe | H | F | H | F | H |
| XXVIII-237 | H | CONMe₂ | H | F | H | F | H |
| XXVIII-238 | H | COMe | H | F | H | F | H |
| XXVIII-239 | H | COOH | H | F | H | F | H |
| XXVIII-240 | H | COOMe | H | F | H | F | H |
| XXVIII-241 | H | CSOMe | H | F | H | F | H |
| XXVIII-242 | H | CSNH₂ | H | F | H | F | H |
| XXVIII-243 | H | CSNMe₂ | H | F | H | F | H |
| XXVIII-244 | H | CSNHMe | H | F | H | F | H |
| XXVIII-245 | H | OMe | H | F | H | F | H |
| XXVIII-246 | H | OEt | H | F | H | F | H |
| XXVIII-247 | H | OCOMe | H | F | H | F | H |
| XXVIII-248 | H | OCOOMe | H | F | H | F | H |
| XXVIII-249 | H | OCONHMe | H | F | H | F | H |
| XXVIII-250 | H | OCONMe₂ | H | F | H | F | H |
| XXVIII-251 | H | OCSMe | H | F | H | F | H |
| XXVIII-252 | H | OCSNMe₂ | H | F | H | F | H |
| XXVIII-253 | H | SMe | H | F | H | F | H |
| XXVIII-254 | H | SEt | H | F | H | F | H |
| XXVIII-255 | H | SCOMe | H | F | H | F | H |
| XXVIII-256 | H | SCSNMe₂ | H | F | H | F | H |
| XXVIII-257 | H | SCSNHMe | H | F | H | F | H |
| XXVIII-258 | H | NHMe | H | F | H | F | H |
| XXVIII-259 | H | NH₂ | H | F | H | F | H |
| XXVIII-260 | H | NMe₂ | H | F | H | F | H |
| XXVIII-261 | H | NHCOMe | H | F | H | F | H |
| XXVIII-262 | H | NHCONH₂ | H | F | H | F | H |
| XXVIII-263 | H | NHCONHMe | H | F | H | F | H |
| XXVIII-264 | H | NHCONMe₂ | H | F | H | F | H |
| XXVIII-265 | H | phenyl | H | F | H | F | H |
| XXVIII-266 | H | 2-chloro- | H | F | H | F | H |
| | | phenyl | | | | | |
| XXVIII-267 | H | 4-nitrophenyl | H | F | H | F | H |
| XXVIII-268 | H | 2-pyridyl | H | F | H | F | H |
| XXVIII-269 | H | 3-pyridyl | H | F | H | F | H |
| XXVIII-270 | H | 4-pyridyl | H | F | H | F | H |
| XXVIII-271 | H | 2-furyl | H | F | H | F | H |
| XXVIII-272 | H | PhO | H | F | H | F | H |
| XXVIII-273 | CH₂ | | H | F | H | F | H |
| XXVIII-274 | H | H | H | F | H | F | H |
| XXVIII-275 | H | Me | H | F | H | F | H |
| XXVIII-276 | H | Me | F | F | H | F | H |
| XXVIII-277 | H | Et | H | F | H | F | H |
| XXVIII-278 | H | Me | Me | F | H | F | H |
| XXVIII-279 | Me | H | Me | F | H | F | H |
| XXVIII-280 | H | F | H | F | H | F | H |
| XXVIII-281 | Me | F | H | F | H | F | H |
| XXVIII-282 | H | H | H | Me | H | H | H |
| XXVIII-283 | H | H | H | Et | H | H | H |
| XXVIII-284 | H | H | H | vinyl | H | H | H |
| XXVIII-285 | H | H | H | allyl | H | H | H |
| XXVIII-286 | H | H | H | cyclopropyl | H | H | H |
| XXVIII-287 | H | H | H | F | H | H | H |
| XXVIII-288 | H | H | H | Cl | H | H | H |
| XXVIII-289 | H | H | H | NO₂ | H | H | H |
| XXVIII-290 | H | H | H | CN | H | H | H |
| XXVIII-291 | H | H | H | CONH₂ | H | H | H |
| XXVIII-292 | H | H | H | CONHMe | H | H | H |
| XXVIII-293 | H | H | H | CONMe₂ | H | H | H |
| XXVIII-294 | H | H | H | COMe | H | H | H |
| XXVIII-295 | H | H | H | COOH | H | H | H |
| XXVIII-296 | H | H | H | COOMe | H | H | H |
| XXVIII-297 | H | H | H | CSOMe | H | H | H |
| XXVIII-298 | H | H | H | CSNH₂ | H | H | H |
| XXVIII-299 | H | H | H | CSNMe₂ | H | H | H |
| XXVIII-300 | H | H | H | CSNHMe | H | H | H |
| XXVIII-301 | H | H | H | OMe | H | H | H |
| XXVIII-302 | H | H | H | OEt | H | H | H |
| XXVIII-303 | H | H | H | OCOMe | H | H | H |
| XXVIII-304 | H | H | H | OCOOMe | H | H | H |
| XXVIII-305 | H | H | H | OCONHMe | H | H | H |
| XXVIII-306 | H | H | H | OCONMe₂ | H | H | H |
| XXVIII-307 | H | H | H | OCSMe | H | H | H |
| XXVIII-308 | H | H | H | OCSNMe₂ | H | H | H |
| XXVIII-309 | H | H | H | SMe | H | H | H |
| XXVIII-310 | H | H | H | SEt | H | H | H |
| XXVIII-311 | H | H | H | SCOMe | H | H | H |
| XXVIII-312 | H | H | H | SCSNMe₂ | H | H | H |
| XXVIII-313 | H | H | H | SCSNHMe | H | H | H |
| XXVIII-314 | H | H | H | NHMe | H | H | H |
| XXVIII-315 | H | H | H | NH₂ | H | H | H |
| XXVIII-316 | H | H | H | NMe₂ | H | H | H |
| XXVIII-317 | H | H | H | NHCOMe | H | H | H |
| XXVIII-318 | H | H | H | NHCONH₂ | H | H | H |
| XXVIII-319 | H | H | H | NHCONHMe | H | H | H |
| XXVIII-320 | H | H | H | NHCONMe₂ | H | H | H |
| XXVIII-321 | H | H | H | phenyl | H | H | H |
| XXVIII-322 | H | H | H | 2-chloro- | H | H | H |
| | | | | phenyl | | | |
| XXVIII-323 | H | H | H | 4-nitrophenyl | H | H | H |
| XXVIII-324 | H | H | H | 2-pyridyl | H | H | H |
| XXVIII-325 | H | H | H | 3-pyridyl | H | H | H |
| XXVIII-326 | H | H | H | 4-pyridyl | H | H | H |
| XXVIII-327 | H | H | H | 2-furyl | H | H | H |
| XXVIII-328 | H | H | H | PhO | H | H | H |
| XXVIII-329 | H | H | H | H | H | Me | H |
| XXVIII-330 | H | H | H | H | H | Et | H |
| XXVIII-331 | H | H | H | H | H | vinyl | H |
| XXVIII-332 | H | H | H | H | H | allyl | H |
| XXVIII-333 | H | H | H | H | H | cyclopropyl | H |
| XXVIII-334 | H | H | H | H | H | F | H |
| XXVIII-335 | H | H | H | H | H | Cl | H |
| XXVIII-336 | H | H | H | H | H | NO₂ | H |
| XXVIII-337 | H | H | H | H | H | CN | H |
| XXVIII-338 | H | H | H | H | H | CONH₂ | H |
| XXVIII-339 | H | H | H | H | H | CONHMe | H |
| XXVIII-340 | H | H | H | H | H | CONMe₂ | H |
| XXVIII-341 | H | H | H | H | H | COMe | H |
| XXVIII-342 | H | H | H | H | H | COOH | H |
| XXVIII-343 | H | H | H | H | H | COOMe | H |
| XXVIII-344 | H | H | H | H | H | CSOMe | H |
| XXVIII-345 | H | H | H | H | H | CSNH₂ | H |
| XXVIII-346 | H | H | H | H | H | CSNMe₂ | H |
| XXVIII-347 | H | H | H | H | H | CSNHMe | H |
| XXVIII-348 | H | H | H | H | H | OMe | H |
| XXVIII-349 | H | H | H | H | H | OEt | H |
| XXVIII-350 | H | H | H | H | H | OCOMe | H |
| XXVIII-351 | H | H | H | H | H | OCOOMe | H |
| XXVIII-352 | H | H | H | H | H | OCONHMe | H |
| XXVIII-353 | H | H | H | H | H | OCONMe₂ | H |
| XXVIII-354 | H | H | H | H | H | OCSMe | H |
| XXVIII-355 | H | H | H | H | H | OCSNMe₂ | H |
| XXVIII-356 | H | H | H | H | H | SMe | H |
| XXVIII-357 | H | H | H | H | H | SEt | H |
| XXVIII-358 | H | H | H | H | H | SCOMe | H |
| XXVIII-359 | H | H | H | H | H | SCSNMe₂ | H |
| XXVIII-360 | H | H | H | H | H | SCSNHMe | H |
| XXVIII-361 | H | H | H | H | H | NHMe | H |
| XXVIII-362 | H | H | H | H | H | NH₂ | H |
| XXVIII-363 | H | H | H | H | H | NMe₂ | H |
| XXVIII-364 | H | H | H | H | H | NHCOMe | H |
| XXVIII-365 | H | H | H | H | H | NHCONH₂ | H |
| XXVIII-366 | H | H | H | H | H | NHCONHMe | H |
| XXVIII-367 | H | H | H | H | H | NHCONMe₂ | H |
| XXVIII-368 | H | H | H | H | H | phenyl | H |
| XXVIII-369 | H | H | H | H | H | 2-chloro- | H |
| | | | | | | phenyl | |
| XXVIII-370 | H | H | H | H | H | 4-nitrophenyl | H |
| XXVIII-371 | H | H | H | H | H | 2-pyridyl | H |
| XXVIII-372 | H | H | H | H | H | 3-pyridyl | H |
| XXVIII-373 | H | H | H | H | H | 4-pyridyl | H |
| XXVIII-374 | H | H | H | H | H | 2-furyl | H |
| XXVIII-375 | H | H | H | H | H | PhO | H |
| XXVIII-376 | H | H | H | H | H | H | Me |
| XXVIII-377 | H | H | H | H | H | H | Et |
| XXVIII-378 | H | H | H | H | H | H | Vinyl |
| XXVIII-379 | H | H | H | H | H | H | Allyl |
| XXVIII-380 | H | H | H | H | H | H | Cyclopropyl |
| XXVIII-381 | H | H | H | H | H | H | F |
| XXVIII-382 | H | H | H | H | H | H | Cl |
| XXVIII-383 | H | H | H | H | H | H | NO₂ |
| XXVIII-384 | H | H | H | H | H | H | CN |
| XXVIII-385 | H | H | H | H | H | H | CONH₂ |
| XXVIII-386 | H | H | H | H | H | H | CONHMe |
| XXVIII-387 | H | H | H | H | H | H | CONMe₂ |
| XXVIII-388 | H | H | H | H | H | H | COMe |
| XXVIII-389 | H | H | H | H | H | H | COOH |
| XXVIII-390 | H | H | H | H | H | H | COOMe |
| XXVIII-391 | H | H | H | H | H | H | CSOMe |
| XXVIII-392 | H | H | H | H | H | H | CSNH₂ |
| XXVIII-393 | H | H | H | H | H | H | CSNMe₂ |
| XXVIII-394 | H | H | H | H | H | H | CSNHMe |
| XXVIII-395 | H | H | H | H | H | H | OMe |
| XXVIII-396 | H | H | H | H | H | H | OEt |
| XXVIII-397 | H | H | H | H | H | H | OCOMe |
| XXVIII-398 | H | H | H | H | H | H | OCOOMe |
| XXVIII-399 | H | H | H | H | H | H | OCONHMe |
| XXVIII-400 | H | H | H | H | H | H | OCONMe₂ |
| XXVIII-401 | H | H | H | H | H | H | OCSMe |
| XXVIII-402 | H | H | H | H | H | H | OCSNMe₂ |
| XXVIII-403 | H | H | H | H | H | H | SMe |
| XXVIII-404 | H | H | H | H | H | H | SEt |
| XXVIII-405 | H | H | H | H | H | H | SCOMe |
| XXVIII-406 | H | H | H | H | H | H | SCSNMe₂ |
| XXVIII-407 | H | H | H | H | H | H | SCSNHMe |
| XXVIII-408 | H | H | H | H | H | H | NHMe |
| XXVIII-409 | H | H | H | H | H | H | NH₂ |
| XXVIII-410 | H | H | H | H | H | H | NMe₂ |
| XXVIII-411 | H | H | H | H | H | H | NHCOMe |
| XXVIII-412 | H | H | H | H | H | H | NHCONH₂ |
| XXVIII-413 | H | H | H | H | H | H | NHCONHMe |
| XXVIII-414 | H | H | H | H | H | H | NHCONMe₂ |
| XXVIII-415 | H | H | H | H | H | H | phenyl |
| XXVIII-416 | H | H | H | H | H | H | 2-chloro- |
| | | | | | | | phenyl |
| XXVIII-417 | H | H | H | H | H | H | 4-nitrophenyl |
| XXVIII-418 | H | H | H | H | H | H | 2-pyridyl |
| XXVIII-419 | H | H | H | H | H | H | 3-pyridyl |
| XXVIII-420 | H | H | H | H | H | H | 4-pyridyl |
| XXVIII-421 | H | H | H | H | H | H | 2-furyl |
| XXVIII-422 | H | H | H | H | H | H | PhO |

Table XXIX provides 422 compounds of formula Iac

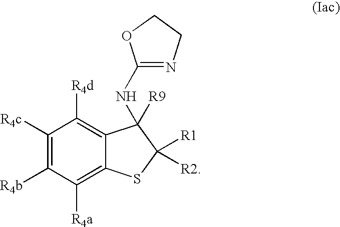

wherein the values of R¹, R², R^{4a}, R^{4b}, R^{4c}, R^{4d} and R⁹ are given in Table 4.

Table XXX provides 422 compounds of formula Iad

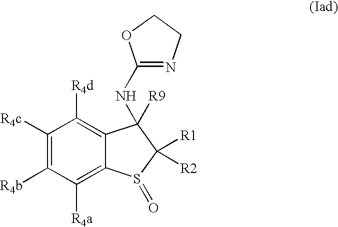

wherein the values of R¹, R², R^{4a}, R^{4b}, R^{4c}, R^{4d} and R⁹ are given in Table 4.

Table XXXI provides 422 compounds of formula Iae

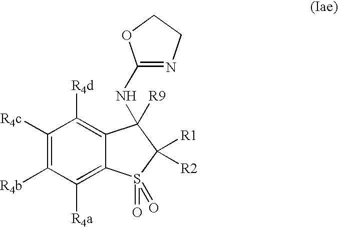

wherein the values of R¹, R², R^{4a}, R^{4b}, R^{4c}, R^{4d} and R⁹ are given in Table 4.

Table XXXII provides 422 compounds of formula Iaf

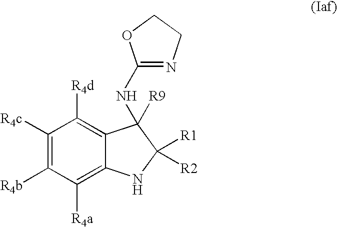

wherein the values of R¹, R², R^{4a}, R^{4b}, R^{4c}, R^{4d} and R⁹ are given in Table 4.

Table XXXIII provides 422 compounds of formula Iag

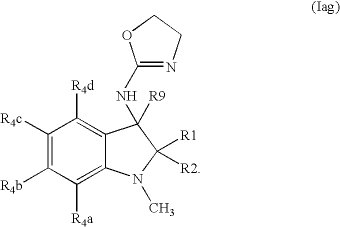

wherein the values of R¹, R², R^{4a}, R^{4b}, R^{4c}, R_{4d} and R⁹ are given in Table 4.

Table XXXIV provides 422 compounds of formula Iah

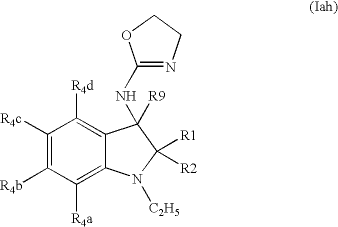

wherein the values of R¹, R², R^{4a}, R^{4b}, R^{4c}, R^{4d} and R⁹ are given in Table 4.

Table XXXV provides 422 compounds of formula Iai

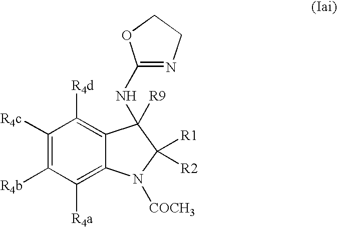

wherein the values of R¹, R², R^{4a}, R^{4b}, R^{4c}, R^{4d} and R⁹ are given in Table 4.

Table XXXVI provides 422 compounds of formula Iaj

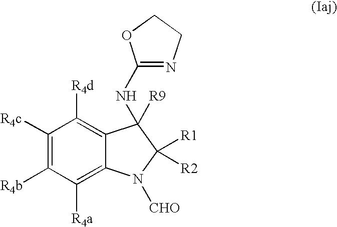

wherein the values of R¹, R², R^{4a}, R^{4b}, R^{4c}, R^{4d} and R⁹ are given in Table 4.

Table XXXVII provides 422 compounds of formula Iak

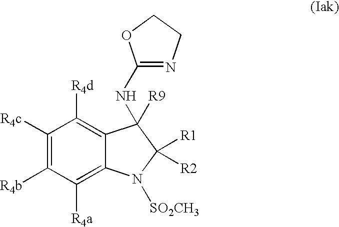

wherein the values of R¹, R², R^{4a}, R^{4b}, R^{4c}, R^{4d} and R⁹ are given in Table 4.

Table XXXVIII provides 391 compounds of formula Ial

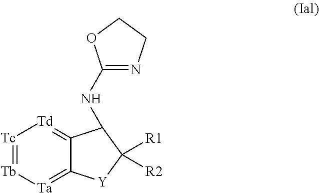

wherein the values of Y, R¹, R², T^{a}, T^{b}, T^{c} and T^{d} are given in Table 5.

TABLE 5

| Compound No | Y | R1 | R2 | Ta | Tb | Tc | Td |
|---|---|---|---|---|---|---|---|
| XXXVIII-1 | CH₂ | H | H | N | CH | CH | CH |
| XXXVIII-2 | CHMe | H | H | N | CH | CH | CH |
| XXXVIII-3 | CHEt | H | H | N | CH | CH | CH |
| XXXVIII-4 | CHF | H | H | N | CH | CH | CH |
| XXXVIII-5 | C═CH₂ | H | H | N | CH | CH | CH |
| XXXVIII-6 | O | H | H | N | CH | CH | CH |
| XXXVIII-7 | S | H | H | N | CH | CH | CH |
| XXXVIII-8 | NH | H | H | N | CH | CH | CH |
| XXXVIII-9 | NMe | H | H | N | CH | CH | CH |
| XXXVIII-10 | NCHO | H | H | N | CH | CH | CH |
| XXXVIII-11 | NCOMe | H | H | N | CH | CH | CH |
| XXXVIII-12 | CH₂ | Me | H | N | CH | CH | CH |
| XXXVIII-13 | O | Me | H | N | CH | CH | CH |
| XXXVIII-14 | CHMe | Me | H | N | CH | CH | CH |
| XXXVIII-15 | CH₂ | ═O | | N | CH | CH | CH |
| XXXVIII-16 | CH₂ | ═CH₂ | | N | CH | CH | CH |
| XXXVIII-17 | CH₂ | ═CHMe | | N | CH | CH | CH |
| XXXVIII-18 | CH₂ | H | H | N | CH | CF | CH |
| XXXVIII-19 | CHMe | H | H | N | CH | CF | CH |
| XXXVIII-20 | CHEt | H | H | N | CH | CF | CH |
| XXXVIII-21 | CHF | H | H | N | CH | CF | CH |
| XXXVIII-22 | C═CH₂ | H | H | N | CH | CF | CH |
| XXXVIII-23 | O | H | H | N | CH | CF | CH |
| XXXVIII-24 | S | H | H | N | CH | CF | CH |
| XXXVIII-25 | NH | H | H | N | CH | CF | CH |
| XXXVIII-26 | NMe | H | H | N | CH | CF | CH |
| XXXVIII-27 | NCHO | H | H | N | CH | CF | CH |
| XXXVIII-28 | NCOMe | H | H | N | CH | CF | CH |
| XXXVIII-29 | CH₂ | Me | H | N | CH | CF | CH |
| XXXVIII-30 | O | Me | H | N | CH | CF | CH |
| XXXVIII-31 | CHMe | Me | H | N | CH | CF | CH |
| XXXVIII-32 | CH₂ | ═O | | N | CH | CF | CH |
| XXXVIII-33 | CH₂ | ═CH₂ | | N | CH | CF | CH |
| XXXVIII-34 | CH₂ | ═CHMe | | N | CH | CF | CH |
| XXXVIII-35 | CH₂ | H | H | N | CH | CH | CF |
| XXXVIII-36 | CHMe | H | H | N | CH | CH | CF |
| XXXVIII-37 | CHEt | H | H | N | CH | CH | CF |
| XXXVIII-38 | CHF | H | H | N | CH | CH | CF |
| XXXVIII-39 | C═CH₂ | H | H | N | CH | CH | CF |
| XXXVIII-40 | O | H | H | N | CH | CH | CF |
| XXXVIII-41 | S | H | H | N | CH | CH | CF |
| XXXVIII-42 | NH | H | H | N | CH | CH | CF |
| XXXVIII-43 | NMe | H | H | N | CH | CH | CF |
| XXXVIII-44 | NCHO | H | H | N | CH | CH | CF |
| XXXVIII-45 | NCOMe | H | H | N | CH | CH | CF |
| XXXVIII-46 | CH₂ | Me | H | N | CH | CH | CF |
| XXXVIII-47 | O | Me | H | N | CH | CH | CF |
| XXXVIII-48 | CHMe | Me | H | N | CH | CH | CF |
| XXXVIII-49 | CH₂ | ═O | | N | CH | CH | CF |
| XXXVIII-50 | CH₂ | ═CH₂ | | N | CH | CH | CF |
| XXXVIII-51 | CH₂ | ═CHMe | | N | CH | CH | CF |
| XXXVIII-52 | CH₂ | H | H | CH | N | CH | CH |
| XXXVIII-53 | CHMe | H | H | CH | N | CH | CH |
| XXXVIII-54 | CHEt | H | H | CH | N | CH | CH |
| XXXVIII-55 | CHF | H | H | CH | N | CH | CH |
| XXXVIII-56 | C═CH₂ | H | H | CH | N | CH | CH |
| XXXVIII-57 | O | H | H | CH | N | CH | CH |
| XXXVIII-58 | S | H | H | CH | N | CH | CH |
| XXXVIII-59 | NH | H | H | CH | N | CH | CH |
| XXXVIII-60 | NMe | H | H | CH | N | CH | CH |
| XXXVIII-61 | NCHO | H | H | CH | N | CH | CH |
| XXXVIII-62 | NCOMe | H | H | CH | N | CH | CH |
| XXXVIII-63 | CH₂ | Me | H | CH | N | CH | CH |
| XXXVIII-64 | O | Me | H | CH | N | CH | CH |
| XXXVIII-65 | CHMe | Me | H | CH | N | CH | CH |
| XXXVIII-66 | CH₂ | ═O | | CH | N | CH | CH |
| XXXVIII-67 | CH₂ | ═CH₂ | | CH | N | CH | CH |
| XXXVIII-68 | CH₂ | ═CHMe | | CH | N | CH | CH |
| XXXVIII-69 | CH₂ | H | H | CF | N | CH | CH |
| XXXVIII-70 | CHMe | H | H | CF | N | CH | CH |
| XXXVIII-71 | CHEt | H | H | CF | N | CH | CH |
| XXXVIII-72 | CHF | H | H | CF | N | CH | CH |
| XXXVIII-73 | C═CH₂ | H | H | CF | N | CH | CH |
| XXXVIII-74 | O | H | H | CF | N | CH | CH |
| XXXVIII-75 | S | H | H | CF | N | CH | CH |
| XXXVIII-76 | NH | H | H | CF | N | CH | CH |
| XXXVIII-77 | NMe | H | H | CF | N | CH | CH |
| XXXVIII-78 | NCHO | H | H | CF | N | CH | CH |
| XXXVIII-79 | NCOMe | H | H | CF | N | CH | CH |
| XXXVIII-80 | CH₂ | Me | H | CF | N | CH | CH |
| XXXVIII-81 | O | Me | H | CF | N | CH | CH |
| XXXVIII-82 | CHMe | Me | H | CF | N | CH | CH |
| XXXVIII-83 | CH₂ | ═O | | CF | N | CH | CH |
| XXXVIII-84 | CH₂ | ═CH₂ | | CF | N | CH | CH |
| XXXVIII-85 | CH₂ | ═CHMe | | CF | N | CH | CH |
| XXXVIII-86 | CH₂ | H | H | CMe | N | CH | CH |
| XXXVIII-87 | CHMe | H | H | CMe | N | CH | CH |
| XXXVIII-88 | CHEt | H | H | CMe | N | CH | CH |
| XXXVIII-89 | CHF | H | H | CMe | N | CH | CH |
| XXXVIII-90 | C═CH₂ | H | H | CMe | N | CH | CH |
| XXXVIII-91 | O | H | H | CMe | N | CH | CH |
| XXXVIII-92 | S | H | H | CMe | N | CH | CH |
| XXXVIII-93 | NH | H | H | CMe | N | CH | CH |
| XXXVIII-94 | NMe | H | H | CMe | N | CH | CH |
| XXXVIII-95 | NCHO | H | H | CMe | N | CH | CH |
| XXXVIII-96 | NCOMe | H | H | CMe | N | CH | CH |
| XXXVIII-97 | CH₂ | Me | H | CMe | N | CH | CH |
| XXXVIII-98 | O | Me | H | CMe | N | CH | CH |
| XXXVIII-99 | CHMe | Me | H | CMe | N | CH | CH |
| XXXVIII-100 | CH₂ | ═O | | CMe | N | CH | CH |
| XXXVIII-101 | CH₂ | ═CH₂ | | CMe | N | CH | CH |
| XXXVIII-102 | CH₂ | ═CHMe | | CMe | N | CH | CH |
| XXXVIII-103 | CH₂ | H | H | CH | N | CF | CH |
| XXXVIII-104 | CHMe | H | H | CH | N | CF | CH |
| XXXVIII-105 | CHEt | H | H | CH | N | CF | CH |
| XXXVIII-106 | CHF | H | H | CH | N | CF | CH |
| XXXVIII-107 | C═CH₂ | H | H | CH | N | CF | CH |
| XXXVIII-108 | O | H | H | CH | N | CF | CH |
| XXXVIII-109 | S | H | H | CH | N | CF | CH |
| XXXVIII-110 | NH | H | H | CH | N | CF | CH |
| XXXVIII-111 | NMe | H | H | CH | N | CF | CH |
| XXXVIII-112 | NCHO | H | H | CH | N | CF | CH |
| XXXVIII-113 | NCOMe | H | H | CH | N | CF | CH |
| XXXVIII-114 | CH₂ | Me | H | CH | N | CF | CH |
| XXXVIII-115 | O | Me | H | CH | N | CF | CH |
| XXXVIII-116 | CHMe | Me | H | CH | N | CF | CH |
| XXXVIII-117 | CH₂ | ═O | | CH | N | CF | CH |
| XXXVIII-118 | CH₂ | ═CH₂ | | CH | N | CF | CH |
| XXXVIII-119 | CH₂ | ═CHMe | | CH | N | CF | CH |
| XXXVIII-120 | CH₂ | H | H | CH | N | CH | CF |
| XXXVIII-121 | CHMe | H | H | CH | N | CH | CF |
| XXXVIII-122 | CHEt | H | H | CH | N | CH | CF |
| XXXVIII-123 | CHF | H | H | CH | N | CH | CF |
| XXXVIII-124 | C═CH₂ | H | H | CH | N | CH | CF |
| XXXVIII-125 | O | H | H | CH | N | CH | CF |
| XXXVIII-126 | S | H | H | CH | N | CH | CF |
| XXXVIII-127 | NH | H | H | CH | N | CH | CF |
| XXXVIII-128 | NMe | H | H | CH | N | CH | CF |
| XXXVIII-129 | NCHO | H | H | CH | N | CH | CF |
| XXXVIII-130 | NCOMe | H | H | CH | N | CH | CF |
| XXXVIII-131 | CH₂ | Me | H | CH | N | CH | CF |
| XXXVIII-132 | O | Me | H | CH | N | CH | CF |
| XXXVIII-133 | CHMe | Me | H | CH | N | CH | CF |
| XXXVIII-134 | CH₂ | ═O | | CH | N | CH | CF |
| XXXVIII-135 | CH₂ | ═CH₂ | | CH | N | CH | CF |
| XXXVIII-136 | CH₂ | ═CHMe | | CH | N | CH | CF |
| XXXVIII-137 | CH₂ | H | H | CH | CH | N | CH |
| XXXVIII-138 | CHMe | H | H | CH | CH | N | CH |
| XXXVIII-139 | CHEt | H | H | CH | CH | N | CH |
| XXXVIII-140 | CHF | H | H | CH | CH | N | CH |
| XXXVIII-141 | C═CH₂ | H | H | CH | CH | N | CH |
| XXXVIII-142 | O | H | H | CH | CH | N | CH |
| XXXVIII-143 | S | H | H | CH | CH | N | CH |
| XXXVIII-144 | NH | H | H | CH | CH | N | CH |
| XXXVIII-145 | NMe | H | H | CH | CH | N | CH |
| XXXVIII-146 | NCHO | H | H | CH | CH | N | CH |
| XXXVIII-147 | NCOMe | H | H | CH | CH | N | CH |
| XXXVIII-148 | CH₂ | Me | H | CH | CH | N | CH |
| XXXVIII-149 | O | Me | H | CH | CH | N | CH |
| XXXVIII-150 | CHMe | Me | H | CH | CH | N | CH |
| XXXVIII-151 | CH₂ | ═O | | CH | CH | N | CH |
| XXXVIII-152 | CH₂ | ═CH₂ | | CH | CH | N | CH |
| XXXVIII-153 | CH₂ | ═CHMe | | CH | CH | N | CH |
| XXXVIII-154 | CH₂ | H | H | CF | CH | N | CH |
| XXXVIII-155 | CHMe | H | H | CF | CH | N | CH |
| XXXVIII-156 | CHEt | H | H | CF | CH | N | CH |
| XXXVIII-157 | CHF | H | H | CF | CH | N | CH |
| XXXVIII-158 | C═CH₂ | H | H | CF | CH | N | CH |
| XXXVIII-159 | O | H | H | CF | CH | N | CH |
| XXXVIII-160 | S | H | H | CF | CH | N | CH |
| XXXVIII-161 | NH | H | H | CF | CH | N | CH |
| XXXVIII-162 | NMe | H | H | CF | CH | N | CH |
| XXXVIII-163 | NCHO | H | H | CF | CH | N | CH |
| XXXVIII-164 | NCOMe | H | H | CF | CH | N | CH |
| XXXVIII-165 | CH₂ | Me | H | CF | CH | N | CH |
| XXXVIII-166 | O | Me | H | CF | CH | N | CH |
| XXXVIII-167 | CHMe | Me | H | CF | CH | N | CH |
| XXXVIII-168 | CH₂ | ═O | | CF | CH | N | CH |
| XXXVIII-169 | CH₂ | ═CH₂ | | CF | CH | N | CH |
| XXXVIII-170 | CH₂ | ═CHMe | | CF | CH | N | CH |
| XXXVIII-171 | CH₂ | H | H | CMe | CH | N | CH |
| XXXVIII-172 | CHMe | H | H | CMe | CH | N | CH |
| XXXVIII-173 | CHEt | H | H | CMe | CH | N | CH |
| XXXVIII-174 | CHF | H | H | CMe | CH | N | CH |
| XXXVIII-175 | C═CH₂ | H | H | CMe | CH | N | CH |
| XXXVIII-176 | O | H | H | CMe | CH | N | CH |
| XXXVIII-177 | S | H | H | CMe | CH | N | CH |
| XXXVIII-178 | NH | H | H | CMe | CH | N | CH |
| XXXVIII-179 | NMe | H | H | CMe | CH | N | CH |
| XXXVIII-180 | NCHO | H | H | CMe | CH | N | CH |
| XXXVIII-181 | NCOMe | H | H | CMe | CH | N | CH |
| XXXVIII-182 | CH₂ | Me | H | CMe | CH | N | CH |
| XXXVIII-183 | O | Me | H | CMe | CH | N | CH |
| XXXVIII-184 | CHMe | Me | H | CMe | CH | N | CH |
| XXXVIII-185 | CH₂ | ═O | | CMe | CH | N | CH |
| XXXVIII-186 | CH₂ | ═CH₂ | | CMe | CH | N | CH |
| XXXVIII-187 | CH₂ | ═CHMe | | CMe | CH | N | CH |
| XXXVIII-188 | CH₂ | H | H | CH | CH | N | CF |
| XXXVIII-189 | CHMe | H | H | CH | CH | N | CF |
| XXXVIII-190 | CHEt | H | H | CH | CH | N | CF |
| XXXVIII-191 | CHF | H | H | CH | CH | N | CF |
| XXXVIII-192 | C═CH₂ | H | H | CH | CH | N | CF |
| XXXVIII-193 | O | H | H | CH | CH | N | CF |
| XXXVIII-194 | S | H | H | CH | CH | N | CF |
| XXXVIII-195 | NH | H | H | CH | CH | N | CF |
| XXXVIII-196 | NMe | H | H | CH | CH | N | CF |
| XXXVIII-197 | NCHO | H | H | CH | CH | N | CF |
| XXXVIII-198 | NCOMe | H | H | CH | CH | N | CF |
| XXXVIII-199 | CH₂ | Me | H | CH | CH | N | CF |
| XXXVIII-200 | O | Me | H | CH | CH | N | CF |
| XXXVIII-201 | CHMe | Me | H | CH | CH | N | CF |
| XXXVIII-202 | CH₂ | ═O | | CH | CH | N | CF |
| XXXVIII-203 | CH₂ | ═CH₂ | | CH | CH | N | CF |
| XXXVIII-204 | CH₂ | ═CHMe | | CH | CH | N | CF |
| XXXVIII-205 | CH₂ | H | H | CH | CH | CH | N |
| XXXVIII-206 | CHMe | H | H | CH | CH | CH | N |
| XXXVIII-207 | CHEt | H | H | CH | CH | CH | N |
| XXXVIII-208 | CHF | H | H | CH | CH | CH | N |
| XXXVIII-209 | C═CH₂ | H | H | CH | CH | CH | N |
| XXXVIII-210 | O | H | H | CH | CH | CH | N |
| XXXVIII-211 | S | H | H | CH | CH | CH | N |
| XXXVIII-212 | NH | H | H | CH | CH | CH | N |
| XXXVIII-213 | NMe | H | H | CH | CH | CH | N |
| XXXVIII-214 | NCHO | H | H | CH | CH | CH | N |
| XXXVIII-215 | NCOMe | H | H | CH | CH | CH | N |
| XXXVIII-216 | CH₂ | Me | H | CH | CH | CH | N |
| XXXVIII-217 | O | Me | H | CH | CH | CH | N |
| XXXVIII-218 | CHMe | Me | H | CH | CH | CH | N |
| XXXVIII-219 | CH₂ | ═O | | CH | CH | CH | N |
| XXXVIII-220 | CH₂ | ═CH₂ | | CH | CH | CH | N |
| XXXVIII-221 | CH₂ | ═CHMe | | CH | CH | CH | N |
| XXXVIII-222 | CH₂ | H | H | CF | CH | CH | N |
| XXXVIII-223 | CHMe | H | H | CF | CH | CH | N |
| XXXVIII-224 | CHEt | H | H | CF | CH | CH | N |
| XXXVIII-225 | CHF | H | H | CF | CH | CH | N |
| XXXVIII-226 | C═CH₂ | H | H | CF | CH | CH | N |
| XXXVIII-227 | O | H | H | CF | CH | CH | N |
| XXXVIII-228 | S | H | H | CF | CH | CH | N |
| XXXVIII-229 | NH | H | H | CF | CH | CH | N |
| XXXVIII-230 | NMe | H | H | CF | CH | CH | N |
| XXXVIII-231 | NCHO | H | H | CF | CH | CH | N |
| XXXVIII-232 | NCOMe | H | H | CF | CH | CH | N |
| XXXVIII-233 | CH₂ | Me | H | CF | CH | CH | N |
| XXXVIII-234 | O | Me | H | CF | CH | CH | N |
| XXXVIII-235 | CHMe | Me | H | CF | CH | CH | N |
| XXXVIII-236 | CH₂ | ═O | | CF | CH | CH | N |
| XXXVIII-237 | CH₂ | ═CH₂ | | CF | CH | CH | N |
| XXXVIII-238 | CH₂ | ═CHMe | | CF | CH | CH | N |
| XXXVIII-239 | CH₂ | H | H | CMe | CH | CH | N |
| XXXVIII-240 | CHMe | H | H | CMe | CH | CH | N |
| XXXVIII-241 | CHEt | H | H | CMe | CH | CH | N |
| XXXVIII-242 | CHF | H | H | CMe | CH | CH | N |
| XXXVIII-243 | C═CH₂ | H | H | CMe | CH | CH | N |
| XXXVIII-244 | O | H | H | CMe | CH | CH | N |
| XXXVIII-245 | S | H | H | CMe | CH | CH | N |
| XXXVIII-246 | NH | H | H | CMe | CH | CH | N |
| XXXVIII-247 | NMe | H | H | CMe | CH | CH | N |
| XXXVIII-248 | NCHO | H | H | CMe | CH | CH | N |
| XXXVIII-249 | NCOMe | H | H | CMe | CH | CH | N |
| XXXVIII-250 | CH₂ | Me | H | CMe | CH | CH | N |
| XXXVIII-251 | O | Me | H | CMe | CH | CH | N |
| XXXVIII-252 | CHMe | Me | H | CMe | CH | CH | N |
| XXXVIII-253 | CH₂ | ═O | | CMe | CH | CH | N |
| XXXVIII-254 | CH₂ | ═CH₂ | | CMe | CH | CH | N |
| XXXVIII-255 | CH₂ | ═CHMe | | CMe | CH | CH | N |
| XXXVIII-256 | CH₂ | H | H | CH | CH | CF | N |
| XXXVIII-257 | CHMe | H | H | CH | CH | CF | N |
| XXXVIII-258 | CHEt | H | H | CH | CH | CF | N |
| XXXVIII-259 | CHF | H | H | CH | CH | CF | N |
| XXXVIII-260 | C═CH₂ | H | H | CH | CH | CF | N |
| XXXVIII-261 | O | H | H | CH | CH | CF | N |
| XXXVIII-262 | S | H | H | CH | CH | CF | N |
| XXXVIII-263 | NH | H | H | CH | CH | CF | N |
| XXXVIII-264 | NMe | H | H | CH | CH | CF | N |
| XXXVIII-265 | NCHO | H | H | CH | CH | CF | N |
| XXXVIII-266 | NCOMe | H | H | CH | CH | CF | N |
| XXXVIII-267 | CH₂ | Me | H | CH | CH | CF | N |
| XXXVIII-268 | O | Me | H | CH | CH | CF | N |
| XXXVIII-269 | CHMe | Me | H | CH | CH | CF | N |
| XXXVIII-270 | CH₂ | ═O | | CH | CH | CF | N |
| XXXVIII-271 | CH₂ | ═CH₂ | | CH | CH | CF | N |
| XXXVIII-272 | CH₂ | ═CHMe | | CH | CH | CF | N |
| XXXVIII-273 | CH₂ | H | H | N | CH | N | CH |
| XXXVIII-274 | CHMe | H | H | N | CH | N | CH |
| XXXVIII-275 | CHEt | H | H | N | CH | N | CH |
| XXXVIII-276 | CHF | H | H | N | CH | N | CH |
| XXXVIII-277 | C═CH₂ | H | H | N | CH | N | CH |
| XXXVIII-278 | O | H | H | N | CH | N | CH |
| XXXVIII-279 | S | H | H | N | CH | N | CH |
| XXXVIII-280 | NH | H | H | N | CH | N | CH |
| XXXVIII-281 | NMe | H | H | N | CH | N | CH |
| XXXVIII-282 | NCHO | H | H | N | CH | N | CH |
| XXXVIII-283 | NCOMe | H | H | N | CH | N | CH |
| XXXVIII-284 | CH₂ | Me | H | N | CH | N | CH |
| XXXVIII-285 | O | Me | H | N | CH | N | CH |
| XXXVIII-286 | CHMe | Me | H | N | CH | N | CH |
| XXXVIII-287 | CH₂ | ═O | | N | CH | N | CH |
| XXXVIII-288 | CH₂ | ═CH₂ | | N | CH | N | CH |
| XXXVIII-289 | CH₂ | ═CHMe | | N | CH | N | CH |
| XXXVIII-290 | CH₂ | H | H | N | CH | N | CF |
| XXXVIII-291 | CHMe | H | H | N | CH | N | CF |
| XXXVIII-292 | CHEt | H | H | N | CH | N | CF |
| XXXVIII-293 | CHF | H | H | N | CH | N | CF |
| XXXVIII-294 | C═CH₂ | H | H | N | CH | N | CF |
| XXXVIII-295 | O | H | H | N | CH | N | CF |
| XXXVIII-296 | S | H | H | N | CH | N | CF |
| XXXVIII-297 | NH | H | H | N | CH | N | CF |
| XXXVIII-298 | NMe | H | H | N | CH | N | CF |
| XXXVIII-299 | NCHO | H | H | N | CH | N | CF |
| XXXVIII-300 | NCOMe | H | H | N | CH | N | CF |
| XXXVIII-301 | CH₂ | Me | H | N | CH | N | CF |
| XXXVIII-302 | O | Me | H | N | CH | N | CF |
| XXXVIII-303 | CHMe | Me | H | N | CH | N | CF |
| XXXVIII-304 | CH₂ | ═O | | N | CH | N | CF |
| XXXVIII-305 | CH₂ | ═CH₂ | | N | CH | N | CF |
| XXXVIII-306 | CH₂ | ═CHMe | | N | CH | N | CF |
| XXXVIII-307 | CH₂ | H | H | N | N | CH | CH |
| XXXVIII-308 | CHMe | H | H | N | N | CH | CH |
| XXXVIII-309 | CHEt | H | H | N | N | CH | CH |
| XXXVIII-310 | CHF | H | H | N | N | CH | CH |
| XXXVIII-311 | C═CH₂ | H | H | N | N | CH | CH |
| XXXVIII-312 | O | H | H | N | N | CH | CH |
| XXXVIII-313 | S | H | H | N | N | CH | CH |
| XXXVIII-314 | NH | H | H | N | N | CH | CH |
| XXXVIII-315 | NMe | H | H | N | N | CH | CH |
| XXXVIII-316 | NCHO | H | H | N | N | CH | CH |
| XXXVIII-317 | NCOMe | H | H | N | N | CH | CH |
| XXXVIII-318 | CH₂ | Me | H | N | N | CH | CH |
| XXXVIII-319 | O | Me | H | N | N | CH | CH |
| XXXVIII-320 | CHMe | Me | H | N | N | CH | CH |
| XXXVIII-321 | CH₂ | ═O | | N | N | CH | CH |
| XXXVIII-322 | CH₂ | ═CH₂ | | N | N | CH | CH |
| XXXVIII-323 | CH₂ | ═CHMe | | N | N | CH | CH |
| XXXVIII-324 | CH₂ | H | H | N | N | CF | CH |
| XXXVIII-325 | CHMe | H | H | N | N | CF | CH |
| XXXVIII-326 | CHEt | H | H | N | N | CF | CH |
| XXXVIII-327 | CHF | H | H | N | N | CF | CH |
| XXXVIII-328 | C═CH₂ | H | H | N | N | CF | CH |
| XXXVIII-329 | O | H | H | N | N | CF | CH |
| XXXVIII-330 | S | H | H | N | N | CF | CH |
| XXXVIII-331 | NH | H | H | N | N | CF | CH |
| XXXVIII-332 | NMe | H | H | N | N | CF | CH |
| XXXVIII-333 | NCHO | H | H | N | N | CF | CH |
| XXXVIII-334 | NCOMe | H | H | N | N | CF | CH |
| XXXVIII-335 | CH₂ | Me | H | N | N | CF | CH |
| XXXVIII-336 | O | Me | H | N | N | CF | CH |
| XXXVIII-337 | CHMe | Me | H | N | N | CF | CH |
| XXXVIII-338 | CH₂ | ═O | | N | N | CF | CH |
| XXXVIII-339 | CH₂ | ═CH₂ | | N | N | CF | CH |
| XXXVIII-340 | CH₂ | ═CHMe | | N | N | CF | CH |
| XXXVIII-341 | CH₂ | H | H | N | N | CH | CF |
| XXXVIII-342 | CHMe | H | H | N | N | CH | CF |
| XXXVIII-343 | CHEt | H | H | N | N | CH | CF |
| XXXVIII-344 | CHF | H | H | N | N | CH | CF |
| XXXVIII-345 | C═CH₂ | H | H | N | N | CH | CF |
| XXXVIII-346 | O | H | H | N | N | CH | CF |
| XXXVIII-347 | S | H | H | N | N | CH | CF |
| XXXVIII-348 | NH | H | H | N | N | CH | CF |
| XXXVIII-349 | NMe | H | H | N | N | CH | CF |
| XXXVIII-350 | NCHO | H | H | N | N | CH | CF |
| XXXVIII-351 | NCOMe | H | H | N | N | CH | CF |
| XXXVIII-352 | CH₂ | Me | H | N | N | CH | CF |
| XXXVIII-353 | O | Me | H | N | N | CH | CF |
| XXXVIII-354 | CHMe | Me | H | N | N | CH | CF |
| XXXVIII-355 | CH₂ | ═O | | N | N | CH | CF |
| XXXVIII-356 | CH₂ | ═CH₂ | | N | N | CH | CF |
| XXXVIII-357 | CH₂ | ═CHMe | | N | N | CH | CF |
| XXXVIII-358 | CH₂ | H | H | N | CH | CH | N |
| XXXVIII-359 | CHMe | H | H | N | CH | CH | N |
| XXXVIII-360 | CHEt | H | H | N | CH | CH | N |
| XXXVIII-361 | CHF | H | H | N | CH | CH | N |
| XXXVIII-362 | C═CH₂ | H | H | N | CH | CH | N |
| XXXVIII-363 | O | H | H | N | CH | CH | N |
| XXXVIII-364 | S | H | H | N | CH | CH | N |
| XXXVIII-365 | NH | H | H | N | CH | CH | N |
| XXXVIII-366 | NMe | H | H | N | CH | CH | N |
| XXXVIII-367 | NCHO | H | H | N | CH | CH | N |
| XXXVIII-368 | NCOMe | H | H | N | CH | CH | N |
| XXXVIII-369 | CH₂ | Me | H | N | CH | CH | N |
| XXXVIII-370 | O | Me | H | N | CH | CH | N |
| XXXVIII-371 | CHMe | Me | H | N | CH | CH | N |
| XXXVIII-372 | CH₂ | ═O | | N | CH | CH | N |
| XXXVIII-373 | CH₂ | ═CH₂ | | N | CH | CH | N |
| XXXVIII-374 | CH₂ | ═CHMe | | N | CH | CH | N |
| XXXVIII-375 | CH₂ | H | H | CH | N | CH | N |
| XXXVIII-376 | CHMe | H | H | CH | N | CH | N |
| XXXVIII-377 | CHEt | H | H | CH | N | CH | N |
| XXXVIII-378 | CHF | H | H | CH | N | CH | N |
| XXXVIII-379 | C═CH₂ | H | H | CH | N | CH | N |
| XXXVIII-380 | O | H | H | CH | N | CH | N |
| XXXVIII-381 | S | H | H | CH | N | CH | N |
| XXXVIII-382 | NH | H | H | CH | N | CH | N |
| XXXVIII-383 | NMe | H | H | CH | N | CH | N |
| XXXVIII-384 | NCHO | H | H | CH | N | CH | N |
| XXXVIII-385 | NCOMe | H | H | CH | N | CH | N |
| XXXVIII-386 | CH₂ | Me | H | CH | N | CH | N |
| XXXVIII-387 | O | Me | H | CH | N | CH | N |
| XXXVIII-388 | CHMe | Me | H | CH | N | CH | N |

| | | | | | | |
|---|---|---|---|---|---|---|
| XXXVIII-389 | CH₂ | ═O | CH | N | CH | N |
| XXXVIII-390 | CH₂ | ═CH₂ | CH | N | CH | N |
| XXXVIII-391 | CH₂ | ═CHMe | CH | N | CH | N |

Table XXXIX provides 578 compounds of formula Iam

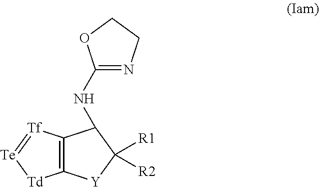

wherein the values of Y, R¹, R², T^{d}, T^{e} and T^{f} are given in Table 6.

TABLE 6

| Compound No | Y | R1 | R2 | Td | Te | Tf |
|---|---|---|---|---|---|---|
| XXXIX-1 | CH₂ | H | H | NH | CH | CH |
| XXXIX-2 | CHMe | H | H | NH | CH | CH |
| XXXIX-3 | CHEt | H | H | NH | CH | CH |
| XXXIX-4 | CHF | H | H | NH | CH | CH |
| XXXIX-5 | C═CH₂ | H | H | NH | CH | CH |
| XXXIX-6 | O | H | H | NH | CH | CH |
| XXXIX-7 | S | H | H | NH | CH | CH |
| XXXIX-8 | NH | H | H | NH | CH | CH |
| XXXIX-9 | NMe | H | H | NH | CH | CH |
| XXXIX-10 | NCHO | H | H | NH | CH | CH |
| XXXIX-11 | NCOMe | H | H | NH | CH | CH |
| XXXIX-12 | CH₂ | Me | H | NH | CH | CH |
| XXXIX-13 | O | Me | H | NH | CH | CH |
| XXXIX-14 | CHMe | Me | H | NH | CH | CH |
| XXXIX-15 | CH₂ | ═O | | NH | CH | CH |
| XXXIX-16 | CH₂ | ═CH₂ | | NH | CH | CH |
| XXXIX-17 | CH₂ | ═CHMe | | NH | CH | CH |
| XXXIX-18 | CH₂ | H | H | NH | CF | CH |
| XXXIX-19 | CHMe | H | H | NH | CF | CH |
| XXXIX-20 | CHEt | H | H | NH | CF | CH |
| XXXIX-21 | CHF | H | H | NH | CF | CH |
| XXXIX-22 | C═CH₂ | H | H | NH | CF | CH |
| XXXIX-23 | O | H | H | NH | CF | CH |
| XXXIX-24 | S | H | H | NH | CF | CH |
| XXXIX-25 | NH | H | H | NH | CF | CH |
| XXXIX-26 | NMe | H | H | NH | CF | CH |
| XXXIX-27 | NCHO | H | H | NH | CF | CH |
| XXXIX-28 | NCOMe | H | H | NH | CF | CH |
| XXXIX-29 | CH₂ | Me | H | NH | CF | CH |
| XXXIX-30 | O | Me | H | NH | CF | CH |
| XXXIX-31 | CHMe | Me | H | NH | CF | CH |
| XXXIX-32 | CH₂ | ═O | | NH | CF | CH |
| XXXIX-33 | CH₂ | ═CH₂ | | NH | CF | CH |
| XXXIX-34 | CH₂ | ═CHMe | | NH | CF | CH |
| XXXIX-35 | CH₂ | H | H | NH | CMe | CH |
| XXXIX-36 | CHMe | H | H | NH | CMe | CH |
| XXXIX-37 | CHEt | H | H | NH | CMe | CH |
| XXXIX-38 | CHF | H | H | NH | CMe | CH |
| XXXIX-39 | C═CH₂ | H | H | NH | CMe | CH |
| XXXIX-40 | O | H | H | NH | CMe | CH |
| XXXIX-41 | S | H | H | NH | CMe | CH |
| XXXIX-42 | NH | H | H | NH | CMe | CH |
| XXXIX-43 | NMe | H | H | NH | CMe | CH |
| XXXIX-44 | NCHO | H | H | NH | CMe | CH |
| XXXIX-45 | NCOMe | H | H | NH | CMe | CH |
| XXXIX-46 | CH₂ | Me | H | NH | CMe | CH |
| XXXIX-47 | O | Me | H | NH | CMe | CH |
| XXXIX-48 | CHMe | Me | H | NH | CMe | CH |
| XXXIX-49 | CH₂ | ═O | | NH | CMe | CH |
| XXXIX-50 | CH₂ | ═CH₂ | | NH | CMe | CH |
| XXXIX-51 | CH₂ | ═CHMe | | NH | CMe | CH |
| XXXIX-52 | CH₂ | H | H | NH | CH | CMe |
| XXXIX-53 | CHMe | H | H | NH | CH | CMe |
| XXXIX-54 | CHEt | H | H | NH | CH | CMe |
| XXXIX-55 | CHF | H | H | NH | CH | CMe |
| XXXIX-56 | C═CH₂ | H | H | NH | CH | CMe |
| XXXIX-57 | O | H | H | NH | CH | CMe |
| XXXIX-58 | S | H | H | NH | CH | CMe |
| XXXIX-59 | NH | H | H | NH | CH | CMe |
| XXXIX-60 | NMe | H | H | NH | CH | CMe |
| XXXIX-61 | NCHO | H | H | NH | CH | CMe |
| XXXIX-62 | NCOMe | H | H | NH | CH | CMe |
| XXXIX-63 | CH₂ | Me | H | NH | CH | CMe |
| XXXIX-64 | O | Me | H | NH | CH | CMe |
| XXXIX-65 | CHMe | Me | H | NH | CH | CMe |
| XXXIX-66 | CH₂ | ═O | | NH | CH | CMe |
| XXXIX-67 | CH₂ | ═CH₂ | | NH | CH | CMe |
| XXXIX-68 | CH₂ | ═CHMe | | NH | CH | CMe |
| XXXIX-69 | CH₂ | H | H | NMe | CH | CH |
| XXXIX-70 | CHMe | H | H | NMe | CH | CH |
| XXXIX-71 | CHEt | H | H | NMe | CH | CH |
| XXXIX-72 | CHF | H | H | NMe | CH | CH |
| XXXIX-73 | C═CH₂ | H | H | NMe | CH | CH |
| XXXIX-74 | O | H | H | NMe | CH | CH |
| XXXIX-75 | S | H | H | NMe | CH | CH |
| XXXIX-76 | NH | H | H | NMe | CH | CH |
| XXXIX-77 | NMe | H | H | NMe | CH | CH |
| XXXIX-78 | NCHO | H | H | NMe | CH | CH |
| XXXIX-79 | NCOMe | H | H | NMe | CH | CH |
| XXXIX-80 | CH₂ | Me | H | NMe | CH | CH |
| XXXIX-81 | O | Me | H | NMe | CH | CH |
| XXXIX-82 | CHMe | Me | H | NMe | CH | CH |
| XXXIX-83 | CH₂ | ═O | | NMe | CH | CH |
| XXXIX-84 | CH₂ | ═CH₂ | | NMe | CH | CH |
| XXXIX-85 | CH₂ | ═CHMe | | NMe | CH | CH |
| XXXIX-86 | CH₂ | H | H | NMe | CF | CH |
| XXXIX-87 | CHMe | H | H | NMe | CF | CH |
| XXXIX-88 | CHEt | H | H | NMe | CF | CH |
| XXXIX-89 | CHF | H | H | NMe | CF | CH |
| XXXIX-90 | C═CH₂ | H | H | NMe | CF | CH |
| XXXIX-91 | O | H | H | NMe | CF | CH |
| XXXIX-92 | S | H | H | NMe | CF | CH |
| XXXIX-93 | NH | H | H | NMe | CF | CH |
| XXXIX-94 | NMe | H | H | NMe | CF | CH |
| XXXIX-95 | NCHO | H | H | NMe | CF | CH |
| XXXIX-96 | NCOMe | H | H | NMe | CF | CH |
| XXXIX-97 | CH₂ | Me | H | NMe | CF | CH |
| XXXIX-98 | O | Me | H | NMe | CF | CH |
| XXXIX-99 | CHMe | Me | H | NMe | CF | CH |
| XXXIX-100 | CH₂ | ═O | | NMe | CF | CH |
| XXXIX-101 | CH₂ | ═CH₂ | | NMe | CF | CH |
| XXXIX-102 | CH₂ | ═CHMe | | NMe | CF | CH |
| XXXIX-103 | CH₂ | H | H | NMe | CMe | CH |
| XXXIX-104 | CHMe | H | H | NMe | CMe | CH |
| XXXIX-105 | CHEt | H | H | NMe | CMe | CH |
| XXXIX-106 | CHF | H | H | NMe | CMe | CH |
| XXXIX-107 | C═CH₂ | H | H | NMe | CMe | CH |
| XXXIX-108 | O | H | H | NMe | CMe | CH |
| XXXIX-109 | S | H | H | NMe | CMe | CH |
| XXXIX-110 | NH | H | H | NMe | CMe | CH |
| XXXIX-111 | NMe | H | H | NMe | CMe | CH |
| XXXIX-112 | NCHO | H | H | NMe | CMe | CH |
| XXXIX-113 | NCOMe | H | H | NMe | CMe | CH |
| XXXIX-114 | CH₂ | Me | H | NMe | CMe | CH |
| XXXIX-115 | O | Me | H | NMe | CMe | CH |
| XXXIX-116 | CHMe | Me | H | NMe | CMe | CH |
| XXXIX-117 | CH₂ | ═O | | NMe | CMe | CH |
| XXXIX-118 | CH₂ | ═CH₂ | | NMe | CMe | CH |
| XXXIX-119 | CH₂ | ═CHMe | | NMe | CMe | CH |
| XXXIX-120 | CH₂ | H | H | NMe | CH | CMe |
| XXXIX-121 | CHMe | H | H | NMe | CH | CMe |
| XXXIX-122 | CHEt | H | H | NMe | CH | CMe |
| XXXIX-123 | CHF | H | H | NMe | CH | CMe |
| XXXIX-124 | C═CH₂ | H | H | NMe | CH | CMe |
| XXXIX-125 | O | H | H | NMe | CH | CMe |
| XXXIX-126 | S | H | H | NMe | CH | CMe |
| XXXIX-127 | NH | H | H | NMe | CH | CMe |
| XXXIX-128 | NMe | H | H | NMe | CH | CMe |
| XXXIX-129 | NCHO | H | H | NMe | CH | CMe |
| XXXIX-130 | NCOMe | H | H | NMe | CH | CMe |
| XXXIX-131 | CH₂ | Me | H | NMe | CH | CMe |
| XXXIX-132 | O | Me | H | NMe | CH | CMe |
| XXXIX-133 | CHMe | Me | H | NMe | CH | CMe |
| XXXIX-134 | CH₂ | ═O | | NMe | CH | CMe |
| XXXIX-135 | CH₂ | ═CH₂ | | NMe | CH | CMe |
| XXXIX-136 | CH₂ | ═CHMe | | NMe | CH | CMe |
| XXXIX-137 | CH₂ | H | H | O | CH | CH |
| XXXIX-138 | CHMe | H | H | O | CH | CH |
| XXXIX-139 | CHEt | H | H | O | CH | CH |
| XXXIX-140 | CHF | H | H | O | CH | CH |
| XXXIX-141 | C═CH₂ | H | H | O | CH | CH |
| XXXIX-142 | O | H | H | O | CH | CH |
| XXXIX-143 | S | H | H | O | CH | CH |
| XXXIX-144 | NH | H | H | O | CH | CH |
| XXXIX-145 | NMe | H | H | O | CH | CH |
| XXXIX-146 | NCHO | H | H | O | CH | CH |
| XXXIX-147 | NCOMe | H | H | O | CH | CH |
| XXXIX-148 | CH₂ | Me | H | O | CH | CH |
| XXXIX-149 | O | Me | H | O | CH | CH |
| XXXIX-150 | CHMe | Me | H | O | CH | CH |
| XXXIX-151 | CH₂ | ═O | | O | CH | CH |
| XXXIX-152 | CH₂ | ═CH₂ | | O | CH | CH |
| XXXIX-153 | CH₂ | ═CHMe | | O | CH | CH |
| XXXIX-154 | CH₂ | H | H | O | CF | CH |
| XXXIX-155 | CHMe | H | H | O | CF | CH |
| XXXIX-156 | CHEt | H | H | O | CF | CH |
| XXXIX-157 | CHF | H | H | O | CF | CH |
| XXXIX-158 | C═CH₂ | H | H | O | CF | CH |
| XXXIX-159 | O | H | H | O | CF | CH |
| XXXIX-160 | S | H | H | O | CF | CH |
| XXXIX-161 | NH | H | H | O | CF | CH |
| XXXIX-162 | NMe | H | H | O | CF | CH |
| XXXIX-163 | NCHO | H | H | O | CF | CH |
| XXXIX-164 | NCOMe | H | H | O | CF | CH |
| XXXIX-165 | CH₂ | Me | H | O | CF | CH |
| XXXIX-166 | O | Me | H | O | CF | CH |
| XXXIX-167 | CHMe | Me | H | O | CF | CH |
| XXXIX-168 | CH₂ | ═O | | O | CF | CH |
| XXXIX-169 | CH₂ | ═CH₂ | | O | CF | CH |
| XXXIX-170 | CH₂ | ═CHMe | | O | CF | CH |
| XXXIX-171 | CH₂ | H | H | O | CMe | CH |
| XXXIX-172 | CHMe | H | H | O | CMe | CH |
| XXXIX-173 | CHEt | H | H | O | CMe | CH |
| XXXIX-174 | CHF | H | H | O | CMe | CH |
| XXXIX-175 | C═CH₂ | H | H | O | CMe | CH |
| XXXIX-176 | O | H | H | O | CMe | CH |
| XXXIX-177 | S | H | H | O | CMe | CH |
| XXXIX-178 | NH | H | H | O | CMe | CH |
| XXXIX-179 | NMe | H | H | O | CMe | CH |
| XXXIX-180 | NCHO | H | H | O | CMe | CH |
| XXXIX-181 | NCOMe | H | H | O | CMe | CH |
| XXXIX-182 | CH₂ | Me | H | O | CMe | CH |
| XXXIX-183 | O | Me | H | O | CMe | CH |
| XXXIX-184 | CHMe | Me | H | O | CMe | CH |
| XXXIX-185 | CH₂ | ═O | | O | CMe | CH |
| XXXIX-186 | CH₂ | ═CH₂ | | O | CMe | CH |
| XXXIX-187 | CH₂ | ═CHMe | | O | CMe | CH |
| XXXIX-188 | CH₂ | H | H | O | CH | CMe |
| XXXIX-189 | CHMe | H | H | O | CH | CMe |
| XXXIX-190 | CHEt | H | H | O | CH | CMe |
| XXXIX-191 | CHF | H | H | O | CH | CMe |
| XXXIX-192 | C═CH₂ | H | H | O | CH | CMe |
| XXXIX-193 | O | H | H | O | CH | CMe |
| XXXIX-194 | S | H | H | O | CH | CMe |
| XXXIX-195 | NH | H | H | O | CH | CMe |
| XXXIX-196 | NMe | H | H | O | CH | CMe |
| XXXIX-197 | NCHO | H | H | O | CH | CMe |
| XXXIX-198 | NCOMe | H | H | O | CH | CMe |
| XXXIX-199 | CH₂ | Me | H | O | CH | CMe |
| XXXIX-200 | O | Me | H | O | CH | CMe |
| XXXIX-201 | CHMe | Me | H | O | CH | CMe |
| XXXIX-202 | CH₂ | ═O | | O | CH | CMe |
| XXXIX-203 | CH₂ | ═CH₂ | | O | CH | CMe |
| XXXIX-204 | CH₂ | ═CHMe | | O | CH | CMe |
| XXXIX-205 | CH₂ | H | H | S | CH | CH |
| XXXIX-206 | CHMe | H | H | S | CH | CH |
| XXXIX-207 | CHEt | H | H | S | CH | CH |
| XXXIX-208 | CHF | H | H | S | CH | CH |
| XXXIX-209 | C═CH₂ | H | H | S | CH | CH |
| XXXIX-210 | O | H | H | S | CH | CH |
| XXXIX-211 | S | H | H | S | CH | CH |
| XXXIX-212 | NH | H | H | S | CH | CH |
| XXXIX-213 | NMe | H | H | S | CH | CH |
| XXXIX-214 | NCHO | H | H | S | CH | CH |
| XXXIX-215 | NCOMe | H | H | S | CH | CH |
| XXXIX-216 | CH₂ | Me | H | S | CH | CH |
| XXXIX-217 | O | Me | H | S | CH | CH |
| XXXIX-218 | CHMe | Me | H | S | CH | CH |
| XXXIX-219 | CH₂ | ═O | | S | CH | CH |
| XXXIX-220 | CH₂ | ═CH₂ | | S | CH | CH |
| XXXIX-221 | CH₂ | ═CHMe | | S | CH | CH |
| XXXIX-222 | CH₂ | H | H | S | CF | CH |
| XXXIX-223 | CHMe | H | H | S | CF | CH |
| XXXIX-224 | CHEt | H | H | S | CF | CH |
| XXXIX-225 | CHF | H | H | S | CF | CH |
| XXXIX-226 | C═CH₂ | H | H | S | CF | CH |
| XXXIX-227 | O | H | H | S | CF | CH |
| XXXIX-228 | S | H | H | S | CF | CH |
| XXXIX-229 | NH | H | H | S | CF | CH |
| XXXIX-230 | NMe | H | H | S | CF | CH |
| XXXIX-231 | NCHO | H | H | S | CF | CH |
| XXXIX-232 | NCOMe | H | H | S | CF | CH |
| XXXIX-233 | CH₂ | Me | H | S | CF | CH |
| XXXIX-234 | O | Me | H | S | CF | CH |
| XXXIX-235 | CHMe | Me | H | S | CF | CH |
| XXXIX-236 | CH₂ | ═O | | S | CF | CH |
| XXXIX-237 | CH₂ | ═CH₂ | | S | CF | CH |
| XXXIX-238 | CH₂ | ═CHMe | | S | CF | CH |
| XXXIX-239 | CH₂ | H | H | S | CMe | CH |
| XXXIX-240 | CHMe | H | H | S | CMe | CH |
| XXXIX-241 | CHEt | H | H | S | CMe | CH |
| XXXIX-242 | CHF | H | H | S | CMe | CH |
| XXXIX-243 | C═CH₂ | H | H | S | CMe | CH |
| XXXIX-244 | O | H | H | S | CMe | CH |
| XXXIX-245 | S | H | H | S | CMe | CH |
| XXXIX-246 | NH | H | H | S | CMe | CH |
| XXXIX-247 | NMe | H | H | S | CMe | CH |
| XXXIX-248 | NCHO | H | H | S | CMe | CH |
| XXXIX-249 | NCOMe | H | H | S | CMe | CH |
| XXXIX-250 | CH₂ | Me | H | S | CMe | CH |
| XXXIX-251 | O | Me | H | S | CMe | CH |
| XXXIX-252 | CHMe | Me | H | S | CMe | CH |
| XXXIX-253 | CH₂ | ═O | | S | CMe | CH |
| XXXIX-254 | CH₂ | ═CH₂ | | S | CMe | CH |
| XXXIX-255 | CH₂ | ═CHMe | | S | CMe | CH |
| XXXIX-256 | CH₂ | H | H | S | CH | CMe |
| XXXIX-257 | CHMe | H | H | S | CH | CMe |
| XXXIX-258 | CHEt | H | H | S | CH | CMe |
| XXXIX-259 | CHF | H | H | S | CH | CMe |
| XXXIX-260 | C═CH₂ | H | H | S | CH | CMe |
| XXXIX-261 | O | H | H | S | CH | CMe |
| XXXIX-262 | S | H | H | S | CH | CMe |
| XXXIX-263 | NH | H | H | S | CH | CMe |
| XXXIX-264 | NMe | H | H | S | CH | CMe |
| XXXIX-265 | NCHO | H | H | S | CH | CMe |
| XXXIX-266 | NCOMe | H | H | S | CH | CMe |
| XXXIX-267 | CH₂ | Me | H | S | CH | CMe |
| XXXIX-268 | O | Me | H | S | CH | CMe |
| XXXIX-269 | CHMe | Me | H | S | CH | CMe |
| XXXIX-270 | CH₂ | ═O | | S | CH | CMe |
| XXXIX-271 | CH₂ | ═CH₂ | | S | CH | CMe |
| XXXIX-272 | CH₂ | ═CHMe | | S | CH | CMe |
| XXXIX-273 | CH₂ | H | H | NH | N | CH |
| XXXIX-274 | CHMe | H | H | NH | N | CH |
| XXXIX-275 | CHEt | H | H | NH | N | CH |
| XXXIX-276 | CHF | H | H | NH | N | CH |
| XXXIX-277 | C═CH₂ | H | H | NH | N | CH |
| XXXIX-278 | O | H | H | NH | N | CH |
| XXXIX-279 | S | H | H | NH | N | CH |
| XXXIX-280 | NH | H | H | NH | N | CH |
| XXXIX-281 | NMe | H | H | NH | N | CH |
| XXXIX-282 | NCHO | H | H | NH | N | CH |
| XXXIX-283 | NCOMe | H | H | NH | N | CH |
| XXXIX-284 | CH₂ | Me | H | NH | N | CH |
| XXXIX-285 | O | Me | H | NH | N | CH |
| XXXIX-286 | CHMe | Me | H | NH | N | CH |
| XXXIX-287 | CH₂ | ═O | | NH | N | CH |
| XXXIX-288 | CH₂ | ═CH₂ | | NH | N | CH |
| XXXIX-289 | CH₂ | ═CHMe | | NH | N | CH |
| XXXIX-290 | CH₂ | H | H | NH | N | CMe |
| XXXIX-291 | CHMe | H | H | NH | N | CMe |
| XXXIX-292 | CHEt | H | H | NH | N | CMe |
| XXXIX-293 | CHF | H | H | NH | N | CMe |
| XXXIX-294 | C═CH₂ | H | H | NH | N | CMe |
| XXXIX-295 | O | H | H | NH | N | CMe |
| XXXIX-296 | S | H | H | NH | N | CMe |
| XXXIX-297 | NH | H | H | NH | N | CMe |
| XXXIX-298 | NMe | H | H | NH | N | CMe |
| XXXIX-299 | NCHO | H | H | NH | N | CMe |
| XXXIX-300 | NCOMe | H | H | NH | N | CMe |
| XXXIX-301 | CH₂ | Me | H | NH | N | CMe |
| XXXIX-302 | O | Me | H | NH | N | CMe |
| XXXIX-303 | CHMe | Me | H | NH | N | CMe |
| XXXIX-304 | CH₂ | ═O | | NH | N | CMe |
| XXXIX-305 | CH₂ | ═CH₂ | | NH | N | CMe |
| XXXIX-306 | CH₂ | ═CHMe | | NH | N | CMe |
| XXXIX-307 | CH₂ | H | H | NMe | N | CH |
| XXXIX-308 | CHMe | H | H | NMe | N | CH |
| XXXIX-309 | CHEt | H | H | NMe | N | CH |
| XXXIX-310 | CHF | H | H | NMe | N | CH |
| XXXIX-311 | C═CH₂ | H | H | NMe | N | CH |
| XXXIX-312 | O | H | H | NMe | N | CH |
| XXXIX-313 | S | H | H | NMe | N | CH |
| XXXIX-314 | NH | H | H | NMe | N | CH |
| XXXIX-315 | NMe | H | H | NMe | N | CH |
| XXXIX-316 | NCHO | H | H | NMe | N | CH |
| XXXIX-317 | NCOMe | H | H | NMe | N | CH |
| XXXIX-318 | CH₂ | Me | H | NMe | N | CH |
| XXXIX-319 | O | Me | H | NMe | N | CH |
| XXXIX-320 | CHMe | Me | H | NMe | N | CH |
| XXXIX-321 | CH₂ | ═O | | NMe | N | CH |
| XXXIX-322 | CH₂ | ═CH₂ | | NMe | N | CH |
| XXXIX-323 | CH₂ | ═CHMe | | NMe | N | CH |
| XXXIX-324 | CH₂ | H | H | NMe | N | CMe |
| XXXIX-325 | CHMe | H | H | NMe | N | CMe |
| XXXIX-326 | CHEt | H | H | NMe | N | CMe |
| XXXIX-327 | CHF | H | H | NMe | N | CMe |
| XXXIX-328 | C═CH₂ | H | H | NMe | N | CMe |
| XXXIX-329 | O | H | H | NMe | N | CMe |
| XXXIX-330 | S | H | H | NMe | N | CMe |
| XXXIX-331 | NH | H | H | NMe | N | CMe |
| XXXIX-332 | NMe | H | H | NMe | N | CMe |
| XXXIX-333 | NCHO | H | H | NMe | N | CMe |
| XXXIX-334 | NCOMe | H | H | NMe | N | CMe |
| XXXIX-335 | CH₂ | Me | H | NMe | N | CMe |
| XXXIX-336 | O | Me | H | NMe | N | CMe |
| XXXIX-337 | CHMe | Me | H | NMe | N | CMe |
| XXXIX-338 | CH₂ | ═O | | NMe | N | CMe |
| XXXIX-339 | CH₂ | ═CH₂ | | NMe | N | CMe |
| XXXIX-340 | CH₂ | ═CHMe | | NMe | N | CMe |
| XXXIX-341 | CH₂ | H | H | O | N | CH |
| XXXIX-342 | CHMe | H | H | O | N | CH |
| XXXIX-343 | CHEt | H | H | O | N | CH |
| XXXIX-344 | CHF | H | H | O | N | CH |
| XXXIX-345 | C═CH₂ | H | H | O | N | CH |
| XXXIX-346 | O | H | H | O | N | CH |
| XXXIX-347 | S | H | H | O | N | CH |
| XXXIX-348 | NH | H | H | O | N | CH |
| XXXIX-349 | NMe | H | H | O | N | CH |
| XXXIX-350 | NCHO | H | H | O | N | CH |
| XXXIX-351 | NCOMe | H | H | O | N | CH |
| XXXIX-352 | CH₂ | Me | H | O | N | CH |
| XXXIX-353 | O | Me | H | O | N | CH |
| XXXIX-354 | CHMe | Me | H | O | N | CH |
| XXXIX-355 | CH₂ | ═O | | O | N | CH |
| XXXIX-356 | CH₂ | ═CH₂ | | O | N | CH |
| XXXIX-357 | CH₂ | ═CHMe | | O | N | CH |
| XXXIX-358 | CH₂ | H | H | O | N | CMe |
| XXXIX-359 | CHMe | H | H | O | N | CMe |
| XXXIX-360 | CHEt | H | H | O | N | CMe |
| XXXIX-361 | CHF | H | H | O | N | CMe |
| XXXIX-362 | C═CH₂ | H | H | O | N | CMe |
| XXXIX-363 | O | H | H | O | N | CMe |
| XXXIX-364 | S | H | H | O | N | CMe |
| XXXIX-365 | NH | H | H | O | N | CMe |
| XXXIX-366 | NMe | H | H | O | N | CMe |
| XXXIX-367 | NCHO | H | H | O | N | CMe |
| XXXIX-368 | NCOMe | H | H | O | N | CMe |
| XXXIX-369 | CH₂ | Me | H | O | N | CMe |
| XXXIX-370 | O | Me | H | O | N | CMe |
| XXXIX-371 | CHMe | Me | H | O | N | CMe |
| XXXIX-372 | CH₂ | ═O | | O | N | CMe |
| XXXIX-373 | CH₂ | ═CH₂ | | O | N | CMe |
| XXXIX-374 | CH₂ | ═CHMe | | O | N | CMe |
| XXXIX-375 | CH₂ | H | H | S | N | CH |
| XXXIX-376 | CHMe | H | H | S | N | CH |
| XXXIX-377 | CHEt | H | H | S | N | CH |
| XXXIX-378 | CHF | H | H | S | N | CH |
| XXXIX-379 | C═CH₂ | H | H | S | N | CH |
| XXXIX-380 | O | H | H | S | N | CH |
| XXXIX-381 | S | H | H | S | N | CH |
| XXXIX-382 | NH | H | H | S | N | CH |
| XXXIX-383 | NMe | H | H | S | N | CH |
| XXXIX-384 | NCHO | H | H | S | N | CH |
| XXXIX-385 | NCOMe | H | H | S | N | CH |
| XXXIX-386 | CH₂ | Me | H | S | N | CH |
| XXXIX-387 | O | Me | H | S | N | CH |
| XXXIX-388 | CHMe | Me | H | S | N | CH |
| XXXIX-389 | CH₂ | ═O | | S | N | CH |
| XXXIX-390 | CH₂ | ═CH₂ | | S | N | CH |
| XXXIX-391 | CH₂ | ═CHMe | | S | N | CH |
| XXXIX-392 | CH₂ | H | H | S | N | CMe |
| XXXIX-393 | CHMe | H | H | S | N | CMe |
| XXXIX-394 | CHEt | H | H | S | N | CMe |
| XXXIX-395 | CHF | H | H | S | N | CMe |
| XXXIX-396 | C═CH₂ | H | H | S | N | CMe |
| XXXIX-397 | O | H | H | S | N | CMe |
| XXXIX-398 | S | H | H | S | N | CMe |
| XXXIX-399 | NH | H | H | S | N | CMe |
| XXXIX-400 | NMe | H | H | S | N | CMe |
| XXXIX-401 | NCHO | H | H | S | N | CMe |
| XXXIX-402 | NCOMe | H | H | S | N | CMe |
| XXXIX-403 | CH₂ | Me | H | S | N | CMe |
| XXXIX-404 | O | Me | H | S | N | CMe |
| XXXIX-405 | CHMe | Me | H | S | N | CMe |
| XXXIX-406 | CH₂ | ═O | | S | N | CMe |
| XXXIX-407 | CH₂ | ═CH₂ | | S | N | CMe |
| XXXIX-408 | CH₂ | ═CHMe | | S | N | CMe |
| XXXIX-409 | CH₂ | H | H | NH | CH | N |
| XXXIX-410 | CHMe | H | H | NH | CH | N |
| XXXIX-411 | CHEt | H | H | NH | CH | N |
| XXXIX-412 | CHF | H | H | NH | CH | N |
| XXXIX-413 | C═CH₂ | H | H | NH | CH | N |
| XXXIX-414 | O | H | H | NH | CH | N |
| XXXIX-415 | S | H | H | NH | CH | N |
| XXXIX-416 | NH | H | H | NH | CH | N |
| XXXIX-417 | NMe | H | H | NH | CH | N |
| XXXIX-418 | NCHO | H | H | NH | CH | N |
| XXXIX-419 | NCOMe | H | H | NH | CH | N |
| XXXIX-420 | CH₂ | Me | H | NH | CH | N |
| XXXIX-421 | O | Me | H | NH | CH | N |
| XXXIX-422 | CHMe | Me | H | NH | CH | N |
| XXXIX-423 | CH₂ | ═O | | NH | CH | N |
| XXXIX-424 | CH₂ | ═CH₂ | | NH | CH | N |
| XXXIX-425 | CH₂ | ═CHMe | | NH | CH | N |
| XXXIX-426 | CH₂ | H | H | NH | CMe | N |
| XXXIX-427 | CHMe | H | H | NH | CMe | N |
| XXXIX-428 | CHEt | H | H | NH | CMe | N |
| XXXIX-429 | CHF | H | H | NH | CMe | N |
| XXXIX-430 | C═CH₂ | H | H | NH | CMe | N |
| XXXIX-431 | O | H | H | NH | CMe | N |
| XXXIX-432 | S | H | H | NH | CMe | N |
| XXXIX-433 | NH | H | H | NH | CMe | N |
| XXXIX-434 | NMe | H | H | NH | CMe | N |
| XXXIX-435 | NCHO | H | H | NH | CMe | N |
| XXXIX-436 | NCOMe | H | H | NH | CMe | N |
| XXXIX-437 | CH₂ | Me | H | NH | CMe | N |
| XXXIX-438 | O | Me | H | NH | CMe | N |
| XXXIX-439 | CHMe | Me | H | NH | CMe | N |
| XXXIX-440 | CH₂ | ═O | | NH | CMe | N |
| XXXIX-441 | CH₂ | ═CH₂ | | NH | CMe | N |
| XXXIX-442 | CH₂ | ═CHMe | | NH | CMe | N |
| XXXIX-443 | CH₂ | H | H | O | CH | N |
| XXXIX-444 | CHMe | H | H | O | CH | N |
| XXXIX-445 | CHEt | H | H | O | CH | N |
| XXXIX-446 | CHF | H | H | O | CH | N |
| XXXIX-447 | C═CH₂ | H | H | O | CH | N |
| XXXIX-448 | O | H | H | O | CH | N |
| XXXIX-449 | S | H | H | O | CH | N |
| XXXIX-450 | NH | H | H | O | CH | N |
| XXXIX-451 | NMe | H | H | O | CH | N |
| XXXIX-452 | NCHO | H | H | O | CH | N |
| XXXIX-453 | NCOMe | H | H | O | CH | N |
| XXXIX-454 | CH₂ | Me | H | O | CH | N |
| XXXIX-455 | O | Me | H | O | CH | N |
| XXXIX-456 | CHMe | Me | H | O | CH | N |
| XXXIX-457 | CH₂ | ═O | | O | CH | N |
| XXXIX-458 | CH₂ | ═CH₂ | | O | CH | N |
| XXXIX-459 | CH₂ | ═CHMe | | O | CH | N |
| XXXIX-460 | CH₂ | H | H | O | CMe | N |
| XXXIX-461 | CHMe | H | H | O | CMe | N |
| XXXIX-462 | CHEt | H | H | O | CMe | N |
| XXXIX-463 | CHF | H | H | O | CMe | N |
| XXXIX-464 | C═CH₂ | H | H | O | CMe | N |
| XXXIX-465 | O | H | H | O | CMe | N |
| XXXIX-466 | S | H | H | O | CMe | N |
| XXXIX-467 | NH | H | H | O | CMe | N |
| XXXIX-468 | NMe | H | H | O | CMe | N |
| XXXIX-469 | NCHO | H | H | O | CMe | N |
| XXXIX-470 | NCOMe | H | H | O | CMe | N |
| XXXIX-471 | CH₂ | Me | H | O | CMe | N |
| XXXIX-472 | O | Me | H | O | CMe | N |
| XXXIX-473 | CHMe | Me | H | O | CMe | N |
| XXXIX-474 | CH₂ | ═O | | O | CMe | N |
| XXXIX-475 | CH₂ | ═CH₂ | | O | CMe | N |
| XXXIX-476 | CH₂ | ═CHMe | | O | CMe | N |
| XXXIX-477 | CH₂ | H | H | S | CH | N |
| XXXIX-478 | CHMe | H | H | S | CH | N |
| XXXIX-479 | CHEt | H | H | S | CH | N |
| XXXIX-480 | CHF | H | H | S | CH | N |
| XXXIX-481 | C═CH₂ | H | H | S | CH | N |
| XXXIX-482 | O | H | H | S | CH | N |
| XXXIX-483 | S | H | H | S | CH | N |
| XXXIX-484 | NH | H | H | S | CH | N |
| XXXIX-485 | NMe | H | H | S | CH | N |
| XXXIX-486 | NCHO | H | H | S | CH | N |
| XXXIX-487 | NCOMe | H | H | S | CH | N |
| XXXIX-488 | CH₂ | Me | H | S | CH | N |
| XXXIX-489 | O | Me | H | S | CH | N |
| XXXIX-490 | CHMe | Me | H | S | CH | N |
| XXXIX-491 | CH₂ | ═O | | S | CH | N |
| XXXIX-492 | CH₂ | ═CH₂ | | S | CH | N |
| XXXIX-493 | CH₂ | ═CHMe | | S | CH | N |
| XXXIX-494 | CH₂ | H | H | S | CMe | N |
| XXXIX-495 | CHMe | H | H | S | CMe | N |
| XXXIX-496 | CHEt | H | H | S | CMe | N |
| XXXIX-497 | CHF | H | H | S | CMe | N |
| XXXIX-498 | C═CH₂ | H | H | S | CMe | N |
| XXXIX-499 | O | H | H | S | CMe | N |
| XXXIX-500 | S | H | H | S | CMe | N |
| XXXIX-501 | NH | H | H | S | CMe | N |
| XXXIX-502 | NMe | H | H | S | CMe | N |
| XXXIX-503 | NCHO | H | H | S | CMe | N |
| XXXIX-504 | NCOMe | H | H | S | CMe | N |
| XXXIX-505 | CH₂ | Me | H | S | CMe | N |
| XXXIX-506 | O | Me | H | S | CMe | N |
| XXXIX-507 | CHMe | Me | H | S | CMe | N |
| XXXIX-508 | CH₂ | ═O | | S | CMe | N |
| XXXIX-509 | CH₂ | ═CH₂ | | S | CMe | N |
| XXXIX-510 | CH₂ | ═CHMe | | S | CMe | N |
| XXXIX-511 | CH₂ | H | H | NH | N | N |
| XXXIX-512 | CHMe | H | H | NH | N | N |
| XXXIX-513 | CHEt | H | H | NH | N | N |
| XXXIX-514 | CHF | H | H | NH | N | N |
| XXXIX-515 | C═CH₂ | H | H | NH | N | N |
| XXXIX-516 | O | H | H | NH | N | N |
| XXXIX-517 | S | H | H | NH | N | N |
| XXXIX-518 | NH | H | H | NH | N | N |
| XXXIX-519 | NMe | H | H | NH | N | N |
| XXXIX-520 | NCHO | H | H | NH | N | N |
| XXXIX-521 | NCOMe | H | H | NH | N | N |
| XXXIX-522 | CH₂ | Me | H | NH | N | N |
| XXXIX-523 | O | Me | H | NH | N | N |
| XXXIX-524 | CHMe | Me | H | NH | N | N |
| XXXIX-525 | CH₂ | ═O | | NH | N | N |
| XXXIX-526 | CH₂ | ═CH₂ | | NH | N | N |
| XXXIX-527 | CH₂ | ═CHMe | | NH | N | N |
| XXXIX-528 | CH₂ | H | H | NMe | N | N |
| XXXIX-529 | CHMe | H | H | NMe | N | N |
| XXXIX-530 | CHEt | H | H | NMe | N | N |
| XXXIX-531 | CHF | H | H | NMe | N | N |
| XXXIX-532 | C═CH₂ | H | H | NMe | N | N |
| XXXIX-533 | O | H | H | NMe | N | N |
| XXXIX-534 | S | H | H | NMe | N | N |
| XXXIX-535 | NH | H | H | NMe | N | N |
| XXXIX-536 | NMe | H | H | NMe | N | N |
| XXXIX-537 | NCHO | H | H | NMe | N | N |
| XXXIX-538 | NCOMe | H | H | NMe | N | N |
| XXXIX-539 | CH₂ | Me | H | NMe | N | N |
| XXXIX-540 | O | Me | H | NMe | N | N |
| XXXIX-541 | CHMe | Me | H | NMe | N | N |
| XXXIX-542 | CH₂ | ═O | | NMe | N | N |
| XXXIX-543 | CH₂ | ═CH₂ | | NMe | N | N |
| XXXIX-544 | CH₂ | ═CHMe | | NMe | N | N |
| XXXIX-545 | CH₂ | H | H | O | N | N |
| XXXIX-546 | CHMe | H | H | O | N | N |
| XXXIX-547 | CHEt | H | H | O | N | N |
| XXXIX-548 | CHF | H | H | O | N | N |
| XXXIX-549 | C═CH₂ | H | H | O | N | N |
| XXXIX-550 | O | H | H | O | N | N |
| XXXIX-551 | S | H | H | O | N | N |
| XXXIX-552 | NH | H | H | O | N | N |
| XXXIX-553 | NMe | H | H | O | N | N |
| XXXIX-554 | NCHO | H | H | O | N | N |
| XXXIX-555 | NCOMe | H | H | O | N | N |
| XXXIX-556 | CH₂ | Me | H | O | N | N |
| XXXIX-557 | O | Me | H | O | N | N |
| XXXIX-558 | CHMe | Me | H | O | N | N |
| XXXIX-559 | CH₂ | ═O | | O | N | N |
| XXXIX-560 | CH₂ | ═CH₂ | | O | N | N |
| XXXIX-561 | CH₂ | ═CHMe | | O | N | N |
| XXXIX-562 | CH₂ | H | H | S | N | N |
| XXXIX-563 | CHMe | H | H | S | N | N |
| XXXIX-564 | CHEt | H | H | S | N | N |
| XXXIX-565 | CHF | H | H | S | N | N |
| XXXIX-566 | C═CH₂ | H | H | S | N | N |
| XXXIX-567 | O | H | H | S | N | N |
| XXXIX-568 | S | H | H | S | N | N |
| XXXIX-569 | NH | H | H | S | N | N |
| XXXIX-570 | NMe | H | H | S | N | N |
| XXXIX-571 | NCHO | H | H | S | N | N |
| XXXIX-572 | NCOMe | H | H | S | N | N |
| XXXIX-573 | CH₂ | Me | H | S | N | N |
| XXXIX-574 | O | Me | H | S | N | N |
| XXXIX-575 | CHMe | Me | H | S | N | N |

| | | | | | |
|---|---|---|---|---|---|
| XXXIX-576 | CH₂ | ═O | S | N | N |
| XXXIX-577 | CH₂ | ═CH₂ | S | N | N |
| XXXIX-578 | CH₂ | ═CHMe | S | N | N |

Table XL provides 392 compounds of formula Ian

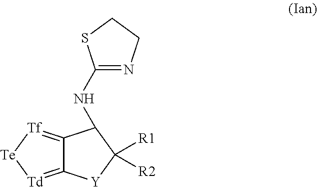

wherein the values of Y, R¹, R², T^{d}, T^{e} and T^{f} are given in Table 7.

TABLE 7

| Compound No | Y | R1 | R2 | Td | Te | Tf |
|---|---|---|---|---|---|---|
| XL-1 | CH₂ | H | H | CH | NH | CH |
| XL-2 | CHMe | H | H | CH | NH | CH |
| XL-3 | CHEt | H | H | CH | NH | CH |
| XL-4 | CHF | H | H | CH | NH | CH |
| XL-5 | C═CH₂ | H | H | CH | NH | CH |
| XL-6 | O | H | H | CH | NH | CH |
| XL-7 | S | H | H | CH | NH | CH |
| XL-8 | NH | H | H | CH | NH | CH |
| XL-9 | NMe | H | H | CH | NH | CH |
| XL-10 | NCHO | H | H | CH | NH | CH |
| XL-11 | NCOMe | H | H | CH | NH | CH |
| XL-12 | CH₂ | Me | H | CH | NH | CH |
| XL-13 | O | Me | H | CH | NH | CH |
| XL-14 | CHMe | Me | H | CH | NH | CH |
| XL-15 | CH₂ | H | H | CMe | NH | CH |
| XL-16 | CHMe | H | H | CMe | NH | CH |
| XL-17 | CHEt | H | H | CMe | NH | CH |
| XL-18 | CHF | H | H | CMe | NH | CH |
| XL-19 | C═CH₂ | H | H | CMe | NH | CH |
| XL-20 | O | H | H | CMe | NH | CH |
| XL-21 | S | H | H | CMe | NH | CH |
| XL-22 | NH | H | H | CMe | NH | CH |
| XL-23 | NMe | H | H | CMe | NH | CH |
| XL-24 | NCHO | H | H | CMe | NH | CH |
| XL-25 | NCOMe | H | H | CMe | NH | CH |
| XL-26 | CH₂ | Me | H | CMe | NH | CH |
| XL-27 | O | Me | H | CMe | NH | CH |
| XL-28 | CHMe | Me | H | CMe | NH | CH |
| XL-29 | CH₂ | H | H | CH | NH | CMe |
| XL-30 | CHMe | H | H | CH | NH | CMe |
| XL-31 | CHEt | H | H | CH | NH | CMe |
| XL-32 | CHF | H | H | CH | NH | CMe |
| XL-33 | C═CH₂ | H | H | CH | NH | CMe |
| XL-34 | O | H | H | CH | NH | CMe |
| XL-35 | S | H | H | CH | NH | CMe |
| XL-36 | NH | H | H | CH | NH | CMe |
| XL-37 | NMe | H | H | CH | NH | CMe |
| XL-38 | NCHO | H | H | CH | NH | CMe |
| XL-39 | NCOMe | H | H | CH | NH | CMe |
| XL-40 | CH₂ | Me | H | CH | NH | CMe |
| XL-41 | O | Me | H | CH | NH | CMe |
| XL-42 | CHMe | Me | H | CH | NH | CMe |
| XL-43 | CH₂ | H | H | CMe | NH | CMe |
| XL-44 | CHMe | H | H | CMe | NH | CMe |
| XL-45 | CHEt | H | H | CMe | NH | CMe |
| XL-46 | CHF | H | H | CMe | NH | CMe |
| XL-47 | C═CH₂ | H | H | CMe | NH | CMe |
| XL-48 | O | H | H | CMe | NH | CMe |
| XL-49 | S | H | H | CMe | NH | CMe |
| XL-50 | NH | H | H | CMe | NH | CMe |
| XL-51 | NMe | H | H | CMe | NH | CMe |
| XL-52 | NCHO | H | H | CMe | NH | CMe |
| XL-53 | NCOMe | H | H | CMe | NH | CMe |
| XL-54 | CH₂ | Me | H | CMe | NH | CMe |
| XL-55 | O | Me | H | CMe | NH | CMe |
| XL-56 | CHMe | Me | H | CMe | NH | CMe |
| XL-57 | CH₂ | H | H | CH | NMe | CH |
| XL-58 | CHMe | H | H | CH | NMe | CH |
| XL-59 | CHEt | H | H | CH | NMe | CH |
| XL-60 | CHF | H | H | CH | NMe | CH |
| XL-61 | C═CH₂ | H | H | CH | NMe | CH |
| XL-62 | O | H | H | CH | NMe | CH |
| XL-63 | S | H | H | CH | NMe | CH |
| XL-64 | NH | H | H | CH | NMe | CH |
| XL-65 | NMe | H | H | CH | NMe | CH |
| XL-66 | NCHO | H | H | CH | NMe | CH |
| XL-67 | NCOMe | H | H | CH | NMe | CH |
| XL-68 | CH₂ | Me | H | CH | NMe | CH |
| XL-69 | O | Me | H | CH | NMe | CH |
| XL-70 | CHMe | Me | H | CH | NMe | CH |
| XL-71 | CH₂ | H | H | CMe | NMe | CH |
| XL-72 | CHMe | H | H | CMe | NMe | CH |
| XL-73 | CHEt | H | H | CMe | NMe | CH |
| XL-74 | CHF | H | H | CMe | NMe | CH |
| XL-75 | C═CH₂ | H | H | CMe | NMe | CH |
| XL-76 | O | H | H | CMe | NMe | CH |
| XL-77 | S | H | H | CMe | NMe | CH |
| XL-78 | NH | H | H | CMe | NMe | CH |
| XL-79 | NMe | H | H | CMe | NMe | CH |
| XL-80 | NCHO | H | H | CMe | NMe | CH |
| XL-81 | NCOMe | H | H | CMe | NMe | CH |
| XL-82 | CH₂ | Me | H | CMe | NMe | CH |
| XL-83 | O | Me | H | CMe | NMe | CH |
| XL-84 | CHMe | Me | H | CMe | NMe | CH |
| XL-85 | CH₂ | H | H | CH | NMe | CMe |
| XL-86 | CHMe | H | H | CH | NMe | CMe |
| XL-87 | CHEt | H | H | CH | NMe | CMe |
| XL-88 | CHF | H | H | CH | NMe | CMe |
| XL-89 | C═CH₂ | H | H | CH | NMe | CMe |
| XL-90 | O | H | H | CH | NMe | CMe |
| XL-91 | S | H | H | CH | NMe | CMe |
| XL-92 | NH | H | H | CH | NMe | CMe |
| XL-93 | NMe | H | H | CH | NMe | CMe |
| XL-94 | NCHO | H | H | CH | NMe | CMe |
| XL-95 | NCOMe | H | H | CH | NMe | CMe |
| XL-96 | CH₂ | Me | H | CH | NMe | CMe |
| XL-97 | O | Me | H | CH | NMe | CMe |
| XL-98 | CHMe | Me | H | CH | NMe | CMe |
| XL-99 | CH₂ | H | H | CMe | NMe | CMe |
| XL-100 | CHMe | H | H | CMe | NMe | CMe |
| XL-101 | CHEt | H | H | CMe | NMe | CMe |
| XL-102 | CHF | H | H | CMe | NMe | CMe |
| XL-103 | C═CH₂ | H | H | CMe | NMe | CMe |
| XL-104 | O | H | H | CMe | NMe | CMe |
| XL-105 | S | H | H | CMe | NMe | CMe |
| XL-106 | NH | H | H | CMe | NMe | CMe |
| XL-107 | NMe | H | H | CMe | NMe | CMe |
| XL-108 | NCHO | H | H | CMe | NMe | CMe |
| XL-109 | NCOMe | H | H | CMe | NMe | CMe |
| XL-110 | CH₂ | Me | H | CMe | NMe | CMe |
| XL-111 | O | Me | H | CMe | NMe | CMe |
| XL-112 | CHMe | Me | H | CMe | NMe | CMe |
| XL-113 | CH₂ | H | H | CH | O | CH |
| XL-114 | CHMe | H | H | CH | O | CH |
| XL-115 | CHEt | H | H | CH | O | CH |
| XL-116 | CHF | H | H | CH | O | CH |
| XL-117 | C═CH₂ | H | H | CH | O | CH |
| XL-118 | O | H | H | CH | O | CH |
| XL-119 | S | H | H | CH | O | CH |
| XL-120 | NH | H | H | CH | O | CH |
| XL-121 | NMe | H | H | CH | O | CH |
| XL-122 | NCHO | H | H | CH | O | CH |
| XL-123 | NCOMe | H | H | CH | O | CH |
| XL-124 | CH₂ | Me | H | CH | O | CH |
| XL-125 | O | Me | H | CH | O | CH |
| XL-126 | CHMe | Me | H | CH | O | CH |
| XL-127 | CH₂ | H | H | CMe | O | CH |
| XL-128 | CHMe | H | H | CMe | O | CH |
| XL-129 | CHEt | H | H | CMe | O | CH |
| XL-130 | CHF | H | H | CMe | O | CH |
| XL-131 | C═CH₂ | H | H | CMe | O | CH |
| XL-132 | O | H | H | CMe | O | CH |
| XL-133 | S | H | H | CMe | O | CH |
| XL-134 | NH | H | H | CMe | O | CH |
| XL-135 | NMe | H | H | CMe | O | CH |
| XL-136 | NCHO | H | H | CMe | O | CH |
| XL-137 | NCOMe | H | H | CMe | O | CH |
| XL-138 | CH₂ | Me | H | CMe | O | CH |
| XL-139 | O | Me | H | CMe | O | CH |
| XL-140 | CHMe | Me | H | CMe | O | CH |
| XL-141 | CH₂ | H | H | CH | O | CMe |
| XL-142 | CHMe | H | H | CH | O | CMe |
| XL-143 | CHEt | H | H | CH | O | CMe |
| XL-144 | CHF | H | H | CH | O | CMe |
| XL-145 | C═CH₂ | H | H | CH | O | CMe |
| XL-146 | O | H | H | CH | O | CMe |
| XL-147 | S | H | H | CH | O | CMe |
| XL-148 | NH | H | H | CH | O | CMe |
| XL-149 | NMe | H | H | CH | O | CMe |
| XL-150 | NCHO | H | H | CH | O | CMe |
| XL-151 | NCOMe | H | H | CH | O | CMe |
| XL-152 | CH₂ | Me | H | CH | O | CMe |
| XL-153 | O | Me | H | CH | O | CMe |
| XL-154 | CHMe | Me | H | CH | O | CMe |
| XL-155 | CH₂ | H | H | CMe | O | CMe |
| XL-156 | CHMe | H | H | CMe | O | CMe |
| XL-157 | CHEt | H | H | CMe | O | CMe |
| XL-158 | CHF | H | H | CMe | O | CMe |
| XL-159 | C═CH₂ | H | H | CMe | O | CMe |
| XL-160 | O | H | H | CMe | O | CMe |
| XL-161 | S | H | H | CMe | O | CMe |
| XL-162 | NH | H | H | CMe | O | CMe |
| XL-163 | NMe | H | H | CMe | O | CMe |
| XL-164 | NCHO | H | H | CMe | O | CMe |
| XL-165 | NCOMe | H | H | CMe | O | CMe |
| XL-166 | CH₂ | Me | H | CMe | O | CMe |
| XL-167 | O | Me | H | CMe | O | CMe |
| XL-168 | CHMe | Me | H | CMe | O | CMe |
| XL-169 | CH₂ | H | H | CH | S | CH |
| XL-170 | CHMe | H | H | CH | S | CH |
| XL-171 | CHEt | H | H | CH | S | CH |
| XL-172 | CHF | H | H | CH | S | CH |
| XL-173 | C═CH₂ | H | H | CH | S | CH |
| XL-174 | O | H | H | CH | S | CH |
| XL-175 | S | H | H | CH | S | CH |
| XL-176 | NH | H | H | CH | S | CH |
| XL-177 | NMe | H | H | CH | S | CH |
| XL-178 | NCHO | H | H | CH | S | CH |
| XL-179 | NCOMe | H | H | CH | S | CH |
| XL-180 | CH₂ | Me | H | CH | S | CH |
| XL-181 | O | Me | H | CH | S | CH |
| XL-182 | CHMe | Me | H | CH | S | CH |
| XL-183 | CH₂ | H | H | CMe | S | CH |
| XL-184 | CHMe | H | H | CMe | S | CH |
| XL-185 | CHEt | H | H | CMe | S | CH |
| XL-186 | CHF | H | H | CMe | S | CH |
| XL-187 | C═CH₂ | H | H | CMe | S | CH |
| XL-188 | O | H | H | CMe | S | CH |
| XL-189 | S | H | H | CMe | S | CH |
| XL-190 | NH | H | H | CMe | S | CH |
| XL-191 | NMe | H | H | CMe | S | CH |
| XL-192 | NCHO | H | H | CMe | S | CH |
| XL-193 | NCOMe | H | H | CMe | S | CH |
| XL-194 | CH₂ | Me | H | CMe | S | CH |
| XL-195 | O | Me | H | CMe | S | CH |
| XL-196 | CHMe | Me | H | CMe | S | CH |
| XL-197 | CH₂ | H | H | CH | S | CMe |
| XL-198 | CHMe | H | H | CH | S | CMe |
| XL-199 | CHEt | H | H | CH | S | CMe |
| XL-200 | CHF | H | H | CH | S | CMe |
| XL-201 | C═CH₂ | H | H | CH | S | CMe |
| XL-202 | O | H | H | CH | S | CMe |
| XL-203 | S | H | H | CH | S | CMe |
| XL-204 | NH | H | H | CH | S | CMe |
| XL-205 | NMe | H | H | CH | S | CMe |
| XL-206 | NCHO | H | H | CH | S | CMe |
| XL-207 | NCOMe | H | H | CH | S | CMe |
| XL-208 | CH₂ | Me | H | CH | S | CMe |
| XL-209 | O | Me | H | CH | S | CMe |
| XL-210 | CHMe | Me | H | CH | S | CMe |
| XL-211 | CH₂ | H | H | CMe | S | CMe |
| XL-212 | CHMe | H | H | CMe | S | CMe |
| XL-213 | CHEt | H | H | CMe | S | CMe |
| XL-214 | CHF | H | H | CMe | S | CMe |
| XL-215 | C═CH₂ | H | H | CMe | S | CMe |
| XL-216 | O | H | H | CMe | S | CMe |
| XL-217 | S | H | H | CMe | S | CMe |
| XL-218 | NH | H | H | CMe | S | CMe |
| XL-219 | NMe | H | H | CMe | S | CMe |
| XL-220 | NCHO | H | H | CMe | S | CMe |
| XL-221 | NCOMe | H | H | CMe | S | CMe |
| XL-222 | CH₂ | Me | H | CMe | S | CMe |
| XL-223 | O | Me | H | CMe | S | CMe |
| XL-224 | CHMe | Me | H | CMe | S | CMe |
| XL-225 | CH₂ | H | H | N | NMe | CH |
| XL-226 | CHMe | H | H | N | NMe | CH |
| XL-227 | CHEt | H | H | N | NMe | CH |
| XL-228 | CHF | H | H | N | NMe | CH |
| XL-229 | C═CH₂ | H | H | N | NMe | CH |
| XL-230 | O | H | H | N | NMe | CH |
| XL-231 | S | H | H | N | NMe | CH |
| XL-232 | NH | H | H | N | NMe | CH |
| XL-233 | NMe | H | H | N | NMe | CH |
| XL-234 | NCHO | H | H | N | NMe | CH |
| XL-235 | NCOMe | H | H | N | NMe | CH |
| XL-236 | CH₂ | Me | H | N | NMe | CH |
| XL-237 | O | Me | H | N | NMe | CH |
| XL-238 | CHMe | Me | H | N | NMe | CH |
| XL-239 | CH₂ | H | H | N | NMe | CMe |
| XL-240 | CHMe | H | H | N | NMe | CMe |
| XL-241 | CHEt | H | H | N | NMe | CMe |
| XL-242 | CHF | H | H | N | NMe | CMe |
| XL-243 | C═CH₂ | H | H | N | NMe | CMe |
| XL-244 | O | H | H | N | NMe | CMe |
| XL-245 | S | H | H | N | NMe | CMe |
| XL-246 | NH | H | H | N | NMe | CMe |
| XL-247 | NMe | H | H | N | NMe | CMe |
| XL-248 | NCHO | H | H | N | NMe | CMe |
| XL-249 | NCOMe | H | H | N | NMe | CMe |
| XL-250 | CH₂ | Me | H | N | NMe | CMe |
| XL-251 | O | Me | H | N | NMe | CMe |
| XL-252 | CHMe | Me | H | N | NMe | CMe |
| XL-253 | CH₂ | H | H | N | O | CH |
| XL-254 | CHMe | H | H | N | O | CH |
| XL-255 | CHEt | H | H | N | O | CH |
| XL-256 | CHF | H | H | N | O | CH |
| XL-257 | C═CH₂ | H | H | N | O | CH |
| XL-258 | O | H | H | N | O | CH |
| XL-259 | S | H | H | N | O | CH |
| XL-260 | NH | H | H | N | O | CH |
| XL-261 | NMe | H | H | N | O | CH |
| XL-262 | NCHO | H | H | N | O | CH |
| XL-263 | NCOMe | H | H | N | O | CH |
| XL-264 | CH₂ | Me | H | N | O | CH |
| XL-265 | O | Me | H | N | O | CH |
| XL-266 | CHMe | Me | H | N | O | CH |
| XL-267 | CH₂ | H | H | N | O | CMe |
| XL-268 | CHMe | H | H | N | O | CMe |
| XL-269 | CHEt | H | H | N | O | CMe |
| XL-270 | CHF | H | H | N | O | CMe |
| XL-271 | C═CH₂ | H | H | N | O | CMe |
| XL-272 | O | H | H | N | O | CMe |
| XL-273 | S | H | H | N | O | CMe |
| XL-274 | NH | H | H | N | O | CMe |
| XL-275 | NMe | H | H | N | O | CMe |
| XL-276 | NCHO | H | H | N | O | CMe |
| XL-277 | NCOMe | H | H | N | O | CMe |
| XL-278 | CH₂ | Me | H | N | O | CMe |
| XL-279 | O | Me | H | N | O | CMe |
| XL-280 | CHMe | Me | H | N | O | CMe |
| XL-281 | CH₂ | H | H | N | S | CH |
| XL-282 | CHMe | H | H | N | S | CH |
| XL-283 | CHEt | H | H | N | S | CH |
| XL-284 | CHF | H | H | N | S | CH |
| XL-285 | C═CH₂ | H | H | N | S | CH |
| XL-286 | O | H | H | N | S | CH |
| XL-287 | S | H | H | N | S | CH |
| XL-288 | NH | H | H | N | S | CH |
| XL-289 | NMe | H | H | N | S | CH |
| XL-290 | NCHO | H | H | N | S | CH |
| XL-291 | NCOMe | H | H | N | S | CH |
| XL-292 | CH₂ | Me | H | N | S | CH |
| XL-293 | O | Me | H | N | S | CH |
| XL-294 | CHMe | Me | H | N | S | CH |
| XL-295 | CH₂ | H | H | N | S | CMe |
| XL-296 | CHMe | H | H | N | S | CMe |
| XL-297 | CHEt | H | H | N | S | CMe |
| XL-298 | CHF | H | H | N | S | CMe |
| XL-299 | C═CH₂ | H | H | N | S | CMe |
| XL-300 | O | H | H | N | S | CMe |
| XL-301 | S | H | H | N | S | CMe |
| XL-302 | NH | H | H | N | S | CMe |
| XL-303 | NMe | H | H | N | S | CMe |
| XL-304 | NCHO | H | H | N | S | CMe |
| XL-305 | NCOMe | H | H | N | S | CMe |
| XL-306 | CH₂ | Me | H | N | S | CMe |
| XL-307 | O | Me | H | N | S | CMe |
| XL-308 | CHMe | Me | H | N | S | CMe |
| XL-309 | CH₂ | H | H | CH | NMe | N |
| XL-310 | CHMe | H | H | CH | NMe | N |
| XL-311 | CHEt | H | H | CH | NMe | N |
| XL-312 | CHF | H | H | CH | NMe | N |
| XL-313 | C═CH₂ | H | H | CH | NMe | N |
| XL-314 | O | H | H | CH | NMe | N |
| XL-315 | S | H | H | CH | NMe | N |
| XL-316 | NH | H | H | CH | NMe | N |
| XL-317 | NMe | H | H | CH | NMe | N |
| XL-318 | NCHO | H | H | CH | NMe | N |
| XL-319 | NCOMe | H | H | CH | NMe | N |
| XL-320 | CH₂ | Me | H | CH | NMe | N |
| XL-321 | O | Me | H | CH | NMe | N |
| XL-322 | CHMe | Me | H | CH | NMe | N |
| XL-323 | CH₂ | H | H | CMe | NMe | N |
| XL-324 | CHMe | H | H | CMe | NMe | N |
| XL-325 | CHEt | H | H | CMe | NMe | N |
| XL-326 | CHF | H | H | CMe | NMe | N |
| XL-327 | C═CH₂ | H | H | CMe | NMe | N |
| XL-328 | O | H | H | CMe | NMe | N |
| XL-329 | S | H | H | CMe | NMe | N |
| XL-330 | NH | H | H | CMe | NMe | N |
| XL-331 | NMe | H | H | CMe | NMe | N |
| XL-332 | NCHO | H | H | CMe | NMe | N |
| XL-333 | NCOMe | H | H | CMe | NMe | N |
| XL-334 | CH₂ | Me | H | CMe | NMe | N |
| XL-335 | O | Me | H | CMe | NMe | N |
| XL-336 | CHMe | Me | H | CMe | NMe | N |
| XL-337 | CH₂ | H | H | CH | O | N |
| XL-338 | CHMe | H | H | CH | O | N |
| XL-339 | CHEt | H | H | CH | O | N |
| XL-340 | CHF | H | H | CH | O | N |
| XL-341 | C═CH₂ | H | H | CH | O | N |
| XL-342 | O | H | H | CH | O | N |
| XL-343 | S | H | H | CH | O | N |
| XL-344 | NH | H | H | CH | O | N |
| XL-345 | NMe | H | H | CH | O | N |
| XL-346 | NCHO | H | H | CH | O | N |
| XL-347 | NCOMe | H | H | CH | O | N |
| XL-348 | CH₂ | Me | H | CH | O | N |
| XL-349 | O | Me | H | CH | O | N |
| XL-350 | CHMe | Me | H | CH | O | N |
| XL-351 | CH₂ | H | H | CMe | O | N |
| XL-352 | CHMe | H | H | CMe | O | N |
| XL-353 | CHEt | H | H | CMe | O | N |
| XL-354 | CHF | H | H | CMe | O | N |
| XL-355 | C═CH₂ | H | H | CMe | O | N |
| XL-356 | O | H | H | CMe | O | N |
| XL-357 | S | H | H | CMe | O | N |
| XL-358 | NH | H | H | CMe | O | N |
| XL-359 | NMe | H | H | CMe | O | N |
| XL-360 | NCHO | H | H | CMe | O | N |
| XL-361 | NCOMe | H | H | CMe | O | N |
| XL-362 | CH₂ | Me | H | CMe | O | N |
| XL-363 | O | Me | H | CMe | O | N |
| XL-364 | CHMe | Me | H | CMe | O | N |
| XL-365 | CH₂ | H | H | CH | S | N |
| XL-366 | CHMe | H | H | CH | S | N |
| XL-367 | CHEt | H | H | CH | S | N |
| XL-368 | CHF | H | H | CH | S | N |
| XL-369 | C═CH₂ | H | H | CH | S | N |
| XL-370 | O | H | H | CH | S | N |
| XL-371 | S | H | H | CH | S | N |
| XL-372 | NH | H | H | CH | S | N |
| XL-373 | NMe | H | H | CH | S | N |
| XL-374 | NCHO | H | H | CH | S | N |
| XL-375 | NCOMe | H | H | CH | S | N |
| XL-376 | CH₂ | Me | H | CH | S | N |
| XL-377 | O | Me | H | CH | S | N |
| XL-378 | CHMe | Me | H | CH | S | N |
| XL-379 | CH₂ | H | H | CMe | S | N |
| XL-380 | CHMe | H | H | CMe | S | N |
| XL-381 | CHEt | H | H | CMe | S | N |
| XL-382 | CHF | H | H | CMe | S | N |
| XL-383 | C═CH₂ | H | H | CMe | S | N |
| XL-384 | O | H | H | CMe | S | N |
| XL-385 | S | H | H | CMe | S | N |
| XL-386 | NH | H | H | CMe | S | N |
| XL-387 | NMe | H | H | CMe | S | N |
| XL-388 | NCHO | H | H | CMe | S | N |
| XL-389 | NCOMe | H | H | CMe | S | N |
| XL-390 | CH₂ | Me | H | CMe | S | N |
| XL-391 | O | Me | H | CMe | S | N |
| XL-392 | CHMe | Me | H | CMe | S | N |

Table XLI provides 336 compounds of formula Iao

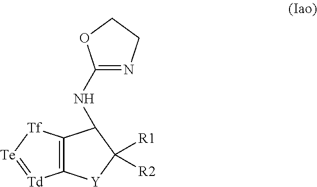

wherein the values of Y, R¹, R², T^{d}, T^{e} and T^{f} are given in Table 8.

TABLE 8

| Compound No | Y | R1 | R2 | Td | Te | Tf |
|---|---|---|---|---|---|---|
| XLI-1 | CH₂ | H | H | CH | CH | NH |
| XLI-2 | CHMe | H | H | CH | CH | NH |
| XLI-3 | CHEt | H | H | CH | CH | NH |
| XLI-4 | CHF | H | H | CH | CH | NH |
| XLI-5 | C═CH₂ | H | H | CH | CH | NH |
| XLI-6 | O | H | H | CH | CH | NH |
| XLI-7 | S | H | H | CH | CH | NH |
| XLI-8 | NH | H | H | CH | CH | NH |
| XLI-9 | NMe | H | H | CH | CH | NH |
| XLI-10 | NCHO | H | H | CH | CH | NH |
| XLI-11 | NCOMe | H | H | CH | CH | NH |
| XLI-12 | CH₂ | Me | H | CH | CH | NH |
| XLI-13 | O | Me | H | CH | CH | NH |
| XLI-14 | CHMe | Me | H | CH | CH | NH |
| XLI-15 | CH₂ | H | H | CMe | CH | NH |
| XLI-16 | CHMe | H | H | CMe | CH | NH |
| XLI-17 | CHEt | H | H | CMe | CH | NH |
| XLI-18 | CHF | H | H | CMe | CH | NH |
| XLI-19 | C═CH₂ | H | H | CMe | CH | NH |
| XLI-20 | O | H | H | CMe | CH | NH |
| XLI-21 | S | H | H | CMe | CH | NH |
| XLI-22 | NH | H | H | CMe | CH | NH |
| XLI-23 | NMe | H | H | CMe | CH | NH |
| XLI-24 | NCHO | H | H | CMe | CH | NH |
| XLI-25 | NCOMe | H | H | CMe | CH | NH |
| XLI-26 | CH₂ | Me | H | CMe | CH | NH |
| XLI-27 | O | Me | H | CMe | CH | NH |
| XLI-28 | CHMe | Me | H | CMe | CH | NH |
| XLI-29 | CH₂ | H | H | CH | CMe | NH |
| XLI-30 | CHMe | H | H | CH | CMe | NH |
| XLI-31 | CHEt | H | H | CH | CMe | NH |
| XLI-32 | CHF | H | H | CH | CMe | NH |
| XLI-33 | C═CH₂ | H | H | CH | CMe | NH |
| XLI-34 | O | H | H | CH | CMe | NH |
| XLI-35 | S | H | H | CH | CMe | NH |
| XLI-36 | NH | H | H | CH | CMe | NH |
| XLI-37 | NMe | H | H | CH | CMe | NH |
| XLI-38 | NCHO | H | H | CH | CMe | NH |
| XLI-39 | NCOMe | H | H | CH | CMe | NH |
| XLI-40 | CH₂ | Me | H | CH | CMe | NH |
| XLI-41 | O | Me | H | CH | CMe | NH |
| XLI-42 | CHMe | Me | H | CH | CMe | NH |
| XLI-43 | CH₂ | H | H | CH | CH | NMe |
| XLI-44 | CHMe | H | H | CH | CH | NMe |
| XLI-45 | CHEt | H | H | CH | CH | NMe |
| XLI-46 | CHF | H | H | CH | CH | NMe |
| XLI-47 | C═CH₂ | H | H | CH | CH | NMe |
| XLI-48 | O | H | H | CH | CH | NMe |
| XLI-49 | S | H | H | CH | CH | NMe |
| XLI-50 | NH | H | H | CH | CH | NMe |
| XLI-51 | NMe | H | H | CH | CH | NMe |
| XLI-52 | NCHO | H | H | CH | CH | NMe |
| XLI-53 | NCOMe | H | H | CH | CH | NMe |
| XLI-54 | CH₂ | Me | H | CH | CH | NMe |
| XLI-55 | O | Me | H | CH | CH | NMe |
| XLI-56 | CHMe | Me | H | CH | CH | NMe |
| XLI-57 | CH₂ | H | H | CMe | CH | NMe |
| XLI-58 | CHMe | H | H | CMe | CH | NMe |
| XLI-59 | CHEt | H | H | CMe | CH | NMe |
| XLI-60 | CHF | H | H | CMe | CH | NMe |
| XLI-61 | C═CH₂ | H | H | CMe | CH | NMe |
| XLI-62 | O | H | H | CMe | CH | NMe |
| XLI-63 | S | H | H | CMe | CH | NMe |
| XLI-64 | NH | H | H | CMe | CH | NMe |
| XLI-65 | NMe | H | H | CMe | CH | NMe |
| XLI-66 | NCHO | H | H | CMe | CH | NMe |
| XLI-67 | NCOMe | H | H | CMe | CH | NMe |
| XLI-68 | CH₂ | Me | H | CMe | CH | NMe |
| XLI-69 | O | Me | H | CMe | CH | NMe |
| XLI-70 | CHMe | Me | H | CMe | CH | NMe |
| XLI-71 | CH₂ | H | H | CH | CMe | NMe |
| XLI-72 | CHMe | H | H | CH | CMe | NMe |
| XLI-73 | CHEt | H | H | CH | CMe | NMe |
| XLI-74 | CHF | H | H | CH | CMe | NMe |
| XLI-75 | C═CH₂ | H | H | CH | CMe | NMe |
| XLI-76 | O | H | H | CH | CMe | NMe |
| XLI-77 | S | H | H | CH | CMe | NMe |
| XLI-78 | NH | H | H | CH | CMe | NMe |
| XLI-79 | NMe | H | H | CH | CMe | NMe |
| XLI-80 | NCHO | H | H | CH | CMe | NMe |
| XLI-81 | NCOMe | H | H | CH | CMe | NMe |
| XLI-82 | CH₂ | Me | H | CH | CMe | NMe |
| XLI-83 | O | Me | H | CH | CMe | NMe |
| XLI-84 | CHMe | Me | H | CH | CMe | NMe |
| XLI-85 | CH₂ | H | H | CH | CH | O |
| XLI-86 | CHMe | H | H | CH | CH | O |
| XLI-87 | CHEt | H | H | CH | CH | O |
| XLI-88 | CHF | H | H | CH | CH | O |
| XLI-89 | C═CH₂ | H | H | CH | CH | O |
| XLI-90 | O | H | H | CH | CH | O |
| XLI-91 | S | H | H | CH | CH | O |
| XLI-92 | NH | H | H | CH | CH | O |
| XLI-93 | NMe | H | H | CH | CH | O |
| XLI-94 | NCHO | H | H | CH | CH | O |
| XLI-95 | NCOMe | H | H | CH | CH | O |
| XLI-96 | CH₂ | Me | H | CH | CH | O |
| XLI-97 | O | Me | H | CH | CH | O |
| XLI-98 | CHMe | Me | H | CH | CH | O |
| XLI-99 | CH₂ | H | H | CMe | CH | O |
| XLI-100 | CHMe | H | H | CMe | CH | O |
| XLI-101 | CHEt | H | H | CMe | CH | O |
| XLI-102 | CHF | H | H | CMe | CH | O |
| XLI-103 | C═CH₂ | H | H | CMe | CH | O |
| XLI-104 | O | H | H | CMe | CH | O |
| XLI-105 | S | H | H | CMe | CH | O |
| XLI-106 | NH | H | H | CMe | CH | O |
| XLI-107 | NMe | H | H | CMe | CH | O |
| XLI-108 | NCHO | H | H | CMe | CH | O |
| XLI-109 | NCOMe | H | H | CMe | CH | O |
| XLI-110 | CH₂ | Me | H | CMe | CH | O |
| XLI-111 | O | Me | H | CMe | CH | O |
| XLI-112 | CHMe | Me | H | CMe | CH | O |
| XLI-113 | CH₂ | H | H | CH | CMe | O |
| XLI-114 | CHMe | H | H | CH | CMe | O |
| XLI-115 | CHEt | H | H | CH | CMe | O |
| XLI-116 | CHF | H | H | CH | CMe | O |
| XLI-117 | C═CH₂ | H | H | CH | CMe | O |
| XLI-118 | O | H | H | CH | CMe | O |
| XLI-119 | S | H | H | CH | CMe | O |
| XLI-120 | NH | H | H | CH | CMe | O |
| XLI-121 | NMe | H | H | CH | CMe | O |
| XLI-122 | NCHO | H | H | CH | CMe | O |
| XLI-123 | NCOMe | H | H | CH | CMe | O |
| XLI-124 | CH₂ | Me | H | CH | CMe | O |
| XLI-125 | O | Me | H | CH | CMe | O |
| XLI-126 | CHMe | Me | H | CH | CMe | O |
| XLI-127 | CH₂ | H | H | CH | CH | S |
| XLI-128 | CHMe | H | H | CH | CH | S |
| XLI-129 | CHEt | H | H | CH | CH | S |
| XLI-130 | CHF | H | H | CH | CH | S |
| XLI-131 | C═CH₂ | H | H | CH | CH | S |
| XLI-132 | O | H | H | CH | CH | S |
| XLI-133 | S | H | H | CH | CH | S |
| XLI-134 | NH | H | H | CH | CH | S |
| XLI-135 | NMe | H | H | CH | CH | S |
| XLI-136 | NCHO | H | H | CH | CH | S |
| XLI-137 | NCOMe | H | H | CH | CH | S |
| XLI-138 | CH₂ | Me | H | CH | CH | S |
| XLI-139 | O | Me | H | CH | CH | S |
| XLI-140 | CHMe | Me | H | CH | CH | S |
| XLI-141 | CH₂ | H | H | CMe | CH | S |
| XLI-142 | CHMe | H | H | CMe | CH | S |
| XLI-143 | CHEt | H | H | CMe | CH | S |
| XLI-144 | CHF | H | H | CMe | CH | S |
| XLI-145 | C═CH₂ | H | H | CMe | CH | S |
| XLI-146 | O | H | H | CMe | CH | S |
| XLI-147 | S | H | H | CMe | CH | S |
| XLI-148 | NH | H | H | CMe | CH | S |
| XLI-149 | NMe | H | H | CMe | CH | S |
| XLI-150 | NCHO | H | H | CMe | CH | S |
| XLI-151 | NCOMe | H | H | CMe | CH | S |
| XLI-152 | CH₂ | Me | H | CMe | CH | S |
| XLI-153 | O | Me | H | CMe | CH | S |
| XLI-154 | CHMe | Me | H | CMe | CH | S |
| XLI-155 | CH₂ | H | H | CH | CMe | S |
| XLI-156 | CHMe | H | H | CH | CMe | S |
| XLI-157 | CHEt | H | H | CH | CMe | S |
| XLI-158 | CHF | H | H | CH | CMe | S |
| XLI-159 | C═CH₂ | H | H | CH | CMe | S |
| XLI-160 | O | H | H | CH | CMe | S |
| XLI-161 | S | H | H | CH | CMe | S |
| XLI-162 | NH | H | H | CH | CMe | S |
| XLI-163 | NMe | H | H | CH | CMe | S |
| XLI-164 | NCHO | H | H | CH | CMe | S |
| XLI-165 | NCOMe | H | H | CH | CMe | S |
| XLI-166 | CH₂ | Me | H | CH | CMe | S |
| XLI-167 | O | Me | H | CH | CMe | S |
| XLI-168 | CHMe | Me | H | CH | CMe | S |
| XLI-169 | CH₂ | H | H | N | CH | NMe |
| XLI-170 | CHMe | H | H | N | CH | NMe |
| XLI-171 | CHEt | H | H | N | CH | NMe |
| XLI-172 | CHF | H | H | N | CH | NMe |
| XLI-173 | C═CH₂ | H | H | N | CH | NMe |
| XLI-174 | O | H | H | N | CH | NMe |
| XLI-175 | S | H | H | N | CH | NMe |
| XLI-176 | NH | H | H | N | CH | NMe |
| XLI-177 | NMe | H | H | N | CH | NMe |
| XLI-178 | NCHO | H | H | N | CH | NMe |
| XLI-179 | NCOMe | H | H | N | CH | NMe |
| XLI-180 | CH₂ | Me | H | N | CH | NMe |
| XLI-181 | O | Me | H | N | CH | NMe |
| XLI-182 | CHMe | Me | H | N | CH | NMe |
| XLI-183 | CH₂ | H | H | N | CMe | NMe |
| XLI-184 | CHMe | H | H | N | CMe | NMe |
| XLI-185 | CHEt | H | H | N | CMe | NMe |
| XLI-186 | CHF | H | H | N | CMe | NMe |
| XLI-187 | C═CH₂ | H | H | N | CMe | NMe |
| XLI-188 | O | H | H | N | CMe | NMe |
| XLI-189 | S | H | H | N | CMe | NMe |
| XLI-190 | NH | H | H | N | CMe | NMe |
| XLI-191 | NMe | H | H | N | CMe | NMe |
| XLI-192 | NCHO | H | H | N | CMe | NMe |
| XLI-193 | NCOMe | H | H | N | CMe | NMe |
| XLI-194 | CH₂ | Me | H | N | CMe | NMe |
| XLI-195 | O | Me | H | N | CMe | NMe |
| XLI-196 | CHMe | Me | H | N | CMe | NMe |
| XLI-197 | CH₂ | H | H | N | CH | O |
| XLI-198 | CHMe | H | H | N | CH | O |
| XLI-199 | CHEt | H | H | N | CH | O |
| XLI-200 | CHF | H | H | N | CH | O |
| XLI-201 | C═CH₂ | H | H | N | CH | O |
| XLI-202 | O | H | H | N | CH | O |
| XLI-203 | S | H | H | N | CH | O |
| XLI-204 | NH | H | H | N | CH | O |
| XLI-205 | NMe | H | H | N | CH | O |
| XLI-206 | NCHO | H | H | N | CH | O |
| XLI-207 | NCOMe | H | H | N | CH | O |
| XLI-208 | CH₂ | Me | H | N | CH | O |
| XLI-209 | O | Me | H | N | CH | O |
| XLI-210 | CHMe | Me | H | N | CH | O |
| XLI-211 | CH₂ | H | H | N | CMe | O |
| XLI-212 | CHMe | H | H | N | CMe | O |
| XLI-213 | CHEt | H | H | N | CMe | O |
| XLI-214 | CHF | H | H | N | CMe | O |
| XLI-215 | C═CH₂ | H | H | N | CMe | O |
| XLI-216 | O | H | H | N | CMe | O |
| XLI-217 | S | H | H | N | CMe | O |
| XLI-218 | NH | H | H | N | CMe | O |
| XLI-219 | NMe | H | H | N | CMe | O |
| XLI-220 | NCHO | H | H | N | CMe | O |
| XLI-221 | NCOMe | H | H | N | CMe | O |
| XLI-222 | CH₂ | Me | H | N | CMe | O |
| XLI-223 | O | Me | H | N | CMe | O |
| XLI-224 | CHMe | Me | H | N | CMe | O |
| XLI-225 | CH₂ | H | H | N | CH | S |
| XLI-226 | CHMe | H | H | N | CH | S |
| XLI-227 | CHEt | H | H | N | CH | S |
| XLI-228 | CHF | H | H | N | CH | S |
| XLI-229 | C═CH₂ | H | H | N | CH | S |
| XLI-230 | O | H | H | N | CH | S |
| XLI-231 | S | H | H | N | CH | S |
| XLI-232 | NH | H | H | N | CH | S |
| XLI-233 | NMe | H | H | N | CH | S |
| XLI-234 | NCHO | H | H | N | CH | S |
| XLI-235 | NCOMe | H | H | N | CH | S |
| XLI-236 | CH₂ | Me | H | N | CH | S |
| XLI-237 | O | Me | H | N | CH | S |
| XLI-238 | CHMe | Me | H | N | CH | S |
| XLI-239 | CH₂ | H | H | N | CMe | S |
| XLI-240 | CHMe | H | H | N | CMe | S |
| XLI-241 | CHEt | H | H | N | CMe | S |
| XLI-242 | CHF | H | H | N | CMe | S |
| XLI-243 | C═CH₂ | H | H | N | CMe | S |
| XLI-244 | O | H | H | N | CMe | S |
| XLI-245 | S | H | H | N | CMe | S |
| XLI-246 | NH | H | H | N | CMe | S |
| XLI-247 | NMe | H | H | N | CMe | S |
| XLI-248 | NCHO | H | H | N | CMe | S |
| XLI-249 | NCOMe | H | H | N | CMe | S |
| XLI-250 | CH₂ | Me | H | N | CMe | S |
| XLI-251 | O | Me | H | N | CMe | S |
| XLI-252 | CHMe | Me | H | N | CMe | S |
| XLI-253 | CH₂ | H | H | CH | N | NMe |
| XLI-254 | CHMe | H | H | CH | N | NMe |
| XLI-255 | CHEt | H | H | CH | N | NMe |
| XLI-256 | CHF | H | H | CH | N | NMe |
| XLI-257 | C═CH₂ | H | H | CH | N | NMe |
| XLI-258 | O | H | H | CH | N | NMe |
| XLI-259 | S | H | H | CH | N | NMe |
| XLI-260 | NH | H | H | CH | N | NMe |
| XLI-261 | NMe | H | H | CH | N | NMe |
| XLI-262 | NCHO | H | H | CH | N | NMe |
| XLI-263 | NCOMe | H | H | CH | N | NMe |
| XLI-264 | CH₂ | Me | H | CH | N | NMe |
| XLI-265 | O | Me | H | CH | N | NMe |
| XLI-266 | CHMe | Me | H | CH | N | NMe |
| XLI-267 | CH₂ | H | H | CMe | N | NMe |
| XLI-268 | CHMe | H | H | CMe | N | NMe |
| XLI-269 | CHEt | H | H | CMe | N | NMe |
| XLI-270 | CHF | H | H | CMe | N | NMe |
| XLI-271 | C═CH₂ | H | H | CMe | N | NMe |
| XLI-272 | O | H | H | CMe | N | NMe |
| XLI-273 | S | H | H | CMe | N | NMe |
| XLI-274 | NH | H | H | CMe | N | NMe |
| XLI-275 | NMe | H | H | CMe | N | NMe |
| XLI-276 | NCHO | H | H | CMe | N | NMe |
| XLI-277 | NCOMe | H | H | CMe | N | NMe |
| XLI-278 | CH₂ | Me | H | CMe | N | NMe |
| XLI-279 | O | Me | H | CMe | N | NMe |
| XLI-280 | CHMe | Me | H | CMe | N | NMe |
| XLI-281 | CH₂ | H | H | CH | N | O |
| XLI-282 | CHMe | H | H | CH | N | O |
| XLI-283 | CHEt | H | H | CH | N | O |
| XLI-284 | CHF | H | H | CH | N | O |
| XLI-285 | C═CH₂ | H | H | CH | N | O |
| XLI-286 | O | H | H | CH | N | O |
| XLI-287 | S | H | H | CH | N | O |
| XLI-288 | NH | H | H | CH | N | O |
| XLI-289 | NMe | H | H | CH | N | O |
| XLI-290 | NCHO | H | H | CH | N | O |
| XLI-291 | NCOMe | H | H | CH | N | O |
| XLI-292 | CH₂ | Me | H | CH | N | O |
| XLI-293 | O | Me | H | CH | N | O |
| XLI-294 | CHMe | Me | H | CH | N | O |
| XLI-295 | CH₂ | H | H | CMe | N | O |
| XLI-296 | CHMe | H | H | CMe | N | O |
| XLI-297 | CHEt | H | H | CMe | N | O |
| XLI-298 | CHF | H | H | CMe | N | O |
| XLI-299 | C═CH₂ | H | H | CMe | N | O |
| XLI-300 | O | H | H | CMe | N | O |
| XLI-301 | S | H | H | CMe | N | O |
| XLI-302 | NH | H | H | CMe | N | O |
| XLI-303 | NMe | H | H | CMe | N | O |
| XLI-304 | NCHO | H | H | CMe | N | O |
| XLI-305 | NCOMe | H | H | CMe | N | O |
| XLI-306 | CH₂ | Me | H | CMe | N | O |
| XLI-307 | O | Me | H | CMe | N | O |
| XLI-308 | CHMe | Me | H | CMe | N | O |
| XLI-309 | CH₂ | H | H | CH | N | S |
| XLI-310 | CHMe | H | H | CH | N | S |
| XLI-311 | CHEt | H | H | CH | N | S |
| XLI-312 | CHF | H | H | CH | N | S |
| XLI-313 | C═CH₂ | H | H | CH | N | S |
| XLI-314 | O | H | H | CH | N | S |
| XLI-315 | S | H | H | CH | N | S |
| XLI-316 | NH | H | H | CH | N | S |
| XLI-317 | NMe | H | H | CH | N | S |
| XLI-318 | NCHO | H | H | CH | N | S |
| XLI-319 | NCOMe | H | H | CH | N | S |
| XLI-320 | CH₂ | Me | H | CH | N | S |
| XLI-321 | O | Me | H | CH | N | S |
| XLI-322 | CHMe | Me | H | CH | N | S |
| XLI-323 | CH₂ | H | H | CMe | N | S |
| XLI-324 | CHMe | H | H | CMe | N | S |
| XLI-325 | CHEt | H | H | CMe | N | S |
| XLI-326 | CHF | H | H | CMe | N | S |
| XLI-327 | C═CH₂ | H | H | CMe | N | S |
| XLI-328 | O | H | H | CMe | N | S |
| XLI-329 | S | H | H | CMe | N | S |
| XLI-330 | NH | H | H | CMe | N | S |
| XLI-331 | NMe | H | H | CMe | N | S |
| XLI-332 | NCHO | H | H | CMe | N | S |
| XLI-333 | NCOMe | H | H | CMe | N | S |
| XLI-334 | CH₂ | Me | H | CMe | N | S |
| XLI-335 | O | Me | H | CMe | N | S |
| XLI-336 | CHMe | Me | H | CMe | N | S |

Table XLIII provides 612 compounds of formula Iap

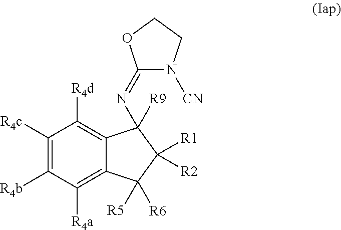

wherein the values of R¹, R², R^{4a}, R^{4b}, R^{4c}, R^{4d}, R⁵, R⁶ and R⁹ are given in Table 3.

Table XLIII provides 612 compounds of formula Iaq

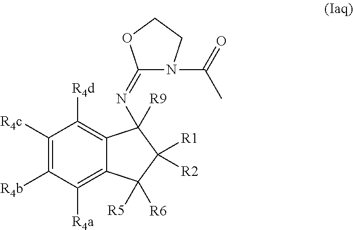

wherein the values of R¹, R², R^{4a}, R^{4b}, R^{4c}, R^{4d}, R⁵, R⁶ and R⁹ are given in Table 3.

The following tables provide characterising data for some of the compounds in Tables I-XLIII; other compounds are only described in these tables.

Characterising Data - 1

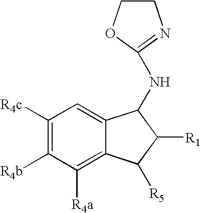

| Cpd. No | R1 | R5 | R4a | R4b | R4c | isomer | Mp [° C.] |
|---|---|---|---|---|---|---|---|
| 1.001 | CH₃ | H | H | H | H | cis | solid |
| 1.002 | H | CH₃ | H | H | H | cis | 119-122 |
| 1.003 | H | H | H | OCH₃ | H | | 115-118 |
| 1.004 | H | H | CH₃ | H | H | | 78-82 |
| 1.005 | H | H | H | F | H | | 110-114 |
| 1.006 | H | H | H | Br | H | | 100-125 |
| | | | | | | | decomp. |
| 1.007 | H | H | H | Cl | H | | 110-114 |
| 1.008 | H | H | H | OCH₃ | OCH₃ | | 109-113 |
| 1.009 | H | H | H | H | OCH₃ | | 84-88 |
| 1.010 | H | H | H | CH₃ | H | | 116-120 |
| 1.011 | H | Ph | H | H | H | 10:1 ratio | >120 |
| | | | | | | | decomp. |
| 1.012 | H | H | H | H | CH₃ | | 144-147 |
| 1.013 | OH | H | H | H | H | 1R2S | 139-141 |
| 1.014 | OH | H | H | H | H | 1S2R | 140-142 |
| 1.015 | CH₃ | H | H | H | H | trans | 80-86 |
| 1.016 | H | H | F | H | H | | 102-103 |
| 1.017 | H | H | H | H | H | <S>-form | 72-73 |

Characterising Data - 2

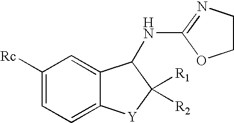

| Cpd. No. | Y | CR1R2 | Rc | | Mp [° C.] |
|---|---|---|---|---|---|
| 2.001 | O | CH₂ | H | | 106-110 |
| 2.002 | S | C(═O) | H | | beige crystals |
| 2.003 | O | CH₂ | CH₃ | •HCl | solid |
| 2.004 | NH | C(═O) | H | •HCl | solid |

Characterising Data - 3
Error! Objects cannot be created from editing field codes.

| Cpd. | | | | | | Mp |
|---|---|---|---|---|---|---|
| No. | Y | R1 | R4a | R4c | Isomer | [° C.] |
| 3.001 | —CH₂—S— | H | H | H | | 98-104 |
| 3.002 | —N(CH₃)—SO₂— | H | H | H | | 150-154 |
| 3.003 | —O—CHPh— | H | H | H | c:t 1:1 | 169-175 |
| 3.004 | —CH₂—CH₂— | CH₃ | H | H | c:t 1:4 | 108-112 |
| 3.005 | —S—CH₂— | H | H | Cl | | 119-126 |
| 3.006 | —O—CH₂— | H | Cl | H | | 142-144 |
| 3.007 | —CH₂—CH₂— | H | H | H | | 112-115 |
| 3.008 | —CH₂—CH₂— | H | OCH₃ | H | | 133-136 |
| 3.009 | —CH₂—CH₂— | H | H | OCH₃ | | 126-129 |
| 3.010 | —O—CH₂— | H | H | H | HCl | solid |

Characterising Data - 4

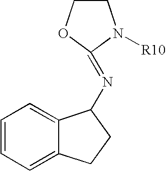

| Cpd. No. | R10 | Mp [° C.] |
|---|---|---|
| 4.001 | —CH₂Ph | oil |

The compounds of the invention may be made by a variety of methods. Various methods useful for the preparation of 2-amino-1,3-oxazolines can be found for example in Chem. Rev. 1949, 447-476 and in Chem. Rev. 1971, 71, 483-505.

Compounds of formula (I) can be prepared by treatment of an amine of formula (IIa) with chloroethylisocyanate to afford an intermediate compound of formula (IIIa), which is then cyclised as outlined in the reaction scheme below. This cyclisation can take place in the presence of base (see for example Bioorg. Med. Chem. Lett. 1994, 4, 2317-2322), or simply on heating (see for example Ber. 1895, 28, 2929-2938). Leaving groups other than chloride can be used (see for example Synth. 1987, 853-854).

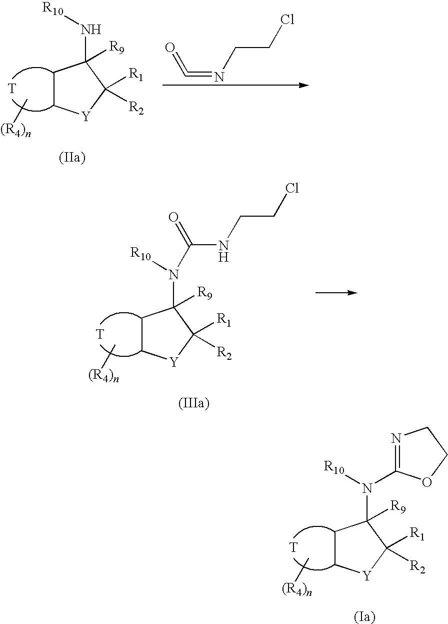

Reaction of amines with 2-methylthio-1,3-oxazoline can lead to compounds of the formula (1a) (see J. Med. Chem. 1982, 25, 735-742). Leaving groups other than methylthio can also be used (see Carb. Res. 1994, 1124-1126; Tet. Lett. 1991, 32, 5363).

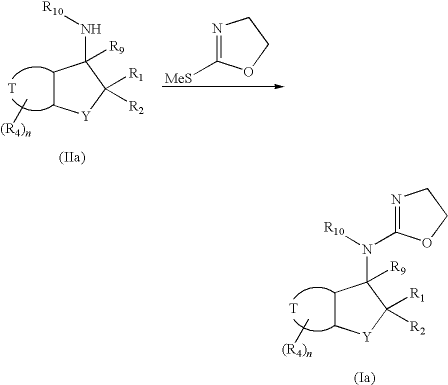

Other methods for the preparation of 2-alkylamino-1,3-oxazolines include the following. Treatment of alkylamino-aziridinyl ureas with iodide (see U.S. Pat. No. 4,064,348). Treatment of hydroxyethylureas with chlorinating agents to compounds of the formula (III) (see DE 2 362 754), and then cyclisation as described above.

The amines (IIa) can be used to make 2-hydroxyethyl-thioureas (VIIᵢ), according to methods found in Houben-Weyl E4, (1983) 484, for example using in a first step thiophosgene, and then in a second step 2-hydroxyethylamine. The thioureas (VIIᵢᵢ) can be prepared from the isothiocyanates (VI). Each of these thioureas can be cyclised to the corresponding oxazolines (I), for example with methyl iodide as described for example in J. Chem. Soc. (C) 1966, 65-67, with oxidising agents as described for example in Synlett. 1997, 1324-1326, with sulfonyl chlorides as described for example in Tet. 2001, 57, 7137-7141, or with strong bases as described in DE 2800062. If R¹⁰ is H, then (IIa) and (IIb) are identical, (VII₁) and VIIᵢᵢ) are identical, and (Iᵢ), (Iᵢᵢ) and (Iᵢᵢᵢ) are tautomers of the same compound.

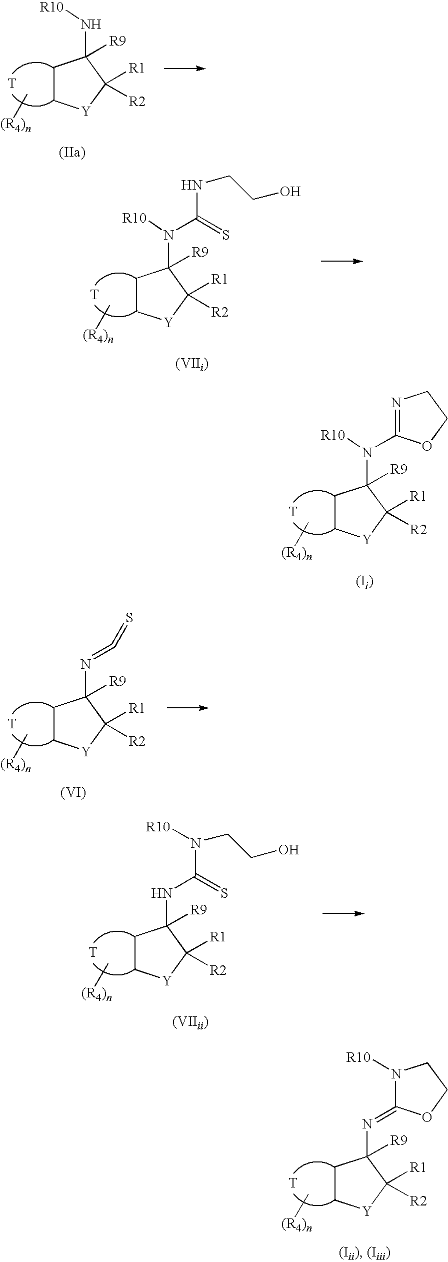

Amines of the formula (IIb), wherein R₉ is hydrogen, can be prepared in various ways. One method starts from ketones (IV). Transformation involves reduction of the ketone with NaBH₄/MeOH in the known manner and conversion to the corresponding azide according to WO 95/01970. Subsequent reduction with PPh₃/H₂O (e.g. J. Med. Chem. 2005, 48, 485) or in the presence of SnCl₂/MeOH (e.g. Synthetic Commun. 1991, 21, 733) leads to the desired amines (IIb).

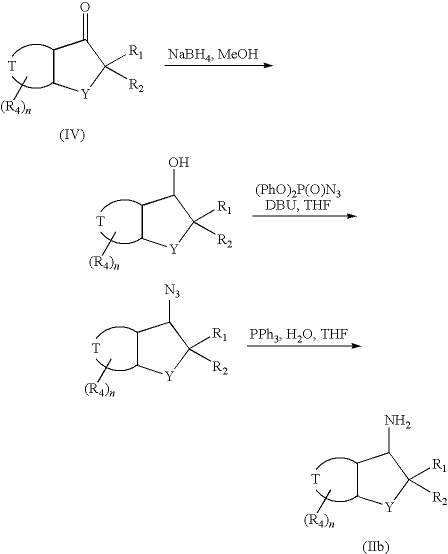

Amines of the formula (IIb) can also be prepared from carboxylic acids (V) by a Curtius degradation using (PhO)₂P(O)N₃ as reagent (e.g. Tetrahedron Letters 1997, 38, 1681) as shown below.

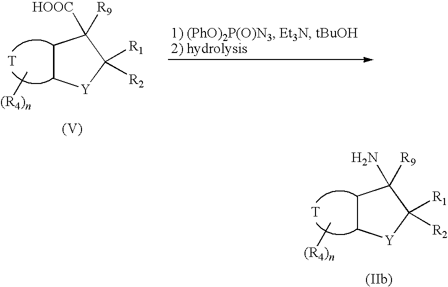

Amines of formula IIb, wherein Y is oxygen are partially known or may be prepared by the known methods as described in Chimica Acta Turica, 13(3), 403-412 (1985) and Farmaco, Edizione Scientifica, 43(7-8), 643-655 (1988).

With the exception of the following compounds: 1-(2-chloroethyl)-3-(1-indanyl)urea, 1-(2-bromoethyl)-3-(1-indanyl)urea, 1-(2-iodoethyl)-3-(1-indanyl)urea, 1-(2,4-diiodo-indany-1-yl)-3-(2-iodoethyl)-urea, 1-(2-bromo-ethyl)-3-(2-ethyl-indan-1-yl) urea, methanesulfonic acid 2-[3-(2-propoxy-indan-1-yl)-ureido]-ethyl ester, 1-(2-bromo-thyl)-3-(2,4,5-trichloro-indan-1-yl)-urea, 1-(2-chloroethyl)-3-(2-chloro-1-indanyl)urea, 1-(2-chloroethyl)-3-(3,4-dimethyl-1-indanyl)urea, Methanesulfonic acid 2-[3-(2-methyl-indan-1-yl)-ureido]-ethyl ester, Toluene-4-sulfonic acid 2-[3-(2,5-diiodo-indan-1-yl)-ureido]-ethyl ester, 1-(2-chloroethyl)-3-(3,4-dihydro-2H-1-benzothiopyran-4-yl) urea, 1-(2-chloroethyl)-3-(1,2,3,4-tetrahydro-1-naphthyl)urea, 1-(2-bromoethyl)-3-(1,2,3,4-tetrahydro-1-naphthyl)urea, 1-(2-iodoethyl)-3-(1,2,3,4-tetrahydro-1-naphthyl)urea, 1-(2-chloroethyl)-3-(2-chloro-1,2,3,4-tetrahydro-1-naphthyl)urea, 1-(2-chloroethyl)-3-(4,5,6,7-tetrahydrobenzo[b]thien-4-yl) urea, N-(2-chloroethyl)-N′-(2,3,3a,4,5,9b-hexahydro-1H-benz[e]inden-5-yl)urea, (4α,5α,6α)-N-(2-chloroethyl)-N′-(5,6-dihydro-5,6-dihydroxy-4H-cyclopenta[b]thien-4-yl) urea, (4α,5α,6α)-N-[5,6-bis(acetyloxy)-5,6-dihydro-4H-cyclopenta[b]thien-4-yl]-N′-(2-chloroethyl)urea, (4α,5α,6α)-N-(2-chloroethyl)-N′-(5,6-dihydro-5,6-dihydroxy-4H-cyclopenta[b]thien-4-yl) urea and (4α,5α,6α)-N-[5,6-bis(acetyloxy)-5,6-dihydro-4H-cyclopenta[b]thien-4-yl]-N′-(2-chloroethyl)urea, the ureas of formula (III)

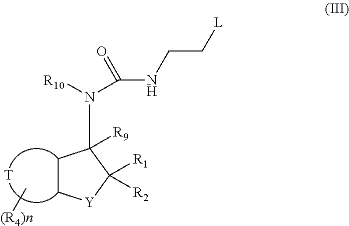

wherein T, X, Y, R¹, R², R⁴, R⁹ and n are as defined above with respect to formula I, and L is a leaving group e.g. chloro, bromo, iodo, C₁₋₄alkylsulfonyloxy or phenylsulfonyloxy (where phenyl is optionally substituted by halogen, C₁₋₄alkyl, C₁₋₄alkoxy or nitro) are novel and can be used to form a compound of formula I as defined herein. Such ureas form yet a further aspect of the invention.

Furthermore it has been found that the ureas of formula (III) per se also have pronounced pesticidal activity too. The present invention therefore also provides a method of combating and controlling insects, acarines, nematodes or molluscs, which comprises applying to a pest, to a locus of a pest, or to a plant susceptible to attack by a pest an insecticidally, acaricidally, nematicidally or molluscicidally an effective amount of a compound of formula (III).

In preferred compounds L is chlorine (these compounds are referred to herein as compounds of formula IIIa). Characterising data for exemplary compounds of formula IIIa are shown in the following tables.

Characterising Data - IIIa1

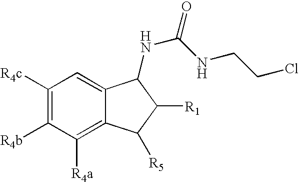

| Cpd. No | R1 | R5 | R4a | R4b | R4c | isomer | Mp [° C.] |
|---|---|---|---|---|---|---|---|
| 31.001 | CH₃ | H | H | H | H | | |
| 31.002 | H | CH₃ | H | H | H | Cis | |
| 31.012 | H | H | H | H | CH₃ | | solid |
| 31.016 | H | H | F | H | H | | 86-87 |
| 31.017 | H | H | H | H | H | Rac | 146-147 |
| 31.018 | H | H | H | H | H | <S>-form | 160-161 |
| 31.019 | H | H | H | H | H | <R>-form | 160-161 |

Characterising Data - IIIa2

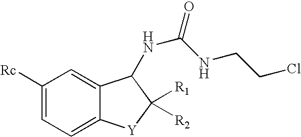

| Cpd. No. | Y | CR1R2 | Rc | Mp [° C.] |
|---|---|---|---|---|
| 32.002 | S | C(═O) | H | Solid |
| 32.004 | NH | C(═O) | H | |

Characterising Data - IIIa3

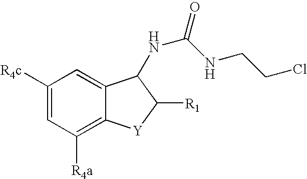

| Cpd. | | | | | | Mp |
|---|---|---|---|---|---|---|
| No. | Y | R1 | R4a | R4c | Isomer | [° C.] |
| 33.002 | —N(CH₃)—SO₂— | H | H | H | | |
| 33.007 | —CH₂—CH₂— | H | H | H | | solid |
| 33.010 | —O—CH₂— | H | H | H | | |
| 33.011 | —C(Et, CONH₂)CH₂— | H | H | H | | |

The compounds of formula (I) and/or formula (III) can be used to combat and control infestations of insect pests such as Lepidoptera, Diptera, Hemiptera, Thysanoptera, Orthoptera, Dictyoptera, Coleoptera, Siphonaptera, Hymenoptera and Isoptera and also other invertebrate pests, for example, acarine, nematode and mollusc pests. Insects, acarines, nematodes and molluscs are hereinafter collectively referred to as pests. The pests which may be combated and controlled by the use of the invention compounds include those pests associated with agriculture (which term includes the growing of crops for food and fibre products), horticulture and animal husbandry, companion animals, forestry and the storage of products of vegetable origin (such as fruit, grain and timber); those pests associated with the damage of man-made structures and the transmission of diseases of man and animals; and also nuisance pests (such as flies).

Examples of pest species which may be controlled by the compounds of formula (I) or formula (III) include: Myzus persicae (aphid), Aphis gossypii (aphid), Aphis fabae (aphid), Lygus spp. (capsids), Dysdercus spp. (capsids), Nilaparvata lugens (planthopper), Nephotettixc incticeps (leafhopper), Nezara spp. (stinkbugs), Euschistus spp. (stinkbugs), Leptocorisa spp. (stinkbugs), Frankliniella occidentalis (thrip), Thrips spp. (thrips), Leptinotarsa decemlineata (Colorado potato beetle), Anthonomus grandis (boll weevil), Aonidiella spp. (scale insects), Trialeurodes spp. (white flies), Bemisia tabaci (white fly), Ostrinia nubilalis (European corn borer), Spodoptera littoralis (cotton leafworm), Heliothis virescens (tobacco budworm), Helicoverpa armigera (cotton bollworm), Helicoverpa zea (cotton bollworm), Sylepta derogata (cotton leaf roller), Pieris brassicae (white butterfly), Plutella xylostella (diamond back moth), Agrotis spp. (cutworms), Chilo suppressalis (rice stem borer), Locusta migratoria (locust), Chortiocetes terminifera (locust), Diabrotica spp. (rootworms), Panonychus ulmi (European red mite), Panonychus citri (citrus red mite), Tetranychus urticae (two-spotted spider mite), Tetranychus cinnabarinus (carmine spider mite), Phyllocoptruta oleivora (citrus rust mite), Polyphagotarsonemus latus (broad mite), Brevipalpus spp. (flat mites), Boophilus microplus (cattle tick), Dermacentor variabilis (American dog tick), Ctenocephalides fells (cat flea), Liriomyza spp. (leafminer), Musca domestica (housefly), Aedes aegypti (mosquito), Anopheles spp. (mosquitoes), Culex spp. (mosquitoes), Lucillia spp. (blowflies), Blattella germanica (cockroach), Periplaneta americana (cockroach), Blatta orientalis (cockroach), termites of the Mastotermitidae (for example Mastotermes spp.), the Kalotermitidae (for example Neotermes spp.), the Rhinotermitidae (for example Coptotermes formosanus, Reticulitermes flavipes, R. speratu, R. virginicus, R. hesperus, and R. santonensis) and the Termitidae (for example Globitermes sulphureus), Solenopsis geminata (fire ant), Monomorium pharaonis (pharaoh's ant), Damalinia spp. and Linognathus spp. (biting and sucking lice), and Deroceras reticulatum (slug).

The invention therefore provides a method of combating and controlling insects, acarines, or molluscs which comprises applying an insecticidally, acaricidally, nematicidally or molluscicidally effective amount of a compound of formula (I) or formula (III), or a composition containing a compound of formula (I) and/or formula (III), to a pest, a locus of pest, or to a plant susceptible to attack by a pest, The compounds of formula (I) or formula (III) are preferably used against insects or acarines.

The term “plant” as used herein includes seedlings, bushes and trees.

In order to apply a compound of formula (I) or formula (III) as an insecticide, acaricide, nematicide or molluscicide to a pest, a locus of pest, or to a plant susceptible to attack by a pest, a compound of formula (I) or formula (III) is usually formulated into a composition which includes, in addition to the compound of formula (I) or formula (III), a suitable inert diluent or carrier and, optionally, a surface active agent (SFA). SFAs are chemicals which are able to modify the properties of an interface (for example, liquid/solid, liquid/air or liquid/liquid interfaces) by lowering the interfacial tension and thereby leading to changes in other properties (for example dispersion, emulsification and wetting). It is preferred that all compositions (both solid and liquid formulations) comprise, by weight, 0.0001 to 95%, more preferably 1 to 85%, for example 5 to 60%, of a compound of formula (I) or formula (III). The composition is generally used for the control of pests such that a compound of formula (I) or formula (III) is applied at a rate of from 0.1 g to 10 kg per hectare, preferably from 1 g to 6 kg per hectare, more preferably from 1 g to 1 kg per hectare.

When used in a seed dressing, a compound of formula (I) or formula (III) is used at a rate of 0.0001 g to 10 g (for example 0.001 g or 0.05 g), preferably 0.005 g to 10 g, more preferably 0.005 g to 4 g, per kilogram of seed.

In another aspect the present invention provides an insecticidal, acaricidal, nematicidal or molluscicidal composition comprising an insecticidally, acaricidally, nematicidally or molluscicidally effective amount, of a compound of formula (I) and/or formula (III) and a suitable carrier or diluent therefor. The composition is preferably an insecticidal, acaricidal, nematicidal or molluscicidal composition.

In a still further aspect the invention provides a method of combating and controlling pests at a locus which comprises treating the pests or the locus of the pests with an insecticidally, acaricidally, nematicidally or molluscicidally effective amount of a composition comprising a compound of formula (I) and/or formula (III). The compounds of formula (I) and/or formula (III) are preferably used against insects, acarines or nematodes.

The compositions can be chosen from a number of formulation types, including dustable powders (DP), soluble powders (SP), water soluble granules (SG), water dispersible granules (WG), wettable powders (WP), granules (GR) (slow or fast release), soluble concentrates (SL), oil miscible liquids (OL), ultra low volume liquids (UL), emulsifiable concentrates (EC), dispersible concentrates (DC), emulsions (both oil in water (EW) and water in oil (EO)), micro-emulsions (ME), suspension concentrates (SC), aerosols, fogging/smoke formulations, capsule suspensions (CS) and seed treatment formulations. The formulation type chosen in any instance will depend upon the particular purpose envisaged and the physical, chemical and biological properties of the compound of formula (I) and/or formula (III).

Dustable powders (DP) may be prepared by mixing a compound of formula (I) with one or more solid diluents (for example natural clays, kaolin, pyrophyllite, bentonite, alumina, montmorillonite, kieselguhr, chalk, diatomaceous earths, calcium phosphates, calcium and magnesium carbonates, sulphur, lime, flours, talc and other organic and inorganic solid carriers) and mechanically grinding the mixture to a fine powder.

Soluble powders (SP) may be prepared by mixing a compound of formula (I) and/or formula (III) with one or more water-soluble inorganic salts (such as sodium bicarbonate, sodium carbonate or magnesium sulphate) or one or more water-soluble organic solids (such as a polysaccharide) and, optionally, one or more wetting agents, one or more dispersing agents or a mixture of said agents to improve water dispersibility/solubility. The mixture is then ground to a fine powder. Similar compositions may also be granulated to form water soluble granules (SG).

Wettable powders (WP) may be prepared by mixing a compound of formula (I) and/or formula (III) with one or more solid diluents or carriers, one or more wetting agents and, preferably, one or more dispersing agents and, optionally, one or more suspending agents to facilitate the dispersion in liquids. The mixture is then ground to a fine powder. Similar compositions may also be granulated to form water dispersible granules (WG).

Granules (GR) may be formed either by granulating a mixture of a compound of formula (I) and one or more powdered solid diluents or carriers, or from pre-formed blank granules by absorbing a compound of formula (I) and/or formula (III) (or a solution thereof, in a suitable agent) in a porous granular material (such as pumice, attapulgite clays, fuller's earth, kieselguhr, diatomaceous earths or ground corn cobs) or by adsorbing a compound of formula (I) and/or formula (III)(or a solution thereof, in a suitable agent) on to a hard core material (such as sands, silicates, mineral carbonates, sulphates or phosphates) and drying if necessary. Agents which are commonly used to aid absorption or adsorption include solvents (such as aliphatic and aromatic petroleum solvents, alcohols, ethers, ketones and esters) and sticking agents (such as polyvinyl acetates, polyvinyl alcohols, dextrins, sugars and vegetable oils). One or more other additives may also be included in granules (for example an emulsifying agent, wetting agent or dispersing agent).

Dispersible Concentrates (DC) may be prepared by dissolving a compound of formula (I) and/or formula (III) in water or an organic solvent, such as a ketone, alcohol or glycol ether. These solutions may contain a surface active agent (for example to improve water dilution or prevent crystallisation in a spray tank).

Emulsifiable concentrates (EC) or oil-in-water emulsions (EW) may be prepared by dissolving a compound of formula (I) and/or formula (III) in an organic solvent (optionally containing one or more wetting agents, one or more emulsifying agents or a mixture of said agents). Suitable organic solvents for use in ECs include aromatic hydrocarbons (such as alkylbenzenes or alkylnaphthalenes, exemplified by SOLVESSO 100, SOLVESSO 150 and SOLVESSO 200; SOLVESSO is a Registered Trade Mark), ketones (such as cyclohexanone or methylcyclohexanone) and alcohols (such as benzyl alcohol, furfuryl alcohol or butanol),

N-alkylpyrrolidones (such as N-methylpyrrolidone or N-octylpyrrolidone), dimethyl amides of fatty acids (such as C₈-C₁₀ fatty acid dimethylamide) and chlorinated hydrocarbons. An EC product may spontaneously emulsify on addition to water, to produce an emulsion with sufficient stability to allow spray application through appropriate equipment. Preparation of an EW involves obtaining a compound of formula (I) and/or formula (III) either as a liquid (if it is not a liquid at room temperature, it may be melted at a reasonable temperature, typically below 70° C.) or in solution (by dissolving it in an appropriate solvent) and then emulsifiying the resultant liquid or solution into water containing one or more SFAs, under high shear, to produce an emulsion. Suitable solvents for use in EWs include vegetable oils, chlorinated hydrocarbons (such as chlorobenzenes), aromatic solvents (such as alkylbenzenes or alkylnaphthalenes) and other appropriate organic solvents which have a low solubility in water.

Microemulsions (ME) may be prepared by mixing water with a blend of one or more solvents with one or more SFAs, to produce spontaneously a thermodynamically stable isotropic liquid formulation. A compound of formula (I) and/or formula (III) is present initially in either the water or the solvent/SFA blend. Suitable solvents for use in MEs include those hereinbefore described for use in ECs or in EWs. An ME may be either an oil-in-water or a water-in-oil system (which system is present may be determined by conductivity measurements) and may be suitable for mixing water-soluble and oil-soluble pesticides in the same formulation. An ME is suitable for dilution into water, either remaining as a microemulsion or forming a conventional oil-in-water emulsion.

Suspension concentrates (SC) may comprise aqueous or non-aqueous suspensions of finely divided insoluble solid particles of a compound of formula (I) and/or formula (III). SCs may be prepared by ball or bead milling the solid compound of formula (I) and/or formula (III) in a suitable medium, optionally with one or more dispersing agents, to produce a fine particle suspension of the compound(s). One or more wetting agents may be included in the composition and a suspending agent may be included to reduce the rate at which the particles settle. Alternatively, a compound of formula (I) and/or formula (III) may be dry milled and added to water, containing agents hereinbefore described, to produce the desired end product.

Aerosol formulations comprise a compound of formula (I) and/or formula (III) and a suitable propellant (for example n-butane). A compound of formula (I) and/or formula (III) may also be dissolved or dispersed in a suitable medium (for example water or a water miscible liquid, such as n-propanol) to provide compositions for use in non-pressurised, hand-actuated spray pumps.

A compound of formula (I) and/or formula (III) may be mixed in the dry state with a pyrotechnic mixture to form a composition suitable for generating, in an enclosed space, a smoke containing the compound.

Capsule suspensions (CS) may be prepared in a manner similar to the preparation of EW formulations but with an additional polymerisation stage such that an aqueous dispersion of oil droplets is obtained, in which each oil droplet is encapsulated by a polymeric shell and contains a compound of formula (I) and/or formula (III) and, optionally, a carrier or diluent therefor. The polymeric shell may be produced by either an interfacial polycondensation reaction or by a coacervation procedure. The compositions may provide for controlled release of the compound of formula (I) and/or formula (III) and they may be used for seed treatment. A compound of formula (I) and/or formula (III) may also be formulated in a biodegradable polymeric matrix to provide a slow, controlled release of the compound.

A composition may include one or more additives to improve the biological performance of the composition (for example by improving wetting, retention or distribution on surfaces; resistance to rain on treated surfaces; or uptake or mobility of a compound of formula (I) and/or formula (III)). Such additives include surface active agents, spray additives based on oils, for example certain mineral oils or natural plant oils (such as soy bean and rape seed oil), and blends of these with other bio-enhancing adjuvants (ingredients which may aid or modify the action of a compound of formula (I) and/or formula (III)).

A compound of formula (I) and/or formula (III) may also be formulated for use as a seed treatment, for example as a powder composition, including a powder for dry seed treatment (DS), a water soluble powder (SS) or a water dispersible powder for slurry treatment (WS), or as a liquid composition, including a flowable concentrate (FS), a solution (LS) or a capsule suspension (CS). The preparations of DS, SS, WS, FS and LS compositions are very similar to those of, respectively, DP, SP, WP, SC and DC compositions described above. Compositions for treating seed may include an agent for assisting the adhesion of the composition to the seed (for example a mineral oil or a film-forming barrier).

Wetting agents, dispersing agents and emulsifying agents may be surface SFAs of the cationic, anionic, amphoteric or non-ionic type.

Suitable SFAs of the cationic type include quaternary ammonium compounds (for example cetyltrimethyl ammonium bromide), imidazolines and amine salts.

Suitable anionic SFAs include alkali metals salts of fatty acids, salts of aliphatic monoesters of sulphuric acid (for example sodium lauryl sulphate), salts of sulphonated aromatic compounds (for example sodium dodecylbenzenesulphonate, calcium dodecylbenzenesulphonate, butylnaphthalene sulphonate and mixtures of sodium di-isopropyl- and tri-isopropyl-naphthalene sulphonates), ether sulphates, alcohol ether sulphates (for example sodium laureth-3-sulphate), ether carboxylates (for example sodium laureth-3-carboxylate), phosphate esters (products from the reaction between one or more fatty alcohols and phosphoric acid (predominately mono-esters) or phosphorus pentoxide (predominately di-esters), for example the reaction between lauryl alcohol and tetraphosphoric acid; additionally these products may be ethoxylated), sulphosuccinamates, paraffin or olefine sulphonates, taurates and lignosulphonates.

Suitable SFAs of the amphoteric type include betaines, propionates and glycinates.

Suitable SFAs of the non-ionic type include condensation products of alkylene oxides, such as ethylene oxide, propylene oxide, butylene oxide or mixtures thereof, with fatty alcohols (such as oleyl alcohol or cetyl alcohol) or with alkylphenols (such as octylphenol, nonylphenol or octylcresol); partial esters derived from long chain fatty acids or hexitol anhydrides; condensation products of said partial esters with ethylene oxide; block polymers (comprising ethylene oxide and propylene oxide); alkanolamides; simple esters (for example fatty acid polyethylene glycol esters); amine oxides (for example lauryl dimethyl amine oxide); and lecithins.

Suitable suspending agents include hydrophilic colloids (such as polysaccharides, polyvinylpyrrolidone or sodium carboxymethylcellulose) and swelling clays (such as bentonite or attapulgite).

A compound of formula (I) and/or formula (III) may be applied by any of the known means of applying pesticidal compounds. For example, it may be applied, formulated or unformulated, to the pests or to a locus of the pests (such as a habitat of the pests, or a growing plant liable to infestation by the pests) or to any part of the plant, including the foliage, stems, branches or roots, to the seed before it is planted or to other media in which plants are growing or are to be planted (such as soil surrounding the roots, the soil generally, paddy water or hydroponic culture systems), directly or it may be sprayed on, dusted on, applied by dipping, applied as a cream or paste formulation, applied as a vapour or applied through distribution or incorporation of a composition (such as a granular composition or a composition packed in a water-soluble bag) in soil or an aqueous environment.

A compound of formula (I) and/or formula (III) may also be injected into plants or sprayed onto vegetation using electrodynamic spraying techniques or other low volume methods, or applied by land or aerial irrigation systems.

Compositions for use as aqueous preparations (aqueous solutions or dispersions) are generally supplied in the form of a concentrate containing a high proportion of the active ingredient, the concentrate being added to water before use. These concentrates, which may include DCs, SCs, ECs, EWs, MEs SGs, SPs, WPs, WGs and CSs, are often required to withstand storage for prolonged periods and, after such storage, to be capable of addition to water to form aqueous preparations which remain homogeneous for a sufficient time to enable them to be applied by conventional spray equipment. Such aqueous preparations may contain varying amounts of a compound of formula (I) and/or formula (III) (for example 0.0001 to 10%, by weight) depending upon the purpose for which they are to be used.

A compound of formula (I) and/or formula (III) may be used in mixtures with fertilisers (for example nitrogen-, potasium- or phosphorus-containing fertilisers). Suitable formulation types include granules of fertiliser. The mixtures suitably contain up to 25% by weight of the compound of formula (I) and/or formula (III).

The invention therefore also provides a fertiliser composition comprising a fertiliser and a compound of formula (I) and/or formula (III).

The compositions of this invention may contain other compounds having biological activity, for example micronutrients or compounds having fungicidal activity or which possess plant growth regulating, herbicidal, insecticidal, nematicidal or acaricidal activity.

The compound of formula (I) and/or formula (III) may be the sole active ingredient of the composition or it may be admixed with one or more additional active ingredients such as a pesticide, fungicide, synergist, herbicide or plant growth regulator where appropriate. An additional active ingredient may: provide a composition having a broader spectrum of activity or increased persistence at a locus; synergise the activity or complement the activity (for example by increasing the speed of effect or overcoming repellency) of the compound of formula (I) and/or formula (III); or help to overcome or prevent the development of resistance to individual components. The particular additional active ingredient will depend upon the intended utility of the composition. Examples of suitable pesticides include the following:

a) Pyrethroids, such as permethrin, cypermethrin, fenvalerate, esfenvalerate, deltamethrin, cyhalothrin (in particular lambda-cyhalothrin), bifenthrin, fenpropathrin, cyfluthrin, tefluthrin, fish safe pyrethroids (for example ethofenprox), natural pyrethrin, tetramethrin, s-bioallethrin, fenfluthrin, prallethrin or 5-benzyl-3-furylmethyl-(E)-(1R,3S)-2,2-dimethyl-3-(2-oxothiolan-3-ylidenemethyl)cyclopropane carboxylate;
b) Organophosphates, such as, profenofos, sulprofos, acephate, methyl parathion, azinphos-methyl, demeton-s-methyl, heptenophos, thiometon, fenamiphos, monocrotophos, profenofos, triazophos, methamidophos, dimethoate, phosphamidon, malathion, chlorpyrifos, phosalone, terbufos, fensulfothion, fonofos, phorate, phoxim, pirimiphos-methyl, pirimiphos-ethyl, fenitrothion, fosthiazate or diazinon;
c) Carbamates (including aryl carbamates), such as pirimicarb, triazamate, cloethocarb, carbofuran, furathiocarb, ethiofencarb, aldicarb, thiofurox, carbosulfan, bendiocarb, fenobucarb, propoxur, methomyl or oxamyl;
d) Benzoyl ureas, such as diflubenzuron, triflumuron, hexaflumuron, flufenoxuron or chlorfluazuron;
e) Organic tin compounds, such as cyhexatin, fenbutatin oxide or azocyclotin;
f) Pyrazoles, such as tebufenpyrad and fenpyroximate;
g) Macrolides, such as avermectins or milbemycins, for example abamectin, emamectin benzoate, ivermectin, milbemycin, spinosad or azadirachtin;
h) Hormones or pheromones;
i) Organochlorine compounds such as endosulfan, benzene hexachloride, DDT, chlordane or dieldrin;
j) Amidines, such as chiordimeform or amitraz;
k) Fumigant agents, such as chloropicrin, dichloropropane, methyl bromide or metam;
l) Chloronicotinyl compounds such as imidacloprid, thiacloprid, acetamiprid, nitenpyram or thiamethoxam;
m) Diacylhydrazines, such as tebufenozide, chromafenozide or methoxyfenozide;
n) Diphenyl ethers, such as diofenolan or pyriproxifen;
o) lndoxacarb;

### p) Chlorfenapyr; or

### q) Pymetrozine.

In addition to the major chemical classes of pesticide listed above, other pesticides having particular targets may be employed in the composition, if appropriate for the intended utility of the composition. For instance, selective insecticides for particular crops, for example stemborer specific insecticides (such as cartap) or hopper specific insecticides (such as buprofezin) for use in rice may be employed. Alternatively insecticides or acaricides specific for particular insect species/stages may also be included in the compositions (for example acaricidal ovo-larvicides, such as clofentezine, flubenzimine, hexythiazox or tetradifon; acaricidal motilicides, such as dicofol or propargite; acaricides, such as bromopropylate or chlorobenzilate; or growth regulators, such as hydramethylnon, cyromazine, methoprene, chlorfluazuron or diflubenzuron).

Examples of fungicidal compounds which may be included in the composition of the invention are (E)-N-methyl-2-[2-(2,5-dimethylphenoxymethyl)phenyl]-2-methoxy-iminoacetamide (SSF-129), 4-bromo-2-cyano-N,N-dimethyl-6-trifluoromethylbenzimidazole-1-sulphonamide, α-[N-(3-chloro-2,6-xylyl)-2-methoxyacetamido]-γ-butyrolactone, 4-chloro-2-cyano-N,N-dimethyl-5-p-tolylimidazole-1-sulfonamide (IKF-916, cyamidazosulfamid), 3-5-dichloro-N-(3-chloro-1-ethyl-1-methyl-2-oxopropyl)-4-methylbenzamide (RH-7281, zoxamide), N-allyl-4,5,-dimethyl-2-trimethylsilylthiophene-3-carboxamide (MON65500), N-(1-cyano-1,2-dimethylpropyl)-2-(2,4-dichlorophenoxy)propionamide (AC382042), N-(2-methoxy-5-pyridyl)-cyclopropane carboxamide, acibenzolar (CGA245704), alanycarb, aldimorph, anilazine, azaconazole, azoxystrobin, benalaxyl, benomyl, biloxazol, bitertanol, blasticidin S, bromuconazole, bupirimate, captafol, captan, carbendazim, carbendazim chlorhydrate, carboxin, carpropamid, carvone, CGA41396, CGA41397, chinomethionate, chlorothalonil, chlorozolinate, clozylacon, copper containing compounds such as copper oxychloride, copper oxyquinolate, copper sulphate, copper tallate and Bordeaux mixture, cymoxanil, cyproconazole, cyprodinil, debacarb, di-2-pyridyl disulphide 1,1′-dioxide, dichlofluanid, diclomezine, dicloran, diethofencarb, difenoconazole, difenzoquat, diflumetorim, O,O-di-iso-propyl-5-benzyl thiophosphate, dimefluazole, dimetconazole, dimethomorph, dimethirimol, diniconazole, dinocap, dithianon, dodecyl dimethyl ammonium chloride, dodemorph, dodine, doguadine, edifenphos, epoxiconazole, ethirimol, ethyl(Z)-N-benzyl-N([methyl(methyl-thioethylideneaminooxycarbonyl)amino]thio)-β-alaninate, etridiazole, famoxadone, fenamidone (RPA407213), fenarimol, fenbuconazole, fenfuram, fenhexamid (KBR2738), fenpiclonil, fenpropidin, fenpropimorph, fentin acetate, fentin hydroxide, ferbam, ferimzone, fluazinam, fludioxonil, flumetover, fluoroimide, fluquinconazole, flusilazole, flutolanil, flutriafol, folpet, fuberidazole, furalaxyl, furametpyr, guazatine, hexaconazole, hydroxyisoxazole, hymexazole, imazalil, imibenconazole, iminoctadine, iminoctadine triacetate, ipconazole, iprobenfos, iprodione, iprovalicarb (SZX0722), isopropanyl butyl carbamate, isoprothiolane, kasugamycin, kresoxim-methyl, LY186054, LY211795, LY248908, mancozeb, maneb, mefenoxam, mepanipyrim, mepronil, metalaxyl, metconazole, metiram, metiram-zinc, metominostrobin, myclobutanil, neoasozin, nickel dimethyldithiocarbamate, nitrothal-isopropyl, nuarimol, ofurace, organomercury compounds, oxadixyl, oxasulfuron, oxolinic acid, oxpoconazole, oxycarboxin, pefurazoate, penconazole, pencycuron, phenazin oxide, phosetyl-Al, phosphorus acids, phthalide, picoxystrobin (ZA1963), polyoxin D, polyram, probenazole, prochloraz, procymidone, propamocarb, propiconazole, propineb, propionic acid, pyrazophos, pyrifenox, pyrimethanil, pyroquilon, pyroxyfur, pyrrolnitrin, quaternary ammonium compounds, quinomethionate, quinoxyfen, quintozene, sipconazole (F-155), sodium pentachlorophenate, spiroxamine, streptomycin, sulphur, tebuconazole, tecloftalam, tecnazene, tetraconazole, thiabendazole, thifluzamid, 2-(thiocyanomethylthio)benzothiazole, thiophanate-methyl, thiram, timibenconazole, tolclofos-methyl, tolylfluanid, triadimefon, triadimenol, triazbutil, triazoxide, tricyclazole, tridemorph, trifloxystrobin (CGA279202), triforine, triflumizole, triticonazole, validamycin A, vapam, vinclozolin, zineb and ziram.

The compounds of formula (I) and/or formula (III) may be mixed with soil, peat or other rooting media for the protection of plants against seed-borne, soil-borne or foliar fungal diseases.

Examples of suitable synergists for use in the compositions include piperonyl butoxide, sesamex, safroxan and dodecyl imidazole.

Suitable herbicides and plant-growth regulators for inclusion in the compositions will depend upon the intended target and the effect required.

An example of a rice selective herbicide which may be included is propanil. An example of a plant growth regulator for use in cotton is PIX™.

Some mixtures may comprise active ingredients which have significantly different physical, chemical or biological properties such that they do not easily lend themselves to the same conventional formulation type. In these circumstances other formulation types may be prepared. For example, where one active ingredient is a water insoluble solid and the other a water insoluble liquid, it may nevertheless be possible to disperse each active ingredient in the same continuous aqueous phase by dispersing the solid active ingredient as a suspension (using a preparation analogous to that of an SC) but dispersing the liquid active ingredient as an emulsion (using a preparation analogous to that of an EW). The resultant composition is a suspoemulsion (SE) formulation.

The invention is illustrated by the following Examples:

### EXAMPLE 1

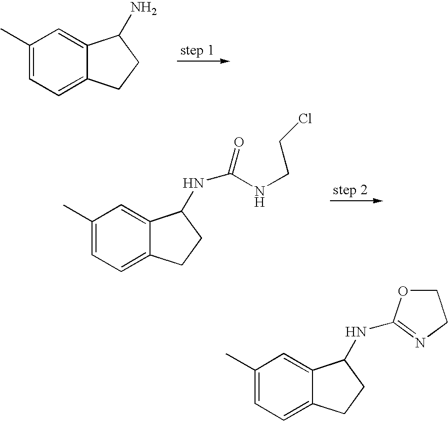

### Step 1. 1-(2-Chloro-ethyl)-3-(6-methyl-indan-1-yl)-urea

2-Chloroethylisocyanate (0.610 ml, 6.79 mmol) was added to a solution of 6-methyl-1-indanamine (1 g, 6.79 mmol) in EtOAc (16 ml) at 0° C. with stirring to give a white precipitate. After stirring for 2 hours at room temperature the mixture was cooled to 0° C. and the product filtered off and washed with EtOAc and Et₂O to give 1.55 g of a white solid.

¹H-NMR (ppm, D₆-DMSO): 7.11 (d, arom); 7.02 (s, arom); 7.00 (d, arom); 6.41 (d, NH); 6.11 (t, NH); 5.06 (m, H—C(1)); 3.62 (t, CH₂Cl); 3.38 (m, CH₂N); 2, 38 (m, H—C(3)); 2.70 (m, H—C(3)); 2.48 (m, H—C(2)); 2.39 (s, Me); 1.68 (m, H—C(2)).

### Step 2. (4,5-Dihydro-oxazol-2-yl)-(6-methyl-indan-1-yl)-amine

A solution of the product of step 1 (800 mg, 3.16 mmol) and DBU (0.940 ml, 6.33 mmol) in DMF (6 ml) was heated at 100° C. for 3 hours then cooled and poured onto 25 ml ice and stirred for 30 minutes. The product was filtered off and washed twice with water, and dried overnight in the air to give 587 mg of a white solid.

¹H-NMR (ppm, D₆-DMSO): 6.92 (s, arom); 6.80 (s, arom); 6,47 (br s, NH); 4.78 (br s, H—C(1)); 4.01 and 3.43 (2×br s, oxazoline); 2,64 and 2.50 (2 m, 2H—C(3)); 2.16 and 1.48 (2 m, 2H—C(2)); 2.04 (s, Me)

### EXAMPLE 2

This Example illustrates the pesticidal/insecticidal properties of compounds of formula (I). The compounds numbers are those of the characterising data tables. Test against were performed as follows:

Heliothis virescens (Tobacco budworm):
Eggs (0-24 h old) were placed in 24-well microtiter plate on artificial diet and treated with test solutions at an application rate of 200 ppm by pipetting. After an incubation period of 4 days, samples were checked for egg mortality, larval mortality, and growth regulation. The following compounds gave at least 80% control of Heliothis virescens: 1.004, 1.005, 1.012, 1.016, 4.001, 31.016, 31.018.
Myzus persicae (Green peach aphid):
Sunflower leaf discs were placed on agar in a 24-well microtiter plate and sprayed with test solutions at an application rate of 200 ppm. After drying, the leaf discs were infested with an aphid population of mixed ages. After an incubation period of 6 DAT, samples were checked for mortality.

The following compounds gave at least 80% control of Myzus persicae: 1.001, 1.002, 1.004, 1.005, 1.006, 1.007, 1.009, 1.010, 1.012, 1.015, 1.016, 1.017, 2.001, 2.003, 3.001, 3.004, 3.005, 3.006, 3.007, 3.009, 3.010, 31.001, 31.002, 31.016, 31.017, 31.018, 33.010, 33.011.

Myzus persicae (Green peach aphid):
Roots of pea seedlings, infested with an aphid population of mixed ages, were placed directly in the test solutions of 24 ppm. 6 days after introduction, samples were checked for mortality.

The following compounds gave at least 80% control of Myzus persicae: 1.001, 1.002, 1.004, 1.010, 1.012, 1.015, 1.016, 1.017, 2.001, 3.007, 3.008, 31.001, 31.002, 31.016, 31.017, 31.018.

Tetranychus urticae (Two-spotted spider mite):
Bean leaf discs on agar in 24-well microtiter plates wer sprayed with test solutions at an application rate of 200 ppm. After drying, the leaf discs are infested with mite populations of mixed ages. 8 days later, discs are checked for egg mortality, larval mortality, and adult mortality.

The following compounds gave at least 80% control of Tetranychus urticae: 1.004, 1.016, 2.001, 3.006, 31.016, 31.018.

1. A method of combating and controlling insects, acarines, or molluscs which comprises applying to a pest, to a locus of a pest, or to a plant susceptible to attack by a pest an insecticidally, acaricidally, nematicidally or molluscicidally effective amount of a compound of formula (I): 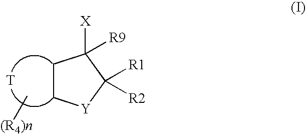
wherein
X is (i), (ii) or (iii) 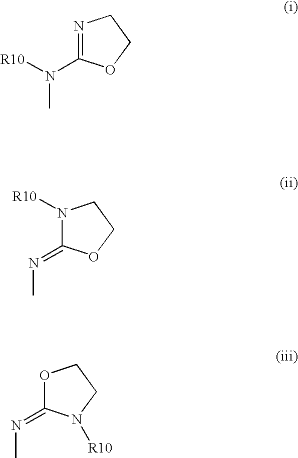
R¹⁰ is hydrogen, hydroxy, cyano, formyl, G-, G-O—, G-S—, G-S—S—, G-A-, R²⁴R²⁵N—, G-A-NR¹⁷—, R²⁴R²⁵N—S—, R²⁴R²⁵N-A-, R¹⁸N═C(R¹⁹)—, G-O-A- or G-S-A-; where R²⁴ and R²⁵ are independently H or G-, or R²⁴ and R²⁵ together with the N atom to which they are attached, form a group N═CRaRb where Ra and Rb are H, C₁₋₆ alkyl or phenyl; or R²⁴ and R²⁵ together with the N atom to which they are attached form a five, six or seven membered ring which may contain one or two further hetero atoms selected from O, N or S and which may be optionally substituted by one to four C₁₋₆ alkyl groups or phenyl; R¹⁷ is H, G-, G-C(O)— or G-OC(O); R¹⁸ is H, OH, cyano, nitro, G-, G-O— or R³⁸R³⁹N—, where R³⁸ and R³⁹ are independently H or G-, or R³⁸ and R³⁹ together with the N atom to which they are attached, form a group N═CRaRb where Ra and Rb are H, C₁₋₆ alkyl or phenyl; or R³⁸ and R³⁹ together with the N atom to which they are attached form a five, six or seven membered ring which may contain one or two further hetero atoms selected from O, N or S and which may be optionally substituted by one to four C₁₋₆ alkyl groups; R¹⁹ is H, cyano, G-, G-O—, G-S— or R⁴²R⁴³N—, where R⁴² and R⁴³ are independently H or G; or R⁴² and R⁴³ together with the N atom to which they are attached, form a group N═CRaRb where Ra and Rb are H, C₁₋₆ alkyl or phenyl; or R⁴² and R⁴³ together with the N atom to which they are attached form a five, six or seven membered ring which may contain one or two further hetero atoms selected from O, N or S and which may be optionally substituted by one to four C₁₋₆ alkyl groups or phenyl;
Y is O, S(O)ₘ, where m is 0, 1 or 2, NR³, SO₂—NR³, NR³—SO₂, NR³—O or O—NR³, where R³ is H, OH, cyano, formyl, G-, G-O—, G-S—, G-A-, R²⁷R²⁸N—, R²⁷R²⁸N-A-, G-O-A-, G-S-A-, G-A-NR²⁹—, R²⁷R²⁸N-A-NR²⁹—, G-O-A-NR²⁹— or G-S-A-NR²⁹—, where R²⁷ and R²⁸ are independently H or G-, or R²⁷ and R²⁸ together with the N atom to which they are attached, form a group N═CRaRb where Ra and Rb are H, C₁₋₆ alkyl or phenyl; or R²⁷ and R²⁸ together with the N atom to which they are attached form a five, six or seven membered ring which may contain one or two further hetero atoms selected from O, N or S and which may be optionally substituted by one to four C₁₋₆ alkyl groups or phenyl; R²⁹ is H or G-; or Y is CR⁵R⁶, CR⁵R⁶—CR⁷R⁸, O—CR⁷R⁸, S(O)ₘ—CR⁷R⁸, NR³—CR⁷R⁸, CR⁵R⁶—O, CR⁵R⁶—S(O)ₘ, CR⁵R⁶—NR³, where R³ and m have the meanings assigned to them above, and R⁵, R⁶, R⁷ and R⁸ are each independently H, OH, halogen, nitro, cyano, rhodano, carboxy, formyl, formyloxy, G-, G-O—, G-S—, G-A-, R²¹R²²N—, R²¹R²²N-A-, G-O-A-, G-S-A-, G-A-O—, G-A-S—, G-A-NR²³—, R²¹R²²N-A-O—, R²¹R²²N-A-S—, R²¹R²²N-A-NR²³—, G-O-A-O—, G-O-A-S—, G-O-A-NR²³—, G-S-A-O—, G-S-A-NR²³— or R²⁰S(O)(═NR¹⁷)—, where R²¹ and R²² are independently H or G-, or R²¹ and R²² together with the N atom to which they are attached, form a group N═CRaRb where Ra and Rb are H, C₁₋₆ alkyl or phenyl; or R²¹ and R²² together with the N atom to which they are attached form a five, six or seven membered ring which may contain one or two further hetero atoms selected from O, N or S and which may be optionally substituted by one to four C₁₋₆ alkyl groups or phenyl; R²³ is H or G- and R¹⁷ is as defined above; R²⁰ is C₁₋₆ alkyl, optionally substituted phenyl, optionally substituted benzyl; or two of the groups R⁵, R⁶, R⁷ and R⁸ attached to the same carbon atom are ═O, ═S, ═NR¹¹ or ═CR¹²R¹³, where R¹¹ is H, OH, nitro, cyano, formyl, formyloxy, G-, G-O—, G-A-, R³⁶R³⁷N—, G-C(O)—O—, G-C(O)—NR²⁶—, R³⁶R³⁷N—C(O)O—, G-O—C(O)O—, G-O—C(O)—NR²⁶—, where R³⁶, R³⁷ and R²⁶ are independently H or G-, or R³⁶ and R³⁷ together with the N atom to which they are attached, form a group N═CRaRb where Ra and Rb are H, C₁₋₆ alkyl or phenyl; or R³⁶ and R³⁷ together with the N atom to which they are attached form a five, six or seven membered ring which may contain one or two further hetero atoms selected from O, N or S and which may be optionally substituted by one to four C₁₋₆ alkyl groups or phenyl, and R¹² and R¹³ are each independently H, halogen, nitro, cyano, formyl, formyloxy, G-, G-O—, G-S—, G-A-, R⁴⁰R⁴¹N—, R⁴⁰R⁴¹N-A-, G-O-A-, G-A-O—, R⁴⁰R⁴¹N-A-O—, R⁴⁰R⁴¹N-A-S—, G-O-A-O—, G-O-A-S—, G-O-A-NR³⁰—, where R⁴⁰, R⁴¹ and R³⁰ are independently H or G-, or R⁴⁰ and R⁴¹ together with the N atom to which they are attached, form a group N═CRaRb where Ra and Rb are H, C₁₋₆ alkyl or phenyl; or R⁴⁹ and R⁴¹ together with the N atom to which they are attached form a five, six or seven membered ring which may contain one or two further hetero atoms selected from O, N or S and which may be optionally substituted by one to four C₁₋₆ alkyl groups or phenyl, or R¹² and R¹³ together with the carbon atom to which they are attached form a 3 to 6 membered carbocyclic ring; or the groups R⁵ and R⁶ or R⁷ and R⁸ together with the carbon atom to which they are attached form a three to six membered ring, containing at least 2 carbon atoms and optionally containing one or two sulfur and/or one or two non-adjacent oxygen atoms or a group NR¹⁴, where R¹⁴ is H, OH, cyano, formyl, G-, G-O—, G-S—, G-A-, R²⁷R²⁸N—, R²⁷R²⁸N-A-, G-O-A-, G-S-A-, G-A-NR²⁹—, R²⁷R²⁸N-A-NR²⁹—, G-O-A-NR²⁹— or G-S-A-NR²⁹—, where R²⁷, R²⁸ and R²⁹ have the meanings assigned to them above, the ring being optionally substituted by one to four C₁₋₆ alkyl groups or phenyl; or two of the groups R⁵, R⁶, R⁷ and R⁸ attached to different atoms together with the atoms they are attached form a three to seven membered ring, that optionally contains one or two sulfur and/or one or two non-adjacent oxygen atoms or a group NR¹⁴, where R¹⁴ is as defined above, or two of the groups R⁵, R⁶, R⁷ and R⁸ attached to adjacent atoms combine to form a bond;
the ring (T) 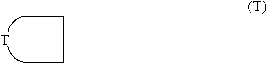
is a 5- or 6-membered aromatic or heteroaromatic ring;
R¹ and R² are each independently H, OH, halogen, nitro, cyano, rhodano, carboxy, formyl, formyloxy, G-, G-O—, G-S—, G-A-, R²¹R²²N—, R²¹R²²N-A-, G-O-A-, G-S-A-, G-A-O—, G-A-S—, G-A-NR²³—, R²¹R²²N-A-O—, R²¹R²²N-A-S—, R²¹R²²N-A-NR²³—, G-O-A-O—, G-O-A-S—, G-O-A-NR²³—, G-S-A-O—, G-S-A-NR²³— or R²⁰S(O)(═NR¹⁷)—, where R¹⁷, R²⁰, R²¹, R²² and R²³ are as defined above, or two of the groups R¹ and R² attached to the same carbon atom are ═O, ═S, ═NR¹¹ or ═CR¹²R¹³, where R¹¹, R¹² and R¹³ are defined as above, or the groups R¹ and R² together with the same carbon atom to which they are attached form a three to six membered ring, containing at least 2 carbon atoms and optionally containing one or two sulfur and/or one or two non-adjacent oxygen atoms or a group NR¹⁴, where R¹⁴ is as defined above, the ring being optionally substituted by C₁₋₆ alkyl; or two of the groups R¹, R² and R⁷, R⁸ attached to different atoms together with the atoms they are attached form a three to seven membered ring, that optionally contains one or two sulfur and/or one or two non-adjacent oxygen atoms or a group NR¹⁴, where R¹⁴ is defined as above, the ring being optionally substituted by one or four C₁₋₆ alkyl groups or phenyl; or two of the groups R¹, R², R⁵, R⁶, R⁷ and R⁸ attached to adjacent atoms combine to form a bond;
each R⁴ is independently OH, halogen, nitro, cyano, azido, rhodano, isothiocyanato, carboxy, formyl, formyloxy, G-, G-O—, G-S—, G-A-, R³¹R³²N—, R³¹R³²N-A-, G-O-A-, G-S-A-, G-A-O—, G-A-S—, G-A-NR³³—, R³¹R³²N-A-O—, R³¹R³²N-A-S—, R³¹R³²N-A-NR³³—, G-O-A-O—, G-O-A-S—, G-O-A-NR³³—, G-S-A-O, G-S-A-NR³³—, R²⁰S(O)(═NR¹⁷)—, R¹⁸N═C(R¹⁹)—, R⁴⁴R⁴⁵P(O)— or R⁴⁴R⁴⁵P(S)—, where R¹⁷, R¹⁸, R¹⁹ and R²⁰ have the meanings assigned to them above, and R³¹, R³² and R³³ are independently H or G-, or R³¹ and R³² together with the N atom to which they are attached, form a group N═CRaRb where Ra and Rb are H, C₁₋₆ alkyl or phenyl; or R³¹ and R³² together with the N atom to which they are attached form a five, six or seven membered ring which may contain one or two further hetero atoms selected from O, N or S and which may be optionally substituted by one to four C₁₋₆ alkyl groups or phenyl, and R⁴⁴ and R⁴⁵ are independently H, C₁₋₆ alkyl, C₁₋₆ alkoxy, phenyl, phenoxy; or 2 adjacent groups R⁴ together with the carbon atoms to which they are attached form a 4, 5, 6 or 7 membered carbocyclic or heterocyclic ring which may be optionally substituted by C₁₋₆alkyl or halogen; or a group R⁴ together with a group R³, R⁵, R⁶ or R⁹ and the atoms to which they are attached form a 5-7 membered ring optionally containing an NR¹⁵ group where R¹⁵ is H, OH, cyano, formyl, G-, G-O—, G-S—, G-A-, R²⁷R²⁸N—, R²⁷R²⁸N-A-, G-O-A-, G-S-A-, G-A-NR²⁹—, R²⁷R²⁸N-A-NR²⁹—, G-O-A-NR²⁹— or G-S-A-NR²⁹—, where R²⁷, R²⁸ and R²⁹ have the meanings assigned to them above, or containing an S or O atom, the ring being optionally substituted by one to four C₁₋₆ alkyl groups or phenyl;
n is 0, 1, 2, 3 or 4;
R⁹ is H, formyl, G-, G-A-, G-O-A-, R³⁴R³⁵N-A-, where R³⁴ and R³⁵ are independently H or G-, or R³⁴ and R³⁵ together with the N atom to which they are attached, form a group N═CRaRb where Ra and Rb are H, C₁₋₆ alkyl or phenyl; or R³⁴ and R³⁵ together with the N atom to which they are attached form a five, six or seven membered ring which may contain one or two further hetero atoms selected from O, N or S and which may be optionally substituted by one to four C₁₋₆ alkyl groups or phenyl; or R⁹ is G-O-A- or G-S-A-; or R⁹ together with a group R¹, R², R³, R⁵, R⁶, R⁷ or R⁸ and the atoms to which they are attached may form a three to seven membered ring, that optionally may contain one or two sulfur and/or one or two non-adjacent oxygen atoms or a group NR¹⁶; where R¹⁶ is H, OH, cyano, formyl, G-, G-O—, G-S—, G-A-, R²⁷R²⁸N—, R²⁷R²⁸N-A-, G-O-A-, G-S-A-, G-A-NR²⁹—, R²⁷R²⁸N-A-NR²⁹—, G-O-A-NR²⁹— or G-S-A-NR²⁹—, where R²⁷, R²⁸ and R²⁹ have the meanings assigned to them above,
G is optionally substituted C₁₋₁₂ alkyl, optionally substituted C₂₋₁₂ alkenyl, optionally substituted C₂₋₁₂ alkynyl, optionally substituted C₃₋₈ cycloalkyl, optionally substituted C₃₋₈ cycloalkenyl, optionally substituted aryl, optionally substituted heteroaryl or optionally substituted heterocyclyl;
A is S(O), SO₂, C(O) or C(S);
or salts or N-oxides thereof, and where at least one R¹, R², R⁴, R⁵, R⁶, R⁹ is different to hydrogen when R¹⁰ is hydrogen or methyl, T is benzene and Y is CR⁵R⁶.

2. The method according to claim 1, wherein when the compound of formula I is a compound of formula Iα or Iβ 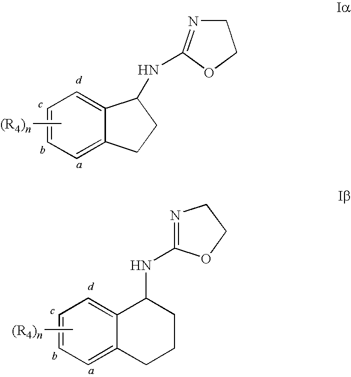
wherein R⁴ is as defined above, and (i) n is 2 or 3, and R⁴ at position “c” is selected from CH₃, Cl, F, Br, CF₃ and OCF₃, then at least one further R⁴ is different to that at position “c”, and when a further R⁴ is present at position “a”, then said R⁴ is not selected from CH₃, Cl, F, Br, CF₃ or OCF₃, or (ii) when the compound of formula I is a compound of formula Iα or Iβ as defined above, and n is 1, then R⁴ is not CH₃, Cl, F, Br, CF₃ or OCF₃.

3. The method according to claim 1, wherein the compound of formula I is a compound of formula (IA) 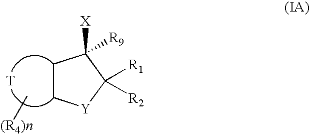
wherein the absolute chirality on the X and R⁹ bearing carbon atom is as shown, and T, X, Y, R¹, R², R⁴, R⁹, and n are as defined in claim 1.

4. The method according to claim 1, wherein the ring (T) 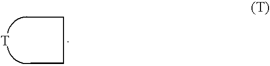
is a benzene ring.

5. The method according to claim 1, wherein the ring (T) 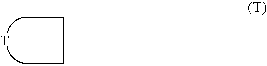
is a 5 or 6 membered heteroaromatic ring, wherein the ring members are each independently CH, S, N, NR⁴, O or CR⁴, wherein R⁴ is as defined in claim 1, provided that there is no more than one O or S atom present in the ring.

6. The method according to claim 1, wherein Y is O, S(O)ₘ, NR³, SO₂—NR³, NR³—SO₂, NR³—O, O—NR³, O—CR⁷R⁸, S(O)ₘ—CR⁷R⁸, NR³—CR⁷R⁸, CR⁵R⁶—O, CR⁵R⁶—S(O)ₘ or CR⁵R⁶—NR³, and wherein R³, R⁵, R⁶, R⁷, R⁸ and m are as defined in claim 1.

7. The method according to claim 1, wherein Y is O or CR⁵R⁶ and R⁵ and R⁶ are as defined in claim 1.

8. The method according to claim 1, wherein Y is CR⁵R⁶ or CR⁵R⁶CR⁷R⁸, and R⁵, R⁶, R⁷ and R⁸ are as R⁶, defined in claim 1, and where at least one of R¹, R², R⁵, R⁶, R⁷, R⁸ or R⁹ is not hydrogen.

9. The method according to claim 1, wherein each of R⁵, R⁶, R⁷ and R⁸ is independently: hydrogen; halogen; hydroxyl; amino; nitro; cyano; C₁₋₆ alkyl; C₁₋₆ alkenyl; C₁₋₆ haloalkyl; C₁₋₆ alkoxy(C₁₋₆)alkyl; phenyl(C₁₋₃)alkyl, wherein the phenyl group is optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino, dialkylamino, C₁₋₆ alkylsulfonyl or C₁₋₆ alkoxycarbonyl, or two adjacent positions on the phenyl ring may be cyclised to form a 5, 6 or 7 membered carbocyclic or heterocyclic ring and said ring is optionally substituted with halogen); C₃₋₅ cycloalkyl; 1,3-dioxolan-2-yl; phenyl, optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino, dialkylamino, C₁₋₆ alkylsulfonyl or C₁₋₆ alkoxycarbonyl, or two adjacent positions on the phenyl ring may be cyclised to form a 5, 6 or 7 membered carbocyclic or heterocyclic ring and said ring is optionally substituted with halogen; heteroaryl optionally substituted by halo, nitro, cyano, C₁₋₆ alkyl, C₁₋₆ haloalkyl, C₁₋₆ alkoxy or C₁₋₆ haloalkoxy; C₁₋₆ alkoxy; C₁₋₆ haloalkoxy; C₁₋₆ alkenyloxy; C₁₋₆ alkynyloxy; C₁₋₃ alkoxy(C₁₋₃)alkoxy; benzyloxy, wherein the phenyl is optionally substituted by halogen, C₁₋₄ alkyl, C₁₋₄ alkoxy, C₁₋₄ haloalkyl, C₁₋₄ haloalkoxy, CN, or NO₂; phenoxy, wherein the phenyl is optionally substituted by halogen, nitro, cyano, C₁₋₆ alkyl, C₁₋₆ haloalkyl, C₁₋₆ alkoxy or C₁₋₆ haloalkoxy); C₁₋₆ alkylthio; C₁₋₆ haloalkylthio; formyl; C₁₋₆ alkylcarbonyl; C₁₋₆ alkoxycarbonyl; carboxy; C₁₋₆ alkoxythionocarbonyl; carbamoyl; C₁₋₆ alkylaminocarbonyl; di-C₁₋₆ alkylaminocarbonyl; thiocarbamoyl; C₁₋₆ alkylaminothionocarbonyl; di-C₁₋₆ alkylaminothionocarbonyl; C₁₋₆ alkylcarbonyloxy; C₁₋₆ alkylthionocarbonyloxy; C₁₋₆ alkoxycarbonyloxy; C₁₋₆ alkylaminocarbonyloxy; di-C₁₋₆ alkylaminocarbonyloxy; C₁₋₆ alkylcarbonylthio; C₁₋₆ alkylaminothionocarbonylthio; di-(C₁₋₆)-alkylaminothionocarbonylthio; C₁₋₆ alkoxycarbonylamino; aminocarbonylamino; C₁₋₆ alkylaminocarbonylamino; di-C₁₋₆alkylaminocarbonylamino; NR⁵⁴R⁵⁵, where R⁵⁴ and R⁵⁵ are independently hydrogen, C₁₋₆ alkyl, C₁₋₆ haloalkyl, C₁₋₄ alkoxy(C₁₋₄)alkyl, formyl, C₁₋₆ alkylcarbonyl, or optionally substituted phenylcarbonyl wherein said substitution on the phenyl is selected from halogen, C₁₋₄ alkyl, C₁₋₄ alkoxy, C₁₋₄ haloalkyl, C₁₋₄ haloalkoxy, CN and NO₂); or R⁵ and R⁶ or R⁷ and R⁸ together with the carbon atom to which they are attached form a three to six membered ring, said ring optionally comprising one or two sulfur atoms or one or two non-adjacent oxygen atoms or a group NR⁵⁶, wherein R⁵⁶ is hydrogen, C₁₋₆ alkyl, C₁₋₆ haloalkyl, C₁₋₄ alkoxy(C₁₋₄)alkyl, formyl, C₁₋₆ alkylcarbonyl); or R⁵ or R⁷ when attached to a carbon atom adjacent to the R¹ bearing carbon atom, together with R¹ form a bond; or R⁵ and R¹, or R⁷ and R¹, together with the carbon atoms to which they are attached form a three to six membered ring optionally comprising one or two non-adjacent oxygen atoms; or R⁵ and R⁶, or R⁷ and R⁸, form ═O, ═S, ═NR⁵⁷ or ═CR⁵⁸R⁵⁹, wherein R⁵⁷ is OH, optionally substituted C₁₋₆ alkoxy or C₁₋₄ alkylcarbonylamino, and R⁵⁸ and R⁵⁹ are independently H or C₁₋₆ alkyl.

10. The method according to claim 1, wherein R¹ and R² are independently: hydrogen; hydroxyl; halogen; amino; nitro; cyano; C₁₋₆ alkyl; C₁₋₆ alkenyl; C₁₋₆ haloalkyl; C₁₋₆ alkoxy(C₁₋₆)alkyl; phenyl(C₁₋₃)alkyl, wherein the phenyl group is optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino, dialkylamino, C₁₋₆ alkylsulfonyl or C₁₋₆ alkoxycarbonyl); C₃₋₅ cycloalkyl; 1,3-dioxolan-2-yl; phenyl optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino, dialkylamino, C₁₋₆ alkylsulfonyl or C₁₋₆ alkoxycarbonyl; heteroaryl optionally substituted by halogen, nitro, cyano, C₁₋₆ alkyl, C₁₋₆ haloalkyl, C₁₋₆ alkoxy or C₁₋₆ haloalkoxy); C₁₋₆ alkoxy; C₁₋₆ haloalkoxy; C₂₋₆ alkenyloxy; C₂₋₆ alkynyloxy; phenoxy, wherein the phenyl group is optionally substituted by halogen, nitro, cyano, C₁₋₆ alkyl, C₁₋₆ haloalkyl, C₁₋₆ alkoxy or C₁₋₆ haloalkoxy; C₁₋₆ alkylthio; C₁₋₆ haloalkylthio; formyl; C₁₋₆ alkylcarbonyl; phenylcarbonyl, wherein the phenyl group is optionally substituted by halogen, C₁₋₄ alkyl, C₁₋₄ alkoxy, C₁₋₄ haloalkyl, C₁₋₄ haloalkoxy, CN, or NO₂; C₁₋₆ alkoxycarbonyl; carboxy; C₁₋₆ alkoxythionocarbonyl; carbamoyl; C₁₋₆ alkylaminocarbonyl; di-C₁₋₆ alkylaminocarbonyl; thiocarbamoyl; C₁₋₆ alkylaminothionocarbonyl; di-C₁₋₆ alkylaminothionocarbonyl; C₁₋₆ alkylcarbonyloxy; C₁₋₆ alkylthionocarbonyloxy; C₁₋₆ alkoxycarbonyloxy; C₁₋₆ alkylaminocarbonyloxy; di-C₁₋₆ alkylaminocarbonyloxy; C₁₋₆ alkylcarbonylthio; C₁₋₆ alkylaminothionocarbonylthio; di-(C₁₋₆)-alkylaminothionocarbonylthio; C₁₋₈ alkylamino; di-(C₁₋₈)-alkylamino; formylamino; C₁₋₆ alkylcarbonylamino; C₁₋₆ alkoxycarbonylamino; aminocarbonylamino; C₁₋₆ alkylaminocarbonylamino; di-C₁₋₆alkylaminocarbonylamino; or R¹ and R² together are ═O, ═S, ═NR¹¹ or ═CR¹²R¹³, wherein R¹¹ is OH, C₁₋₆ alkoxy or C₁₋₆ alkylcarbonylamino, and R¹² and R¹³ are independently H, C₁₋₆ alkyl, or C₁₋₆ haloalkyl; or R¹ and R⁹ together with the carbon atom they are attached form a three to six membered ring optionally comprising one or two non-adjacent oxygen atoms; or R¹ and R² together form a three to six membered ring optionally comprising one or two non-adjacent oxygen atoms.

11. The method according to, claim 1, wherein each R⁴ is independently: halogen; hydroxyl; amino; nitro; cyano; C₁₋₈ alkyl; C₁₋₈ haloalkyl; cyano(C₁₋₆)alkyl; C₁₋₃ alkoxy(C₁₋₃)alkyl; C₂₋₆ alkenyl; C₂₋₆ alkynyl; C₃₋₆ cycloalkyl; C₁₋₃ alkyl-(C₃₋₆)-cycloalkyl; phenyl optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino or dialkylamino; heteroaryl optionally substituted by halogen, nitro, cyano, C₁₋₆ alkyl, C₁₋₆ haloalkyl, C₁₋₆ alkoxy or C₁₋₆ haloalkoxy; heterocyclyl optionally substituted by halogen, nitro, cyano, C₁₋₆ alkyl, C₁₋₆ haloalkyl, C₁₋₆ alkoxy or C₁₋₆ haloalkoxy; formyl; C₁₋₆ alkylcarbonyl; carboxy; C₁₋₆ alkoxycarbonyl; C₁₋₆ alkylthiocarbonyl; C₁₋₆ alkoxythionocarbonyl; carbamoyl; C₁₋₆ alkylaminocarbonyl; di-C₁₋₆ alkylaminocarbonyl; thiocarbamoyl; C₁₋₆ alkylaminothionocarbonyl; di-C₁₋₆ alkylaminothionocarbonyl; C₁₋₈ alkoxy; C₁₋₆ haloalkoxy; phenoxy, wherein the phenyl group is optionally substituted by halogen, C₁₋₄ alkyl, C₁₋₄ alkoxy, C₁₋₄ haloalkyl, C₁₋₄ haloalkoxy, CN, NO₂, or phenyl; heteroaryloxy optionally substituted by halo, nitro, cyano, C₁₋₃ alkyl, C₁₋₃ haloalkyl, C₁₋₃ alkoxy or C₁₋₃ haloalkoxy; C₁₋₆ alkylcarbonyloxy; C₁₋₆ alkylthionocarbonyloxy; C₁₋₆ alkoxycarbonyloxy; C₁₋₆ alkylaminocarbonyloxy; di-C₁₋₆ alkylaminocarbonyloxy; C₁₋₆ alkylaminothionocarbonyloxy; di-C₁₋₆ alkylaminothionocarbonyloxy; C₁₋₈ alkylthio; C₁₋₆ haloalkylthio; arylthio or heteroarylthio where the aryl or heteroaryl is optionally substituted by halogen, C₁₋₄ alkyl, C₁₋₄ alkoxy, C₁₋₄ haloalkyl, C₁₋₄ haloalkoxy, CN, NO₂ or phenyl; C₁₋₆ alkylcarbonylthio; C₁₋₆ alkylaminocarbonylthio; di-C₁₋₆ alkylaminocarbonylthio; C₁₋₆ alkylaminothionocarbonylthio; di-(C₁₋₆)-alkylaminothionocarbonylthio; C₁₋₈ alkylamino; di-(C₁₋₈)-alkylamino, formylamino; C₁₋₆ alkylcarbonylamino; C₁₋₆ alkoxycarbonylamino, aminocarbonylamino; C₁₋₆ alkylaminocarbonylamino; di-C₁₋₆alkylaminocarbonylamino; aminothionocarbonylamino; C₁₋₆ alkylaminothionocarbonylamino; di-C₁₋₆ alkylaminothionocarbonylamino; or 2 adjacent groups R⁴ together with the carbon atoms to which they are attached form a 4, 5, 6 or 7 membered carbocylic or heterocyclic ring optionally substituted by halogen; n is 1, 2 or 3.

12. The method according to claim 1, wherein R⁹ is: hydrogen; C₁₋₆ alkyl; C₁₋₆ cyanoalkyl; C₁₋₆ haloalkyl; C₃₋₇ cycloalkyl(C₁₋₄alkyl; C₁₋₆ alkoxy(C₁₋₆)alkyl; aryl(C₁₋₆)alkyl, wherein the aryl group is optionally substituted by halogen, nitro, cyano, C₁₋₆ alkyl, C₁₋₆ haloalkyl, C₁₋₆ alkoxy, C₁₋₆ haloalkoxy, C₁₋₆ alkylsulfonyl, C₁₋₆ alkylsulfinyl, C₁₋₆ alkylthio, C₁₋₆ alkoxycarbonyl, C₁₋₆ alkylcarbonylamino, or arylcarbonyl; C₁₋₆ alkylcarbonyl; phenylcarbonyl, wherein the phenyl is optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino or dialkylamino; C₁₋₆ alkoxycarbonyl; carboxy; alkylaminothionocarbonyl; C(O)NR³⁴R³⁵, wherein R³⁴ and R³⁵ are independently hydrogen, C₁₋₆ alkyl or C₁₋₆ haloalkyl or C₁₋₆ alkoxy(C₁₋₆)alkyl or R³⁴ and R³⁵ together with the N atom to which they are attached form a five, six or seven-membered ring containing an O or S atom; or R⁹ and R¹ together with the carbon atoms to which they are attached form a three to six membered ring, optionally comprising one or two sulphur and/or one or two non-adjacent oxygen atoms.

13. The method according to claim 1, wherein R¹⁹ is: hydrogen; hydroxyl; amino; cyano; C₁₋₆ alkyl; C₁₋₆ haloalkyl; C₁₋₆ alkoxy(C₁₋₆)alkyl; C₃₋₆ alkenyl; C₃₋₆ alkinyl; phenyl(C₁₋₃)alkyl, wherein the phenyl group is optionally substituted by halogen, C₁₋₄ alkyl, C₁₋₄ alkoxy, C₁₋₄ haloalkyl, C₁₋₄ haloalkoxy, cyano, nitro, C₁₋₆ alkylsulfonyl or C₁₋₆ alkoxycarbonyl; C₃₋₅ cycloalkyl-C₁₋₃alkyl; C₃₋₅ cycloalkyl; aryl or heteroaryl wherein the aryl or heteroaryl is optionally substituted by halogen, C₁₋₄ alkyl, C₁₋₄ alkoxy, C₁₋₄ haloalkyl, C₁₋₄ haloalkoxy, cyano, nitro, C₁₋₆ alkylsulfonyl or C₁₋₆ alkoxycarbonyl; C₁₋₆ alkylthio; C₁₋₆ haloalkylthio; C₁₋₆ alkyldithio; C₁₋₆ alkylthiosulfinyl; formyl; C₁₋₆ alkylcarbonyl; aryl- or heteroarylcarbonyl, wherein the aryl or heteroaryl is optionally substituted by halogen, C₁₋₄ alkyl, C₁₋₄ alkoxy, C₁₋₄ haloalkyl, C₁₋₄ haloalkoxy, cyano or nitro; C₁₋₆ alkoxycarbonyl; C₁₋₆ alkylthiocarbonyl; C₁₋₆ alkylcarbonylamino; or R²⁴R²⁵N—, R²⁴R²⁵N—S—, or R²⁴R²⁵N-A- wherein R²⁴ and R²⁵ are independently hydrogen or C₁₋₆alkyl and A is SO₂, C(O) or C(S).

14. A compound of formula IB 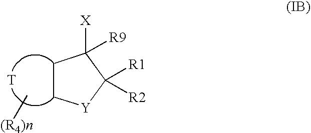
wherein T, X, Y, R¹, R², R⁴, R⁹, and n are as defined in claim 1 or salts or N-oxides thereof, with the proviso that the following compounds (IC1) to (IC5) are excluded: 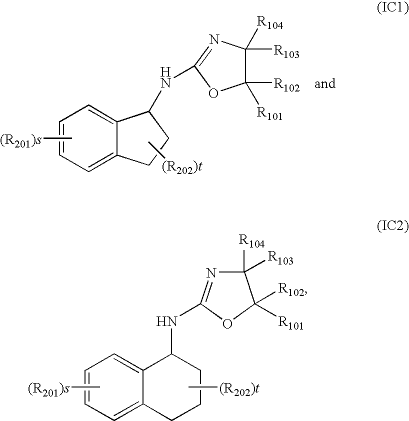
wherein R¹⁰¹ R¹⁰², R¹⁰³ and R¹⁰⁴ are hydrogen, R²⁰¹ and R²⁰² independently of each other are halogen, C₁₋₄ alkyl, C₁₋₄ alkoxy, C₁₋₄ alkylthio, trifluoromethyl or trifluoromethoxy, where the sum of s and t is 0, 1, 2 or 3. 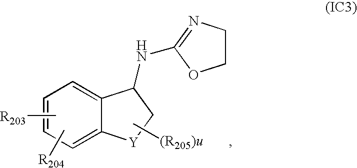
wherein Y is OCH₂, SCH₂, N(Me)CH₂, CH₂O, CH₂S or CH₂N(Me), R²⁰³ is hydrogen or C₁₋₄ alkyl, R²⁰⁴ is hydrogen, fluorine, chlorine, bromine, C₁₋₄ alkyl, C₁₋₄ alkoxy, C₁₋₄ alkylthio or dimethylamino, and R²⁰⁵ is C₁₋₄ alkyl, where u is 0, 1 or 2. 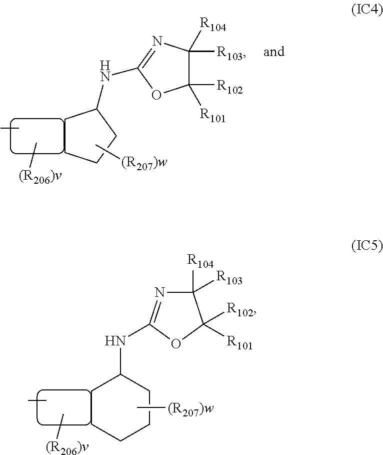
wherein T is thienyl or furyl, R¹⁰¹ R¹⁰², R¹⁰³ and R¹⁰⁴ are hydrogen, R²⁰⁶ and R²⁰⁷ independently of each other are hydrogen, chlorine or C₁₋₃ alkyl, and the sum of v and w is 0, 1, 2 or 3.

15. The compound according to claim 14, wherein the ring T is a 5- or 6-membered heteroaromatic ring, wherein the ring members are each independently CH, S, N, NR⁴, O, or CR⁴, provided that there is no more than one O or S atom present in the ring.

16. The compound according to claim 14, wherein Y is O, S(O)ₘ, NR³, SO₂—NR³, NR³—SO₂, NR³—O, O—NR³, O—CR⁷R⁸, S(O)ₘ—CR⁷R⁸, NR³—CR⁷R⁸, CR⁵R⁶—O, CR⁵R⁶—S(O)ₘ or CR⁵R⁶—NR³, and where R³, R⁵, R⁶, R⁷, R⁸ and m are as defined in claim 1.

17. The compound according to claim 14, wherein each R⁴ is independently: halogen; hydroxyl; amino; nitro; cyano; C₁₋₈ alkyl; C₁₋₈ haloalkyl; cyano(C₁₋₆)alkyl; C₁₋₃ alkoxy(C₁₋₃)alkyl; C₂₋₆ alkenyl; C₂₋₆ alkynyl; C₃₋₆ cycloalkyl; C₁₋₃ alkyl-(C₃₋₆)-cycloalkyl; phenyl optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino or dialkylamino; heteroaryl optionally substituted by halogen, nitro, cyano, C₁₋₆ alkyl, C₁₋₆ haloalkyl, C₁₋₆ alkoxy or C₁₋₆ haloalkoxy; heterocyclyl optionally substituted by halogen, nitro, cyano, C₁₋₆ alkyl, C₁₋₆ haloalkyl, C₁₋₆ alkoxy or C₁₋₆ haloalkoxy; formyl; C₁₋₆ alkylcarbonyl; carboxy; C₁₋₆ alkoxycarbonyl; C₁₋₆ alkylthiocarbonyl; C₁₋₆ alkoxythionocarbonyl; carbamoyl; C₁₋₆ alkylaminocarbonyl; di-C₁₋₆ alkylaminocarbonyl; thiocarbamoyl; C₁₋₆ alkylaminothionocarbonyl; di-C₁₋₆ alkylaminothionocarbonyl; C₁₋₈ alkoxy; C₁₋₆ haloalkoxy; phenoxy, wherein the phenyl group is optionally substituted by halogen, C₁₋₄ alkyl, C₁₋₄ alkoxy, C₁₋₄ haloalkyl, C₁₋₄ haloalkoxy, CN, NO₂, or phenyl; heteroaryloxy optionally substituted by halo, nitro, cyano, C₁₋₃ alkyl, C₁₋₃ haloalkyl, C₁₋₃ alkoxy or C₁₋₃ haloalkoxy; C₁₋₆ alkylcarbonyloxy; C₁₋₆ alkylthionocarbonyloxy; C₁₋₆ alkoxycarbonyloxy; C₁₋₆ alkylaminocarbonyloxy; di-C₁₋₆ alkylaminocarbonyloxy; C₁₋₆ alkylaminothionocarbonyloxy; di-C₁₋₆ alkylaminothionocarbonyloxy; C₁₋₈ alkylthio; C₁₋₆ haloalkylthio; arylthio or heteroarylthio where the aryl or heteroaryl is optionally substituted by halogen, C₁₋₄ alkyl, C₁₋₄ alkoxy, C₁₋₄ haloalkyl, C₁₋₄ haloalkoxy, CN, NO₂ or phenyl; C₁₋₆ alkylcarbonylthio; C₁₋₆ alkylaminocarbonylthio; di-C₁₋₆ alkylaminocarbonylthio; C₁₋₆ alkylaminothionocarbonylthio; di-(C₁₋₆)-alkylaminothionocarbonylthio; C₁₋₈ alkylamino; di-(C₁₋₈)-alkylamino, formylamino; C₁₋₆ alkylcarbonylamino; C₁₋₆ alkoxycarbonylamino, aminocarbonylamino; C₁₋₆ alkylaminocarbonylamino; di-C₁₋₆alkylaminocarbonylamino; aminothionocarbonylamino; C₁₋₆ alkylaminothionocarbonylamino; di-C₁₋₆ alkylaminothionocarbonylamino; or 2 adjacent groups R⁴ together with the carbon atoms to which they are attached form a 4, 5, 6 or 7 membered carbocylic or heterocyclic ring optionally substituted by halogen; n is 1, 2 or 3.

18. A method of combating and controlling insects, acarines, or molluscs which comprises applying to a pest, to a locus of a pest, or to a plant susceptible to attack by a pest an insecticidally, acaricidally, nematicidally or molluscicidally effective amount of a compound of formula (III) 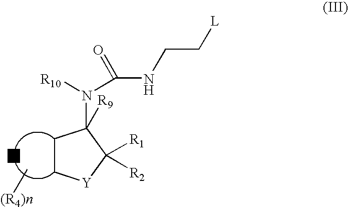
wherein T, Y, R¹, R², R⁴, R⁹, R¹⁹ and n are as defined in claim 1, and L is chlorine, bromine, iodine, C₁₋₄alkylsulfonyloxy or, phenylsulfonyloxy wherein the phenyl is optionally substituted by halogen, C₁₋₄alkyl, C₁₋₄alkoxy or nitro.

19. A compound of formula (III) 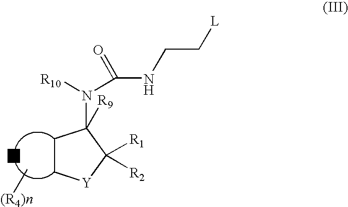
wherein T, Y, R¹, R², R⁴, R⁹, R¹⁹ and n are as defined in claim 1, and L is chlorine, bromine, iodine, C₁₋₄alkylsulfonyloxy or, phenylsulfonyloxy wherein the phenyl is optionally substituted by halogen, C₁₋₄alkyl, C₁₋₄alkoxy or nitro), with the proviso that compound of formula (III) is not:
1-(2-chloroethyl)-3-(1-indanyl)urea,
1-(2-bromoethyl)-3-(1-indanyl)urea,
1-(2-iodoethyl)-3-(1-indanyl)urea,
1-(2,4-diiodo-indany-1-yl)-3-(2-iodoethyl)-urea,
1-(2-bromo-ethyl)-3-(2-ethyl-indan-1-yl)urea,
Methanesulfonic acid 2-[3-(2-propoxy-indan-1-yl)-ureido]-ethyl ester,
1-(2-bromo-thyl)-3-(2,4,5-trichloro-indan-1-yl)urea,
1-(2-chloroethyl)-3-(2-chloro-1-indanyl)urea,
1-(2-chloroethyl)-3-(3,4-dimethyl-1-indanyl)urea,
Methanesulfonic acid 2-[3-(2-methyl-indan-1-yl)-ureido]-ethyl ester,
Toluene-4-sulfonic acid 2-[3-(2,5-diiodo-indan-1-yl)-ureido]-ethyl ester,
1-(2-chloroethyl)-3-(3,4-dihydro-2H-1-benzothiopyran-4-yl)urea,
1-(2-chloroethyl)-3-(1,2,3,4-tetrahydro-1-naphthyl)urea,
1-(2-bromoethyl)-3-(1,2,3,4-tetrahydro-1-naphthyl)urea,
1-(2-iodoethyl)-3-(1,2,3,4-tetrahydro-1-naphthyl)urea,
1-(2-chloroethyl)-3-(2-chloro-1,2,3,4-tetrahydro-1-naphthyl)urea,
1-(2-chloroethyl)-3-(4,5,6,7-tetrahydrobenzo[b]thien-4-yl)urea,
N-(2-chloroethyl)-N′-(2,3,3a,4,5,9b-hexahydro-1H-benz[e]inden-5-yl)urea,
(4α,5α,6α)-N-(2-chloroethyl)-N′-(5,6-dihydro-5,6-dihydroxy-4H-cyclopenta[b]thien-4-yl)urea,
(4α,5α,6α)-N[5,6-bis(acetyloxy)-5,6-dihydro-4H-cyclopenta[b]thien-4-yl]-N′-(2-chloroethyl)urea,
(4α,5α,6α)-N-(2-chloroethyl)-N′-(5,6-dihydro-5,6-dihydroxy-4H-cyclopenta[b]thien-4-yl)urea, or
(4α,5α,6α)-N-[5,6-bis(acetyloxy)-5,6-dihydro-4H-cyclopenta[b]thien-4-yl]-N′-(2-chloroethyl)urea.

20. An insecticidal, acaricidal, nematicidal or molluscicidal composition comprising an effective amount of a compound of formula I: 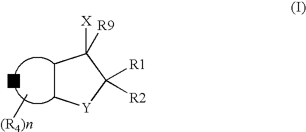
wherein
X is (i), (ii) or (iii) 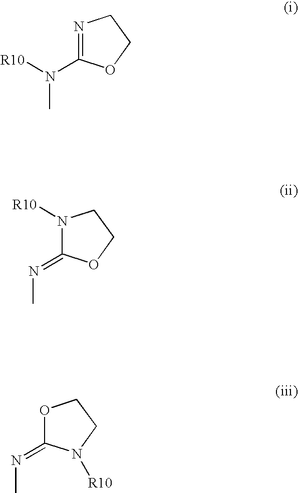
R¹⁰ is hydrogen, hydroxy, cyano, formyl, G-, G-O—, G-S—, G-S—S—, G-A-, R²⁴R²⁵N—, G-A-NR¹⁷—, R²⁴R²⁵N—S—, R²⁴R²⁵N-A-, R¹⁸N═C(R¹⁹)—, G-O-A- or G-S-A-; where R²⁴ and R²⁵ are independently H or G-, or R²⁴ and R²⁵ together with the N atom to which they are attached, form a group N═CRaRb where Ra and Rb are H, C₁₋₆ alkyl or phenyl; or R²⁴ and R²⁵ together with the N atom to which they are attached form a five, six or seven membered ring which may contain one or two further hetero atoms selected from O, N or S and which may be optionally substituted by one to four C₁₋₆ alkyl groups or phenyl; R¹⁷ is H, G-, G-C(O)— or G-OC(O); R¹⁸ is H, OH, cyano, nitro, G-, G-O— or R³⁸R³⁹N—, where R³⁸ and R³⁹ are independently H or G-, or R³⁸ and R³⁹ together with the N atom to which they are attached, form a group N═CRaRb where Ra and Rb are H, C₁₋₆ alkyl or phenyl; or R³⁸ and R³⁹ together with the N atom to which they are attached form a five, six or seven membered ring which may contain one or two further hetero atoms selected from O, N or S and which may be optionally substituted by one to four C₁₋₆ alkyl groups; R¹⁹ is H, cyano, G-, G-O—, G-S— or R⁴²R⁴³N—, where R⁴² and R⁴³ are independently H or G; or R⁴² and R⁴³ together with the N atom to which they are attached form a group N═CRaRb where Ra and Rb are H, C₁₋₆ alkyl or phenyl; or R⁴² and R⁴³ together with the N atom to which they are attached form a five, six or seven membered ring which may contain one or two further hetero atoms selected from O, N or S and which may be optionally substituted by one to four C₁₋₆ alkyl groups or phenyl;
Y is O, S(O)ₘ, where m is 0, 1 or 2, NR³, SO₂—NR³, NR³—SO₂, NR³—O or O—NR³, where R³ is H, OH, cyano, formyl, G-, G-O—, G-S—, G-A-, R²⁷R²⁸N—, R²⁷R²⁸N-A-, G-O-A-, G-S-A-, G-A-NR²⁹—, R²⁷R²⁸N-A-NR²⁹—, G-O-A-NR²⁹— or G-S-A-NR²⁹—, where R²⁷ and R²⁸ are independently H or G-, or R²⁷ and R²⁸ together with the N atom to which they are attached, form a group N═CRaRb where Ra and Rb are H, C₁₋₆ alkyl or phenyl; or R²⁷ and R²⁸ together with the N atom to which they are attached form a five, six or seven membered ring which may contain one or two further hetero atoms selected from O, N or S and which may be optionally substituted by one to four C₁₋₆ alkyl groups or phenyl; 0 is H or G-; or Y is CR⁵R⁶, CR⁵R⁶—CR⁷R⁸, O—CR⁷R⁸, S(O)ₘ—CR⁷R⁸, NR³—CR⁷R⁸, CR⁵R⁶—O, CR⁵R⁶—S(O)ₘ, CR⁵R⁶—NR³ where R³ and m have the meanings assigned to them above, and R⁵, R⁶, R⁷ and R⁸ are each independently H, OH, halogen, nitro, cyano, rhodano, carboxy, formyl, formyloxy, G-, G-O—, G-S—, G-A-, R²¹R²²N—, R²¹R²²N-A-, G-O-A-, G-S-A-, G-A-O—, G-A-S—, G-A-NR²³—, R²¹R²²N-A-O—, R²¹R²²N-A-S—, R²¹R²²N-A-NR²³—, G-O-A-O—, G-O-A-S—, G-O-A-NR²³—, G-S-A-O—, G-S-A-NR²³— or R²⁰S(O)(═NR¹⁷)—, where R²¹ and R²² are independently H or G-, or R²¹ and R²² together with the N atom to which they are attached, form a group N═CRaRb where Ra and Rb are H, C₁₋₆ alkyl or phenyl; or R²¹ and R²² together with the N atom to which they are attached form a five, six or seven membered ring which may contain one or two further hetero atoms selected from O, N or S and which may be optionally substituted by one to four C₁₋₆ alkyl groups or phenyl; R²³ is H or G- and R¹⁷ is as defined above; R²⁰ is C₁₋₆ alkyl, optionally substituted phenyl, optionally substituted benzyl; or two of the groups R⁵, R⁶, R⁷ and R⁸ attached to the same carbon atom are ═O, ═S, ═NR¹¹ or ═CR¹²R¹³, where R¹¹ is H, OH, nitro, cyano, formyl, formyloxy, G-, G-O—, G-A-, R³⁶R³⁷N—, G-C(O)—O—, G-C(O)—NR²⁶—, R³⁶R³⁷N—C(O)O—, G-O—C(O)O—NR²⁶—, where R³⁶, R³⁷ and R²⁶ are independently H or G-, or R³⁶ and R³⁷ together with the N atom to which they are attached, form a group N═CRaRb where Ra and Rb are H, C₁₋₆ alkyl or phenyl; or R³⁶ and R³⁷ together with the N atom to which they are attached form a five, six or seven membered ring which may contain one or two further hetero atoms selected from O, N or S and which may be optionally substituted by one to four C₁₋₆ alkyl groups or phenyl, and R¹² and R¹³ are each independently H, halogen, nitro, cyano, formyl, formyloxy, G-, G-O—, G-S—, G-A-, R⁴⁰R⁴¹N—, R⁴⁰R⁴¹N-A-, G-O-A-, G-A-O—, R⁴⁰R⁴¹N-A-O—, R⁴⁰R⁴¹N-A-S—, G-O-A-O—, G-O-A-S—, G-O-A-NR³⁰—, where R⁴⁰, R⁴¹ and R³⁰ are independently H or G-, or R⁴⁰ and R⁴¹ together with the N atom to which they are attached, form a group N═CRaRb where Ra and Rb are H, C₁₋₆ alkyl or phenyl; R⁴⁰ or R⁴¹ together with the N atom to which they are attached form a five, six or seven membered ring which may contain one or two further hetero atoms selected from O, N or S and which may be optionally substituted by one to four C₁₋₆ alkyl groups or phenyl, or R¹² and R¹³ together with the carbon atom to which they are attached form a 3 to 6 membered carbocyclic ring; or the groups R⁵ and R⁶ or R⁷ and R⁸ together with the carbon atom to which they are attached form a three to six membered ring, containing at least 2 carbon atoms and optionally containing one or two sulfur and/or one or two non-adjacent oxygen atoms or a group NR¹⁴, where R¹⁴ is H, OH, cyano, formyl, G-, G-O—, G-S—, G-A-, R²⁷R²⁸N—, R²⁷R²⁸N-A-, G-O-A-, G-S-A-, G-A-NR²⁹—, R²⁷R²⁸N-A-NR²⁹—, G-O-A-NR²⁹— or G-S-A-NR²⁹—, where R²⁷, R²⁸ and R²⁹ have the meanings assigned to them above, the ring being optionally substituted by one to four C₁₋₆ alkyl groups or phenyl; or two of the groups R⁵, R⁶, R⁷ and R⁸ attached to different atoms together with the atoms they are attached form a three to seven membered ring, that optionally contains one or two sulfur and/or one or two non-adjacent oxygen atoms or a group NR¹⁴, where R¹⁴ is as defined above, or two of the groups R⁵, R⁶, R⁷ and R⁸ attached to adjacent atoms combine to form a bond;
the ring (T) 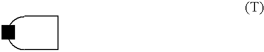
is a 5- or 6-membered aromatic or heteroaromatic ring;
R¹ and R² are each independently H, OH, halogen, nitro, cyano, rhodano, carboxy, formyl, formyloxy, G-, G-O—, G-S—, G-A-, R²¹R²²N—, R²¹R²²N-A-, G-O-A-, G-S-A-, G-A-O—, G-A-S—, G-A-NR²³—, R²¹R²²N-A-O—, R²¹R²²N-A-S—, R²¹R²²N-A-NR²³—, G-O-A-O—, G-O-A-S—, G-O-A-NR²³—, G-S-A-O—, G-S-A-NR²³—, or R²⁰S(O)(═NR¹⁷)—, where R¹⁷, R²⁰, R²¹, R²² and R²³ are as defined above, or two of the groups R¹ and R² attached to the same carbon atom are ═O, ═S, ═NR¹¹ or ═CR¹²R¹³, where R¹¹, R¹² and R¹³ are defined as above, or the groups R¹ and R² together with the same carbon atom to which they are attached form a three to six membered ring, containing at least 2 carbon atoms and optionally containing one or two sulfur and/or one or two non-adjacent oxygen atoms or a group NR¹⁴, where R¹⁴ is as defined above, the ring being optionally substituted by C₁₋₆ alkyl; or two of the groups R¹, R² and R⁷, R⁸ attached to different atoms together with the atoms they are attached form a three to seven membered ring, that optionally contains one or two sulfur and/or one or two non-adjacent oxygen atoms or a group NR¹⁴, where R¹⁴ is defined as above, the ring being optionally substituted by one or four C₁₋₆ alkyl groups or phenyl; or two of the groups R¹, R², R⁵, R⁶, R⁷ and R⁸ attached to adjacent atoms combine to form a bond;
each R⁴ is independently OH, halogen, nitro, cyano, azido, rhodano, isothiocyanato, carboxy, formyl, formyloxy, G-, G-O—, G-S—, G-A-, R³¹R³²N—, R³¹R³¹N-A-, G-O-A-, G-S-A-, G-A-O—, G-A-S—, G-A-NR³³—, R³¹R³²N-A-O—, R³¹R³¹N-A-S—, R³¹R³²N-A-NR³²—, G-O-A-O—, G-O-A-S—, G-O-A-NR³³—, G-S-A-O, G-S-A-NR³³—, R²⁰S(O)(═NR¹⁷)—, R¹⁸═C(R¹⁹)—, R⁴⁴R⁴⁵P(O)— or R⁴⁴R⁴⁵P(S)—, where R¹⁷, R¹⁸, R¹⁹ and R²⁰ have the meanings assigned to them above, and R³¹, R³² and R³³ are independently H or G-, or R³¹ and R³² together with the N atom to which they are attached, form a group N═CRaRb where Ra and Rb are H, C₁₋₆ alkyl or phenyl; or R³¹ and R³² together with the N atom to which they are attached form a five, six or seven membered ring which may contain one or two further hetero atoms selected from O, N or S and which may be optionally substituted by one to four C₁₋₆ alkyl groups or phenyl, and R⁴⁴ and R⁴⁵ are independently H, C₁₋₆ alkyl, C₁₋₆ alkoxy, phenyl, phenoxy; or 2 adjacent groups R⁴ together with the carbon atoms to which they are attached form a 4, 5, 6 or 7 membered carbocyclic or heterocyclic ring which may be optionally substituted by C₁₋₆alkyl or halogen; or a group R⁴ together with a group R³, R⁵, R⁶ or R⁹ and the atoms to which they are attached form a 5-7 membered ring optionally containing an NR¹⁵ group where R¹⁵ is H, OH, cyano, formyl, G-, G-O—, G-S—, G-A-, R²⁷R²⁸N—, R²⁷R²⁸N-A-, G-O-A-, G-S-A-, G-O-A-NR²⁹—, R²⁷R²⁸N-A-NR²⁹— or G-S-A-NR²⁹—, where R²⁷, R²⁸ and R²⁹ have the meanings assigned to them above, or containing an S or O atom, the ring being optionally substituted by one to four C₁₋₆ alkyl groups or phenyl;
n is 0, 1, 2, 3 or 4;
R⁹ is H, formyl, G-, G-A-, G-O-A-, R³⁴R³⁵N-A-, where R³⁴ and R³⁵ are independently H or G-, or R³⁴ and R³⁵ with the N atom to which they are attached, form a group N═CRaRb where Ra and Rb are H, C₁₋₆ alkyl or phenyl; or R³⁴ and R³⁵ with the N atom to which they are attached form a five, six or seven membered ring which may contain one or two further hetero atoms selected from O, N or S and which may be optionally substituted by one to four C₁₋₆ alkyl groups or phenyl; or R⁹ is G-O-A- or G-S-A-; or R⁹ together with a group R¹, R², R³, R⁵, R⁶, R⁷ or R⁸ and the atoms to which they are attached may form a three to seven membered ring, that optionally may contain one or two sulfur and/or one or two non-adjacent oxygen atoms or a group NR¹⁶; where R¹⁶ is H, OH, cyano, formyl, G-, G-O—, G-S—, G-A-, R²⁷R²⁸N—, R²⁷R²⁸N-A-, G-O-A-, G-S-A-, G-A-NR²⁹—, R²⁷R²⁸N-A-NR²⁹—, G-O-A-NR²⁹— or G-S-A-NR²⁹—, where R²⁷, R²⁸ and R²⁹ have the meanings assigned to them above,
G is optionally substituted C₁₋₁₂ alkyl, optionally substituted C₂₋₁₂ alkenyl, optionally substituted C₂₋₁₂ alkynyl, optionally substituted C₃₋₈ cycloalkyl, optionally substituted C₃₋₈ cycloalkenyl, optionally substituted aryl, optionally substituted heteroaryl or optionally substituted heterocyclyl;
A is S(O), SO₂, C(O) or C(S);
or salts or N-oxides thereof, and where at least one R¹, R², R⁴, R⁵, R⁶, R⁹ is different to hydrogen when R¹⁰ is hydrogen or methyl, T is benzene and Y is CR⁵R⁶.

21. An insecticidal, acaricidal, nematicidal or molluscicidal composition comprising an effective amount of a compound of formula (III) as defined in claim 19.
